(19) United States
(12) Patent Application Publication
(10) Pub. No.: US 2023/0329294 A1
(43) Pub. Date: Oct. 19, 2023

(54) PROTEIN COMPOSITIONS AND CONSUMABLE PRODUCTS THEREOF

(71) Applicant: Clara Foods Co., Daly City, CA (US)

(72) Inventors: Kritika MAHADEVAN, South San Francisco, CA (US); Joel Andrew KREPS, Daly City, CA (US); Isha JOSHI, Daly City, CA (US); Farnoosh AYOUGHI, Daly City, CA (US); Weixi ZHONG, Daly City, CA (US); Harshal KSHIRSAGAR, Daly City, CA (US); Alexandre CHAPEAUX, Daly City, CA (US); Wesley RUTHERFORD-JENKINS, Daly City, CA (US); Ranjan PATNAIK, Daly City, CA (US); Frank Douglas IVEY, Daly City, CA (US); Eric LIN, Daly City, CA (US); Sridharan GOVIND, Daly City, CA (US)

(21) Appl. No.: 18/296,654

(22) Filed: Apr. 6, 2023

Related U.S. Application Data
(63) Continuation of application No. PCT/US21/53850, filed on Oct. 6, 2021.
(60) Provisional application No. 63/088,290, filed on Oct. 6, 2020.
(60) Provisional application No. 63/109,137, filed on Nov. 3, 2020.

(51) Int. Cl. A23L 15/00; C12N 1/16; A23J 3/20; A23P 30/40; A23L 33/195; A23J 1/00; A23L 27/60; A21D 2/26; A21D 13/50; A21D 13/22; A21D 13/28

(52) U.S. Cl. CPC A23L 15/35; C12N 1/16; A23J 3/20; A23P 30/40; A23L 33/195; A23J 1/008; A23L 27/60; A21D 2/267; A21D 13/50; A21D 13/22; A21D 13/28

(57) ABSTRACT

Provided herein are compositions with enhanced protein content, protein combinations with high solubility and improved functionality, including foam stability and foam capacity, and methods for the preparation thereof.

### CROSS-REFERENCE TO RELATED APPLICATIONS

This application is a continuation of International Patent Application PCT/US21/53850, filed Oct. 6, 2021, which claims priority to US Provisional Patent Application Ser. Nos. 63/088,290, filed Oct. 6, 2020 and 63/109,137, filed Nov. 3, 2020. The entire contents of the aforementioned patent applications are incorporated herein by reference.

### SEQUENCE LISTING

The instant application contains a Sequence Listing which has been submitted electronically in XML format and is hereby incorporated by reference in its entirety. Said XML copy, created on Apr. 6, 2023, is named 49160-725,301SL.xml and is 193,800 bytes in size.

### BACKGROUND

Proteins are important dietary nutrients. They can serve as a fuel source or as sources of amino acids, including the essential amino acids that cannot be synthesized by the body. The daily recommended intake of protein for healthy adults is 10% to 35% of a person's total calorie needs, and currently the majority of protein intake for most humans is from animal-based sources. In addition, athletes and bodybuilders may rely upon increased protein consumption to build muscle mass and improve performance. With the world population growth and the coinciding growth in global food demand, there is a need to provide alternative sustainable, non-animal-based sources of proteins as useful source of protein for daily diet, dietary supplementation and sports nutrition.

### SUMMARY

An aspect of the present disclosure is a foam composition comprising a protein component, wherein the protein component comprises a mixture of recombinantly produced ovomucoid (rOVD) protein and recombinantly produced ovalbumin (rOVA) protein, wherein the foam composition has a foam capacity and a foam stability comparable to or higher than the foam capacity and the foam stability of a control composition that comprises similar contents h identity and quantity as the foam composition except the control composition's protein component is one of: chicken egg-white or an egg white substitute; ovomucoid alone; or ovalbumin alone.

In embodiments, the protein component consists essentially of a mixture of the rOVD and rOVA.

In some embodiments, the protein component comprises from about 2% to about 30% w/w of the foam composition, e.g., from about 4% to about 25% w/w of the foam composition from about 4% to about 20% w/w of the foam composition, from about 3% to about 20% w/w of the foam composition. In some cases, the protein component comprises from about 0.1% to about 99.5% rOVD w/w of the protein component and/or the protein component comprises from about 0.1% to about 99.5% rOVA w/w of the protein component. In some cases, the rOVD is from about 0.1% to about 20% w/w of the foam composition, e.g., from about 0.1% to about 10% w/w of the foam composition, from about 0.1% to about 5% w/w of the foam composition, from about 0.1% to about 2% w/w of the foam composition, and from about 0.1% to about 1% why of the foam composition. In some cases, the rOVA is from about 0.1% to about 20% w/w of the foam composition, e.g., from about 0.1% to about 10% w/w of the foam composition, from about 0.1% to about 5% w/w of the foam composition, from about 0.1% to about 2% why of the foam composition, or from about 0.1% to about 1% why of the foam composition. In various cases, the foam composition comprises at least 1% rOVD w/w and/or the foam composition comprises at least 1% rOVA w/w. In some cases, a ratio of rOVD to rOVA in the protein component is from 1:20 to 20:1, 1:15 to 15:1, 1:10 to 10:1, 1:5 to 5:1, 1:3 to 3:1, 1:2 to 2:1, or 1:1.

In various embodiments, the foam composition comprises a solvent.

In embodiments, the solvent is water or another consumable liquid.

In some embodiments, the foam composition consists essentially of water or of another consumable liquid and the protein component, e.g., the other consumable liquid is a beverage.

In various embodiments, the foam composition comprises a solvent, a protein component, and one or more components selected from a preservative, flavorant, salt, sweetener, acid, alcohol, fat or oil, stabilizer, and colorant.

In embodiments, the rOVD has a glycosylation pattern different from the glycosylation pattern of an ovomucoid obtained from a chicken egg.

In some embodiments, the rOVD protein comprises at least one glycosylated asparagine residue and the rOVD is substantially devoid of N-linked mannosylation, e.g., each glycosylated asparagine comprises a single N-acetylglucosamine. In some cases, the rOVD comprises at least three glycosylated asparagine residues.

In various embodiments, the rOVD provides protein fortification to the foam composition and provides an improvement to at least one additional feature selected from the group consisting of solubility, mouthfeel, texture, thickness, stability to heat treatment, and stability to pH relative to the control composition.

In embodiments, the foam composition has sensory properties comparable to those of the control composition.

In some embodiments, the rOVA has a glycosylation pattern different from an ovalbumin obtained from a chicken egg.

In various embodiments, the pH of the rOVA when solubilized is from about 3.5 to about 7.0.

In embodiments, the rOVD and/or the rOVA is produced by a microbial host cell, e.g., the microbial host cell is a yeast cell, a filamentous fungal cell, or a bacterial cell. In some cases, the microbial host cell is from a Pichia species, a Saccharomyces species, a Trichoderma species, a Pseudomonas species or an E. coli species.

In some embodiments, the protein component comprises one or more non-egg white proteins.

In various embodiments, the protein component does not comprise any egg white proteins other than rOVD and rOVA.

In embodiments, the rOVD has an amino acid sequence selected from any one of SEQ ID NOs: 1-44.

In some embodiments, the rOVA has an amino acid sequence selected from any one of SEQ ID NOs: 45-118.

Another aspect of the present disclosure is an edible composition comprising any herein disclosed foam composition. In various embodiments, the edible composition comprises at least 0.1% of the foam composition w/w. In embodiments, the composition is selected from: a coffee-drink, an alcoholic drink, a whipped cream composition, a frozen composition, or a dessert composition.

Yet another aspect of the present disclosure is a method for making a foam composition. The method comprises steps of combining a solvent with any-herein disclosed protein component as recited to obtain a liquid composition; and aerating the liquid composition to obtain the foam composition.

A further aspect of the present disclosure is a bilayer composition comprising a liquid fraction and a foam fraction, wherein the liquid fraction and the foam fraction each comprise a solvent and a protein component, wherein the protein component comprises a recombinantly-produced ovomucoid (rOVD) and a recombinantly-produced ovalbumin (rOVA), wherein the foam fraction has a larger volume when aerated for at least 1 minute as compared to a control fraction that comprises similar contents by identity and quantity as the foam fraction except the control fraction's protein component is one of: chicken egg-white or an egg white substitute; ovomucoid alone; or ovalbumin alone.

In some embodiments, the protein component is at least 0.5% of the fluid composition.

In various embodiments, the protein component is at least 1% of the fluid composition.

In embodiments, the protein component comprises from 0.1% to 99.5% rOVD w/w.

In some embodiments, the protein component comprises from 0.1% to 99.5% rOVA w/w.

In various embodiments, when aerated for at least 10 seconds a density of the foam fraction is comparable or less than a density of the control composition.

In embodiments, the liquid fraction and the foam fraction have identical contents by identity and quantity.

In an aspect, the present disclosure provides a solid or semi-solid consumable composition comprising a protein component wherein the protein component comprises a recombinantly-produced ovomucoid (rOVD) and a recombinantly-produced ovalbumin (rOVA), wherein the solid or semi-solid consumable composition has a larger volume when aerated for at least 1 minute as compared to a control composition that comprises similar contents by identity and quantity as the solid or semi-solid consumable composition except the control composition's protein component is one of chicken egg-white or an egg white substitute; ovomucoid alone; or ovalbumin alone. In some embodiments, the solid or semi-solid composition is a baked food, a dessert, a frozen dessert, or an egg-white like composition.

In another aspect, the present disclosure provides an ingredient composition for producing an egg-less food item, the composition comprising a recombinant ovalbumin (rOVA); wherein the pH of the rOVA when solubilized is from about 3.5 to about 7.0; wherein the rOVA when solubilized in an amount from about 2% to about 15% (w/w); has a foaming capacity higher than a foaming capacity of a natural egg white.

In yet another aspect, the present disclosure provides an ingredient composition for producing an egg-less food item, the composition comprising a recombinant ovomucoid (rOVD); wherein the rOVD has a glycosylation pattern different than an ovomucoid obtained from a chicken egg; wherein the ingredient composition comprises at most 20% w/w rOVD and wherein when the rOVD is solubilized and aerated to produce a foam the resulting foam capacity is higher than a foam capacity of a control foam produced by aerating a natural egg white.

In a further aspect, the present disclosure provides an animal-free egg-white like composition having a protein component comprising a recombinantly-produced ovomucoid (rOVD) and a recombinantly-produced ovalbumin (rOVA), wherein the composition has a higher foam stability than a control composition that comprises similar contents by identity and quantity as the animal-free egg-like composition except the control composition's protein component is one of: chicken egg-white or an egg white substitute; ovomucoid alone; or ovalbumin alone.

And, an aspect of the present disclosure is a powder composition comprising a mixture of a recombinantly produced ovomucoid (rOVD) protein and a recombinantly produced ovalbumin (OVA) protein, wherein the powder composition is capable of being solubilized and aerated to produce a foam composition that has a foam capacity and a foam stability comparable to or higher than the foam capacity and the foam stability of a control composition that comprises similar contents by identity and quantity as a control composition except the control composition's protein component is one of: chicken egg-white or an egg white substitute; ovomucoid alone; or ovalbumin alone.

In various embodiments, the foam composition has a protein concentration of less than 20% w/w.

In embodiments, the powder has a protein component that consists essentially of rOVD and rOVA.

In some embodiments, the powder comprises one or more additives, e.g., selected from: a filler or bulking agent, a flavorant, colorant, preservative, adjuster, powdered beverage mix, powdered juice mix, a sweetener, an amino acid, a protein, acidulant, dehydrated soup mix, dehydrated nutritional mix, dehydrated milk powder, caffeinated powder, or any combination thereof.

In various embodiments, the protein content of the powder is at least 1% w/w, e.g., at least 5% w/w of the protein component, at least 8% w/w of the protein component, at least 10% w/w of the protein component, at least 20% w/w of the protein component, at least 30% w/w of the protein component, at least 50% w/w of the protein component, at least 80% w/w of the protein component, and at least 90% w/w of the protein component.

In embodiments, the rOVA is at least 5% w/w of the protein component, e.g., at least 8% w/w of the protein component, at least 10% w/w of the protein component, at least 20% w/w of the protein component, at least 30% w/w of the protein component, at least 50% w/w of the protein component, at least 80% w/w of the protein component, and at least 90% w/w of the protein component.

In some embodiments, the ratio of rOVD to rOVA in the protein component is from 1:20 to 20:1, 1:15 to 15:1, 1:10 to 10:1, 1:5 to 5:1, 1:3 to 3:1, 1:2 to 2:1, or 1:1.

In various embodiments, the rOVD has a glycosylation pattern different from the glycosylation pattern of an ovomucoid obtained from a chicken egg.

In embodiments, the rOVD protein comprises at least one glycosylated asparagine residue and the rOVD is substantially devoid of N-linked mannosylation. In some cases, each glycosylated asparagine comprises a single N-acetylglucosamine.

In some embodiments, the rOVD comprises at least three glycosylated asparagine residues.

In various embodiments, the powder composition has sensory properties comparable to those of the control composition.

In embodiments, the rOVA has a glycosylation pattern different from an ovalbumin obtained from a chicken egg.

In some embodiments, the pH of the rOVA when solubilized is from about 3.5 to about 7.0.

In various embodiments, the rOVD and/or the rOVA is produced by a microbial host cell. In some cases, the microbial host cell is a yeast cell, a filamentous fungal cell, or a bacterial cell. In various cases, the microbial host cell is from a Pichia species, a Saccharomyces species, a Trichoderma species, a Pseudomonas species or an E. coli species.

In embodiments, the protein component does not comprise any egg white proteins other than rOVD and rOVA.

In some embodiments, the rOVD has an amino acid sequence selected from any one of SEQ ID NOs: 1-44.

In various embodiments, the rOVA has an amino acid sequence selected from any one of SEQ ID NOs: 45-118.

In some aspects, described herein is a foam composition, wherein the foam composition has a foam density that is less than a foam density of a control composition that comprises similar contents by identity and quantity as a control composition except the control composition's protein component is one of: chicken egg-white or an egg white substitute; ovomucoid alone; or ovalbumin alone.

In some embodiments, the foam density of an aerated product is less than about 30 g/ml.

In some embodiments, the foam density of an aerated product is less than about 25 g/ml.

In some embodiments, the foam density of an aerated product is less than about 20 g/ml.

In some aspects, described herein are foam compositions comprising a protein component comprising recombinant ovalbumin (rOVA) and recombinant ovomucoid (rOVD) and having a foam density that is less than about 30 g/ml.

Any aspect or embodiment described herein can be combined with any other aspect or embodiment as disclosed herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 14 illustrates a comparison in the glycosylation pattern of native ovomucoid and a recombinant ovomucoid produced in P. pastoris and according to the present disclosure. Shown is a lack of the complex branched glycosylation (including a lack of mannose residues) on the recombinant ovomucoid when produced in a strain of P. pastoris comprising endoglycosidases.

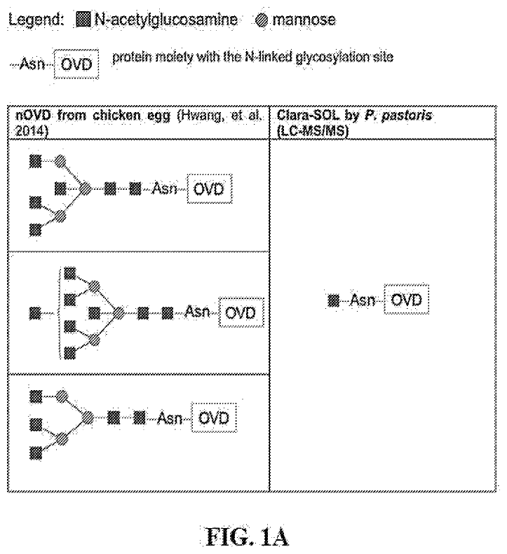

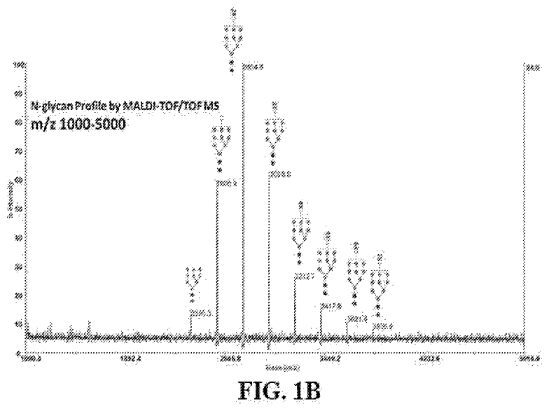

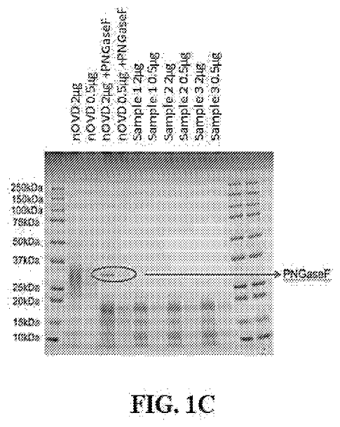

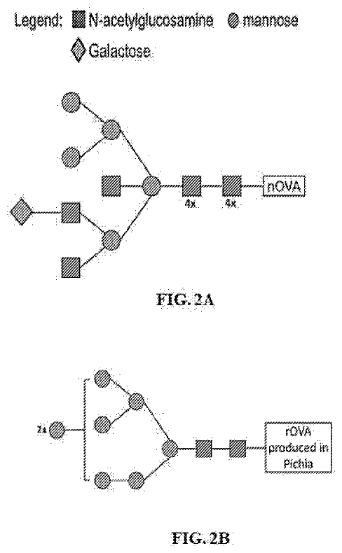

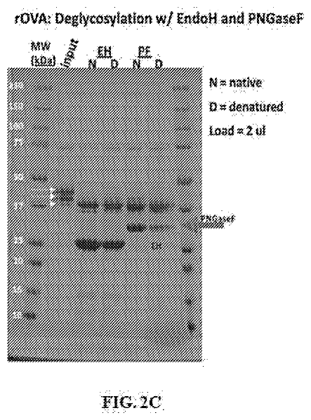

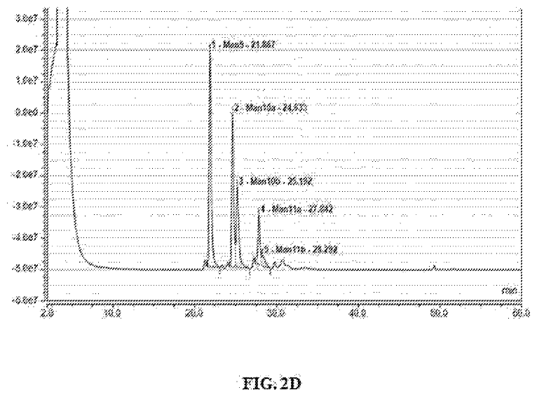

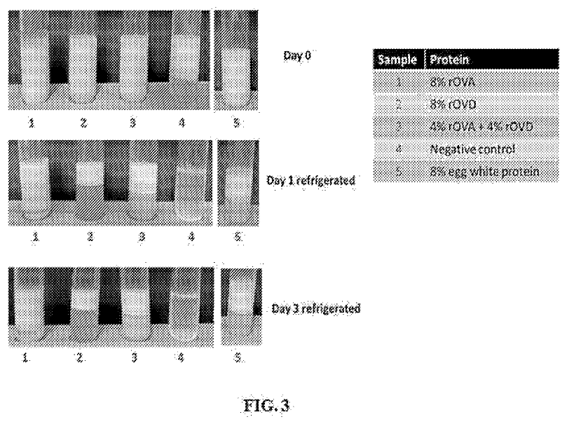

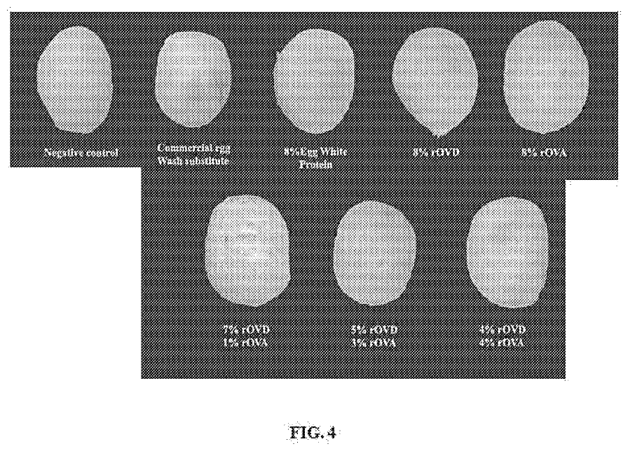

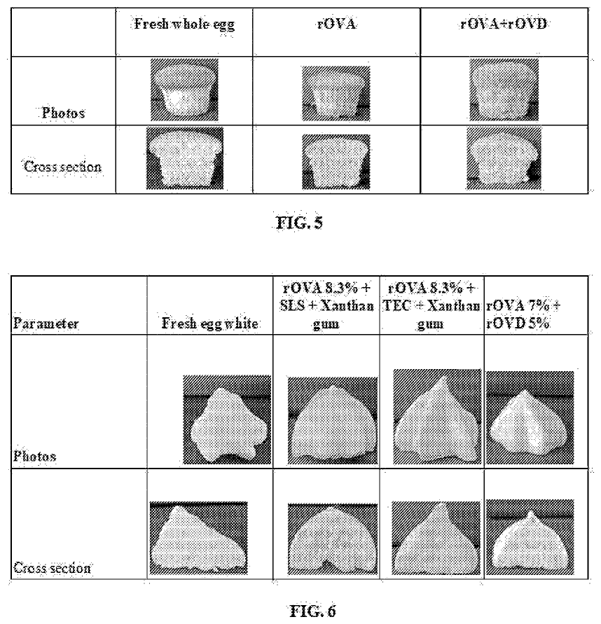

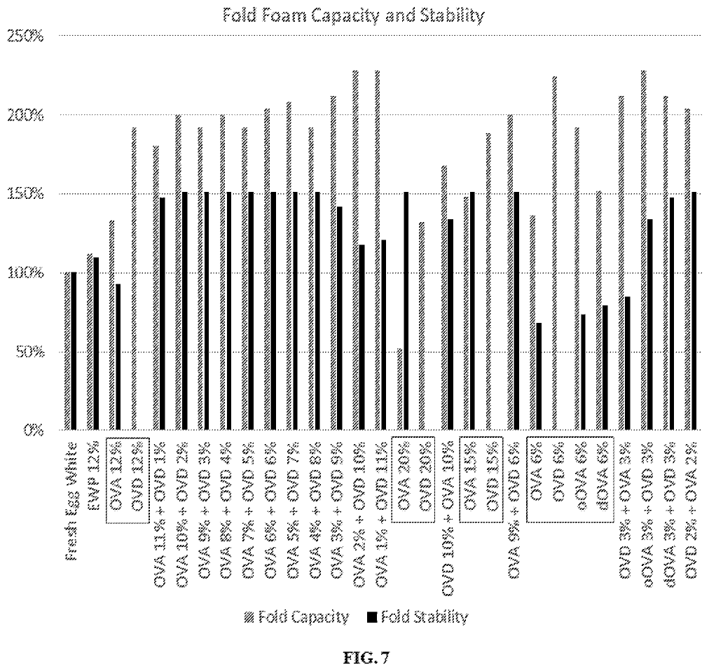

FIG. 1B illustrates the glycosylation patterns of the recombinant OVD produced by P. pastoris without an endoglycosidase treatment. rOVD thus produced have complex branched glycosylation patterns.

FIG. 1C compares the molecular weight of native OVD, native OVD treated with an endoglycosidase, and recombinant OVD samples.

FIGS. 2A-B illustrate glycosylation patterns of native OVA and rOVA produced in P. pastoris respectively.

FIG. 2C illustrates gel electrophoresis migration of glycosylated native and recombinant OVA. Also shown are deglycosylated recombinant OVA treated with EndoH and PNGaseF enzymes.

FIG. 2D illustrates a chromatogram depicting glycosylation patterns of rOVA produced in P. pastoris.

FIG. 3 illustrates a salad dressing composition made using various protein contents such as rOVA, rOVD, a combination of rOVA and rOVD, egg white protein and a negative control with no protein content.

FIG. 4 shows illustrative samples for comparing film forming agents in a bread dough application.

FIG. 5 shows illustrative samples of pound cakes made using various protein compositions.

FIG. 6 shows illustrative samples of meringues made using various protein compositions.

FIG. 7 illustrates foam capacity and fold stability of various protein compositions.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Provided herein are compositions and methods of making compositions including non-animal-based sources of proteins for ingestion by an animal, including a human, such as for daily diet, dietary supplementation, consumer foods and beverages, and enhanced nutrition. Illustrative compositions include foam compositions, edible compositions, bilayer compositions (comprising a liquid fraction and a foam fraction), solid or semi-solid consumable compositions, ingredient compositions for producing an egg-less food item, animal-free egg-white like compositions, and powder compositions.

Compositions of the present disclosure comprise a combination of egg-white proteins such as ovomucoid (OVD) and ovalbumin (OVA). These compositions can be used in a food product, drink product, nutraceutical, pharmaceutical, cosmetic, or as an ingredient in a final product. They can serve as the food product, drink product, and the like. In embodiments herein, the composition is in a liquid form or a semi-solid form. In embodiments herein, the composition is provided in a powdered form; this powder may be used to produce a liquid, solid, or semi-solid composition. Preferably, the OVD and OVA in such compositions is made recombinantly, and may be referred to herein as a recombinant OVD (rOVD) and recombinant OVA (rOVA), respectively.

Methods for manufacturing rOVD and illustrative uses for rOVD are disclosed in WO2021/007565 and methods for manufacturing rOVA and illustrative uses for rOVA are disclosed in WO2021/034980. The contents of the aforementioned applications are incorporated herein by reference in their entirety.

Unless indicated otherwise, the term OVD includes both native OVD (nOVD) and rOVD. Further, the term OVD includes an ovomucoid from any egg-laying animal, e.g., poultry, fowl, waterfowl, game bird, chicken, quail, turkey, turkey vulture, hummingbird, duck, ostrich, goose, gull, guineafowl, pheasant, emu, and any combination thereof. Unless indicated otherwise, the term OVA includes both native OVA (nOVA) and rOVA. Further, the term OVA includes an ovalbumin from any egg-laying animal, e.g., poultry, fowl, waterfowl, game bird, chicken, quail, turkey, turkey vulture, hummingbird, duck, ostrich, goose, gull, guineafowl, pheasant, emu, and any combination thereof. The nOVD or rOVD in the compositions herein is provided in concentrations that both increase the protein content of the composition or food ingredient while maintaining one or more additional characteristics such as high clarity, high solubility, reduced turbidity, or substantial sensory neutrality. The rOVA or nOVA in the compositions herein is provided in concentrations that both increase the protein content of the composition or food ingredient while providing desirable functional features to food ingredients and products.

In some embodiments, the rOVD has an amino acid sequence selected from any one of SEQ ID NOs: 1-44 and/or the rOVA has an amino acid sequence selected from any one of SEQ ID NOs: 45-118.

The use of rOVD and rOVA in any of the compositions herein allows for a non-animal-based source of protein, while providing additional features such as solubility, clarity, hardness, texture, thickness, mouthfeel, compatibility with heat treatment, compatibility with pH ranges and maintaining a consumer-favorable sensory profile. Various embodiments of such compositions, methods of making them, and methods of using them are provided herein. In some embodiments, the rOVD and/or rOVA provide one or more functional characteristics, and especially an improvement in the functional characteristic, such as of gelling, foaming (capacity and stability and time to generate foam), whipping, fluffing, binding, springiness, aeration, coating, film forming, emulsification (including emulsion stability), browning, thickening, texturizing, humectant, clarification, and cohesiveness. The protein combination with such feature(s) can be a food ingredient that provides for production of an egg-less or animal-free food ingredient or consumable food product for animal and/or human ingestion.

In some embodiments, the compositions and methods for making compositions herein increase the protein content of a consumable, and also provide additional features such as compatibility with other ingredients (such as, for example, compatibility with gluten, vitamins, minerals, and carbonation), coloration, smell, taste and compatibility with food and beverage preparation and/or storage conditions.

Native ovomucoid (nOVD), such as isolated from a chicken or another avian egg, has a highly complex branched form of glycosylation. The glycosylation pattern comprises N-linked glycan structures such as N-acetylglucosamine units and N-linked mannose units. See, e.g., FIG. 1B (left-hand column). In some cases, the rOVD for use in a herein-disclosed composition and produced using the methods described herein has a glycosylation pattern which is different than the glycosylation pattern of nOVD. For example, when rOVD is produced in a Pichia sp., the protein may be highly glycosylated. FIG. 1C illustrates the glycosylation patterns of rOVD produced by P. pastoris, showing a complex branched glycosylation pattern. In some embodiments of the compositions and methods herein, rOVD is treated such that the glycosylation pattern is modified from that of nOVD and also modified as compared to an rOVD produced by a Pichia sp. without such treatment. In some cases, the rOVD has no glycosylation. In some cases, the rOVD is substantially devoid of glycosylation (for example, as shown in FIG. 1A, right box). In other cases, the rOVD has reduced glycosylation. In some cases, the rOVD is modified by N-acetylglucosamine at one or more asparagine residues of the protein and lacks or is substantially devoid of N-linked mannosylation. See, e.g., FIG. 1A (right hand column). The changes in glycosylation described herein may lead to an increase in the solubility and clarity of rOVD as compared to proteins such as whey proteins, soy proteins, pea proteins, and nOVD. The modifications in glycosylation of rOVD may lead to a change in the nitrogen to carbon ratio of the protein, such that reducing or removing substantially all of the mannose residues, the nitrogen to carbon ratio is increased (such as compared to nOVD or to rOVD produced without the modification to the glycosylation pattern). The modifications in the glycosylation of rOVD may lead to a comparable or increased solubility and clarity as compared to nOVD even with the reduced glycosylation. The modifications in glycosylation of rOVD may lead a greater amino acid content per unit weight of a protein relative to the weight of a glycosylated rOVD or nOVD which has increased weight due to the carbohydrate chains.

In some embodiments, the composition is a consumable food product. In some embodiments, the consumable food product is a finished product. In some embodiments, the composition is an ingredient of a finished product, e.g., a powder comprising rOVD and rOVA or consisting essentially of rOVD and rOVA or a foam composition that is added to a food product to provide airiness and lightness to the finished product, such as a baked good. In some embodiments, a powder comprises rOVD and rOVA as the only protein component.

As used herein, the term “consumable food composition” refers to a composition, which comprises a protein component of the present disclosure and may be consumed by an animal, including but not limited to humans and other mammals. Consumable food compositions include food products, beverage products, dietary supplements, food additives, and nutraceuticals, as non-limiting examples.

Consumable food compositions also include compositions as an ingredient of a food or beverage, or a product ingested as part of an animal's diet.

Since the rOVD and/or rOVA of the present disclosure is not obtained from an animal source, a composition comprising the rOVD and/rOVA is considered non-animal-derived, animal-free, sustainable, vegetarian and/or vegan.

Provided herein are compositions and methods of making compositions for non-animal-derived sources of proteins which provide nutritional as well as functional properties to food ingredients and consumable products for ingestion by an animal, including a human.

As used herein, a “finished product” refers to a consumable food composition directed to or suitable itself as a food or beverage for animal consumption. As used herein, an “ingredient” or “component” in reference to a consumable food composition refers to a composition that is used with other ingredient(s) or component(s) to create a finished product.

Compositions with rOVD and rOVA

Provided herein are compositions, e.g., consumable food compositions, and methods of making such compositions that increase the protein content of the composition through the addition of a recombinant ovomucoid protein (rOVD) and a recombinant ovalbumin (rOVA). In some embodiments, rOVD and/or rOVA is added to a composition to increase the protein content, such as for added nutritional value. In some embodiments, rOVD or rOVA alone may be added to compositions.

An aspect of the present disclosure is a foam composition comprising a protein component, wherein the protein component comprises a mixture of recombinantly produced ovomucoid (rOVD) protein and recombinantly produced ovalbumin (rOVA) protein, wherein the foam composition has a foam capacity and a foam stability comparable to or higher than the foam capacity and the foam stability of a control composition that comprises similar contents by identity and quantity as the foam composition except the control composition's protein component is one of: chicken egg-white or an egg white substitute; ovomucoid alone; or ovalbumin alone.

Another aspect of the present disclosure is an edible composition comprising any herein disclosed foam composition. In various embodiments, the edible composition comprises at least 0.1% of the foam composition w/w. In embodiments, the composition is selected from: a coffee-drink, an alcoholic drink, a whipped cream composition, a frozen composition, or a dessert composition.

A further aspect of the present disclosure is a bilayer composition comprising a liquid fraction and a foam fraction, wherein the liquid fraction and the foam fraction each comprise a solvent and a protein component, wherein the protein component comprises a recombinantly-produced ovomucoid (rOVD) and a recombinantly-produced ovalbumin (rOVA), wherein the foam fraction has a larger volume when aerated for at least 10 seconds as compared to a control fraction that comprises similar contents by identity and quantity as the foam fraction except the control fraction's protein component is one of: chicken egg-white or an egg white substitute; ovomucoid alone; or ovalbumin alone.

In an aspect, the present disclosure provides a solid or semi-solid consumable composition comprising a protein component wherein the protein component comprises a recombinantly-produced ovomucoid (rOVD) and a recombinantly-produced ovalbumin (rOVA), wherein the solid or semi-solid consumable composition has a larger volume when aerated for at least 1 minute as compared to a control composition that comprises similar contents by identity and quantity as the solid or semi-solid consumable composition except the control composition's protein component is one of: chicken egg-white or an egg white substitute; ovomucoid alone; or ovalbumin alone. In some embodiments, the solid or semi-solid composition is a baked food, a dessert, a frozen dessert, or an egg-white like composition.

In another aspect, the present disclosure provides an ingredient composition for producing an egg-less food item, the composition comprising a recombinant ovalbumin (rOVA); wherein the pH of the rOVA when solubilized is from about 3.5 to about 7.0; wherein the rOVA when solubilized in an amount from about 2% to about 15% (w/w); has a foaming capacity higher than a foaming capacity of a natural egg white.

In yet another aspect, the present disclosure provides an ingredient composition for producing an egg-less food item, the composition comprising a recombinant ovomucoid (rOVD); wherein the rOVD has a glycosylation pattern different than an ovomucoid obtained from a chicken egg; wherein the ingredient composition comprises at most 20% w/w rOVD and wherein when the rOVD is solubilized and aerated to produce a foam the resulting foam capacity is higher than a foam capacity of a control foam produced by aerating a natural egg white.

In a further aspect, the present disclosure provides an animal-free egg-white like composition having a protein component comprising a recombinantly-produced ovomucoid (rOVD) and a recombinantly-produced ovalbumin (rOVA), wherein the composition has a higher foam stability than a control composition that comprises similar contents by identity and quantity as the animal-free egg-like composition except the control composition's protein component is one of: chicken egg-white or an egg white substitute; ovomucoid alone; or ovalbumin alone.

And, an aspect of the present disclosure is a powder composition comprising a mixture of a recombinantly produced ovomucoid (rOVD) protein and a recombinantly produced ovalbumin (rOVA) protein, wherein the powder composition is capable of being solubilized and aerated to produce a foam composition that has a foam capacity and a foam stability comparable to or higher than the foam capacity and the foam stability of a control composition that comprises similar contents by identity and quantity as a control composition except the control composition's protein component is one of: chicken egg-white or an egg white substitute; ovomucoid alone; or ovalbumin alone.

In some embodiments, a composition comprises a protein component comprising a mixture of the rOVD and rOVA, e.g., consisting essentially of a mixture of the rOVD and rOVA.

In some embodiments, the rOVD in the compositions (comprising rOVD and rOVA) and methods for making the same increases the protein content of the composition and the rOVD is substantially soluble in the composition.

### Aerated Compositions

An aspect of the present disclosure is an aerated composition such as a foam composition comprising a protein component, wherein the protein component comprises a mixture of rOVD protein and rOVA protein. In some cases, an aerated composition may comprise rOVD or rOVA alone. In some cases, an aerated composition such as a foam composition does not comprise any, proteins other than rOVD and rOVA. In some embodiments, the foam composition comprises one or more non-egg white proteins. In various embodiments, the foam composition does not comprise any egg white proteins other than rOVD and rOVA.

In some cases, an aerated composition, such as a foam composition comprises one or more solvents. In some embodiments, the solvent is water or another consumable liquid. The solvent or consumable liquid may be a beverage, a juice, a broth, a soup, a soda, a soft drink, a flavored water, a protein water, a fortified water, a carbonated water, a nutritional drink, an energy drink, a sports drink, a recovery drink, an alcoholic drink, a heated drink, a coffee-based drink, a tea-based drink, a plant-based milk, a milk based drink, a non-dairy, plant based milk drink, infant formula drink, a meal replacement drink. In some embodiments, the foam composition comprises a solvent, a protein component, and one or more components selected from a preservative, flavorant, salt, sweetener, acid, alcohol, fat or oil, stabilizer, and colorant. In some embodiments, the foam composition consists essentially of water or of another consumable liquid and the protein component, e.g., the other consumable liquid is a beverage.

In aspects and embodiments, the foam composition has a foam capacity and a foam stability comparable to or higher than the foam capacity and the foam stability of a control composition that comprises similar contents by identity and quantity as the foam composition except the control composition's protein component is one of: chicken egg-white or an egg white substitute; ovomucoid alone; or ovalbumin alone.

In some embodiments, the protein component comprises from about 2% to about 30% w/w of the foam composition. The protein component may comprise from about 2% to about 30% w/w or w/v of the foam composition. The protein component may comprise at least 2% w/w or w/v of the foam composition. The protein component may comprise at most 30% w/w or w/v of the foam composition. The protein component may comprise from 2% to 4%, 2% to 6%, 2% to 8%, 2% to 10%, 2% to 12%, 2% to 14%, 2% to 16%, 2% to 18%, 2% to 20%, 2% to 25%, 2% to 30%, 4% to 6%, 4% to 8%, 4% to 10%, 4% to 12%, 4% to 14%, 4% to 16%, 4% to 18%, 4% to 20%, 4% to 25%, 4% to 30%, 6% to 8%, 6% to 10%, 6% to 12%, 6% to 14%, 6% to 16%, 6% to 18%, 6% to 20%, 6% to 25%, 6% to 30%, 8% to 10%, 8% to 17%, 8% to 14%, 8% to 16%, 8% to 18%, 8% to 20%, 8% to 25%, 8% to 30%, 10% to 12%, 10% to 14%, 10% to 16%, 10% to 18%, 10% to 20%, 10% to 25%, 10% to 30%, 12% to 14%, 12% to 16%, 12% to 18%, 12% to 20%, 12% to 25%, 12% to 30%, 14% to 16%, 14% to 18%, 14% to 20%, 14% to 25%, 14% to 30%, 16% to 18%, 16% to 20%, 16% to 25% 16% to 30%, 18% to 20%, 18% to 25%, 18% to 30%, 20% to 25%, 20% to 30%, or 25% to 30% w/w or w/v of the foam composition. The protein component may comprise about 2%, 4%, 6%, 8%, 10%, 12%, 14%, 16%, 18%, 20%, 25%, or 30% w/w or w/v of the foam composition. The protein component may comprise at least 2%, 4%, 6%, 8%, 10%, 12%, 14%, 16%, 18%, 20%, or 25% w/w or w/v of the foam composition. The protein component may comprise at most 4%, %, 8%, 10%, 12%, 14%, 16%, 18%, 20%, 25%, or 30% w/w or w/v of the foam composition.

A liquid composition comprising a solvent, one or more proteins (such as rOVD and rOVA) and optionally one or more additives may be aerated to produce a foam composition. The concentration of rOVD in the foam composition may be from about 0.1% to about 20% w/w or w/v. The concentration of rOVD in the foam composition may be at least 0.1% w/w or w/v. The concentration of rOVD in the foam composition may be at most 20% w/w or w/v. The concentration of rOVD in the foam composition may be 0.1% to 0.5%, 0.1% to 1%, 0.1% to 2%, 0.1% to 5%, 0.1% to 8%, 0.1% to 10%, 0.1% to 12%, 0.1% to 15%, 0.1% to 18%, 0.1% to 19%, 0.1% to 20%, 0.5% to 1%, 0.3% to 2%, 0.5% to 5%, 0.5% to 8%, 0.5% to 10%, 0.5% to 12%, 0.5% to 15%, 0.5% to 18%, 0.5% to 19%, 0.5% to 20%, 1% to 2%, 1% to 3%, 1% to 8%, 1% to 10%, 1% to 12%, 1% to 15%, 1% to 18%, 1% to 19%, 10% to 20%, 7% to 5%, 2% to 8%, 2% to 10%, 2% to 12%, 2% to 15%, 2% to 18%, 2% to 19%, 2% to 20%, 5% to 8%, 5% to 10%, 5% to 12%, 5% to 15%, 5% to 18%, 5% to 19%, 5% to 20%, 8% to 10%, 8% to 12%, 8% to 15%, 8% to 18%, 8% to 19%, 8% to 20%, 10% to 12%, 10% to 15%, 10% to 18%, 10% to 19%, 10% to 20%, 12% to 15%, 12% to 18%, 12% to 19%, 12% to 20%, 15% to 18%, 15% to 19%, 18% to 19% or 18% to 20% w/w or wily. The concentration of rOVD in the foam composition may be about 0.1%, 0.5%, 1%, 2%, 5%, 8%, 10%, 12%, 15%, 18%, 19%, or 20% w/w or w/v. The concentration of rOVD in the foam composition may be at least 0.1%, 0.5%, 1%, 2%, 5%, 8%, 10%, 12%, 15%, 18% or 19% w/w or w/v. The concentration of rOVD in the foam composition may be at most 0.5%, 1%, 2%, 5%, 8%, 10%, 12%, 15%, 18%, or 19% w/w or w/v. The concentration of rOVA in the foam composition may be from about 0.1% to about 20% w/w or w/v. The concentration of rOVA in the foam composition may be at least 0.1% w/w or w/v. The concentration of rOVA in the foam composition may be at most 20% w/w or w/v. The concentration of rOVA in the foam composition may be 0.1% to 0.5%, 0.1% to 1%, 0.1% to 2%, 0.1% to 5%, 0.1% to 8%, 0.1% to 10%, 0.1% to 12%, 0.1% to 15%, 0.1% to 18%, 0.1% to 1.9%, 01% to 20%, 0.5% to 1%, 0.5% to 2%, 0.5% to 5%, 0.5% to 8%, 0.5% to 10%, 0.5% to 12%, 0.5% to 15%, 0.5% to 18%, 0.5% to 19%, 0.5% to 20%, 1% to 2%, 1% to 5%, 1% to 8%, 1% to 10%, 1% to 12%, 1% to 15%, 1% to 18%, 1% to 19%, 1% to 20%, 2% to 5%, 2% to 8%, 2% to 10%, 2% to 12%, 2% to 15%, 2% to 18%, 2% to 19%, 2% to 20%, 5% to 8%, 5% to 10%, 5% to 12%, 5% to 15%, 5% to 18%, 5% to 19%, 5% to 20%, 8% to 10%, 8% to 12%, 8% to 15%, 8% to 18%, 8% to 19%, 8% to 20%, 10% to 12%, 10% to 15%, 10% to 18%, 10% to 19%, 10% to 20%, 12% to 15%, 12% to 18%, 12% to 19%, 12% to 20%, 15% to 18%, 15% to 19%, 18% to 19% or 18% to 20% w/w or w/v. The concentration of rOVA in the foam composition may be about 0.1%, 0.5%, 1%, 2%, 5%, 8%, 10%, 12%, 15%, 18%, 19%, or 20% w/w or w/v. The concentration of rOVA in the foam composition may be at least 0.1%, 0.5%, 1%, 2%, 5%, 8%, 10%, 12%, 15%, 18% or 19% w/w or w/v. The concentration of rOVA in the foam composition may be at most 0.5% 1%, 2%, 5%, 8%, 10%, 12%, 15%, 18%, or 19% w/w or w/v.

A liquid composition can be transformed into an aerated composition, such as a foam composition, by providing aeration to the liquid composition. Aeration may be provided by blowing gas (e.g., air, N₂, CO₂, O₂, or another inert gas) into the liquid composition. Aeration may be provided by agitating the liquid composition, e.g., with a whisk, impeller blades, mixing blade, or the like. The whisk or blade may be a component of a blender, handheld blender (including a drill-like device such as a Dremel®) or handheld mixer, or a stand mixer. Alternately, aeration may occur by shaking or vibrating a closed container holding the liquid composition. In some cases, aeration may occur by infusing a liquid with steam, as generated by an espresso machine. Depending on the type of liquid composition, the amount of time needed to aerate the composition may vary. In some cases, it may take more than one minute of aeration to form an aerated composition, e.g., 1 minute, 2 minutes, 3 minutes, 4 minutes, 5 minutes, 10 minutes, or more and any amount of time therebetween; in other cases, it may take less than one minute of aeration to form an aerated composition, e.g., 1 second, 10 seconds, 20 seconds, 30 seconds, 40 seconds, 50 seconds or more and any amount of time therebetween.

Any herein-disclosed liquid composition may be aerated to become an aerated composition, such as a foam composition, of the present disclosure.

### Fluid Compositions

In some embodiments, the rOVD and rOVA composition is a fluid composition comprising a liquid and a foam composition. Such a composition may be produced by aerating a liquid composition. A liquid composition comprising a solvent, one or more proteins and optionally one or more additives may be aerated to produce a fluid composition or a composition with a bilayer of foam and liquid. For instance, an aerated composition may be a beverage that comprises foam e.g., caffeinated drinks such as cappuccinos, lattes, alcoholic drinks, etc. In some cases, the fluid composition does not comprise any proteins other than rOVD and rOVA. In some embodiments, the fluid composition comprises one or more non-egg white proteins. In various embodiments, the fluid composition does not comprise any egg white proteins other than rOVD and rOVA.

The fluid composition may comprise a protein component, such as a protein mixture described herein. The fluid composition may comprise from about 1% to about 30% w/w or w/v protein. The fluid composition may comprise at least 1% w/w or w/v protein. The fluid composition may comprise at most 20% w/w or w/v protein. The fluid composition may comprise 1% to 2%, 1% to 5%, 1% to 8%, 1% to 10% 1% to 12%, 1% to 15%, 1% to 18%, 1% to 20%, 1% to 25%, 1% to 30%, 2% to 5%, 2% to 8%, 2% to 10%, 2% to 12%, 2% to 15%, 2% to 18%, 2% to 20%, 2% to 25%, 2% to 30%, 5% to 8%, 5% to 10%, 5% to 12%, 5% to 15%, 5% to 18%, 5% to 20%, 5% to 25%, 5% to 30%, 8% to 10%, 8% to 12%, 8% to 15%, 8% to 18%, 8% to 20%, 8% to 25%, 8% to 30%, 10% to 12%, 10% to 15%, 10% to 18%, 10% to 20%, 10% to 25%, 10% to 30%, 12% to 15%, 12% to 18%, 12% to 20%, 12% to 25%, 12% to 30%, 15% to 18%, 15% to 20%, 15% to 25%, 15% to 30%, 18% to 20%, 18% to 25%, 18% to 30%, 20% to 25%, 20% to 30%, or 25% to 30% w/w or v/v protein. The fluid composition may comprise 1%, 2%, 5%, 8%, 10%, 12%, 15%, 18%, 20%, 25%, or 30% w/w or w/v protein. A preferred embodiment of the fluid composition may comprise from about 1% to about 20% w/w or w/v protein. The protein content in a fluid composition, such as described herein may be a protein mixture comprising rOVD and rOVA.

A fluid composition may comprise from about 0.1% to about 20% rOVD w/w or w/v. A fluid composition may comprise from about 0.1% to about 20% rOVD w/w or wry. A fluid composition may comprise at least 0.1% rOVD w/w or w/v. A fluid composition may comprise at most 20% rOVD w/w or w/v. A fluid composition may comprise 0.1% to 0.5%, 0.1% to 1%, 0.1% to 2%, 0.1% to 5%, 0.1% to 8%, 0.1% to 10%, 0.1% to 12%, 0.1% to 15%, 0.1% to 18%, 0.1% to 20%, 0.5% to 1%, 0.5% to 2%, 0.5% to 5%, 0.5% to 8%, 0.5% to 10%, 0.5% to 12%, 0.5% to 15%, 0.5% to 18%, 0.5% to 20%, 1% to 2%, 1% to 5%, 1% to 8%, 1% to 10%, 1% to 12%, 1% to 15%, 1% to 18%, 1% to 20%, 2% to 5%, 2% to 8%, 2% to 10%, 2% to 12%, 2% to 15%, 2% to 18%, 2% to 20%, 5% to 8%, 5% to 10%, 5% to 12%, 5% to 15%, 5% to 18%, 5% to 20%, 8% to 10%, 8% to 12%, 8% to 15%, 8% to 18%, 8% to 20%, 10% to 12%, 10% to 15%, 10% to 18%, 10% to 20%, 12% to 15%, 12% to 18%, 12% to 20%, 15% to 18%, 15% to 20%, or 18% to 20% rOVD w/w or w/v. A fluid composition may comprise about 0.1%, 0.5%, 1%, 2%, 5%, 8%, 10%, 12%, 15%, 18%, or 20% rOVD w/w or w/v. A fluid composition may comprise at least 0.1%, 0.5%, 1%, 2%, 5%, 8%, 10%, 12%, 15%, or 18% rOVD w/w or w/v. A fluid composition may comprise at most 0.5%, 1%, 2%, 5%, 8%, 10%, 12%, 15%, 18%, or 20% rOVD w/w or w/v. A fluid composition may comprise from about 0.1% to about 20% rOVA w/w or w/v. A fluid composition may comprise from about 0.1% to about 20% rOVA w/w or w/v. A fluid composition may comprise at least 0.1% rOVA w/w or w/v. A fluid composition may comprise at most 20% rOVA w/w or w/v. A fluid composition may comprise 0.1% to 0.5%, 0.1% to 1%, 0.1% to 2%, 0.1% to 5%, 0.1% to 8%, 0.1% to 10%, 0.1% to 12%, 0.1% to 15%, 0.1% to 18%, 0.1% to 20%, 0.5% to 1%, 0.5% to 2%, 0.5% to 5%, 0.5% to 8%, 0.5% to 10%, 0.5% to 12%, 0.5% to 15%, 0.5% to 18%, 0.5% to 20%, 1% to 2%, 1% to 5%, 1% to 8%, 1% to 10%, 1% to 12%, 1% to 15%, 1% to 18%, 1% to 20%, 2% to 5%, 2% to 8%, 2% to 10%, 2% to 12%, 2% to 15%, 2% to 18%, 2% to 20%, 5% to 8%, 5% to 10%, 5% to 12%, 5% to 15%, 5% to 18%, 5% to 20%, 8% to 10%, 8% to 12%, 8% to 15%, 8% to 18%, 8% to 20%, 10% to 12%, 10% to 15%, 10% to 18%, 10% to 20%, 12% to 15%, 12% to 18%, 12% to 20%, 15% to 18%, 15% to 20%, or 18% to 20% rOVA w/w or w/v. A fluid composition may comprise 0.1%, 0.5%, 1%, 2%, 5%, 8%, 10%, 12%, 15%, 18%, or 20% rOVA w/w or w/v. A fluid composition may comprise at least 0.1%, 0.5%, 1%, 2%, 5%, 8%, 10%, 12%, 15%, or 18% rOVA w/w or w/v. A fluid composition may comprise at most 0.5%, 1%, 2%, 5%, 8%, 10%, 12%, 15%, 18%, or 20% rOVA w/w ear w/v.

### Liquid Compositions

In some aspects, described herein are liquid compositions produced using one or more recombinant proteins. The liquid compositions may be any consumable compositions such as beverages, foam based drinks, liquid ingredients used to make consumable compositions, concentrated liquids (such as concentrated syrups) or other liquids described elsewhere herein. A liquid composition may comprise a protein component, such as a protein mixture described herein. A protein mixture may be added to a liquid composition to thicken the liquid composition or to provide airiness/lightness (when aerated), for e.g., in a smoothie. In some cases, the protein mixture consists essentially of rOVD and rOVA. In some cases, the liquid composition comprises one or more proteins in addition to rOVD and rOVA. In some cases, the only proteins in a liquid composition are rOVD and rOVA. In some cases, the liquid composition comprises no egg-white proteins other than rOVD and rOVA.

A liquid composition may comprise a protein component such as a protein mixture described herein. The liquid composition may comprise about 0.1% to about 45% w/w or w/v protein. The liquid composition may comprise at least 0.1% w/w or w/v protein. The liquid composition may comprise at most 45% w/w or w/v protein. The liquid composition may comprise 0.1% to 1%, 0.1% to 5%, 0.1% to 10%, 0.1% to 15%, 0.1% to 20%, 0.1% to 25%, 0.1% to 30%, 0.1% to 35%, 0.1% to 40%, 0.1% to 45%, 1% to 5%, 1% to 10%, 1% to 15%, 1% to 20%, 1% to 25%, 1% to 30%, 1% to 35%, 1% to 40%, 1% to 45%, 5% to 10%, 5% to 15%, 5% to 20%, 5% to 25%, 5% to 30%, 5% to 35%, 5% to 40%, 5% to 45%, 10% to 15%, 10% to 20%, 10% to 25%, 10% to 30%, 10% to 35%, 10% to 40%, 10% to 45%, 15% to 20%, 15% to 25%, 15% to 30%, 15% to 35%, 15% to 40%, 15% to 45%, 20% to 25%, 20% to 30%, 20% to 35%, 2.0% to 40%, 20% to 45%, 25% to 30%, 25% to 35%, 25% to 40%, 25% to 45%, 30% to 35%, 30% to 40%, 30% to 45%, 35% to 40%, 35% to 45%, or 40% to 45% w/w or w/v protein. The liquid composition may comprise 0.1%, 1%, 5%, 10%, 15%, 20%, 25%, 30%, 35%, 40%, or 45% w/w or w/v protein. The liquid composition may comprise at least 0.1%, 1%, 5%, 10%, 15%, 20%, 25%, 30%, 35%, or 40% w/w or w/v protein. The liquid composition may comprise at most 1%, 5%, 10%, 15%, 20%, 25%, 30%, 35% 40%, or 45% w/w or w/v protein. In a preferred embodiment, the liquid composition may comprise at most 35% protein w/w or w/v.

In some cases, the concentration of rOVD in the liquid composition may be from about 0.1% to about 40% in weight per total volume (w/v). The concentration of rOVD in the liquid composition may be at least 0.1% w/v. The concentration of rOVD in the liquid composition may be at most 40% w/v. The concentration of rOVD in the liquid composition may be from 0.1% to 1%, 0.1% to 5%, 0.1% to 10%, 0.1% to 15%, 0.1% to 20%, 0.1% to 25%, 01% to 30% 0.1% to 35%, 0.1% to 40%, 1% to 5%, 1% to 10%, 1% to 15%, 1% to 20%, 1% to 25%, 1% to 30%, 1% to 35%, 1% to 40%, 5% to 10%, 5% to 15%, 5% to 20%, 5% to 25%, 5% to 30%, 5% to 35%, 5% to 40%, 10% to 15%, 10% to 20%, 10% to 25%, 10% to 30%, 10% to 35%, 10% to 40%, 15% to 20%, 15% to 25%, 15% to 30%, 15% to 35%, 15% to 40%, 20% to 25%, 20% to 30%, 20% to 35%, 2.0% to 40%, 2.5% to 30%, 25% to 35%, 25% to 40%, 30% to 35%, 30% to 40%, or 35% to 40% w/v. The concentration of rOVD in the liquid composition may be about 0.1%, 1%, 5%, 10%, 15%, 20%, 25%, 30%, 35%, or 40% w/v. The concentration of rOVD in the liquid composition may be at least 0.1%, 1%, 5%, 10%, 15%, 20%, 25%, 30% or 35% w/v. The concentration of rOVD in the liquid composition may be at most 1%, 5%, 10%, 15%, 20%, 25%, 30%, 35%, or 40% w/v. In some cases, the concentration of rOVA in the liquid composition may be from about 0.1% to about 40% in weight per total volume w/v). The concentration of rOVA in the liquid composition may be at least 0.1% w/v. The concentration of rOVA in the liquid composition may be at most 40% w/v. The concentration of rOVA in the liquid composition may be from 0.1% to 1%, 0.1% to 5%, 0.1% to 10%, 0.1% to 15%, 0.1% to 20%, 0.1% to 25%, 0.1% to 30%, 0.1% to 35%, 0.1% to 40%, 1% to 5%, 1% to 10% 1% to 15%, 1% to 20%, 1% to 25%, 1% to 30%, 1% to 35%, 1% to 40%, 5% to 10%, 5% to 15%, 5% to 20%, 5% to 25%, 5% to 30%, 5% to 35%, 5% to 40%, 10% to 15%, 10% to 20%, 10% to 25%, 10% to 30%, 10% to 35%, 10% to 40%, 15% to 20%, 15% to 25%, 15% to 30%, 15% to 35%, 15% to 40%, 20% to 25%, 20% to 30%, 20% to 35%, 20% to 40%, 25% to 30%, 25% to 35%, 25% to 40%, 30% to 35%, 30% to 40%, or 35% to 40% w/v. The concentration of rOVA in the liquid composition may be about 0.1%, 1%, 5%, 10%, 15%, 20%, 25%, 30%, 35% or 40% w/v. The concentration of rOVA in the liquid composition may be at least 0.1%, 1%, 5%, 10%, 15%, 20%, 25%, 30% or 35% w/v. The concentration of rOVA in the liquid composition may be at most 1%, 5%, 10%, 15%, 20%, 25%, 30%, 35%, or 40% w/v.

### Solid Compositions

In some aspects, described herein are solid compositions. Proteins or protein components described herein may be used as ingredients to produce solid or semi-solid compositions. For example, the protein mixtures described herein can be used as an ingredient for the production of protein fortified gluten-free products including baked goods, a bread, a cookie, a cracker, a biscuit, a frozen dairy product, a frozen “dairy-like” product, a prepared meal, a meat product, a meatless product, a burger, a patty, a protein supplement, a snack bar, a protein bar, a nutrition bar, an energy bar, a dessert, or an “egg-like” product, pastries, cakes and noodles. In some cases, a protein mixture in the solid or semi-solid composition consists essentially of rOVD and rOVA. In some cases, the solid or semi-solid composition comprises one or more proteins in addition to rOVD and rOVA. In some cases, the only proteins in a solid or semi-solid composition are rOVD and rOVA. In some cases, the solid or semi-solid composition comprises no egg-white proteins other than rOVD and rOVA.

A solid or semi-solid composition may comprise one or more proteins. The solid or semi-solid composition may comprise from about 1% to about 30% w/w or w/v protein. The solid or semi-solid composition may comprise at least 1% w/w or w/v protein. The solid or semi-solid composition may comprise at most 30% w/w or wily protein. The solid or semi-solid composition may comprise 1% to 2%, 1% to 5%, 1% to 8%, 1% to 10%, 1% to 12%, 1% to 15%, 1% to 18%, 1% to 20%, 1% to 25%, 1% to 30%, 2% to 5%, 2% to 8%, 2% to 10%, 2% to 12%, 2% to 15%, 2% to 18%, 2% to 20%, 2% to 25%, 2% to 30%, 5% to 8%, 5% to 10%, 5% to 12%, 5% to 15%, 5% to 18%, 5% to 20%, 5% to 25%, 5% to 30%, 8% to 10%, 8% to 12%, 8% to 15%, 8% to 18%, 8% to 20%, 8% to 25%, 8% to 30%, 10% to 12%, 10% to 15%, 10% to 18%, 10% to 20%, 10% to 25%, 10% to 30%, 12% to 15%, 12% to 18%, 12% to 20%, 12% to 25%, 12% to 30%, 15% to 18%, 15% to 20%, 15% to 25%, 15% to 30%, 18% to 20%, 18% to 25%, 18% to 30%, 20% to 25%, 20% to 30%, or 25% to 30% w/w or wily protein. The solid or semi-solid composition may, comprise 1%, 2%, 5%, 8%, 10%, 12%, 15%, 18%, 20%, 25%, or 30% w/w or w/v protein. A preferred embodiment of the solid or semi-solid composition may comprise from 1% to 20% w/w or w/v protein. The protein in such compositions may be a protein mixture comprising rOVD and rOVA. Alternatively, in some cases the protein in such compositions may be rOVD or rOVA alone.

A solid or semi-solid composition may comprise from about 0.1% to about 28% rOVD w/w. A solid or semi-solid composition may comprise at least 0.1% rOVD w/w. A solid or semi-solid composition may comprise at most 28% rOVD w/w. A solid or semi-solid composition may comprise 0.1% to 0.5%, 0.1% to 1%, 0.1% to 5%, 0.1% to 10%, 0.1% to 15%, 0.1% to 20%, 0.1% to 25%, 0.1% to 28%, 0.5% to 1%, 0.5% to 5%, 0.5% to 10%, 0.5% to 15%, 0.5% to 20%, 0.5% to 25%, 0.5% to 28%, 1% to 5%, 1% to 10%, 1% to 15%, 1% to 20%, 1% to 25%, 1% to 28%, 5% to 10%, 5% to 15%, 5% to 20%, 5% to 25%, 5% to 28%, 10% to 15%, 10% to 20%, 10% to 25%, 10% to 28%, 15% to 20%, 15% to 25%, 15% to 28%, 20% to 25%, 20% to 28%, or 25% to 28% rOVD w/w. A solid or semi-solid composition may comprise about 0.1%, 0.5%, 1%, 5%, 10%, 15%, 20%, 25%, or 28% rOVD w/w. A solid or semi-solid composition may comprise at least 0.1%, 0.5%, 1%, 5%, 10%, 15%, 20%, or 25% rOVD w/w. A solid or semi-solid composition may comprise at most 0.5%, 1%, 5%, 10%, 15%, 20%, 25%, or 28% rOVD w/w. A solid or semi-solid composition may comprise from about 0.1% to about 28% rOVA w/w. A solid or semi-solid composition may comprise at least 0.1% rOVA w/w. A solid or semi-solid composition may comprise at most 28% rOVA w/w. A solid or semi-solid composition may comprise 0.1% to 0.5%, 0.1% to 1%, 0.1% to 5%, 0.1% to 10%, 0.1% to 15%, 0.1% to 20%, 0.1% to 25%, 0.1% to 28%, 0.5% to 1%, 0.5% to 5%, 0.5% to 10%, 0.5% to 15%, 0.5% to 20%, 0.5% to 25%, 0.5% to 28%, 1% to 5%, 1% to 10%, 1% to 15%, 1% to 20%, 1% to 25%, 1% to 28%, 5% to 10%, 5% to 15%, 5% to 20%, 5% to 25%, 5% to 28%, 10% to 15%, 10% to 20%, 10% to 25%, 10% to 28%, 15% to 20%, 15% to 25%, 15% to 28%, 20% to 25%, 20% to 28%, or 25% to 28% rOVA w/w. A solid or semi-solid composition may comprise about 0.1%, 0.5%, 1%, 5%, 10%, 15%, 20%, 25%, or 28% rOVA w/w. A solid or semi-solid composition may comprise at least 0.1%, 0.5%, 1%, 5%, 10%, 15%, 20%, or 25% rOVA w/w. A solid or semi-solid composition may comprise at most 0.5%, 1%, 5%, 10%, 15%, 20%, 25%, or 28% rOVA w/w.

### Powder Compositions

In some aspects, described herein are powdered compositions. The powder compositions described herein may be purified protein powders, protein powders mixed with other ingredients such as a filler or hulking agent, a flavorant, colorant, preservative, pH adjuster, powdered beverage mix, powdered juice mix, a sweetener, an amino acid, a protein, acidulant, dehydrated soup mix, dehydrated nutritional mix, dehydrated milk powder, caffeinated powder, coffee or any combination thereof. The powder composition may comprise a protein mixture. In some cases, a protein mixture in the powder composition consists essentially of rOVD and rOVA. In some cases, the powder composition comprises one or more proteins in addition to rOVD and rOVA. In some cases, the powder composition comprises no egg-white proteins other than rOVD and rOVA.

A powder composition may comprise from about 1% to about 98% w/w protein. A powder composition may comprise at least 1% w/w protein. A powder composition may comprise at most 98% w/w protein. A powder composition may comprise 1% to 5%, 1% to 10%, 1% to 20%, 1% to 30%, % to 40%, 1% to 50%, 1% to 60%, 1% to 70%, 1% to 80%, 1% to 90%, 1% to 98%, 5% to 10%, 5% to 20%, 5% to 30%, 5% to 40%, 5% to 50%, 5% to 60%, 5% to 70%, 5% to 80%, 5% to 90%, 5% to 98%, 10% to 20%, 10% to 30%, 10% to 40%, 10% to 50%, 10% to 60%, 10% to 70%, 10% to 80%, 10% to 90%, 10% to 98%, 20% to 30%, 20% to 40%, 20% to 50%, 20% to 60%, 20% to 70%, 20% to 80%, 20% to 90%, 20% to 98%, 30% to 40%, 30% to 50%, 30% to 60%, 30% to 70%, 30% to 80%, 30% to 90%, 30% to 98%, 40% to 50%, 40% to 60%, 40% to 70%, 40% to 80%, 40% to 90%, 40% to 98%, 50% to 60%, 50% to 70%, 50% to 80%, 50% to 90%, 50% to 98%, 60% to 70%, 60% to 80%, 60% to 90%, 60% to 98%, 70% to 80%, 70% to 90%, 70% to 98%, 80% to 90%, 80% to 98%, or 90% to 98% w/w protein. A powder composition may comprise about 1%, 5%, 10%, 20%, 30%, 40%, 50%, 60%, 70%, 80%, 90%, or 98% w/w protein. A powder composition may comprise at least 1%, 5%, 10%, 20%, 30%, 40%, 50%, 60%, 70%, 80%, or 95% w/w protein. A powder composition may comprise at most 5%, 10%, 20%, 30%, 40%, 50%, 60%, 70%, 80%, 90%, or 98% w/w protein. The protein in such powder compositions may be a protein mixture comprising rOVD and rOVA. Alternatively, in some cases the protein in such powder compositions may be rOVD or rOVA alone. For instance, a consumer may be able to combine two separate powders of rOVD and rOVA and combine them in a desired ratio.

In some cases, the concentration of rOVD in the powder composition may be from about 15% to about 99% weight per total weight (w/w). The concentration of rOVD in the powder composition may be at least 15% w % w. In embodiments, the concentration of rOVD in the powder composition may be at most 99% w/w. The concentration of rOVD in the powder composition may be 15% to 30%, 15% to 45%, 15% to 60%, 15% to 75%, 15% to 80%, 15% to 85%, 15% to 90%, 15% to 95%, 15% to 99%, 30% to 45%, 30% to 60%, 30% to 75%, 30% to 80%, 30% to 85%, 30% to 90%, 30% to 95%, 30% to 99%, 45% to 60%, 45% to 75%, 45% to 80%, 45% to 85%, 45% to 90%, 45% to 95%, 45% to 99%, 60% to 75%, 60% to 80%, 60% to 85%, 60% to 90%, 60% to 95%, 60% to 99%, 75% to 80%, 75% to 85%, 75% to 90%, 75% to 95%, 75% to 99%, 80% to 85%, 80% to 90%, 80% to 95%, 80% to 99%, 85% to 90%, 85% to 95%, 85% to 99%, 90% to 95%, 90% to 99%, or 95% to 99% w/w. The concentration of rOVD in the powder composition may be about 15%, 30%, 45%, 60%, 75%, 80%, 85%, 90%, 95%, or 99% w/w. The concentration of rOVD in the powder composition may be at least 15%, 30%, 45%, 60%, 75% 80%, 85%, 90% or 95% w/w. The concentration of rOVD in the powder composition may be at most 30%, 45%, 60%, 75%, 80%, 85%, 90%, 95%, or 99% w/w. In some cases, the concentration of rOVA in the powder composition may be from about 15% to about 99% weight per total weight (w/w). The concentration of rOVA in the powder composition may be at least 15% w/w. In embodiments, the concentration of rOVA in the powder composition may be at most 99% w/w. The concentration of rOVA in the powder composition may be 15% to 30%, 15% to 45%, 15% to 60%, 15% to 75%, 15% to 80%, 15% to 85%, 15% to 90%, 15% to 95%, 15% to 99%, 30% to 45%, 30% to 60%, 30% to 75%, 30% to 80%, 30% to 85%, 30% to 90%, 30% to 95%, 30% to 99%, 45% to 60%, 45% to 75%, 45% to 80%, 45% to 85%, 45% to 90%, 45% to 95%, 45% to 99%, 60% to 75%, 60% to 80%, 60% to 85%, 60% to 90%, 60% to 95%, 60% to 99%, 75% to 80%, 75% to 85%, 75% to 90%, 75% to 95%, 75% to 99%, 80% to 85%, 80% to 90%, 80% to 95%, 80% to 99%, 85% to 90%, 85% to 95%, 85% to 99%, 90% to 95%, 90% to 99%, or 95% to 99% w/w. The concentration of rOVA in the powder composition may be about 15% 30%, 45%, 60%, 75%, 80%, 85%, 90%, 95%, or 99% w/w. The concentration of rOVA in the powder composition may be at least 15%, 30%, 45%, 60%, 75%, 80%, 85%, 90% or 95% w/w. The concentration of rOVA in the powder composition may be at most 30%, 45%, 60%, 75%, 80%, 85%, 90%, 95% or 99% w/w.

### Protein Mixtures in Compositions

A protein component in the compositions described herein may be a protein mixture comprising one or more proteins. In some cases, a protein mixture consists essentially of rOVD and rOVA. In some cases, the protein mixture comprises one or more proteins in addition to rOVD and rOVA. In some cases, the protein mixture comprises no egg-white proteins other than rOVD and rOVA.

A protein mixture may comprise two forms of protein, for example, rOVD and rOVA. A protein mixture may comprise about 5% of an rOVD and about 95% of an rOVA w/w. A protein mixture may comprise about 10% of an rOVD and about 90% of an rOVA w/w. A protein mixture may comprise about 15% of an rOVD and about 85% of an rOVA w/w. A protein mixture may comprise about 20% of an rOVD and about 80% of an rOVA w/w. A protein mixture may comprise about 25% of an rOVD and about 75% of an rOVA w/w. A protein mixture may comprise about 30% of an rOVD and about 70% of an rOVA by/w. A protein mixture may comprise about 35% of an rOVD and about 65% of an rOVA w/w. A protein mixture may comprise about 40% of an rOVD and about 50% of an rOVA w/w. A protein mixture may comprise 45% of an rOVD and 55% of an rOVA w/w. A protein mixture may comprise 50% of an rOVD and 50% of an rOVA w/w. A protein mixture may comprise 55% of an rOVD and 45% of an rOVA w/w. A protein mixture may comprise 60% of an rOVD and 30% of an rOVA w/w. A protein mixture may comprise 65% of an rOVD and 35% of an rOVA w/w. A protein mixture may comprise 70% of an rOVD and 30% of an rOVA w/w. A protein mixture may comprise 75% of an rOVD and 25% of an rOVA w/w. A protein mixture may comprise 80% of an rOVD and 20% of an rOVA w/w. A protein mixture may comprise 85% of an rOVD and 15% of an rOVA w/w. A protein mixture may comprise 90% of an rOVD and 10% of an rOVA w/w. A protein mixture may comprise 95% of an rOVD and 5% of an rOVA

The ratio of an rOVD to an rOVA in the mixture may be about 1:1. The ratio of an rOVD to an rOVA in the mixture may be about 1:2. The ratio of an rOVD to an rOVA in the mixture may be about 1:3. The ratio of an rOVD to an rOVA in the mixture may be about 1:4. The ratio of an rOVD to an rOVA in the mixture may be about 1:5. The ratio of an rOVD to an rOVA in the mixture may be about 1:6. The ratio of an rOVD to an rOVA in the mixture may be about 1:7. The ratio of an rOVD to an rOVA in the mixture may be about 1:8. The ratio of an rOVD to an rOVA in the mixture may be about 1:9.

The ratio of an rOVD to an rOVA in the mixture may be about 2:1. The ratio of an rOVD to an rOVA in the mixture may be about 2:3. The ratio of an rOVD to an rOVA in the mixture may be about 2:5. The ratio of an rOVD to an rOVA in the mixture may be about 2:7. The ratio of an rOVD to an rOVA in the mixture may be about 2:9.

The ratio of an rOVD to an rOVA in the mixture may be about 3:1. The ratio of an rOVD to an rOVA in the mixture may be about 3:2. The ratio of an rOVD to an rOVA in the mixture may be about 3:4. The ratio of an rOVD to an rOVA in the mixture may be about 3:5. The ratio of an rOVD to an rOVA in the mixture may be about 3:7. The ratio of an rOVD to an rOVA in the mixture may be about 3:8.

The ratio of an rOVD to an rOVA in an protein mixture comprising rOVD and rOVA may be from 1:9 to 9:1. The ratio of an rOVD to an rOVA may be from 1:4 to 4:1. The ratio of an rOVD to an rOVA may be from 1:3 to 3:1. The ratio of an rOVD to an rOVA may be from 2:3 to 3:2.

The ratio of an rOVD to an rOVA in a protein mixture comprising rOVD and rOVA may be similar to that found in a chicken egg white, i.e., 1:4 to 1:5. The ratio of an rOVD to an rOVA may be different from that found in a chicken egg white, e.g., not 1:4 or 1:5.

The ratio of an rOVD to an rOVA in an protein mixture comprising rOVD and rOVA may be from 1:20 or 20:1. The ratio of an rOVD to an rOVA in an protein mixture comprising rOVD and rOVA may be 1:20, 1:18, 1:16, 1:14, 1:12, 1:10, 1:8, 1:6, 1:4, 1:2, 1:1, 2:1, 4:1, 6:1, 8:1, 10:1, 12:1, 14:1, 16:1, 18:1 or 20:1.

The total protein in a protein mixture may consist essentially of rOVD and rOVA. In some embodiments, the protein mixture comprises additional proteins other than the combination of rOVD and rOVA.

These protein mixtures may be used as an ingredient or component in a composition and/or a finished product.

### Features and Characteristics of Compositions

Described herein are foam compositions, edible compositions, bilayer compositions (comprising a liquid fraction and a foam fraction), solid or semi-solid consumable compositions, ingredient compositions for producing an egg-less food item, animal-free egg-white like compositions, and powder compositions.

In various embodiments, the protein component of a composition (e.g., comprising at least rOVD and rOVA) provides protein fortification to the composition and provides an improvement to at least one additional feature selected from the group consisting of solubility, mouthfeel, texture, thickness, stability to heat treatment, and stability to pH relative to the control composition.

The compositions herein, e.g., foam compositions, edible compositions, bilayer compositions (comprising a liquid fraction and a foam fraction), solid or semi-solid consumable compositions, ingredient compositions for producing an egg-less food item, animal-free egg-white like compositions, and powder compositions, can provide one or more functional features to food ingredients and food products. In some embodiments, the rOVD and/or rOVA provides a nutritional feature such as protein content, protein fortification, and amino acid content to a food ingredient or food product. The nutritional feature provided by rOVD and/or rOVA in the composition may be comparable or substantially similar to an egg white, native OVD (nOVD), and/or native OVA (nOVA). The nutritional feature provided by rOVD or rOVA in the composition may be better than that provided by a native whole egg or native egg white. In some cases, rOVD and rOVA provide the one or more functional features of egg-white in absence of any other egg-white proteins.

The compositions disclosed herein, e.g., foam compositions, edible compositions, bilayer compositions (comprising a liquid fraction and a foam fraction), solid or semi-solid consumable compositions, ingredient compositions for producing an egg-less food item, animal-free egg-white like compositions, and powder compositions, can provide foaming and foam capacity to a composition. For example, rOVD and rOVA can be used for forming a foam to use in baked products, such as cakes, for meringues and other foods where rOVD and rOVA can replace egg white to provide foam capacity. In some cases, rOVD and rOVA provides foaming and foam capacity of egg-white in absence of any other egg-white proteins. A composition made using a protein mixture comprising rOVD and rOVA may have improved properties as compared to a control composition, e.g., a control composition that comprises similar contents by identity and quantity as the foam composition except the control composition's protein component is one of: chicken egg-white or an egg white substitute; ovomucoid alone; or ovalbumin alone. The control composition in some cases may be a composition that's similar or substantially similar to the composition comprising rOVD and rOVA except the protein content of the control composition is one of natural egg-white (for instance, chicken egg-white), an egg-white substitute composition (for instance, commercially available egg white powders), native OVA, native OVD, rOVD alone, rOVA alone etc. The control composition may be a composition made using the equivalent ingredients, substitutable components, or comparable components as the compositions described herein with the exception of the protein content. The control composition may be a composition where the differences in ingredients as compared to the compositions described herein are insubstantial. As used herein, an “egg white substitute” may include products such as aquafaba, chia seeds, flax seeds, starches, apple sauce, banana puree, condensed milk, and other ingredients that are commonly used as egg white substitutes.

A herein-disclosed composition comprising may have a foam height greater than a foam height of a control composition, e.g., a control composition that comprises similar contents by identity and quantity as the foam composition except the control composition's protein component is one of chicken egg-white or an egg white substitute; ovomucoid alone; or ovalbumin alone. In some cases, a herein-disclosed composition may have a foam height of about or at least 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% greater relative to the control composition. In some cases, a herein-disclosed composition may have a foam height of up to 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% greater relative to the control composition.

A herein-disclosed composition may have a foam stability greater than a foam stability of a control composition, e.g., a control composition that comprises similar contents by identity and quantity as the foam composition except the control composition's protein component is one of: chicken egg-white or an egg white substitute; ovomucoid alone, or ovalbumin alone. In some cases, a herein-disclosed composition may have a foam stability of about or at least 50%, 55%, 60%, 65%, 70%, 75%, 80%, 85%; 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% greater relative to the control composition. In some cases, a herein-disclosed composition may have a foam stability of up to 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% greater relative to the control composition. Foam stability may be calculated by measuring drainage of a foamed solution. In some cases, the drainage may be measured in 10-minute increments for 30 minutes to gather data for foam stability. The drained volume after 30 minutes may be compared to the initial liquid volume (5 mL) for instance, foam Stability (%)=(Initial volume−drained volume) initial volume*100.

A herein-disclosed composition comprising may have a foam capacity greater than a foam capacity of a control composition, e.g., a control composition that comprises similar contents by identity and quantity as the foam composition except the control composition's protein component is one of chicken egg-white or an egg white substitute; ovomucoid alone; or ovalbumin alone. In some cases, a herein-disclosed composition may have a foam capacity of about or at least 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% greater relative to the control composition. In some cases, a herein-disclosed composition comprising may have a foam capacity of up to 50%, 55%, 60%, 65%, 70%, 75%, 80%, 85%, 90%, 95%, 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% greater relative to the control composition. In some embodiments, foam capacity may be determined by measuring the initial volume of foam following the whipping and compare against the initial volume of 5 mL. Foam Capacity (%)=(volume of foam/initial volume)*100.

A herein-disclosed composition may foam faster than a control composition, e.g., a control composition that comprises similar contents by identity and quantity as the foam composition except the control composition's protein component is one of: chicken egg-white or an egg white substitute; ovomucoid alone; or ovalbumin alone. In some cases, a herein-disclosed composition foams at least 1%, 5%, 10%, 15%, 20%, 25%, 30%, 35%, 40%, 45%, 50%, 55%, 60%, 65%, 70%, 75%, 80%, 85%, 90%, 95%, 100%, faster than the control composition, in some cases, a herein-disclosed composition foams up to 1%, 5%, 10%, 15%, 20%, 25%, 30%, 35%, 40%, 45%, 50%, 55%, 60%, 65%, 70%, 75%, 80%, 85%, 90%, 95%, 100% faster than the control composition. A time to measure foaming of a composition may be measured in terms of the time required to aerate a composition to produce a desired level of foam. In one example, the time required to foam a composition comprising rOVD and rOVA may be less than a time required to foam a composition comprising egg-white where both compositions have the same concentration of ingredients and were aerated at the same mixing speed.

A herein-disclosed composition may have a volume higher than a volume of a control composition, e.g., a control composition that comprises similar contents by identity and quantity as the foam composition except the control composition's protein component is one of: chicken egg-white or an egg white substitute; ovomucoid alone; or ovalbumin alone. For instance, a herein-disclosed composition may be an aerated composition (comprising a foam) and in the case where the composition comprises a protein mixture of rOVD and rOVA the aerated composition may have a higher foam than the control composition and therefore produce a composition with higher volume as compared to the control composition, in some cases, a herein-disclosed composition may have a volume of up to 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 350%, 400%, 450%, or 500% greater relative to the control composition. The volume of the herein-disclosed composition may be measured using conventional methods in the art.

The compositions described herein may be able to provide a lighter density composition than a control composition, e.g., a control composition that comprises similar contents by identity and quantity as the foam composition except the control composition's protein component is one of: chicken egg-white or an egg white substitute; ovomucoid alone; or ovalbumin alone. One of the favorable properties of the compositions described herein is an ability to provide a higher foam capacity and stability to the composition. Compositions described herein therefore may be less dense than the control composition.

### Sensory Neutrality and Improved Sensory Properties

In some embodiments, the addition of a protein component of the present disclosure (comprising rOVA and rOVD) to a composition provides sensory neutrality or an improved sensory appeal as compared to other proteins in such compositions. As used herein “sensory neutrality” refers to the absence of a strong or distinctive taste, odor (smell) or combination of taste and smell, as well as texture, mouth-feel, aftertaste and color. A sensory panel such as one described in Kemp et al. 2009 may be used by a panel of trained analysts. Sensory neutrality may provide an improved sensory appeal to a taster, such as a tester of foods or a consumer, when a consumable food composition comprising a protein component of the present disclosure with another like composition that has a different protein such as whey protein, pea protein, soy protein, whole egg or egg white protein at the same concentration. It is well-known in the art that native eggs can provide an unpleasant and undesirable “eggy” smell to a composition; protein components of the present disclosure generally do not provide such an “eggy” smell to a resulting composition.

In some embodiments, the combination of rOVD and rOVA, e.g., as a powder composition, when added to a consumable food composition is substantially odorless, such as measured by a trained sensory panel, in comparison with different solutions with a different protein component present in an equal concentration to the rOVD and rOVA containing solution, for example, in the comparison is whey, soy, collagen, pea, egg white solid isolates, native OVA and/or native OVD. In some embodiments of the rOVD and rOVA compositions described herein, such compositions are essentially odorless at a protein concentration from about 1-5%, 5-10%, 10-15%, 15-20%, 20-25%, 25-30% or greater than 30% rOVD and rOVA weight per total weight (w/w) and/or weight per total volume (w/v) or at a protein concentration of about 1, 2, 5, 8, 10, 11, 12, 13, 14, 15, 16, 17, 18, 19, 20, 21, 22, 23, 24, 25, 26, 27, 28, 29, 30 or more than 30 g of total rOVD and rOVA protein mixture per 100 mL solution (e.g., per 100 mL water).

In some embodiments, the addition of the combination of rOVD and rOVA to a composition also provides a neutral taste in addition to the characteristics such as increased protein nutritional content, solubility, clarity, and/or odorlessness. A neutral taste can be measured for example, by a trained sensory panel in comparison with solutions containing a different protein present in an equal concentration to the combination of rOVD and rOVA, for example, whey, soy, collagen, pea, whole egg, and egg white solid isolates (including native OVD, OVA).

In some embodiments, the addition of the combination of rOVD and rOVA provides a reduction in a certain odor and/or taste that is associated with other proteins used for supplementation. For example, addition of the combination of rOVD and rOVA has less of an “egg-like” odor or taste as compared to the addition of whole egg, fractionated egg or egg-white to a composition. In some embodiments, addition of the combination of rOVD and rOVA has less of a metallic odor or taste as compared to similar compositions yet comprising other proteins.

In some embodiments, the addition of the combination of rOVD and rOVA has an improved mouth-feel as compared to similar compositions yet comprising other proteins. For example, the addition of the combination of rOVD and rOVA is less grainy or has less precipitate or solids as compared to similar compositions yet comprising other proteins.

In various embodiments, the protein component of a composition (e.g., comprising at least rOVD and rOVA) provides protein fortification to the composition and provides an improvement to at least one sensory properties selected from the group consisting of mouthfeel, texture, and thickness, relative to the control composition that comprises similar contents by identity and quantity as the foam composition except the control composition's protein component is one of: chicken egg-white or an egg white substitute; ovomucoid alone; or ovalbumin alone.

In embodiments, a composition of the present disclosure has sensory properties comparable to those of the control composition that comprises similar contents by identity and quantity as the foam composition except the control composition's protein component is one of: chicken egg-white or an egg white substitute; ovomucoid alone; or ovalbumin alone.

In embodiments, a composition of the present disclosure has sensory properties that are improved relative to those of the control composition that comprises similar contents by identity and quantity as the foam composition except the control composition's protein component is one of: chicken egg-white or an egg white substitute; ovomucoid alone; or ovalbumin alone.

In many popular consumable liquids, e.g., milkshakes, coffee-type drinks (such as cappuccino and latte), alcoholic drinks, soups, and smoothies, increased thickness or airiness of the liquid improves a mouthfeel and texture of the liquid. Using a smoothie as an example, without an agent to promote thickening or allowing foam formation and aeration, the liquid composition is merely a thin juice. However, when a protein component of the present disclosure is added, the resulting liquid composition may be transformed into a more preferable consumable product, i.e., which has better mouthfeel, texture, and/or thickness.

The protein component provides improved foaming (at least) which transforms a composition (e.g., a liquid composition) into a foam composition and having the properties associated with a foam composition. In one example, an espresso-type drink can be a foam composition and the protein component allows production of a foam head when the liquid composition (which comprises an espresso based and includes a protein component of the present disclosure) is aerated with steam. The steam aerates the liquid composition and due to favorable properties of the protein component, the resulting foam composition will have a foam height, foam capacity, and/or foam stability when compared to standard espresso-type drink, e.g., cappuccino and latte, and which comprises a dairy or non-dairy milk component.

Even absent aeration, a liquid composition of the present disclosure can have improved mouthfeel, texture, and/or thickness relative to a control composition.

In some embodiments, the addition of the combination of rOVD and rOVA has an improved texture, for example, as compared to similar compositions yet comprising other proteins.

In some embodiments, the addition of the combination of rOVD and rOVA has an improved or appealing color or visual appeal as compared to similar compositions yet comprising other proteins. For example, the addition of the combination of rOVD and rOVA may maintain the clarity of a liquid (such as a carbonated drink, a protein water, sports drink) and provide visual appeal for the consumer.

A composition with the combination of rOVD and rOVA may have an improved sensory appeal as compared to the composition without the combination of rOVD and rOVA or with a different protein present in an equal concentration to the combination of rOVD and rOVA. Such improved sensory appeal may relate to taste and/or smell. Taste and smell can be measured, for example, by a trained sensory panel. In some instances, a sensory panel compares a composition with the combination of rOVD and rOVA to one without it or with a different protein in an equivalent amount.

Compatibility with Additional Ingredients

Provided herein are compositions comprising a protein component of the present disclosure and comprising combination of rOVD and rOVA wherein the rOVD and/or rOVA is compatible with one or more additional ingredients that are used in the preparation of a consumable food composition, including a finished product. Such compatibility provides fortification of protein content to the consumable food composition, while maintaining one or more desired characteristics of the consumable food composition and, in some cases, provides an improvement to at least one sensory properties selected from the group consisting of mouthfeel, texture, and thickness, relative to the control composition that comprises similar contents by identity and quantity as the foam composition except the control composition's protein component is one of: chicken egg-white or an egg white substitute; ovomucoid alone; or ovalbumin alone.

The protein component of the present disclosure (comprising combination of rOVD and rOVA) can be added to any consumable composition that is need of protein fortification; increased foam capacity, foam stability, and foam height, and/or improved sensory properties.

The protein component may be added as a powder composition in which the rOVD and rOVA and other ingredients are dry. Such a powder composition is advantageous in that it remains shelf-stable and may not need refrigeration but can be readily obtained and added to a consumable composition or when forming a consumable composition. Another advantage of use of a powdered compositions is that the powder does not add additional liquid to a consumable composition or when forming a consumable composition. As an example, in baked goods, dry ingredients are added in precise amounts and liquid ingredients are added in precise amounts; thus, a powder composition of the present invention can be added without disrupting the moisture content of a dough (for example). Similarly, by not adding additional moisture to a consumable composition, the additional liquid should further dilute the composition (e.g., a drink) or require extra heating to volatize and extract the additional moisture.

Alternately, the protein component may be added as a liquid composition or as a syrup in which the rOVD and rOVA are in a solution, e.g., comprising the protein component along with a solvent that can be water or another consumable liquid. An advantage of the liquid composition or syrup is that these can be easily mixed into a consumable composition. A syrup, in some embodiments, is a concentrated liquid composition or a concentrated liquid protein component; in a syrup, the amount of protein per unit volume is increased relative to a liquid composition. An advantage of a syrup is that it can be added to a consumable composition or when forming a consumable composition without adding much volume or without substantially diluting the consumable composition.

In some embodiments, a protein component of the present disclosure and comprising combination of rOVD and rOVA is compatible with gluten-containing ingredients. For example, a combination of rOVD and rOVA can be added with a gluten-containing ingredient to achieve protein fortification and maintain gluten-structure necessary for the ingredient and/or finished product. For example, a combination of rOVD and rOVA can be used as an ingredient for the production of protein fortified baked goods, a bread, a cake, a cookie, a cracker, a biscuit, a frozen dairy product, a frozen “dairy-like” product, a prepared meal, a meat product, a meatless product, a burger, a patty, a protein supplement, a snack bar, a protein bar, a nutrition bar, an energy bar, a dessert, a salad dressing, an egg-wash product, or an “egg-like” product, pastries, cakes and noodles. In the finished product, the combination of rOVD and rOVA does not substantially interfere with the gluten structure or has a substantially reduced interference with gluten structure as compared to other protein sources. As discussed throughout the disclosure, the protein component can improve the foam capacity, foam stability, and foam height of an aerated composition/foam composition (including a consumable composition). Thus, a cake which benefits from having additional foaminess would have improved desirable properties by including the protein component of the present disclosure relative to a control cake that comprises similar contents by identity and quantity as the cake of the present disclosure except the control cake's protein component is one of: chicken egg-white or an egg white substitute; ovomucoid alone; or ovalbumin alone.

In some embodiments, comprising a protein component of the present disclosure and comprising combination of rOVD and rOVA is compatible with gluten-free ingredients. For example, a combination of rOVD and rOVA can be added with a gluten-free ingredient mix to achieve protein fortification and provide structure and/or texture to the finished product. Gluten-free ingredients and finished products include such grains and starches (rice, corn, sorghum, and other cereals), root tubers such as potato, and legumes and pulses such as chickpeas and lentils. For example, a combination of rOVD and rOVA can be used as an ingredient for the production of protein fortified gluten-free products including baked goods, a bread, a cake, a cookie, a cracker, a biscuit, a frozen dairy product, a frozen “dairy-like” product, a prepared meal, a meat product, a meatless product, a burger, a patty, a protein supplement, a snack bar, a protein bar, a nutrition bar, an energy bar, a dessert, or an “egg-like” product, pastries, cakes and noodles.

In some embodiments, a combination of rOVD and rOVA is compatible with salts such that a combination of rOVD and rOVA protein does not precipitate out of solution. For example, for use in foods and beverages such as protein smoothies, vegan milk and fruit juices fortified with a combination of rOVD and rOVA, the protein remains substantially in solution. Addition of a combination of rOVD and rOVA does not precipitate in vitamin/mineral fortified environments such as present with fruit juice and juice-like products, and a combination of rOVD and rOVA provides increased protein content and nutrition.

### Consumable Food Compositions

Compositions, e.g., consumable food compositions, for ingestion by an animal, including a human, such as for daily diet, dietary supplementation, consumer foods and beverages, and enhanced nutrition, of the present disclosure comprise, at least, a protein component comprising a combination of rOVD and rOVA. Illustrative compositions include foam compositions, edible compositions, bilayer compositions (comprising a liquid fraction and a foam fraction), solid or semi-solid consumable compositions, ingredient compositions for producing an egg-less food item, animal-free egg-white like compositions, and powder compositions. Examples of consumable food compositions include food products, beverage products, dietary supplements, food additives, and nutraceuticals as non-limiting examples, and also include compositions as an ingredient of a food or beverage or a product ingested as part of an animal's (e.g., human's) diet. In some embodiments, a composition is a finished product, such as a food or beverage for animal consumption or for human consumption, a dietary supplement, or a nutraceutical product. In embodiments, a foam composition is selected from: a coffee-drink, an alcoholic drink, a whipped cream composition, a frozen composition, or a dessert composition. In embodiments, a liquid composition, is selected from: a coffee-drink and an alcoholic drink. In embodiments, a bilayer composition is selected from: a coffee-drink, an alcoholic drink, a whipped cream composition, a frozen composition, or a dessert composition. In embodiments, a solid or semi-solid composition is a baked food, a dessert, a frozen dessert, or an egg-white like composition.

The protein component of the present disclosure and compositions disclosed herein can provide structure, texture or a combination of structure and texture. In some embodiments, a protein component comprising rOVD and rOVA (as described herein) is added to a food ingredient or food product for baking and the protein mixture provides structure, texture or a combination of structure and texture to the baked product. Such protein mixtures can be used in such baked products in place of native egg white, native egg, or native egg protein. The addition of rOVD and rOVA to baked products can also provide protein fortification to improve the nutritional content. In some cases, a protein mixture comprising rOVD and rOVA provides the structure and/or texture of egg-white in absence of any other egg-white proteins. In some cases, rOVD or rOVA alone in a composition may be added to a baked product while providing protein fortification and additional properties to the a consumable composition, including improved solubility, clarity, hardness, texture, thickness, mouthfeel, compatibility with heat treatment, and/or compatibility with pH ranges, while maintaining a consumer-favorable sensory profile.

Compositions comprising protein mixtures disclosed herein can be compatible with gluten formations, such that the protein mixtures comprising rOVD and rOVA can be used where gluten formation provides structure, texture and/or form to a food ingredient or food product.

Exemplary baked products in which a herein-disclosed powder composition (or liquid composition, syrup composition, or foam composition) can be used as an ingredient include, but are not limited to cake, cookie, bread, bagel, biscuits, muffin, cupcake, scone, pancake, macaroon, choux pastry, meringue, and soufflé. For example, the protein components comprising rOVD and rOVA can be used as an ingredient to make cakes such as pound cake, sponge cake, yellow cake, or angel food cake, where such cakes do not contain any native egg white, native whole egg, or native egg protein. Along with the protein mixtures, baked products may contain additional ingredients such as flour, sweetening agents, gums, hydrocolloids, starches, fibers, flavorings (such as flavoring extracts) and other protein sources. In some embodiments, a baked product may include a protein mixture as described herein and at least one fat or oil, at least one grain starch, and optionally at least one sweetener. Grain starch for use in such compositions include flours such as wheat flour, rice flour, corn flour, millet flour, spelt flour, and oat flour, and starches such as from corn, potato, sorghum, and arrowroot. Oil and fat for use in such compositions include plant-derived oils and fats, such as olive oil, corn oil, avocado oil, nut oils (e.g., almond, walnut, and peanut), palm oil, sunflower oil, and safflower oil. The protein component or a composition of the present disclosure may provide such baked goods with at least one characteristic of an egg white such as binding, springiness, aeration, browning, texturizing, humectant, and cohesiveness of the baked product. In some cases, the baked product does not comprise any natural egg white or natural egg, and/or does not include any egg white derived proteins. In some cases, the baked product does not comprise any recombinant proteins other than rOVD and rOVA. In some cases, a protein component comprising rOVD and rOVA is provided to the baked composition as an ingredient, such as starting with a concentrate, isolate or powder form of rOVD and rOVA. In some cases, the protein components comprising rOVD and rOVA provided as an ingredient for baked products is at a pH range from about 3.5 to about 7.0. In some cases, a sweetener is included in the baked product such as a sugar, syrup, molasses, honey, or a sugar-substitute.

Compositions and protein components disclosed herein comprising rOVD and rOVA can also be used to prepare egg-less food products, such as food products made where native whole egg or native egg white is a primary or featured ingredient such as scramble, omelet, patty, soufflé, quiche and frittata. In some embodiments, compositions disclosed herein and/or protein components comprising rOVD and rOVA provides one or more functional features to the preparation including foaming, coagulation, binding, structure, texture, film-formation, nutritional profile, absence of cholesterol (i.e., cholesterol free) and protein fortification. Such egg-less preparations can be vegan, vegetarian, halal, or kosher, or a combination thereof. An egg-less preparation (sometimes referred to as an egg-white substitute) may include the combination of rOVD and rOVA and at least one fat or oil, a polysaccharide or polysaccharide-containing ingredient, and a starch. In some cases, the egg-less preparation may also include a flavoring agent (such as to provide a salty, sulfur-like or umami flavor), and/or a coloring agent (for example to provide yellow-like or off-white color to the baked product). In some cases, the inclusion of rOVD and rOVA in the egg-less preparation provides a characteristic of natural (native) egg white such as hardness, adhesiveness, fracturability, cohesiveness, gumminess and chewiness when the composition is heated or cooked. Exemplary polysaccharide or polysaccharide-containing ingredients for such compositions include gellan gum, sodium alginate, and psyllium. Oil and fat for use in such compositions include plant-derived oils and fats, such as olive oil, coin oil, avocado oil, palm oil, sunflower, and safflower oil.

Compositions disclosed herein can be used for a processed meat product or meat-like product, or for fish-like or shell-fish-like products. In such products, the composition and/or combination of rOVD and rOVA can provide one or more functional characteristics such as protein content and protein supplementations as well as binding, texturizing properties. Exemplary meat and meat-like products include burger, patty, sausage, hot dog, sliced deli meat, jerky, bacon, nugget and ground meat-like mixtures. Meat-like products can resemble beef, pork, chicken, lamb and other edible and consumed meats for humans and for other animals. Fish-like and shell-fish like products can resemble, for example, fish cakes, crab cakes, shrimp, shrimp balls, fish sticks, seafood meat, crab meat, fish fillets and clam strips. In some embodiments, the composition and/or combination of rOVD and rOVA is present in an amount from about 0.1% to about 30% w/w/ or w/v in the meat or meat-like product. In some embodiments, the combination of rOVD and rOVA is used for a meat-like product (also referred to as a meat-analog and includes at least one fat or oil, and a plant-derived protein. Oil and fat for use in such compositions include plant-derived oils and fats, such as olive oil, corn oil, avocado oil, palm oil, sunflower oil, and safflower oil. Plant-derived proteins for use in meat analogs include soy protein, nut proteins, pea protein, lentil and other pulse proteins and whey protein. In some cases, such plant protein is extruded, in other cases, such plant protein is non-extruded protein. In some cases, a meat analog includes the combination of rOVD and rOVA at about 2% to 15% (w/w). In some cases, for meat analog compositions, the combination of rOVD and rOVA acts as a binding agent, a gelling agent or a combination of a binding and gelling agent for such compositions.

Compositions disclosed herein can be employed in coatings for food products. For example, the combination of rOVD and rOVA can provide binding or adhesion characteristics to adhere batter or breading to another food ingredient. The combination of rOVD and rOVA can be used as an “egg-less egg wash” where the rOVD and rOVA proteins provide appearance, color and texture when coated onto other food ingredients or food products, such as baked products. In one example, the “egg-less egg wash” may be used to coat a baked good such that a dry or semi-dry ingredient (e.g., seed, salt, spice, and herb) adheres to the baked good. The addition of rOVD and rOVA as a coating to a food product can provide a crunchy texture or increase the hardness, for example, of the exterior of a food product such as when the product is cooked, baked or fried.

Compositions disclosed herein include sauces and dressings, such as an eggless mayonnaise, commercial mayonnaise substitutes, gravy, sandwich spread, salad dressing or food sauce. Inclusion of the combination of rOVD and rOVA in a sauce or dressing, and the like, can provide one or more characteristics such as binding, emulsifying, thickness, odor neutrality, and mouthfeel. In some embodiments, the combination of rOVD and rOVA is present in such sauces and dressing in an amount from about 0.1% to about 3% or from about 3% to about 5% w/w/ or w/v. In some cases, the amount of rOVD and rOVA in a sauce or dressing may be substantially similar to the amount of whole egg, egg-white, nOVD or nOVA used in a commercially available or commonly used recipe. Exemplary sauces and dressing include mayonnaise, commercial mayonnaise substitutes, alfredo sauce, and hollandaise sauce. In some embodiments, the rOVD and rOVA-containing sauce or dressing does not contain whole egg, egg white, or any other protein extracted from egg. In some cases, the sauce, dressing or other emulsified product made with rOVD and rOVA includes at least one fat or oil and water. Exemplary fats and oils for such compositions include corn oil, safflower oil, nut oils, palm oil, sunflower oil, and avocado oil.

Compositions described herein can be used to prepare confectionaries such as eggless, animal-free, vegetarian, and vegan confectionaries. The combination of rOVD and rOVA can provide one or more functional features to the confectionary including odor neutrality, flavor, mouthfeel, thickness, texture, gelling, cohesiveness, foaming, frothiness, nutritional value and protein fortification. In some embodiments, the prepared confectionary containing rOVD and rOVA does not contain any native egg protein or native egg white. The combination of rOVD and rOVA in such confectionaries can provide a firm or chewy texture. In some embodiments, the combination of rOVD and rOVA is present from about 0.1% to about 15% w/v in a confectionary. Exemplary confectionaries include a gummy, a taffy, a divinity candy, meringue, marshmallow, and a nougat. In some embodiments, a confectionary includes rOVD and rOVA, at least one sweetener and optionally a consumable liquid. Exemplary sweeteners include sugar, honey, sugar-substitutes and plant-derived syrups. In some cases, the combination of rOVD and rOVA is provided as an ingredient for making confectionaries at a from about 3.5 to about 7. In some cases, the combination of rOVD and rOVA is present in the confectionary composition at about 2% to about 15% (w/v). In some embodiments, the confectionary is a food product such as a meringue, a whipped dessert, or a whipped topping. In some embodiments, the combination of rOVD and rOVA in the confectionary provides foaming, whipping, fluffing or aeration to the food product, and/or provides gelation. In some cases, the confectionary is a liquid, such as a foamed drink. In some cases, the liquid may include a consumable alcohol (such w in a sweetened cocktail or after-dinner drink).

Compositions comprising protein components as described herein can be used in dairy products, dairy-like products or dairy containing products. For example, the combination of rOVD and rOVA can be used in preparations of beverages such as a smoothie, milkshake, “egg-nog”, and coffee beverage (e.g., cappuccino and latte). In some embodiments, the combination of rOVD and rOVA is added to additional ingredients where at least one ingredient is a dairy ingredient or dairy-derived ingredient (such as milk, cream, whey, and butter). In some embodiments, the combination of rOVD and rOVA is added to additional ingredients to create a beverage that does not contain any native eggs protein, native eggs white, or native egg. In some embodiments, the combination of rOVD and rOVA is an ingredient in a beverage that does not contain any animal-derived ingredients, such as one that does not contain any native egg-derived or any dairy-derived ingredients. Examples of such non-dairy derived drinks include nut milks, such as soy milk or almond milk. A combination of rOVD and rOVA can also be used to create beverage additions, such as creamer or “non-dairy milk” to provide protein, flavor, texture, thickness, and mouthfeel to a beverage such as a coffee, tea; alcohol-based beverages or cocoa. In some embodiments, the combination of rOVD and rOVA is present in a beverage ingredient or beverage addition in an amount from about 0.1% to about 20% w/w or w/v.

In some embodiments, a herein-disclosed composition and/or protein component comprising a combination of rOVD and rOVA can be used to prepare a dairy-like product such as yogurt, sour cream, cheese, butter, margarine, or whipped topping. Dairy products with a combination of rOVD and rOVA can include other animal-based dairy components or proteins. In some embodiments, such dairy-like products prepared with a combination of rOVD and rOVA do not include any animal-based ingredients. Foam compositions of the present disclosure are especially useful when making whipped dairy-like products, which includes some cheeses (e.g., whipped cream cheese), butters, and whipped toppings (e.g., meringue and a substitute whipped cream).

In dessert products, the combination of rOVD and rOVA can provide one or more characteristics such as creamy texture, low fat content, odor neutrality, flavor, mouthfeel, texture, binding, and nutritional value. A combination of rOVD and rOVA may be present in an ingredient or set of ingredients that is used to prepare a dessert product. Exemplary dessert products suitable for preparation with the combination of rOVD and rOVA include a mousse, a cheesecake, a custard, a meringue, a pudding, a popsicle, a whipped topping, and an ice cream. In some embodiments, dessert products prepared to include rOVD and rOVA are vegan, vegetarian or dairy-free. Dessert products that include a combination of rOVD and rOVA can have an amount of rOVD and rOVA that is from about about 0.1% to about 10% rOVD and rOVA w/w or w/v.

Compositions comprising protein components as described herein and comprising rOVD and rOVA can be used to prepare a snack food, such as a protein bar, an energy bar, a nutrition bar or a granola bar. The combination of rOVD and rOVA can provide characteristics to the snack food including one or more of binding, protein supplementation, flavor neutrality, odor neutrality, coating, texture, thickness, and mouth feel. In some embodiments, the combination of rOVD and rOVA is added to a preparation of a snack food in an amount from about 0.1% to about 30% w/w or w/v.

Compositions comprising protein components as described herein and comprising rOVD and rOVA can be used for nutritional supplements such as in parenteral nutrition, protein drink supplements, protein shakes where the combination of rOVD and rOVA provides a high protein supplement. In some embodiments, the combination of rOVD and rOVA can be added to such compositions in an amount from about 10% to about 30% w/w or w/v.

In some embodiments, compositions of the present disclosure can be used as an egg-replacer and an egg white-replacer. A combination of rOVD and rOVA can be mixed or combined with at least one additional component to form the egg white replacer. The combination of rOVD and rOVA can provide one or more characteristics to the egg-replacer or egg white-replacer, such as gelling, foaming, whipping, fluffing, binding, springiness, aeration, creaminess, cohesiveness, thickness, texture, and mouthfeel. In some embodiments, characteristic is the same or better than a control composition having similar contents by identity and quantity as the composition of the present disclosure except the control composition's protein component is one of: chicken egg-white or an egg white substitute; ovomucoid alone; or ovalbumin alone. In some embodiments, the egg-replacer or egg white-replacer, does not contain any egg, egg white, protein extracted or isolated from egg. In some cases, rOVD or rOVA alone in a composition may be used as an egg-replacer while providing protein fortification and additional properties to the baked composition.

In some embodiments, the compositions of the present disclosure are in powder form and when the powdered composition is formulated into a solution, the rOVD and rOVA is substantially fully soluble. In some embodiments, when the powdered composition is formulated into a solution, the rOVD and/or rOVA is substantially fully soluble and the solution is substantially clear. In some embodiments, when the powdered composition is formulated into a solution, the rOVD and/or rOVA is substantially fully soluble, the solution is substantially clear and the solution is essentially sensory neutral or has an improved sensory appeal as compared to solutions made with other powdered proteins such whey protein, soy protein, pea protein, egg white protein or whole egg proteins. In some embodiments, the powdered composition is solubilized in water where the concentration of rOVD and/or rOVA is or is about 1%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% or 40% weight per total weight (w/w) and/or weight per total volume (w/v) of composition. In embodiments, the powder has a protein component that consists essentially of rOVD and rOVA. In some embodiments, the powder comprises one or more additives, e.g., selected from: a filler or bulking agent, a flavorant, colorant, preservative, pH adjuster, powdered beverage mix, powdered juice mix, a sweetener, an amino acid, a protein, acidulant, dehydrated soup mix, dehydrated nutritional mix, dehydrated milk powder, caffeinated powder, or any combination thereof.

In various embodiments, the protein content of the powder composition is at least 1% w/w. e.g., at least 5% w/w of the protein component, at least 8% w/w of the protein component, at least 10% w/w of the protein component, at least 20% w/w of the protein component, at least 30% w/w of the protein component, at least 50% w/w of the protein component, at least 80% w/w of the protein component, and at least 90% w/w of the protein component.

In some embodiments, a powder composition of the present disclosure and comprising a protein component comprising rOVD and rOVA comprises less than 5% ash. The term “ash” is an art-known term and represents inorganics such as one or more ions, elements, minerals, and/or compounds In some cases, the powder composition comprises less than 5%, 4.5%, 4%, 3.5%, 3%, 2.5%, 2%, 1.5%, 1%, 0.75%, 0.5%, 0.25% or 0.1% ash weight per total weight (w/w) and/or weight per total volume (w/v).

In some embodiments, the moisture content of powder composition of the present disclosure may be less than 15%. The rOVD powder composition may have less than 15%, 12%, 10%, 8%, 6%, 5%, 3%, 2% or 1% moisture weight per total weight (w/w) and/or weight per total volume (w/v). In some embodiments, the carbohydrate content of a powder composition may be less than 30%. The powder composition may have less than 30%, 27%, 25%, 22%, 20%, 17%, 15%, 12%, 10%, 8%, 5%, 3% or 1% carbohydrate content w/w or w/v.

A powder composition may be sprinkled onto another consumable food product to increase protein content. In one example, the powder may be sprinkled onto a yoghurt, a salad, a baked dish, a breakfast cereal, pasta, and so forth. A powder composition may be included in a batter or dry layer for a fried food (e.g., fried meat or fired vegetable).

In some embodiments of the compositions described herein, the composition is essentially free of animal-derived component, whey protein, caseinate, fat, lactose, hydrolyzed lactose, soy protein, collagen, hydrolyzed collagen, or gelatin, or any combination thereof. A composition described herein may be essentially free of cholesterol, glucose, fat, saturated fat, trans fat, or any combination thereof. In some cases, a composition described herein comprises less than 10%, 5%, 4%, 3%, 2%, 1%, or 0.5% fat by dry weight. In some embodiments, the composition may be fat-containing (e.g., such as a mayonnaise) and such composition may include up to about 60% fat or a reduced-fat composition (e.g., reduced fat mayonnaise) and such composition may include lesser percentages of fat. A composition that free of an animal-derived component can be considered vegetarian and/or vegan.

In some embodiments of the compositions described herein, the composition is essentially free of animal-derived components, whey protein, caseinate, fat, lactose, hydrolyzed lactose, soy protein, collagen, hydrolyzed collagen, or gelatin, or any combination thereof. A composition described herein may be essentially free of cholesterol, glucose, fat, saturated fat, trans fat, or any combination thereof. In some cases, a composition described herein comprises less than 10%, 5%, 4%, 3%, 2%, 1%, or 0.5% fat by dry weight. In some embodiments, the composition may be fat-containing (e.g., such as a mayonnaise and commercial mayonnaise substitutes) and such composition may include up to about 60% fat or a reduced-fat composition (e.g., reduced fat mayonnaise and commercial mayonnaise substitutes) and such composition may include lesser percentages of fat. A composition that free of an animal-derived component can be considered vegetarian and/or vegan.

In any of the herein-disclosed compositions, the ratio of rOVD to rOVA in the protein component is from 1:20 to 20:1, 1:15 to 15:1, 1:10 to 10:1, 1:5 to 5:1, 1:3 to 3:1, 1:2 to 2:1, or 1:1.

In any of the herein-disclosed compositions, the rOVD has an amino acid sequence selected from any one of SEQ ID NOs: 1-44 and the rOVA has an amino acid sequence selected from any one of SEQ ID NOs: 45-118.

In any of the herein-disclosed compositions, the rOVD has a glycosylation pattern different from the glycosylation pattern of an ovomucoid obtained from a chicken egg; as examples, the rOVD protein comprises at least one glycosylated asparagine residue and the rOVD is substantially devoid of N-linked mannosylation. In some cases, each glycosylated asparagine comprises a single N-acetylglucosamine. In some embodiments, the rOVD comprises at least three glycosylated asparagine residues.

In any of the herein-disclosed compositions, the rOVD and/or the rOVA is produced by a microbial host cell. In some cases, the microbial host cell is a yeast cell, a filamentous fungal cell, or a bacterial cell. In various cases, the microbial host cell is from a Pichia species, a Saccharomyces species, a Trichoderma species, a Pseudomonas species or an E. coli species.

### Additional Components of Compositions

The compositions, e.g., consumable food compositions, containing a combination of rOVD and rOVA described herein and the methods of making such compositions may including adding or mixing the rOVD and rOVA with one or more ingredients. For example, food additives may be added in or mixed with the compositions. Food additives can add volume and/or mass to a composition. A food additive may improve functional performance and/or physical characteristics. For example, a food additive may prevent gelation or increased viscosity due to the lipid portion of the lipoproteins in the freeze-thaw cycle. An anticaking agent may be added to make a free-flowing composition. Carbohydrates can be added to increase resistance to heat damage, e.g., less protein denaturation during drying and improve stability and flowability of dried compositions. Food additives include, but are not limited to, food coloring, pH adjuster, natural flavoring, artificial flavoring, flavor enhancer, batch marker, food acid, filler, anticaking agent (e.g., sodium silico aluminate), antigreening agent (e.g., citric acid), food stabilizer, foam stabilizer or binding agent, antioxidant, acidity regulatory, bulking agent, color retention agent, whipping agent (e.g., ester-type whipping agent, triethyl citrate, sodium lauryl sulfate), emulsifier (e.g., lecithin), humectant, thickener, excipient, solid diluent, salts, nutrient, sweetener, glazing agent, preservative, vitamin, dietary elements, carbohydrates, polyol, gums, starches, flour, oil, or bran.

Food coloring includes, but is not limited to, FD&C Yellow #5, FD&C Yellow #6, FD&C Red #40, FD&C Red #3, FD&C Blue No. 1, FD&C Blue No. 2, FD&C Green No. 3, carotenoids (e.g., saffron, β-carotene), anthocyanins, annatto; betanin, butterfly pea, caramel coloring; chlorophyllin, elderberry juice, lycopene, carmine, pandan, paprika; turmeric, curcuminoids, quinoline yellow, carmoisine, Ponceau 4R, Patent Blue V. and Green S.

Ingredients for pH adjustment include, but are not limited to, potassium phosphate, sodium hydroxide, potassium hydroxide, citric acid, sodium citrate, sodium bicarbonate, acetic acid, and hydrochloric acid.

Salts include, but are not limited, to acid salts, alkali salts, organic salts, inorganic salts, phosphates, chloride salts, sodium salts, sodium chloride, potassium salts, potassium chloride, magnesium salts, magnesium chloride, magnesium perchlorate; calcium salts, calcium chloride; ammonium chloride, iron salts, iron chlorides, zinc salts, and zinc chloride.

Nutrient includes, but is not limited to, macronutrient, micronutrient, essential nutrient, non-essential nutrient, dietary fiber, amino acid, essential fatty acids, omega-3 fatty acids, and conjugated linoleic acid.

Sweeteners include, but are not limited to, sugar substitute, artificial sweetener, acesulfame potassium, advantame, alitame, aspartame, sodium cyclamate, dulcin, glucin, neohesperidin dihydrochalcone, neotame, P-4000, saccharin, aspartame-acesulfame salt, sucralose, brazzein, curculin; glycyrrhizin, glycerol, inulin, mogroside, mabinlin, malto-oligosaccharide; mannitol, miraculin, monatin, monellin, osladin, pentadin, stevia, trilobatin, and thaumatin.

Carbohydrates include, but are not limited to, sugar, sucrose, glucose, fructose, galactose, lactose, maltose, mannose, allulose, tagatose, xylose, arabinose, high fructose corn syrup, high maltose corn syrup, corn syrup (e.g., glucose-free corn syrup), sialic acid, monosaccharides, disaccharides, and polysaccharides (e.g., polydextrose, maltodextrin).

Polyols include, but are not limited to, xylitol, maltitol, erythritol, sorbitol, threitol, arabitol, hydrogenated starch hydrolysates, isomalt, lactitol, mannitol, and galactitol (dulcitol).

Gums include, but are not limited to, gum arabic, gellan gum, guar gum, locust bean gum, acacia gum, cellulose gum, and xanthan gum.

Vitamins include, but are not limited to, niacin, riboflavin, pantothenic acid, thiamine, folic acid, vitamin A, vitamin 136, vitamin 1312, vitamin D, vitamin E, lutein, zeaxanthin, choline, inositol, and biotin.

Dietary elements include, but are not limited to, calcium, iron, magnesium, phosphorus, potassium, sodium, zinc, copper, manganese, selenium, chlorine, iodine, sulfur, cobalt, molybdenum, nickel, and bromine.

### pH of Compositions

The pH of an rOVD and rOVA composition may be 3.5 to 8. The pH of an rOVD and rOVA composition may be at least 3.5. The pH of an rOVD and rOVA composition may be at most 8. The pH of an rOVD and rOVA composition may be 3.5 to 4, 3.5 to 4.5, 3.5 to 5, 3.5 to 5.5, 3.5 to 6, 3.5 to 6.5, 3.5 to 7, 3.5 to 7.5, 3.5 to 8, 4 to 4.5, 4 to 5, 4 to 5.5, 4 to 6, 4 to 6.5, 4 to 7.4 to 7.5, 4 to 8, 4.5 to 5, 4.5 to 5.5, 4.5 to 6, 4.5 to 6.5, 4.5 to 7, 4.5 to 7.5, 4.5 to 8, 5 to 5.5, 5 to 6, 5 to 6.5, 5 to 7, 5 to 7.5, 5 to 8, 5.5 to 6, 5.5 to 6.5, 5.5 to 7, 5.5 to 7.5, 5.5 to 8, 6 to 6.5, 6 to 7, 6 to 7.5, 6 to 8, 6.5 to 7, 6.5 to 7.5, 6.5 to 8, 7 to 7.5, 7 to 8, or 7.5 to 8. The pH of an rOVD and rOVA composition may be 3.5, 4, 4.5, 5, 5.5, 6, 6.5, 7, 7.5, or 8. An rOVD and rOVA composition with a pH from about 3.5 to about 7 may have one or more improved functionalities as compared to an nOVA composition, an nOVD composition, egg white or egg-white substitute compositions.

The pH of an rOVD and rOVA composition may be 2 to 3.5. The pH of an rOVD and rOVA composition may be at least 2. The pH of an rOVD and rOVA composition may be at most 3.5. The pH of an rOVD and rOVA composition may be 2 to 2.5, 2 to 3, 2 to 3.5, 2.5 to 3, 2.5 to 3.5, or 3 to 3.5. The pH of an rOVD and rOVA composition may be 2, 2.5, 3, or 3.5.

The pH of an rOVD and rOVA composition may be 7 to 12. The pH of an rOVD and rOVA composition may be at least 7. The pH of an rOVD and rOVA composition may be at most 12. The pH of an rOVD and rOVA composition may be 7 to 7.5, 7 to 8, 7 to 8.5, 7 to 9, 7 to 9.5, 7 to 10, 7 to 10.5, 7 to 11, 7 to 11.5, 7 to 12, 7.5 to 8, 7.5 to 8.5, 7, 5 to 9, 7.5 to 9.5, 7, 5 to 10, 7.5 to 10.5, 7.5 to 11, 7.5 to 11.5, 7.5 to 12, 8 to 8.5, 8 to 9, 8 to 9.5, 8 to 10, 8 to 10.5, 8 to 11, 8 to 11.5, 8 to 12, 8.5 to 9, 8.5 to 9.5, 8.5 to 10, 8.5 to 10.5, 8.5 to 11, 8.5 to 11.5, 8.5 to 12, 9 to 9.5, 9 to 10, 9 to 10.5, 9 to 11, 9 to 11.5, 9 to 12, 9.5 to 10, 9.5 to 10.5, 9.5 to 11, 9.5 to 11.5, 9.5 to 12, 10 to 10.5, 10 to 11, 10 to 11.5, 10 to 12, 10.5 to 11, 10.5 to 11.5, 10.5 to 12, 11 to 11.5, 11 to 12, or 11.5 to 12. The pH of an rOVA composition may be 7, 7.5, 8, 8.5, 9, 9.5, 10, 10.5, 11, 11.5, or 12.

In some embodiments, the pH of rOVD and/or rOVA may be adjusted prior to Us inclusion in a composition or its use as an ingredient. In some embodiments, the pH of rOVD and/or rOVA is adjusted during the purification and/or isolation processes. In some embodiments, the pH of the rOVD and/or rOVA for use in an ingredient or in production of a food product composition is adjusted to from about 3.5 to about 7.0. In some cases, the pH of rOVD and/or rOVA may be adjusted to more than one pH during the production process. For example, rOVD and/or rOVA may be expressed in a host cell such as a a microbial cell, and in some cases the rOVA is secreted by the host cell into the growth media (e.g., liquid media). rOVD and/or rOVA may be separated from the host cells and such separation step may be performed at a selected pH, for example at a pH of about 3.5. In some cases, the rOVD and/or rOVA at such separation pH may not be soluble or may not be fully soluble and the pH is adjusted to a higher pH, such as about pH 12. The rOVD and/or rOVA may then be adjusted to a final pH from about 3.5 to about 7.0. Separation of rOVD and/or rOVA from other components of the host cells or other components of the liquid media can include one or more of ion exchange chromatography, such as cation exchange chromatography and/or anion exchange chromatography, filtration and ammonium sulfate precipitation.

### Recombinant OVD and OVA

In any composition described herein, the protein may be recombinantly expressed in a host cell. The recombinant protein may be OVD, a first non-recombinant protein (e.g., OVD) and a second recombinant protein such as ovalbumin (e.g. rOVA), or OVD and at least one second protein may both be recombinantly produced (for example rOVD and rOVA).

rOVD or rOVA can have an amino acid sequence from any species. For example, an rOVD can have an amino acid sequence of OVD native to a bird (avian) or a reptile or platypus and an rOVA can have an amino acid sequence of OVA native to a bird or a reptile or platypus. An rOVD and/or rOVA having an amino acid sequence from an avian OVD and/or OVA can be selected from the group consisting of: poultry, fowl, waterfowl, game bird, chicken, quail, turkey, turkey vulture, hummingbird, duck, ostrich, goose, gull, guineafowl, pheasant, emu, and any combination thereof. An rOVD and/or rOVA can have an amino acid sequence native to a single species, such as Gallus gallus domesticus. Alternatively, an rOVD and/or rOVA can have an amino acid sequence native to two or more species, and as such be a hybrid.

Exemplary OVD and OVA amino acid sequences contemplated herein are provided in Table 1 below as SEQ NOs: 1-44 and 45-118, respectively.

TABLE 1
Sequences

| | SEQ | |
|---|---|---|
| Sequence | ID | |
| Description | NOS | SEQUENCES |
| Ovomucoid | SEQ ID | AEVDCSRFPNATDKEGKDVLVCNKDLRPICGTDGVTYTNDCLLCAYSIEFGT |
| (canonical) | NO: 1 | NISKEHDGECKETVPMNCSSYANTTSEDGKVMVLCNRAFNPVCGTDGVTYD |
| mature chicken | | NECLLCAHKVEQGASVDKRHDGGCRKELAAVSVDCSEYPKPDCTAEDRPLC |
| OVD | | GSDNKTYGNKCNFCNAVVESNGTLTLSHFGKC |
| Ovomucoid | SEQ ID | AEVDCSRFPNATDMEGKDVLVCNKDLRPICGTDGVTYTNDCLLCAYSVEFGT |
| variant of SEQ ID | NO: 2 | NISKEHDGECKETVPMNCSSYANTTSEDGKVMVLCNRAFNPVCGTDGVTYD |
| 1 | | NECLLCAHKVEQGASVDKRHDGGCRKELAAVSVDCSEYPKPDCTAEDRPLC |
| | | GSDNKTYGNKCNFCNAVVESNGTLTLSHFGKC |
| G162M F167A | SEQ ID | AEVDCSRFPNATDMEGKDVLVCNKDLRPICGTDGVTYTNDCLLCAYSVEFGT |
| Ovomucoid | NO: 3 | NISKEHDGECKETVPMNCSSYANTTSEDGKVMVLCNRAFNPVCGTDGVTYD |
| Variant of Chicken | | NECLLCAHKVEQGASVDKRHDGGCRKELAAVSVDCSEYPKPDCTAEDRPLC |
| OVD in Genbank | | GSDNKTYMNKCNACNAVVESNGTLTLSHFGKC |
| Ovomucoid | SEQ ID | MAMAGVFVLFSFVLCGFLPDAAFGAEVDCSRFPNATDKEGKDVLVCNKDLR |
| isoform 1 | NO: 4 | PICGTDGVTYTNDCLLCAYSIEFGTNISKEHDGECKETVPMNCSSYANTTS |
| precursor full | | EDGKVMVLCNRAFNPVCGTDGVTYDNECLLCAHKVEQGASVDKRHDGGCRKE |
| length | | LAAVSVDCSEYPKPDCTAEDRPLCGSDNKTYGNKCNFCNAVVESNGTLTLSH |
| | | FGKC |
| Ovomucoid | SEQ ID | MAMAGVFVLFSFVLCGFLPDAVFGAEVDCSRFPNATDMEGKDVLVCNKDLR |
| [Gallus gallus] | NO: 5 | PICGTDGVTYTNDCLLCAYSVEFGTNISKEHDGECKETVPMNCSSYANTTSE |
| | | DGKVMVLCNRAFNPVCGTDGVTYDNECLLCAHKVEQGASVDKRHDGGCRKE |
| | | LAAVSVDCSEYPKPDCTAEDRPLCGSDNKTYGNKCNFCNAVVESNGTLILSH |
| | | FGKC |
| Ovomucoid | SEQ ID | MAMAGVFVLFSFVLCGFLPDAAFGAEVDCSRFPNATDKEGKDVLVCNKDLR |
| isoform 2 | NO: 6 | PICGTDGVTYTNDCLLCAYSIEFGTNISKEHDGECKETVPMNCSSYANTTSE |
| precursor [Gallus | | DGKVMVLCNRAFNPVCGTDGVTYDNECLLCAHKVEQGASVDKRHDGGCRKE |
| gallus] | | LAAVDCSEYPKPDCTAEDRPLCQSDNKTYGNKCNFCNAVVESNGTLTLSHF |
| | | GKC |
| Ovomucoid | SEQ ID | AEVDCSRFPNATDKEGKDVLVCNKDLRPICGTDGVTYNNECLLCAYSIEFGT |
| [Gallus gallus] | NO: 7 | NISKEHDGECKETVPMNCSSYANTTSEDGKVMVLCNRAFNPVCGTDGVTYD |
| | | NECLLCAHKVEQGASVDKRHDGECRKELAAVSVDCSEYPKPDCTAEDRPLC |
| | | GSDNKTYGNKCNFCNAVVESNGTLILSHFGKC |
| Ovomucoid | SEQ ID | MAMAGVFVIFSFALCGFLPDAAFGVEVDCSRFPNATNEEGKDVLVCTEDLRP |
| [Numida | NO: 8 | ICGTDGVTYSNDCLLCAYNIEYGTNISKEHDGECREAVPVDCSRYPNMTSEEG |
| meleagris] | | KVLILCNKAFNPVCGTDGVTYDNECLLCAHNVEQGTSVGKKHDGECRKELA |
| | | AVDCSEYPKPACTMEYRPLCGSDNKTYDNKCNFCNAVVESNGTLTLSHFGK |
| | | C |
| PREDICTED: | SEQ ID | MQTITWRQPQGDHLRSRAPAATCRAGQYLTMAMAGIFVLFSFALCGFLPDAA |
| Ovomucoid | NO: 9 | FGVEVDCSRFPNTTNEEGKDVLVCTEDLRPICGIDGVTHSECLLCAYNIEYGT |
| isoform X1 | | NISKEHDGECREAVPMDCSRYPNTTNEEGKVMILCNKALNPVCGTDGVTYD |
| [Meleagris | | NECVLCAHNLEQGTSVGKKHDGGCRKELAAVSVDCSEYPKPACTLEYRPLC |
| gallopavo] | | GSDNKTYGNKCNFCNAVVESNGTLTLSHFGKC |
| Ovomucoid | SEQ ID | VEVDCSRFPNTTNEEGKDVLVCTEDLRPICGTDGVTHSECLLCAYNIEYGTNIS |
| [Meleagris | NO: 10 | KEHDGECREAVPMDCSRYPNTTSEEGKVMILCNKALNPVCGTDGVTYDNEC |
| gallopavo] | | VLCAHNLEQGTSVGKKHDGECRKELAAVSVDCSEYPKPACTLEYRPLCGSDN |
| | | KTYGNKCNFCNAVVESNGTLTLSHFGKC |
| PREDICTED: | SEQ ID | MQTITWRQPQGDHLRSRAPAATCRAGQYLTMAMAGIFVLFSFALCGFLPDAA |
| Ovomucoid | NO: 11 | FGVEVDCSRFPNTTNEEGKDVLVCTEDLRPICGTDGVTHSECLLCAYNIEYGT |
| isoform X2 | | NISKEHDGECREAVPMDCSRYPNTTNEEGKVMILCNKALNPVCGTDGVTYD |
| [Meleagris | | NECVLCAHNLEQGTSVGKKHDGGCRKELAAVDCSEYPKPACTLEYRPLCGS |
| gallopavo] | | DNKTYGNKCNFCNAVVESNGTLTLSHFGKC |
| Ovomucoid | SEQ ID | EYGTNISIKHNGECKETVPMDCSRYANMINEEGKVMMPCDRTYNPVCGTDG |
| [Bambusicola | NO: 12 | VTYDNECQLCAHNVEQGTSVDKKHDGVCGKELAAVSVDCSEYPKPECTAEE |
| thoracicus] | | RPICGSDNKTYGNKCNFCNAVVYVQP |
| Ovomucoid | SEQ ID | VDCSRFPNTTNEEGKDVLACTKELHPICGTDGVTYSNECLLCYYNIEYGTNIS |
| [Callipepla | NO: 13 | KEHDGECTEAVPVDCSRYPNTTSEEGKVLIPCNRDFNPVCGSDGVTYENECLL |
| squamata] | | CAHNVEQGTSVGKKHDGGCRKEFAAVSVDCSEYPKPDCTLEYRPLCGSDNK |
| | | TYASKCNFCNAVVIWEQEKNTRHHASHSVFFISARLVC |
| Ovomucoid | SEQ ID | MLPLGLREYGTNTSKEHDGECTEAVPVDCSRYPNTTSEEGKVRILCKKDINPV |
| [Colinus | NO: 14 | CGTDGVTYDNECLLCSHSVGQGASIDKKHDGGCRKEFAAVSVDCSEYPKPAC |
| virginianus] | | MSEYRPLCGSDNKTYVNKCNFCNAVVYVQPWLHSRCRLPPTGTSFLGSEGRE |
| | | TSLLTSRATDLQVAGCTAISAMEATRAAALLGLVLLSSFCELSHLCFSQASCD |
| | | VYRLSGSRNLACPRIFQPVCGTDNVTYPNECSLCRQMLRSRAVYKKHDGRCV |
| | | KVDCTGYMRATGGLGTACSQQYSPLYATNGVIYSNKCTFCSAVANGEDIDLL |
| | | AVKYPEEESWISVSPTPWRMLSAGA |
| Ovomucoid-like | SEQ ID | MSWWGIKPALERPSQEQSTSGQPVDSGSTSTTTMAGIFVLLSLVLCCFPDAAF |
| isoform X2 [Anser | NO: 15 | GVEVDCSRFPNTTNEEGKEVLLCTKDLSPICGTDGVTYSNECLLCAYNIEYGT |
| cygnoides | | NISKDHDGECKEAVPVDCSTYPNMTNEEGKVMLVCNKMFSPVCGTDGVTYD |
| domesticus] | | NECMICAHNVEQGTSVGKKYDGKCKKEVATVDCSDYPKPACTVEYMPLCG |
| | | SDNKTYDNKCNFCNAVVDSNGTLTLSHFGKC |
| Ovomucoid-like | SEQ ID | MSSQNQLHRRRRPLPGGQDLNKYYWPHCTSDRFSWLLHVTAEQFRHCVCIYLQ |
| isoform X1 [Anser | NO: 16 | PALERPSQEQSTSGQPVDSGSTSTTTMAGIFVLLSLVLCCFPDAAFGVEVDCS |
| cygnoides | | RFPNTTNEEGKEVLLCTKDLSPICGTDGVTYSNECLLCAYNIEYGTNISKDHD |
| domesticus] | | GECKEAVPVDCSTYPNMTNEEGKVMLVCNKMFSPVCGTDGVTYDNECMLC |
| | | AHNVEQGTSVGKKYDGKCKKEVATVDCSDYPKPACTVEYMPLCGSDNKTY |
| | | DNKCNFCNAVVDSNGTLTLSHFGKC |
| Ovomucoid | SEQ ID | VEVDCSRFPNTTNEEGKDEVVCPDELRLICGTDGVTYNHECMLCFYNKEYGT |
| [Cotumix | NO: 17 | NISKEQDGECGETVPMDCSRYPNITSEDGKVTILCTKDFSFVCGIDGVTYDNE |
| japonica] | | CMLCAHNVVQGTSVGKKHDGECRKELAAVSVDCSEYPKPACPKDYRPVCGS |
| | | DNKTYSNKCNFCNAVVESNGTLTLNHFGKC |
| Ovomucoid | SEQ ID | MAMAGVFLLFSFALCGFLPDAAFGVEVDCSRFPNTTNEEGKDEVVCPDELRLI |
| [Cotumix | NO: 18 | CGTDGVTYNHECMLCFYNKEYGTNISKEQDGECGETVPMDCSRYPNTTSED |
| japonica] | | GKVTILCTKDFSFVCGTDGVTYDNECMLCAHNIVQGTSVGKKHDGECRKEL |
| | | AAVSVDCSEYPKPACPKDYRPVCGSDNKTYSNKCNFCNAVVESNGTLTLNHF |
| | | GKC |
| Ovomucoid [Anas | SEQ ID | MAGVFVLLSLVLCCFPDAAFGVEVDCSRFPNTTNEEGKDVLLCTKELSPVCG |
| platyrhynchos] | NO: 19 | TDGVTYSNECLLCAYNIEYGTNISKDHDGECKEAVPADCSMYPNMTNEEGK |
| | | MTLLCNKMFSPVCGTDGVTYDNECMLCAHNVEQGTSVGKKYDGKCKKEVA |
| | | TVDCSGYPKPACTMEYMPLCGSDNKTYGNKCNFCNAVVDSNGTLTLSHFGE |
| | | C |
| Ovomucoid, | SEQ ID | QVDCSRFPNTTNEEGKEVLLCTKELSPVCGTDGVTYSNECLLCAYNIEYGTN |
| partial [Anas | NO: 20 | ISKDHDGECKEAVPADCSMYPNMTNEEGKMTLLCNKMFSPVCGTDGVTYDN |
| platyrhynchos] | | ECMLCAHNVEQGTSVGKKYDGKCKKEVATVSVDCSGYPKPACTMEYMPLC |
| | | GSDNKTYGNKCNFCNAVV |
| Ovomucoid-like | SEQ ID | MTMPGAFVVLSFVLCCFPDATFGVEVDCSTYPNTTNEEGKEVLVCSKILSPIC |
| [Tyto alba] | NO: 21 | GTDGVTYSNECLLCANNIEYGTNISKYHDGECKEFVPVNCSRYPNTTNEEGK |
| | | VMLICNKDLSPVCGTDGVTYDNECLLCAHNLEPGTSVGKKYDGECKKEIATV |
| | | DCSDYPKPVCSLESMPLCGSDNKTYSNKCNFCNAVVDSNETLTLSHFGKC |
| Ovomucoid | SEQ ID | MTMAGVFVLLSFALCCFPDAAFGVEVDCSTYPNTTNEEGKEVLVCTKILSPIC |
| [Balearica | NO: 22 | GTDGVTYSNECLLCAYNIEYGTNVSKDHDGECKEVVPVDCSRYPNSTNEEGK |
| regulorum | | VVMLCSKDLNPVCGTDGVTYDNECVLCAHNVESGTSVGKKYDGECKKETA |
| gibbericeps] | | TVDCSDYPKPACTLEYMPFCGSDSKTYSNKCNFCNAVVDSNGTLTLSHFGKC |
| Turkey vulture | SEQ ID | MTTAGVFVLLSFALCSFPDAAFGVEVDCSTYPNTTNEEGKEVLVCTKILSPI |
| [Cathartes aura] | NO: 23 | CGTDGVTYSNECLLCAYNIEYGTNVSKDHDGECKEFVPVDCSRYPNTTNEDG |
| OVD (native | | KVVLLCNKDLSPICGTDGVTYDNECLLCARNLEPGTSVGKKYDGECKKEIAT |
| sequence) | | VDCSDYPKPVCSLEYMPLCGSDSKTYSNKCNFCNAVVDSNGTLTLSHFGKC |
| bolded is native | | |
| signal sequence | | |
| Ovomucoid-like | SEQ ID | MTTAGVFVLLSFILCSFPDAAFGVEVDCSPYPNTTNBEGKEVLVCNKILSPICG |
| [Cuculus canorus] | NO: 24 | TDGVTYSNECLLCAYNLEYGTNISKDYDGECKEVAPVDCSRHPNTTNEEGKV |
| | | SDYPKPVCTLEEMPLCGSDNKTYGNKCNFCNAVVDSNGTLILSHFGKC |
| Ovomucoid | SEQ ID | MTTAVVFVLLSFALCCFPDAAFGVEVDCSTYPNSTNEEGKDVLVCPKILGPIC |
| [Antrostomos | NO: 25 | GTDGVTYSNECLLCAYNIQYGTNVSKDHDGECKEIVPVDCSRYPNTTNEEGK |
| carolinensis] | | VVFLCNKNEDPVCGTDGDTYDNECMLCARSLEPGTTVGKKHDGECKREIAT |
| | | VDCSDYPKPTCSAEDMPLCGSDSKTYSNKCNFCNAVVDSNGTLILSRFGKC |
| Ovomucoid | SEQ ID | MTMTGVFVLLSFAICCFPDAAFGVEVDCSTYPNTTNEEGKEVLVCTKILSPICG |
| [Cariama cristata] | NO: 26 | TDGVTYSNECLLCAYNIEYGTNVSKDHDGECKEVVPVDCSKYPNTTNEEGKV |
| | | VLLCSKDLSPVCGTDGVTYDNECLLCARNLEPGSSVGKKYDGECKKEIATIDC |
| | | SDYPKPVCSLEYMPLCGSDSKTYDNKCNFCNAVVDSNGTLTLSHFGKC |
| Ovomucoid-like | SEQ ID | MTTAGVFVLLSFVLCCFPDAVFGVEVDCSTYPNTTNEEGKEVLVCTKILSPIC |
| isoform X2 | NO: 27 | GTDGVTYSNECLLCAYNIEYGTNVSKDHDGECKEVVPVNCSRYPNTTNEEGK |
| [Py goscelis | | VVLRCSKDLSPVCGTDGVTYDNECLMCARNLEPGAVVGKNYDGECKKEIAT |
| adeliae] | | VDCSDYPKPVCSLEYMPLCGSDSKTYSNKCNFCNAVVDSNGTLILSHFGKC |
| Ovomucoid-like | SEQ ID | MTTAGVFVLLSIALCCFPDAAFGVEVDCSAYSNTTSEEGKEVLSCTKILSPICG |
| [Nipponia nippon] | NO: 28 | TDGVTYSNECLLCAYNIEYGTNISKDHDGECKEVVSVDCSRYPNTTNEEGKA |
| | | VLLCNKDLSPVCGTDGVTYDNECLLCAHNLEPGTSVGKKYDGACKKEIATV |
| | | DCSDYPKPVCTLEYLPLCGSDSKTYSNKCDFCNAVVDSNGTLTLSHFGKC |
| Ovomucoid-like | SEQ ID | MTTAGVFVLLSFALCCFPDAAFGVEVDCSTYPNTTNEEGKEVLVCTKILSPIC |
| [Phaethon | NO: 29 | GTDGTTYSNECLLCAYNIEYGTNVSKDHDGECKVVPVDCSKYPNTTNEDGK |
| lepturus] | | VVLLCNKALSPICGTDRVTYDNECLMCAHNLEPGTSVGKKHDGECQKEVAT |
| | | VDCSDYPKPVCSLEYMPLCGSDGKTYSNKCNFCNAVVNSNGTLTLSHFEKC |
| Ovomucoid-like | SEQ ID | MTTAGVFVLLSFVLCCFFPDAAFGVEVDCSTYPNTTNEEGKEVLVCAKILSPV |
| isoform X1 | NO: 30 | CGTDGVTYSNECLLCAHNIENGINVGKDHDGKCKEAVPVDCSRYPNTTDEE |
| [Melopsittacus | | GKVVLLCNKDVSPVCGTDGVTYDNECLLCAHNLEAGTSVDKKNDSECKTED |
| undulatus] | | TILAAVSVDCSDYPKPVCTLEYLPLCGSDNKTYSNKCRFCNAVVDSNGTLTL |
| | | SRFGKC |
| Ovomucoid | SEQ ID | MTTAGVFVLLSFALCCSPDAAFGVEVDCSTYPNTTNEEGKEVLACTKILSPIC |
| [Podiceps | NO: 31 | GTDGVTYSNECLLCAYNMEYGINVSKDHDGKCKEVVPVDCSRYPNTTNEEG |
| cristatus] | | KVVLLCNKDLSPVCGTDGVTYDNECLLCARNLEPGASVGKKYDGECKKEIA |
| | | TVDCSDYPKPVCSLEHMPLCGSDSKTYSNKCTFCNAVVDSNGILTLSHFGKC |
| Ovomucoid-like | SEQ ID | MTTAGVFVLLSFALCCFPDAAFGVEVDCSTYPNTTNEEGREVLVCTKILSPIC |
| [Fulmarus | NO: 32 | GTDGVTYSNECLLCAYNIEYGTNVSKDHDGECKEVAPVGCSRYPNTTNEEGK |
| glacialis] | | VVLLCNKDLSPVCGTDGVTYDNECLLCARHLEPGTSVGKKYDGECKKEIATV |
| | | DCSDYPKPVCSLEYMPLCGSDSKTYSNKCNFCNAVLDSNGTLTLSHFGKC |
| Ovomucoid | SEQ ID | MTTAGVFVLLSFALCCFPDAVFGVEVDCSTYPNTTNEEGKEVLVCTKILSPIC |
| [Aptenodytes | NO: 33 | GTDGVTYSNECLLCAYNIEYGTNVSKDHDGECKEVVPVDCSRYPNTTNEEGK |
| forsteri] | | VVLRCNKDLSPVCGTDGVTYDNECLMCARNLEPGAIVQKKYDGECKKEIAT |
| | | VDCSDYPKPVCSLEYMPLCGSDSKTYSNKCNFCNAVVDSNGTLILSHFGKC |
| Ovomucoid-like | SEQ ID | MTTAGVFVLLSFVLCCFPDAVFGVEVDCSTYPNTTNEEGKEVLVCTKILSPIC |
| isoform X1 | NO: 34 | GTDGVTYSNECLLCAYNIEYGTNVSKDHDGECKEVVPVDCSRYPNTTNEEGK |
| [Py goscelis | | VVLRCSKDLSPVCGTDGVTYDNECLMCARNLEPGAVVGKNYDGECKKEIAT |
| adeliae] | | VDCSDYPKPVCSLEYMPLCGSDSKTYSNKCNFCNAVVDSNGTLTLSHFGKC |
| Ovomucoid | SEQ ID | MSSQNQLPSRCRPLPGSQDLNKYYQPHCTGDRFCWLFYVTVEQFRHCICIYLQ |
| isoform X1 | NO: 35 | LALERPSHEQSGQPADSRNTSTMTTAGVFVLLSFALCCFPDAVFGVEVDCSTY |
| [Aptenodytes | | PNTTNEEGKEVLVCTKILSPICGTDGVTYSNECLLCAYNIEYGTNVSKDHDGE |
| forsteri] | | CKEVVPVDCSRYPNTTNEEGKVVLRCNKDLSPVCGTDGVTYDNECLMCARN |
| | | LEPGAIVGKKYDGECKKEIATVDCSDYPKPVCSLEYMPLCGSDSKTYSNKCN |
| | | FCNAVVDSNGTLILSHFGKC |
| Ovomucoid, | SEQ ID | MTTAVVFVLLSFALCCFPDAAFGVEVDCSTYPNSTNEEGKDVLVCPKILGPIC |
| partial | NO: 36 | GTDGVTYSNECLLCAYNIQYGTNVSKDHDGECKEIVPVDCSRYPNTTNEEGK |
| [Antrostomus | | VVFLCNKNFDPVCGTDGDTYDNECMLCARSLEPGTTVGKKHDGECKREIAT |
| carolinensis] | | VDCSDYPKPTCSAEDMPLCGSDSKTYSNKCNFCNAVV |
| rOVD as | SEQ ID | EAEAAEVDCSRFPNATDKEGKDVLVCNKDLRPICGTDGVTYTNDCLLCAYSI |
| expressed in pichia | NO: 37 | EFGTNISKEHDGECKETVPMNCSSYANTTSEDGKVMVLCNRAFNPVCGTDGV |
| secreted form 1 | | TYDNECLLCAHKVEQGASVDKRHDGGCRKELAAVSVDCSEYPKPDCTAEDR |
| | | PLCGSDNKTYGNKCNFCNAVVESNGTLTLSHFGKC |
| rOVD as | SEQ ID | EEGVSLEKREAEAAEVDCSRFPNATDKEGKDVLVCNKDLRPICGTDGVTYTN |
| expressed in pichia | NO: 38 | DCLLCAYSIEFGTNISKEHDGECKETVPMNCSSYANTTSEDGKVMVLCNRAF |
| secreted form 2 | | NPVCGTDGVTYDNECLLCAHKVEQGASVDKRHDGGCRKELAAVSVDCSEYP |
| | | KPDCTAEDRPLCGSDNKTYGNKCNFCNAVVESNGTLTLSHFGKC |
| rOVD [gallus] | SEQ ID | MRFPSIFTAVLFAASSALAAPVNTTTEDETAQIPAEAVIGYSDLEGDFDVA |
| coding sequence | NO: 39 | VLPFSNSTNNGLLFINTTIASIAAKEEGVSLEKREAEAAEVDCSRFPNATDK |
| containing an | | EGKDVLVCNKDLRPICGTDGVTYTNDCLLCAYSIEFGTNISKEHDGECKETVP |
| alpha mating | | MNCSSYANTTSEDGKVMVLCNRAFNPVCGTDGVTYDNECLLCAHKVEQGA |
| factor signal | | SVDKRHDGGCRKELAAVSVDCSEYPKPDCTAEDRPLCGSDNKTYGNKCNFC |
| sequence (bolded) | | NAVVESNGTLTLSHFGKC |
| as expressed in | | |
| pichia | | |
| Turkey vulture | SEQ ID | MRFPSIFTAVLFAASSALAAPVNTTTEDETAQIPAEAVIGYSDLEGDFDVA |
| OVD coding | NO: 40 | VLPFSNSTNNGLLFINTTIASIAAKEEGVSLEKREAEAVEVDCSTYPNTTNE |
| sequence | | EGKEVLVCTKILSPICGTDGVTYSNECLLCAYNIEYGTNVSKDHDGECKEFVP |
| containing | | VDCSRYPNTTNEDGKVVLLCNKDLSPICGTDGVTYDNECLLCARNLEPGTSV |
| secretion signals | | GKKYDGECKKEIATVDCSDYPKPVCSLEYMPLCGSDSKTYSNKCNFCNAVV |
| as expressed in | | DSNGTLTLSHFGKC |
| pichia | | |
| bolded is an alpha | | |
| mating factor | | |
| signal sequence | | |
| Turkey vulture | SEQ ID | EAEAVEVDCSTYPNTTNEEGKEVLVCTKILSPICGTDGVTYSNECLLCAYNIE |
| OVD in secreted | NO: 41 | YGTNVSKDHDGECKEFVPVDCSRYPNTTNEDGKVVLLCNKDLSPICGTDGVT |
| form expressed in | | YDNECLLCARNLEPGTSVGKKYDGECKKEIATVDCSDYPKPVCSLEYMPLCG |
| Pichia | | SDSKTYSNKCNFCNAVVDSNGTLTLSHFGKC |
| Humming bird | SEQ ID | MTMAGVFVLLSFILCCFPDTAFGVEVDCSIYPNTTSEEGKEVLVCTETLSPIC |
| OVD (native | NO: 42 | GSDGVTYNNECQLCAYNVEYGINVSKDHDGECKEIVPVDCSRYPNTTEEGR |
| sequence) | | VVMLCNKALSPVCGTDGVTYDNECLLCARNLESGTSVGKKFDGECKKEIAT |
| bolded is the | | VDCTDYPKPVCSLDYMPLCGSDSKTYSNKCNFCNAVMDSNGTLILNHFGKC |
| native signal | | |
| sequence | | |
| Humming bird | SEQ ID | MRFPSIFTAVLFAASSALAAPVNTTTEDETAQIPAEAVIGYSDLEGDFDVA |
| OVD coding | NO: 43 | VLPFSNSTNNGLLFINTTIASIAAKEEGVSLDKREAEAVEVDCSIYPNTTSEE |
| sequence as | | GKEVLVCTETLSPICGSDGVTYNNECQLCAYNVEYGTNVSKDHDGECKEIVP |
| expressed in | | VDCSRYPNTTEEGRVVMLCNKALSPVCGTDGVTYDNECLLCARNLESGTSV |
| Pichia | | GKKFDGECKKEIATVDCTDYPKPVCSLDYMPLCGSDSKTYSNKCNFCNAVM |
| bolded is an alpha | | DSNGTLTLNHFGKC |
| mating factor | | |
| signal sequence | | |
| Humming bird | SEQ ID | EAEAVEVDCSTYPNTTSEEGKEVLVCTETLSPICGSDGVTYNNECQLCAYNVE |
| OVD in secreted | NO: 44 | YGTNVSKDHDGECKEIVPVDCSRYPNTTEEGRVVMLCNKALSPVCGTDGVT |
| form from Pichia | | YDNECLLCARNLESGTSVGKKFDGECKKEIATVDCTDYPKPVCSLDYMPLCG |
| | | SDSKTYSNKCNFCNAVMDSNGTLTLNHFGKC |
| Chicken | SEQ ID | MRFPSIFTAVLFAASSALAAPVNTTTEDETAQIPAEAVIGYSDLEGDFDVA |
| Ovalbumin with | NO: 45 | VLPFSNSTNNGLLFINTTIASIAAKEEGVSLDKREAEAGSIGAASMEFCFDVF |
| bolded signal | | KELKVHHANENIFYCPIAIMSALAMVYLGAKDSTRTQINKVVRFDKLPGFGDS |
| | | IEAQCGTSVNVHSSLRDILNQITKPNDVYSFSLASRLYAEERYPILPEYLQCVK |
| | | ELYRGGLEPINFQTAADQARELINSWVESQTNGHIRNVLQPSSVDSQTAMVLV |
| | | NAIVFKGLWEKAFKDEDTQAMPFRVTEQESKPVQMMYQIGLFRVASMASEK |
| | | MKILELPFASGTMSMLVLLPDEVSGLEQLESIINFEKLTEWTSSNVMEERKIKV |
| | | YLPRMKMEEKYNLTSVLMAMGITDVFSSSANLSGISSAESLKISQAVHAAHAE |
| | | INEAGREVVGSAEAGVDAASVSEEFRADHPFLFCIKHIATNAVLFFGRCVSP |
| Chicken OVA | SEQ ID | EAEAGSIGAASMEFCFDVFKELKVHHANENIFYCPIAIMSALAMVYLGAKDST |
| sequence as | NO: 46 | RTQINKVVRFDKLPGFGDSIEAQCGTSVNVHSSLRDILNQITKPNDVYSFSLAS |
| secreted from | | RLYAFERYPILPEYLQCVKELYRGGLEPINFQTAADQARELINSWVESQTNGII |
| pichia | | RNVLQPSSVDSQTAMVLVNAIVFKGLWEKAFKDEDTQAMPFRVTEQESKPV |
| sequence | | QMMYQIGLFRVASMASEKMKILELPFASGTMSMLVLLPDEVSGLEQLESIINF |
| | | EKLTEWTSSNVMEERKIKVYLPRMKMEEKYNLTSVLMAMGITDVFSSSANLS |
| | | GISSAESLKISQAVHAAHAEINEAGREVVGSAEAGVDAASVSEEFRADHPFLF |
| | | CIKHIATNAVLFFGRCVSP |
| Predicted | SEQ ID | MRVPAQLLGLLLLWLPGARCGSIGAASMEFCFDVFKELKVHHANENIFYCPIA |
| Ovalbumin | NO: 47 | IMSALAMVYLGAKDSTRTQINKVVRFDKLPGFGDSIEAQCGTSVNVHSSLRDI |
| [Achromobacter | | LNQITKPNDVYSFSLASRLYAEERYPILPEYLQCVKELYRGGLEPINFQTAADQ |
| denitrificans] | | ARELINSWVESQTNGIIRNVLQPSSVDSQTAMVLVNAIVFKGLWEKAFKDEDT |
| | | QAMPFRVTEQESKPVQMMYQIGLFRVASMASEKMKILELPFASGIMSMLVLL |
| | | PDEVSGLEQLESIINFEKLTEWTSSNVMEERKIKVYLPRMKMEEKYNLTSVLM |
| | | AMGITDVFSSSANLSGISSAESLKISQAVHAAHAEINEAGREVVGSAEAGVDA |
| | | ASVSEEFRADHPFLFCIKHIATNAVLFFGRCVSPLEIKRAAAHHHHHH |
| OLLAS epitope- | SEQ ID | MTSGFANELGPRLMGKLTMGSIGAASMEFCFDVFKELKVHHANENIFYCPIAI |
| tagged ovalbumin | NO: 48 | MSALAMVYLGAKDSTRTQINKVVRFDKLPGFGDSIEAQCGTSVNVHSSLRDI |
| | | LNQITKPNDVYSFSLASRLYAEERYPILPEYLQCVKELYRGGLEPINFQTAADQ |
| | | ARELINSWVESQTNGHIRNVLQPSSVDSQTAMVLVNAIVFKGLWEKTFKDEDT |
| | | QAMPFRVTEQESKPVQMMYQIGLFRVASMASEKMKILELPFASGTMSMLVLL |
| | | PDEVSGLEQLESIINFEKLTEWTSSNVMEERKIKVYLPRMKMEEKYNLTSVLM |
| | | AMGITDVFSSSANLSGISSAESLKISQAVHAAHAEINEAGREVVGSAEAGVDA |
| | | ASVSEEFRADHPFLFCIKHIATNAVLFFGRCVSPSR |
| Serpin family | SEQ ID | MGGRRVRWEVYISRAGYVNRQIAWRRHHRSLTMRVPAQLLGLLLLWLPGA |
| protein | NO: 49 | RCGSIGAASMEFCFDVFKELKVHHANENIFYCPIAIMSALAMVYLGAKDSTRT |
| [Achromobacter | | QINKVVRFDKLPGFGDSIEAQCGTSVNVHSSLRDILNQITKPNDVYSFSLASRL |
| denitrificans] | | VLQPSSVDSQTAMVLVNAIVFKGLWEKAFKDEDTQAMPFRVTEQESKPVQM |
| | | MYQIGLFRVASMASEKMKILELPFASGTMSMLVLLPDEVSGLEQLESIINFEKL |
| | | TEWTSSNVMEERKIKVYLPRMKMEEKYNLTSVLMAMGITDVFSSSANLSGIS |
| | | SAESLKISQAVHAAHAEINEAGREVVGSAEAGVDAASVSEEFRADHPFLFCIK |
| | | HIATNAVLFFGRCVSPLEIKRAAAHHHHHH |
| PREDICTED: | SEQ ID | MGSIGAVSMEFCFDVFKELKVHHANENIFYSPFTIISALAMVYLGAKDSTRTQI |
| ovalbumin isoform | NO: 50 | NKVVRFDKLPGFGDSVEAQCGTSVNVHSSLRDILNQITKPNDVYSFSLASRLY |
| X1 [Meleagris | | ABETYPILPEYLQCVKELYRGGLESINFQTAADQARGLINSWVESQTNGMIKN |
| gallopavo] | | VLQPSSVDSQTAMVLVNAIVFKGLWEKAFKDEDTQAIPFRVTEQESKPVQMM |
| | | YQIGLFKVASMASEKMKILELPFASGTMSMWVLLPDEVSGLEQLETTISFEKM |
| | | TEWISSNIMEERRIKVYLPRMKMEEKYNLTSVLMAMGITDLFSSSANLSGISSA |
| | | GSLKISQAVHAAYAEIYEAGREVIGSAEAGADATSVSEEFRVDHPFLYCIKHN |
| | | LTNSILFFGRCISP |
| Ovalbumin | SEQ ID | MGSIGAVSMEFCFDVFKELKVHHANENIFYSPFTIISALAMVYLGAKDSTRTQI |
| precursor | NO: 51 | NKVVRFDKLPGFGDSVEAQCGTSVNVHSSLRDILNQITKPNDVYSFSLASRLY |
| [Meleagris | | ABETYPILPEYLQCVKELYRGGLESINFQTAADQARGLINSWVESQINGMIKN |
| gallopavo] | | VLQPSSVDSQTAMVLVNAIVFKGLWEKAFKDEDTQAIPFRVTEQESKPVQMM |
| | | YQIGLFKVASMASEKMKILELPFASGTMSMWVLLPDEVSGLEQLETTISFEKM |
| | | TEWISSNIMEERRIKVYLPRMKMEEKYNLTSVLMAMGITDLFSSSANLSGISSA |
| | | GSLKISQAAHAAYAEIYEAGREVIGSAEAGADATSVSEEFRVDHPFLYCIKHN |
| | | LTNSILFFGRCISP |
| Hypothetical | SEQ ID | YYRVPCMVLCTAFHPYIFIVLLFALDNSEFTMGSIGAVSMEFCFDVFKELRVH |
| protein | NO: 52 | HPNENIFFCPFAIMSAMAMVYLGAKDSTRTQINKVIRFDKLPGFGDSTEAQCG |
| [Bambusicola | | KSANVHSSLKDILNQITKPNDVYSFSLASRLYADETYSIQSEYLQCVNELYRG |
| thoracicus] | | GLESINFQTAADQARELINSWVESQTNGIIRNVLQPSSVDSQTAMVLVNAIVFR |
| | | GLWEKAFKDEDTQTMPFRVTEQESKPVQMMYQIGSFKVASMASEKMKILEL |
| | | PLASGTMSMLVLLPDEVSGLEQLETTISFEKLTEWTSSNVMEERKIKVYLPRM |
| | | KMEEKYNLTSVLMAMGITDLFRSSANLSGISLAGNLKISQAVHAAHAEINEAG |
| | | RKAVSSAEAGVDATSVSEEFRADRPFLFCIKHIATKVVFFFGRYTSP |
| Egg albumin | SEQ ID | MGSIGAASMEFCFDVFKELKVHHANDNMLYSPFAILSTLAMVFLGAKDSTRT |
| | NO: 53 | QINKVVHFDKLPGFGDSIEAQCGTSVNVHSSLRDILNQITKQNDAYSFSLASRL |
| | | YAQETYTVVPEYLQCVKELYRGGLESVNFQTAADQARGLINAWVESQTNGII |
| | | RNILQPSSVDSQTAMVLVNAIAFKGLWEKAFKAEDTQTIPFRVTEQESKPVQM |
| | | MYQIGSFKVASMASEKMKILELPFASGTMSMLVLLPDDVSGLEQLESIISFEKL |
| | | TEWTSSSIMEERKVKVYLPRMKMEEKYNLTSLLMAMGITDLFSSSANLSGISS |
| | | VGSLKISQAVHAAHAEINEAGRDVVGSAEAGVDATEEFRADHPFLFCVKHIET |
| | | NAILLFGRCVSP |
| Ovalbumin | SEQ ID | MASIGAVSTEFCVDVYKELRVHHANENIFYSPFTIISTLAMVYLGAKDSTRTQI |
| isoform X2 | NO: 54 | NKVVRFDKLPGFGDSIEAQCGTSVNVHSSLRDILNQITKPNDVYSFSLASRLY |
| [Numida | | ABETYPILPEYLQCVKELYRGGLESINFQTAADQARELINSWVESQTSGIIKNV |
| meleagris] | | LQPSSVNSQTAMVLVNAIYFKGLWERAFKDEDTQAIPFRVTEQESKPVQMMS |
| | | QIGSFKVASVASEKVKILELPFVSGTMSMLVLLPDEVSGLEQLESTISTEKLTE |
| | | WTSSSIMEERKIKVFLPRMRMEEKYNLTSVLMAMGMTDLFSSSANLSGISSAE |
| | | SLKISQAVHAAYAEIYEAGREVVSSAEAGVDATSVSEEFRVDHPFLLCIKHNP |
| | | TNSILFFGRCISP |
| Ovalbumin | SEQ ID | MALCKAFHPYIFIVLLFDVDNSAFTMASIGAVSTEFCVDVYKELRVHHANENI |
| isoform X1 | NO: 55 | FYSPFTIISTLAMVYLGAKDSTRTQINKVVRFDKLPGFGDSIEAQCGTSVNVHS |
| [Numida | | SLRDILNQITKPNDVYSFSLASRLYAEETYPILPEYLQCVKELYRGGLESINFQT |
| meleagris] | | AADQARELINSWVESQTSGIIKNVLQPSSVNSQTAMVLVNAIYFKGLWERAF |
| | | KDEDTQAIPFRVTEQESKPVQMMSQIGSFKVASVASEKVKILELPFVSGTMSM |
| | | LVLLPDEVSGLEQLESTISTEKLTEWTSSSIMEERKIKVFLPRMRMEEKYNLTS |
| | | VLMAMGMTDLFSSSANLSGISSAESLKISQAVHAAYABIYEAGREVVSSAEAG |
| | | VDATSVSEEFRVDHPFLLCIKHNPTNSILFFGRCISP |
| PREDICTED: | SEQ ID | MGSIGAASMEFCFDVFKELKVHHANDNMLYSPFAILSTLAMVFLGAKDSTRT |
| Ovalbumin | NO: 56 | QINKVVHFDKLPGFGDSIEAQCGTSANVHSSLRDILNQITKQNDAYSFSLASRL |
| isoform X2 | | YAQETYTVVPEYLQCVKELYRGGLESVNFQTAADQARGLINAWVESQTNGII |
| [Coturnix | | RNILQPSSVDSQTAMVLVNAIAFKGLWEKAFKAEDTQTIPFRVTEQESKPVQM |
| Japonica] | | MHQIGSFKVASMASEKMKILELPFASGTMSMI.VLLPDDVSGLEQLESTISFEK |
| | | LTEWTSSSIMEERKVKVYLPRMKMEEKYNLTSLLMAMGITDLFSSSANLSGIS |
| | | SVGSLKISQAVHAAYABINEAGRDVVGSAEAGVDATEEFRADHPFLFCVKHIE |
| | | TNAILLFGRCVSP |
| PREDICTED: | SEQ ID | MGLCTAFHPYIFIVLLFALDNSEFTMGSIGAASMEFCFDVFKELKVHHANDN |
| ovalbumin isoform | NO: 57 | MLYSPFAILSTLAMVFLGAKDSTRTQINKVVHFDKLPGFGDSIEAQCGTSANV |
| X1 [Coturmix | | HSSLRDILNQITKQNDAYSFSLASRLYAQETYTVVPEYLQCVKELYRGGLESV |
| japonica] | | NFQTAADQARGLINAWVESQTNGHIRNILQPSSVDSQTAMVLVNAIAFKGLWE |
| | | KAFKAEDTQTIPFRVTEQESKPVQMMHQIGSFKVASMASEKMKILELPFASGT |
| | | MSMLVLLPDDVSGLEQLESTISFEKLTEWTSSSIMEERKVKVYLPRMKMEEK |
| | | YNLTSLLMAMGITDLFSSSANLSGISSVGSLKISQAVHAAYAEINEAGRDVVG |
| | | SAEAGVDATEEFRADHPFLFCVKHIETNAILLFGRCVSP |
| Egg albumin | SEQ ID | MGSIGAASMEFCFDVFKELKVHHANDNMLYSPFAILSTLAMVFLGAKDSTRT |
| | NO: 58 | QINKVVHFDKLPGFGDSIEAQCGTSANVHSSLRDILNQITKQNDAYSFSLASRL |
| | | YAQETYTVVPEYLQCVKELYRGGLESVNFQTAADQARGLINAWVESQINGII |
| | | RNILQPSSVDSQTAMVLVNAIAFKGLWEKAFKAEDTQTIPFRVTEQESKPVQM |
| | | MHQIGSFKVASMASEKMKILELPFASGTMSMLVLLPDDVSGLEQLESTISFEK |
| | | LTEWTSSSIMEERKVKVYLPRMKMEEKYNLTSLLMAMGITDLFSSSANLSGIS |
| | | SVGSLKIPQAVHAAYAEINEAGRDVVGSAEAGVDATEEFRADHPFLFCVKHIE |
| | | TNAILLFGRCVSP |
| ovalbumin [Anas | SEQ ID | MGSIGAASTEFCFDVFRELRVQHVNENIFYSPFSIISALAMVYLGARDNTRTQI |
| platyrhynchos] | NO: 59 | DKVVHFDKLPGFGESMEAQCGTSVSVHSSLRDILTQITKPSDNFSLSFASRLYA |
| | | EETYAILPEYLQCVKELYKGGLESISFQTAADQARELINSWVESQINGIIKNIL |
| | | QPSSVDSQTTMVLVNAIYFKGMWEKAFKDEDTQAMPFRMTEQESKPVQMM |
| | | YQVGSFKVAMVISEKMKILELPFASGMMSMFVLLPDEVSGLEQLESTISFEKL |
| | | TEWTSSTMMEERRMKVYLPRMKMEEKYNLTSVFMALGMTDLFSSSANMSGI |
| | | SSTVSLKMSEAVHAACVEIFEAGRDVVGSAEAGMDVTSVSEEFRADHPFLFFI |
| | | KHNPTNSILFFGRWMSP |
| PREDICTED: | SEQ ID | MGSIGAASTEFCFDVFRELKVQHVNENIFYSPLSIISALAMVYLGARDNTRTQI |
| ovalbumin-like | NO: 60 | DQVVHFDKIPGFGESMEAQCGTSVSVHSSLRDILTEITKPSDNFSLSFASRLYA |
| [Anser cygnoides | | EETYTILPEYLQCVKELYKGGLESISFQTAADQARELINSWVESQTNGIIKNILQ |
| domesticus] | | PSSVDSQTTMVLVNAIYFKGMWEKAFKDEDTQTMPFRMTEQESKPVQMMY |
| | | QVGSFKLATVTSEKVKILELPFASGMMSMCVLLPDEVSGLEQLETTISFEKLTE |
| | | WTSSTMMEERRMKVYLPRMKMEEKYNLTSVFMALGMTDLFSSSANMSGISS |
| | | TVSLKMSEAVHAACVEIFEAGRDVVGSAEAGMDVTSVSEEFRADHPFLFFIK |
| | | HNPSNSILFFGRWISP |
| PREDICTED: | SEQ ID | MGSIGAASTEFCFDVFKELKVQHVNENIFYSPLTIISALSMVYLGARENTRAQI |
| Ovalbumin-like | NO: 6 | DKVLHFDKMPGFGDTIESQCGTSVSIHTSLKDMFTQITKPSDNYSLSFASRLYA |
| [Aquila chrysaetos | | EETYPILPEYLQCVKELYKGGLETISFQTAAEQARELINSWVESQTNGMIKNIL |
| canadensis] | | QPSSVDPQTKMVLVNAIYFKGVWEKAFKDEDTQEVPFRVTEQESKPVQMMY |
| | | QIGSFKVAVMASEKMKILELPYASGQLSMLVLLPDDVSGLEQLESAITFEKLM |
| | | AWTSSTTMEERKMKVYLPRMKIEEKYNLTSVLMALGVTDLFSSSANLSGISS |
| | | ABSLKISKAVHEAFVEIYEAGSEVVGSTEAGMEVTSVSEEFRADHPFLFLIKHN |
| | | PTNSILFFGRCFSP |
| PREDICTED: | SEQ ID | MGSIGAASTEFCFDVFKELKVQHVNENIFYSPLTIISALSMVYLGARENTRTQI |
| Ovalbumin-like | NO: 62 | DKVLHFDKMTGFGDTVESQCGTSVSIHTSLKDIFTQITKPSDNYSLSLASRLYA |
| [Haliaeetus | | EETYPILPEYLQCVKELYKGGLETVSFQTAABQARELINSWVESQTNGMIKNI |
| albicilla] | | LQPSSVDPQTKMVLVNAIYFKGVWEKAFKDEDTQEVPFRVTEQESKPVQMM |
| | | YQIGSFKVAVMASEKMKILELPYASGQLSMLVLLPDDVSGLEQLESAITSEKL |
| | | MEWTSSTTMEERKMKVYLPRMKIEEKYNLTSVLMALGVTDLFSSSADLSGIS |
| | | SAESLKISKAVHEAFVEIYEAGSEVVGSTEGGMEVTSVSEEFRADHPFLFLIKH |
| | | KPTNSILFFGRCFSP |
| PREDICTED: | SEQ ID | MGSIGAASTEFCFDVFKELKVQHVNENIFYSPLTIISALSMVYLGARENTRTQI |
| Ovalbumin-like | NO: 63 | DKVLHFDKMTGFGDTVESQCGTSVSIHTSLKDIFTQITKPSDNYSLSLASRLYA |
| [Haliaeetus | | EETYPILPEYLQCVKELYKGGLETVSFQTAABQARELINSWVESQTNGMIKNI |
| leucocephalus] | | LQPSSVDPQTKMVLVNAIYFKGVWEKAFKDEDTQEVPFRVTEQESKPVQMM |
| | | YQIGSFKVAVMASEKMKILELPYASGQLSMLVLLPDDVSGLEQLESAITSEKL |
| | | MEWTSSTTMEERKMKVYLPRMKIEEKYNLTSVLMALGVTDLFSSSADLSGIS |
| | | SAESLKISKAVHEAFVEIYEAGSEVVGSTEGGMEVTSFSEEFRADHPFLFLIKH |
| | | KPTNSILFFGRCFSP |
| PREDICTED: | SEQ ID | MGSIGAASTEFCFDVFKELKVQHVNENIFYSPLSIISALSMVYLGARENTRAQI |
| Ovalbumin | NO: 64 | DKVVHFDKITGFGETIESQCGTSVSVHTSLKDMFTQITKPSDNYSLSFASRLYA |
| [Fulmarus | | EETYPILPEYLQCVKELYKGGLETTSFQTAADQARELINSWVESQTNGMIKNI |
| glacialis] | | LQPGSVDPQTEMVLVNAIYFKGMWEKAFKDEDTQAVPFRMTEQESKTVQM |
| | | MYQIGSFKVAVMASEKMKILELPYASGELSMLVMLPDDVSGLEQLETAITFE |
| | | KLMEWTSSNMMEERKMKVYLPRMKMEEKYNLTSVLMALGVTDLFSSSANL |
| | | SGISSAESLKMSEAVHEAFVEIYEAGSEVVGSTGAGMEVTSVSEEFRADHPFL |
| | | FLIKHNPTNSILFFGRCFSP |
| PREDICTED: | SEQ ID | MGSIGAASTEFCFDVFKELRVQHVNENVCYSPLIIISALSLVYLGARENTRAQI |
| Ovalbumin-like | NO: 65 | DKVVHFDKITGFGESIESQCGTSVSVHTSLKDMFNQITKPSDNYSLSVASRLY |
| [Chlamydotis | | AFERYPILPEYLQCVKELYKGGLESISFQTAADQAREAINSWVESQTNGMIKNI |
| macqueenii] | | LQPSSVDPQTEMVLVNAIYFKGMWQKAFKDEDTQAVPFRISEQESKPVQMM |
| | | YQIGSFKVAVMAAEKMKILELPYASGELSMLVLLPDEVSGLEQLENAITVEKL |
| | | MEWTSSSPMEERIMKVYLPRMKIEEKYNLTSVLMALGITDLFSSSANLSGISA |
| | | EESLKMSEAVHQAFAEISEAGSEVVGSSEAGIDATSVSEEFRADHPFLFLIKHN |
| | | ATNSILFFGRCFSP |
| PREDICTED: | SEQ ID | MGSISAASTEFCFDVFKELKVQHVNENIFYSPLSIISALSMVYLGARENTRAQI |
| Ovalbumin like | NO: 66 | EKVVHFDKITGFGESIESQCSTSVSVHTSLKDMFTQITKPSDNYSLSFASRFYA |
| [Nipponia nippon] | | EETYPILPEYLQCVKELYKGGLETINFRTAADQARELINSWVESQTNGMIKNIL |
| | | QPGSVDPQTDMVLVNAIYFKGMWEKAFKDEDTQALPFRVTEQESKPVQMM |
| | | YQIGSFKVAVLASEKVKILELPYASGQLSMLVLLPDDVSGLEQLETAITVEKL |
| | | MEWTSSNNMEERKIKVYLPRIKIEEKYNLTSVLMALGITDLFSSSANLSGISSA |
| | | ESLKVSBATHEAFVEIYEAGSEVAGSTEAGIEVTSVSEEFRADHPFLFLIKHNAT |
| | | NSILFFGRCFSP |
| PREDICTED: | SEQ ID | MVSIGAASTEFCFDVFKELKVQHVNENIFYSPLSIISALSMVYLGARENTRAQI |
| Ovalbumin-like | NO: 67 | DKVVHFDKITGFEETIESQCSTSVSVHTSLKDMFTQITKPSDNYSLSFASRLYA |
| isoform X2 [Gavia | | EETYPILPEYLQCVKELYKGGLETISFQTAADQARELINSWVESQTDGMIKNIL |
| stellata] | | QPGSVDPQTEMVLVNAIYFKQMWEKAFKDEDTQAVPFRMTEQESKPVQMM |
| | | YQIGSFKVAVMASEKMKILELPYASGGMSMLVMLPDDVSGLEQLETAITFEK |
| | | LMEWTSSNMMEERKMKVYLPRMKMEEKYNLTSVLMALGMTDLFSSSANLS |
| | | GISSAESLKMSEAVHEAFVEIYEAGSEAVGSTGAGMEVTSVSEEFRADHPFLF |
| | | LIKHNPTNSILFFGRCFSP |
| PREDICTED: | SEQ ID | MGSIGAASTEFCFDVFKELKVQHVNENIFYSPLSIISALSMVYLGARENTRAQI |
| Ovalbumin | NO: 68 | DKVVHFDKITGFGEPIESQCGISVSVHTSLKDMITQITKPSDNYSLSFASRLYAE |
| [Pelecanus | | ETYPILPEYLQCVKELYKGGLETISFQTAADQARELINSWVENQTNGMIKNIL |
| crispus] | | QPGSVDPQTEMVLVNAVYFKGMWEKAFKDEDTQAVPFRMTEQESKPVQMM |
| | | YQIGSFKVAVMASEKIKILELPYASGELSMLVLLPDDVSGLEQLETAITLDKLT |
| | | EWTSSNAMEERKMKVYLPRMKIEKKYNLTSVLIALGMTDLFSSSANLSGISSA |
| | | ESLKMSEAIHEAFLEIYEAGSEVVGSTEAGMEVTSVSEEFRADHPFLFLIKHNP |
| | | TNSILFFGRCLSP |
| PREDICTED: | SEQ ID | MGSIGAASTEFCFDVFKELKVQHVNENIFYSPLTIISALSMVYLGARENTRAQI |
| Ovalbumin-like | NO: 69 | DKVVHFDKIPGFGDTTESQCGTSVSVHTSLKDMFTQITKPSDNYSVSFASRLY |
| [Charadrius | | LQPGSVDSQTEMVLVNAIYFKGMWEKAFKDEDTQTVPFRMTEQETKPVQM |
| vociferus] | | MYQIGTFKVAVMPSEKMKILELPYASGELCMLVMLPDDVSGLEELESSITVEK |
| | | LMEWTSSNMMEERKMKVFLPRMKIEEKYNLTSVLMALGMIDLFSSSANLSG |
| | | ISSAEPLKMSEAVHEAFIETYEAGSEVVGSTGAGMEITSVSEEFRADHPFLFLIK |
| | | HNPTNSILFFGRCVSP |
| PREDICTED: | SEQ ID | MGSIGAVSTEFCFDVFKELKVQHVNENIFYSPLSIISALSMVYLGARENTRAQI |
| Ovalbumin-like | NO: 70 | DKVVHFDKITGSGETIBAQCGTSVSVHTSLKDMFTQITKPSENYSVGFASRLY |
| [Eurypyga helias] | | ADETYPIIPEYLQCVKELYKGGLEMISFQTAADQARELINSWVESQTNGMIKNI |
| | | LQPGSVDPQTEMILVNAIYFKGVWEKAFKDEDTQAVPFRMTEQESKPVQMM |
| | | YQFGSFKVAAMAAEKMKILELPYASGALSMLVLLPDDVSGLEQLESAITFEKL |
| | | MEWTSSNMMEEKKIKVYLPRMKMEEKYNFTSVLMALGMTDLFSSSANLSGI |
| | | SSADSLKMSEVVHEAFVEIYEAGSEVVGSTGSGMEAASVSEEFRADHPFLFLI |
| | | KHNPTNSILFFGRCFSP |
| PREDICTED: | SEQ ID | MVSIGAASTEFCFDVFKELKVQHVNENIFYSPLSIISALSMVYLGARENTRAQI |
| Ovalbumin-like | NO: 71 | DKVVHFDKITGFEETIESQVQKKQCSTSVSVHTSLKDMFTQITKPSDNYSLSFA |
| isoform X1 [Gavia | | SRLYAEETYPILPEYLQCVKELYKGGLETISFQTAADQARELINSWVESQTDG |
| stellata] | | MIKNILQPGSVDPQTEMVLVNAIYFKGMWEKAFKDEDTQAVPFRMTEQESKP |
| | | VQMMYQIGSFKVAVMASEKMKILELPYASGGMSMLVMLPDDVSGLEQLETA |
| | | ITFEKLMEWTSSNMMEERKMKVYLPRMKMEEKYNLTSVLMALGMTDLFSSS |
| | | ANLSGISSAESLKMSEAVHEAFVEIYEAGSEAVGSTGAGMEVTSVSEEFRADH |
| | | PFLFLIKHNPTNSILFFGRCFSP |
| PREDICTED: | SEQ ID | MGSIGAASGEFCFDVFKELKVQHVNENIFYSPLSIISALSMVYLGARENTRAQI |
| Ovalbumin-like | NO: 72 | DKVVHFDKIIGFGESIESQCGTSVSVHTSLKDMFAQITKPSDNYSLSFASRLYA |
| [Egretta garzetta] | | EETFPILPEYLQCVKELYKGGLETLSFQTAADQARELINSWVESQINGMIKDIL |
| | | QPGSVDPQTEMVLVNAIYFKQVWEKAFKDEDTQTVPFRMTEQESKPVQMMY |
| | | QIGSFKVAVVAAEKIKILELPYASGALSMLVLLPDDVSSLEQLETAITFEKLTE |
| | | WTSSNIMEERKIKVYLPRMKIEEKYNLTSVLMDLGITDLFSSSANLSGISSAES |
| | | LKVSEAIHEAIVDIYEAGSEVVGSSGAGLEGTSVSEEFRADHPFLFLIKHNPTSS |
| | | ILFFGRCFSP |
| PREDICTED: | SEQ ID | MGSIGAASTEFCFDVFKELKVQHVNENIFYSPLSIISALSMVYLGARENTRAQI |
| Ovalbumin-like | NO: 73 | DKVVHFDKITGSGEAIESQCGTSVSVHISLKDMFTQITKPSDNYSLSFASRLYA |
| [Balearica | | EETYPILPEYLQCVKELYKEGLATISFQTAADQAREFINSWVESQTNGMIKNIL |
| regulorum | | QPGSVDPQTQMVLVNAIYFKGVWEKAFKDEDTQAVPFRMTKQESKPVQMM |
| gibbericeps] | | YQIGSFKVAVMASEKMKILELPYASGQLSMLVMLPDDVSGLEQIENAITFEKL |
| | | MEWTNPNMMEERKMKVYLPRMKMEEKYNLTSVLMALGMTDLFSSSANLSG |
| | | ISSAESLKMSEAVHEAFVEIYEAGSEVVGSTGAGIEVTSVSEEFRADHPFLFLIK |
| | | HNPTNSILFFGRCFSP |
| PREDICTED: | SEQ ID | MGSIGEASTEFCIDVFRELKVQHVNENIFYSPLSIISALSMVYLGARENTRAQID |
| Ovalbumin-like | NO: 74 | QVVHFDKITGFGDTVESQCGSSLSVHSSLKDIFAQITQPKDNYSLNFASRLYAE |
| [Nestor notabilis] | | ETYPILPEYLQCVKELYKGGLETISFQTAADQARELINSWVESQTNGMIKNILQ |
| | | PSSVDPQTEMVLVNAIYFKGVWEKAFKDEETQAVPFRITEQENRPVQIMYQF |
| | | GSFKVAVVASEKIKILELPYASGQLSMLVLLPDEVSGLEQLENAITFEKLTEWT |
| | | SSDIMEEKKIKVFLPRMKIEEKYNLTSVLVALGIADLFSSSANLSGISSABSLKM |
| | | SEAVHEAFVEIYEAGSEVVGSSGAGIEAASDSEEFRADHPFLFLIKHKPTNSILF |
| | | FGRCFSP |
| PREDICTED: | SEQ ID | MGSIGAASTEFCFDIFNELKVQHVNENIFYSPLSIISALSMVYLGARENTKAQID |
| Ovalbumin-like | NO: 75 | KVVHFDKITGFGESIESQCSTSASVHTSFKDMFTQITKPSDNYSLSFASRLYAE |
| [Pygoscelis | | PGSVDPQTELVLVNAIYFKGTWEKAFKDKDTQAVPFRVTEQESKPVQMMYQI |
| adeliae] | | GSYKVAVIASEKMKILELPYASGELSMLVLLPDDVSGLEQLETAITFEKLMEW |
| | | TSSNMMEERKVKVYLPRMKIEEKYNLTSVLMALGMTDLFSPSANLSGISSAES |
| | | LKMSEAIHEAFVEIYEAGSEVVGSTEAGMEVTSVSEEFRADHPFLFLIKCNLIN |
| | | SILFFGRCFSP |
| Ovalbumin-like | SEQ ID | MGSISTASTEFCFDVFKELKVQHVNENIFYSPLSIISALSMVYLGARENTRAQIE |
| [Athene | NO: 76 | KVVHFDKITGFGESIESQCGTSVSVHTSLKDMLIQISKPSDNYSLSFASKLYAE |
| cunicularia] | | ETYPILPEYLQCVKELYKGGLESINFQTAADQARQLINSWVESQTNGMIKDIL |
| | | QPSSVDPQTEMVLVNAIYFKGIWEKAFKDEDTQEVPFRITEQESKPVQMMYQI |
| | | GSFKVAVIASEKIKILELPYASGELSMLIVLPDDVSGLEQLETAITFEKLIEWTSP |
| | | SIMEERKTKVYLPRMKIEEKYNLTSVLMALGMTDLFSPSANLSGISSAESLKM |
| | | GRCVSP |
| PREDICTED: | SEQ ID | MGSIGAASTEFCFDVFKELKVQHVNENIFYSPLTIISALSLVYLGARENTRAQI |
| Ovalbumin-like | NO: 77 | DKVFHFDKISGFGETTESQCGTSVSVHTSLKEMFTQITKPSDNYSVSFASRLYA |
| [Calidris pugnax] | | EDTYPILPEYLQCVKELYKGGLETISFQTAADQAREVINSWVESQINGMIKNIL |
| | | QPGSVDSQTEMVLVNAIYFKGMWEKAFKDEDTQTMPFRITEQERKPVQMMY |
| | | QAGSFKVAVMASEKMKILELPYASGEFCMLIMLPDDVSGLEQLENSFSFEKI |
| | | MEWTTSNMMEERKMKVYIPRMKMEEKYNLTSVLMALGMTDLFSSSANLSGI |
| | | SSAETLKMSEAVHEAFMEIYEAGSEVVGSTGSGAEVTGVYEEFRADHPFLFLV |
| | | KHKPTNSILFFGRCVSP |
| PREDICTED: | SEQ ID | MGSIGAASTEFCFDIFNELKVQHVNENIFYSPLSIISALSMVYLGARENTKAQID |
| Ovalbumin | NO: 78 | KVVHFDKITGFGETIESQCSTSVSVHTSLKDTFTQITKPSDNYSLSFASRLYAEE |
| [Aptenodytes | | TYPILPEYSQCVKELYKGGLETISFQTAADQARELINSWVESQTNGMIKNILQP |
| forsteri] | | GSVDPQTELVLVNAIYFKGTWEKAFKDKDTQAVPFRVTEQESKPVQMMYQI |
| | | GSYKVAVIASEKMKILELPYASRELSMLVLLPDDVSGLEQLETAITFEKLMEW |
| | | TSSNMMEERKVKVYLPRMKIEEKYNLTSVLMALGMTDLFSPSANLSGISSAES |
| | | LKMSEAVHEAFVEIYEAGSEVVGSTGAGMEVTSVSEEFRADHPFLFLIKCNPT |
| | | NSILFFGRCFSP |
| PREDICTED: | SEQ ID | MGSISAASAEFCLDVFKELKVQHVNENIFYSPLSIISALSMVYLGARENTRAQI |
| Ovalbumin-like | NO: 79 | DKVVHFDKITGSGETIEFQCGTSANIHPSLKDMFTQITRLSDNYSLSFASRLYA |
| [Pterocles | | EERYPILPEYLQCVKELYKGGLETISFQTAADQARELINSWVESQINGMIKNIL |
| gutturalis] | | QPGSVNPQTEMVLVNAIYFKGLWEKAFKDEDTQTVPFRMTEQESKPVQMMY |
| | | QVGSFKVAVMASDKIKILELPYASGELSMLVLLPDDVTGLBQLETSITFEKLM |
| | | EWTSSNVMEERTMKVYLPHMRMEEKYNLTSVLMALGVIDLFSSSANLSGISS |
| | | AESLKMSEAVHEAFVEIYESGSQVVGSTGAGTEVTSVSEEFRVDHPFLFLIKH |
| | | NPTNSILFFGRCFSP |
| Ovalbumin-like | SEQ ID | MGSIGAASVEFCFDVFKELKVQHVNENIFYSPLSIISALSMVYLGARENTKAQI |
| [Falco peregrinus] | NO: 80 | DKVVHFDKIAGFGEAIESQCVTSASIHSLKDMFTQITKPSDNYSLSFASRLYAE |
| | | EAYSILPEYLQCVKELYKGGLETISFQTAADQARDLINSWVESQTNGMIKNIL |
| | | QPGAVDLETEMVLVNAIYFKGMWEKAFKDEDTQTVPFRMTEQESKPVQMM |
| | | YQVGSFKVAVMASDKIKILELPYASGQLSMVVVLPDDVSGLEQLEASITSEKL |
| | | MEWTSSSIMEEKKIKVYFPHMKIEEKYNLTSVLMALGMTDLFSSSANLSGISS |
| | | AEKLKVSEAVHEAFVEISEAGSEVVGSTEAGTEVTSVSEEFKADHPFLFLIKHN |
| | | PTNSILFFGRCFSP |
| PREDICTED: | SEQ ID | MGSIGAASSEFCFDIFKELKVQHVNENIFYSPLSIISALSMVYLGARENTRAQID |
| Ovalbumin-like | NO: 81 | KVVPFDKITASGESIESQCSTSVSVHTSLKDIFTQITKSSDNHSLSFASRLYABET |
| isoform X2 | | YPILPEYLQCVKELYEGGLETISFQTAADQARELINSWIESQTNGRIKNILQPGS |
| [Phalacrocorax | | VDPQTEMVLVNAIYFKGMWEKAFKDEDTQAVPFRMTEQESKPVQVMHQIGS |
| carbo] | | FKVAVLASEKIKILELPYASGELSMLVLLPDDVSGLEQLETAITFEKLMEWTSP |
| | | NIMEERKIKVFLPRMKIEEKYNLTSVLMALGITDLFSPLANLSGISSAESLKMS |
| | | EAIHEAFVEISEAGSEVIGSTEAEVEVINDPEEFRADHPFLFLIKHNPTNSILFFG |
| | | RCFSP |
| PREDICTED: | SEQ ID | MGSIGAASTEFCFDVFKELKAQYVNENIFYSPMTIITALSMVYLGSKENTRAQI |
| Ovalbumin-like | NO: 82 | AKVAHFDKITGFGESIESQCGASASIQFSLKDLFTQITKPSGNHSLSVASRIYAE |
| [Merops nubicus] | | ETYPILPEYLECMKELYKGGLETINFQTAANQARELINSWVERQTSGMIKNIL |
| | | QPSSVDSQTEMVLVNAIYFRGLWEKAFKVEDTQATPFRITEQESKPVQMMHQ |
| | | IGSFKVAVVASEKIKILELPYASGRLTMLVVLPDDVSGLKQLETTITFEKLME |
| | | WTTSNIMEERKIKVYLPRMKIEEKYNLTSVLMALGLTDLFSSSANLSGISSAES |
| | | LKMSEAVHEAFVEIYEAGSEVVASAEAGMDATSVSEEFRADHPFLFLIKDNTS |
| | | NSILFFGRCFSP |
| PREDICTED: | SEQ ID | MGSIGAASTEFCFDVFKELKGQHVNENIFFCPLSIVSALSMVYLGARENTRAQI |
| Ovalbumin-like | NO: 83 | VKVAHFDKIAGFAESIESQCGTSVSIHTSLKDMFTQITKPSDNYSLNFASRLYA |
| [Tauraco | | EETYPIIPEYLQCVKELYKGGLETISFQTAADQAREIINSWVESQTNGMIKNILR |
| erythrolophus] | | PSSVHPQTELVLVNAVYFKGTWEKAFKDEDTQAVPFRITEQESKPVQMMYQI |
| | | GSFKVAAVTSEKMKILEVPYASGELSMLVLLPDDVSGLEQLETAITAEKLIEW |
| | | TSSTVMEERKLKVYLPRMKIEEKYNLTTVLTALGVTDLFSSSANLSGISSAQG |
| | | LKMSNAVHEAFVEIYEAGSEVVGSKGEGTEVSSVSDEFKADHPFLFLIKHNPT |
| | | NSIVFFGRCFSP |
| PREDICTED: | SEQ ID | MGSIGAASTEFCFDVFKELKVHHVNENILYSPLAIISALSMVYLGAKENTRDQI |
| Ovalbumin-like | NO: 84 | DKVVHFDKITGIGESIESQCSTAVSVHTSLKDVFDQITRPSDNYSLAFASRLYA |
| [Cuculus canorus] | | EKTYPILPEYLQCVKELYKGGLETIDFQTAADQARQLINSWVEDETNGMIKNI |
| | | LRPSSVNPQTKIILVNAIYFKGMWEKAFKDEDTQEVPFRITEQETKSVQMMYQ |
| | | IGSFKVAEVVSDKMKILELPYASGKLSMLVLLPDDVYGLEQLETVITVEKLKE |
| | | WTSSIVMEERITKVYLPRMKIMEKYNLTSVLTAFGITDLFSPSANLSGISSTESL |
| | | KVSEAVHEAFVEIHEAGSEVVGSAGAGIEATSVSEEFKADHPFLFLIKHNPTNS |
| | | ILFFGRCFSP |
| Ovalbumin | SEQ ID | MGSIGAASTEFCLDVFKELKVQHVNENIFYSPLSIISALSMVYLGARENTRAQI |
| [Antrostomus | NO: 85 | DKVVHFDKITGFEDSIESQCGTSVSVHTSLKDMFTQITKPSDNYSVGFASRLY |
| carolinensis] | | AAETYQILPEYSQCVKELYKGGLETINFQKAADQATELINSWVESQTNGMIKN |
| | | ILQPSSVDPQTQIFLVNAIYFKGMWQRAFKEEDTQAVPFRISEKESKPVQMMY |
| | | QIGSFKVAVIPSEKIKILELPYASGLLSMLVILPDDVSGLEQLENAITLEKLMQW |
| | | TSSNMMEERKIKVYLPRMRMEEKYNLTSVFMALGITDLFSSSANLSGISSAES |
| | | LKMSDAVHEASVEIHEAGSEVVGSTGSGTEASSVSEEFRADHPYLFLIKHNPT |
| | | DSIVFFGRCFSP |
| PREDICTED: | SEQ ID | MGSIGAASTEFCFDVFKELKFQHVDENIFYSPLTIISALSMVYLGARENTRAQI |
| Ovalbumin-like | NO: 86 | DKVVHFDKIAGFEETVESQCGTSVSVHTSLKDMFAQITKPSDNYSLSFASRLY |
| [Opisthocomus | | AEETYPILPEYLQCVKELYKGGLETISFQTAADQARDLINSWVESQTNGMIKNI |
| hoazin] | | LQPSSVGPQTELILVNAIYFKGMWQKAFKDEDTQEVPFRMTEQQSKPVQMM |
| | | YQTGSFKVAVVASEKMKILALPYASGQLSLLVMLPDDVSGLKQLESAITSEKL |
| | | IEWTSPSMMEERKIKVYLPRMKIEEKYNLTSVLMALGITDLFSPSANLSGISSA |
| | | ESLKMSQAVHEAFVEIYEAGSEVVGSTGAGMEDSSDSEEFRVDHPFLFFIKHN |
| | | PTNSILFFGRCFSP |
| PREDICTED: | SEQ ID | MGSIGPLSVEFCCDVFKELRIQHPRENIFYSPVTIISALSMVYLGARDNTKAQIE |
| Ovalbumin-like | NO: 87 | KAVHFDKIPGFGESIESQCGTSLSIHTSLKDIFTQITKPSDNYTVGIASRLYAEEK |
| [Lepidothrix | | YPILPEYLQCIKELYKGGLEPINFQTAAEQARELINSWVESQINGMIKNILQPSS |
| coronatal | | VNPETDMVLVNAIYFKGLWEKAFKDEDIQTVPFRITEQESKPVQMMFQIGSFR |
| | | EERKIKVYLPRMKIEEKYNLTSVLTSLGITDLFSSSANLSGISSAESLKVSSAFH |
| | | EASVEIYEAGSKVVGSTGABVEDTSVSEEFRADHPFLFLIKHNPSNSIFFFGRCF |
| | | SP |
| PREDICTED: | SEQ ID | MGSIGTASAEFCFDVFKELKVHHVNENIFYSPLSIISALSMVYLGARENTKTQ |
| Ovalbumin | NO: 88 | MEKVIHFDKITGLGESMESQCGTGVSIHTALKDMLSEITKPSDNYSLSLASRLY |
| [Struthio camelus | | ABQTYAILPEYLQCIKELYKESLETVSFQTAADQARELINSWIESQINGVIKNF |
| australis] | | LQPGSVDSQTELVLVNAIYFKGMWEKAFKDEDTQEVPFRITEQESRPVQMMY |
| | | QAGSFKVATVAAEKIKILELPYASGELSMLVLLPDDISGLEQLETTISFEKLTE |
| | | WTSSNMMEDRNMKVYLPRMKIEEKYNLTSVLIALGMTDLFSPAANLSGISAA |
| | | ESLKMSEAIHAAYVEIYEADSEIVSSAGVQVEVTSDSEEFRVDHPFLFLIKHNP |
| | | TNSVLFFGRCISP |
| PREDICTED: | SEQ ID | MGSIGAVSTEFSCDVFKELRIHHVQENIFYSPVTIISALSMIYLGARDSTKAQIE |
| Ovalbumin-like | NO: 89 | KAVHFDKIPGFGESIESQCGTSLSIHTSIKDMFTKITKASDNYSIGIASRLYAEEK |
| [Acanthisitta | | YPILPEYLQCVKELYKGGLESISFQTAAEQAREIINSWVESQTNGMIKNILQPSS |
| chloris] | | VDPQTDIVLVNAIYFKGLWEKAFRDEDTQTVPFKITEQESKPVQMMYQIGSFK |
| | | VABITSEKIKILEVPYASGQLSLWVLLPDDISGLEKLETAITFENLKEWTSSTKM |
| | | EERKIKVYLPRMKIBEKYNLTSVLTALGITDLFSSSANLSGISSAESLKVSEAFH |
| | | EAIVEISEAGSKVVGSVGAGVDDTSVSEEFRADHPFLFLIKHNPTSSIFFFGRCF |
| | | SP |
| PREDICTED: | SEQ ID | MGSIGAASTEFCFDVFKELKVQHVNENIFYSPLSIISALSMVYLGARENTRAQI |
| Ovalbumin-like | NO: 90 | DKVVHFDKIAGFGESTESQCGTSVSAHTSLKDMSNQITKLSDNYSLSFASRLY |
| [Tyto alba] | | AEETYPILPEYSQCVKELYKGGLESISFQTAAYQARELINAWVESQTNGMIKDI |
| | | LQPGSVDSQTKMVLVNAIYFKGIWEKAFKDEDTQEVPFRMTEQETKPVQMM |
| | | YQIGSFKVAVIAAEKIKILELPYASGQLSMLVILPDDVSGLEQLETAITFEKLTE |
| | | WTSASVMEERKIKVYLPRMSIEEKYNLTSVLIALGVTDLFSSSANLSGISSAES |
| | | LRMSEAIHEAFVETYEAGSTESGTEVTSASEEFRVDHPFLFLIKHKPTNSILFFG |
| | | RCFSP |
| PREDICTED: | SEQ ID | MGSIGAASSEFCFDIFKELKVQHVNENIFYSPLSIISALSMVYLGARENTRAQID |
| Ovalbumin-like | NO: 91 | KVVPFDKITASGESIESQVQKIQCSTSVSVHTSLKDIFTQITKSSDNHSLSFASRL |
| isoform X1 | | YAEETYPILPEYLQCVKELYEGGLETISFQTAADQARELINSWIESQTNGRIKNI |
| [Phalacrocorax | | LQPGSVDPQTEMVLVNAIYFKGMWEKAFKDEDTQAVPFRMTEQESKPVQVM |
| carbo] | | HQIGSFKVAVLASEKIKILELPYASGELSMLVLLPDDVSGLEQLETAITFEKLM |
| | | EWTSPNIMEERKIKVFLPRMKIEEKYNLTSVLMALGITDLFSPLANLSGISSAES |
| | | LKMSEAIHEAFVEISEAGSEVIGSTEAEVEVTNDPEEFRADHPFLFLIKHNPINS |
| | | ILFFGRCFSP |
| Ovalbumin-like | SEQ ID | MGSIGPLSVEFCCDVFKELRIQHARENIFYSPVTIISALSMVYLGARDNTKAQIE |
| [Pipra filicauda] | NO: 92 | KAVHFDKIPGFGESIESQCGTSLSIHTSLKDIFTQITKPSDNYTVGIASRLYAEEK |
| | | YPILPEYLQCIKELYKGGLEPISFQTAABQARELINSWVESQTNGIIKNILQPSSV |
| | | NPETDMVLVNAIYFKQLWEKAFKDEGTQTVPFRITEQESKPVQMMFQIGSFR |
| | | EERKIKVYLPRMKIEEKYNLTSVLTSLGITDLFSSSANLSGISSAERLKVSSAFH |
| | | EASMEINEAGSKVVGAGVDDTSVSEEFRVDRPFLFLIKHNPSNSIFFFGRCFSP |
| Ovalbumin | SEQ ID | MGSIGAASTEFCFDMFKELKVHHVNENIIYSPLSIISILSMVFLGARENTKTQM |
| [Dromaius | NO: 93 | EKVIHFDKITGFGESLESQCGTSVSVHASLKDILSEITKPSDNYSLSLASKLYAE |
| novaehollandiae] | | ETYPVLPEYLQCIKELYKGSLETVSFQTAADQARELINSWVETQTNGVIKNFL |
| | | QPGSVDPQTEMVLVDAIYFKGTWEKAFKDEDTQEVPFRITEQESKPVQMMY |
| | | QAGSFKVATVAAEKMKILELPYASGELSMFVLLPDDISGLEQLETTISIEKLSE |
| | | WTSSNMMEDRKMKVYLPHMKIEEKYNLTSVLVALGMTDLFSPSANLSGISTA |
| | | QTLKMSEAIHGAYVEIYEAGSEMATSTGVLVEAASVSEEFRVDHPFLFLIKHN |
| | | PSNSILFFGRCIFP |
| Chain A, | SEQ ID | MGSIGAASTEFCFDMFKELKVHHVNENIIYSPLSIISILSMVFLGARENTKTQM |
| Ovalbumin | NO: 94 | EKVIHFDKITGFGESLESQCGTSVSVHASLKDILSEITKPSDNYSLSLASKLYAE |
| | | ETYPVLPEYLQCIKELYKGSLETVSFQTAADQARELINSWVETQTNGVIKNFL |
| | | QPGSVDPQTEMVLVDAIYFKGTWEKAFKDEDTQEVPFRITEQESKPVQMMY |
| | | QAGSFKVATVAAEKMKILELPYASGELSMFVLLPDDISGLEQLETTISIEKLSE |
| | | WTSSNMMEDRKMKVYLPHMKIEEKYNLTSVLVALGMTDLFSPSANLSGISTA |
| | | QTLKMSEAIHGAYVEIYEAGSEMATSTGVLVEAASVSEEFRVDHPFLFLIKHN |
| | | PSNSILFFGRCIFPHHHHHH |
| Ovalbumin-like | SEQ ID | MGSIGPLSVEFCCDVFKELRIQHARENIFYSPVTHISALSMVYLGARDNTKAQIE |
| [Corapipo altera] | NO: 95 | KAVHFDKIPGFGESIESQCGTSLSIHTSLKDIFTQITKPSDNYTVGIASRLYAEEK |
| | | YPILPEYLQCIKELYKGGLEPISFQTAABQARELINSWVESQTNGMIKNILQPSA |
| | | VNPETDMVLVNAIYFKGLWEKAFKDEGTQTVPFRITEQESKPVQMMFQIGSF |
| | | RVAEITSEKIRILELPYASGQLSLWVLLPDDISGLEQLETAITFENLKEWTSSTK |
| | | MEERKIKVYLPRMKIEEKYNLTSVLTSLGITDLFSSSANLSGISSAERLKVSSAF |
| | | HEASMEIYEAGSKVVGSTGAGVDDTSVSEEFRVDRPFLFLIKHNPSNSIFFFGR |
| | | CFSP |
| Ovalbumin-like | SEQ ID | MEDQRGNTGFTMGSIGAASTEFCIDVFRELRVQHVNENIFYSPLTHISALSMVY |
| protein [Amazona | NO: 96 | LGARENTRAQIDQVVHFDKIAGFGDTVESQCGSSPSVHNSLKTVXAQITQPRD |
| aestiva] | | NYSLNLASRLYAEESYPILPEYLQCVKELYNGGLETVSFQTAADQARELINSW |
| | | VESQTNGIIKNILQPSSVDPQTEMVLVNAIYFKGLWEKAFKDEETQAVPFRITE |
| | | QENRPVQMMYQFGSFKVAXVASEKIKILELPYASGQLSMLVLLPDEVSGLEQ |
| | | NAITFEKLTEWTSSDLMEERKIKVFFPRVKIEEKYNLTAVLVSLGITDLFSSSA |
| | | NLSGISSAENLKMSEAVHEAXVEIYEAGSEVAGSSGAGIEVASDSEEFRVDHP |
| | | FLFLIXHNPTNSILFFGRCFSP |
| PREDICTED: | SEQ ID | MGSIGAASTEFCIDVFRELRVQHVNENIFYSPLSIISALSMVYLGARENTRAQID |
| Ovalbumin-like | NO: 97 | EVFHFDKIAGFGDTVDPQCGASLSVHKSLQNVFAQITQPKDNYSLNLASRLYA |
| [Melopsittacus | | EESYPILPEYLQCVKELYNEGLETVSFQTGADQARELINSWVENQINGVIKNIL |
| undulatus] | | QPSSVDPQTEMVLVNAIYFKGLWQKAFKDEETQAVPFRITEQENRPVQMMY |
| | | QFGSFKVAVVASEKVKILELPYASGQLSMWVLLPDEVSGLEQLENAITFEKLT |
| | | EWTSSDLTEERKIKVFLPRVKIEEKYNLTAVLMALGVTDLFSSSANFSGISAAE |
| | | NLKMSEAVHEAFVEIYEAGSEVVGSSGAGIEAPSDSEEFRADHPFLFLIKHNPT |
| | | NSILFFGRCFSP |
| Ovalbumin-like | SEQ ID | MGSIGPLSVEFCCDVFKELRIQHARDNIFYSPVTIISALSMVYLGARDNTKAQI |
| [Neopelma | NO: 98 | EKAVHFDKIPGFGESIESQCGTSLSVHTSLKDIFTQITKPRENYTVGIASRLYAE |
| chrysocephalum] | | EKYPILPEYLQCIKELYKGGLEPISFQTAAEQARELINSWVESQTNGMIKNILQP |
| | | SSVNPETDMVLVNAIYFKGLWKKAFKDEGTQTVPFRITEQESKPVQMMFQIG |
| | | SFRVAEITSEKIRILELPYASGQLSLWVLLPDDISGLEQLESAITFENLKEWTSST |
| | | KMEERKIKVYLPRMKIEEKYNLTSVLTSLGITDLFSSSANLSGISSAEKLKVSS |
| | | AFHEASMEIYEAGNKVVGSTGAGVDDTSVSEEFRVDRPFLFLIKHNPSNSIFFF |
| | | GRCFSP |
| PREDICTED: | SEQ ID | MGSIGAASAEFCVDVFKELKDQHVNNIVFSPLMIISALSMVNIGAREDTRAQI |
| Ovalbumin-like | NO: 99 | DKVVHFDKITGYGESIESQCGTSIGIYFSLKDAFTQITKPSDNYSLSFASKLYAE |
| [Buceros | | ETYPILPEYLKCVKELYKGGLETISFQTAADQARELINSWVESQTNGMIKNILQ |
| rhinoceros | | PSSVDPQTEMVLVNAIYFKGLWEKAFKDEDTQAVPFRITEQESKPVQMMYQI |
| silvestris] | | GSFKVAVIASEKIKILELPYASGQLSLLVLLPDDVSGLEQLESAITSEKLLEWTN |
| | | PNIMEERKTKVYLPRMKIEEKYNLTSVLVALGITDLFSSSANLSGISSAEGLKL |
| | | SDAVHEAFVEIYEAGREVVGSSEAGVEDSSVSEEFKADRPFIFLIKHNPTNGIL |
| | | YFGRYISP |
| PREDICTED: | SEQ ID | MGSIGAANTDFCFDVFKELKVHHANENIFYSPLSIVSALAMVYLGARENTRA |
| Ovalbumin-like | NO: | QIDKALHFDKILGFGETVESQCDTSVSVHTSLKDMLIQITKPSDNYSFSFASKIY |
| [Cariama cristata] | 100 | TEETYPILPEYLQCVKELYKGGVETISFQTAADQAREVINSWVESHINGMIKNI |
| | | LQPGSVDPQTKMVLVNAVYFKGIWEKAFKEEDTQEMPFRINEQESKPVQMM |
| | | YQIGSFKLTVAASENLKILEFPYASGQLSMMVILPDEVSGLKQLETSITSEKLIK |
| | | WTSSNTMEERKIRVYLPRMKIEEKYNLKSVLMALGITDLFSSSANLSGISSAES |
| | | LKMSEAVHEAFVEIYEAGSEVTSSTGTEMEAENVSEEFKADHPFLFLIKHNPT |
| | | DSIVFFGRCMSP |
| Ovalbumin | SEQ ID | MGSIGPLSVEFCCDVFKELRIQHARENIFYSPVTIISALSMVYLGARDNTKAQIE |
| [Manacus | NO: | KAVHFDKIPGFGESIESQCGTSLSIHTSLKDIFTQITKPSDNYTVGIASRLYAEEK |
| vitellinus] | 101 | YPILPEYLQCIKELYKGGLEPISFQTAAEQARELINSWVESQTNGMIKNILQPSS |
| | | VNPETDMVLVNAIYFKGLWEKAFKDESTQTVPFRITEQESKPVQMMFQIGSFR |
| | | VABIASEKIRILELPYASGQLSLWVLLPDDISGLEQLETAITFENLKEWTSSTKM |
| | | EERKIKVYLPRMKIBEKYNITSVLTSLGITDLFSSSANLSGISSAERLKVSSAFH |
| | | EASMETYEAGSRVVEAGVDDTSVSEEFRVDRPFLFLIKHNPSNSIFFFGRCFSP |
| Ovalbumin-like | SEQ ID | MGSIGPVSTEFCCDIFKELRIQHARENIIYSPVTIISALSMVYLGARDNTKAQIE |
| [Empidonax | NO: | KAVHFDKIPGFGESIESQCGTSLSIHTSLKDILTQITKPSDNYTVGIASRLYAEE |
| traillii] | 102 | KYPILSEYLQCIKELYKGGLEPISFQTAAEQARELINSWVESQINGMIKNILQPS |
| | | SVNPETDMVLVNAIYFKGLWEKAFKDEGTQTVPFRITEQESKPVQMMFQIGS |
| | | FKVABITSEKIRILELPYASGKLSLWVLLPDDISGLEQLETAITFENLKEWTSST |
| | | RMEERKIKVYLPRMKIBEKYNLTSVLTSLGITDLFSSSANLSGISSAERLKVSSA |
| | | FHEVFVEIYEAGSKVEGSTGAGVDDTSVSEEFRADHPFLFLVKHNPSNSIIFFG |
| | | RCYLP |
| PREDICTED: | SEQ ID | MGSTGAASMEFCFALFRELKVQHVNENIFFSPVTIISALSMVYLGARENTRAQ |
| Ovalbumin-like | NO: | LDKVAPFDKITGFGETIGSQCSTSASSHTSLKDVFTQITKASDNYSLSFASRLY |
| [Leptosomus | 103 | ABETYPILPEYLQCVKELYKGGLESISFQTAADQARELINSWVESQTNGMIKDI |
| discolor] | | LRPSSVDPQTKIILITAIYFKGMWEKAFKEEDTQAVPFRMTEQESKPVQMMYQ |
| | | IGSFKVAVIPSEKLKILELPYASGQLSMLVILPDDVSGLEQLETAITTEKLKEWT |
| | | SPSMMKERKMKVYFPRMRIEEKYNLTSVLMALGITDLFSPSANLSGISSAESL |
| | | KVSEAVHEASVDIDEAGSEVIGSTGVGTEVTSVSEEIRADHPFLFLIKHKPTNSI |
| | | LFFGRCFSP |
| Hypothetical | SEQ ID | MEHAQLTQLVNSNMTSNTCHEADEFENIDFRMDSISVTNTKFCFDVFNEMKV |
| protein | NO: | HHVNENILYSPLSILTALAMVYLGARGNTESQMKKALHFDSITGAGSTTDSQC |
| H355_008077 | 104 | GSSEYIHNLFKEFLTEITRTNATYSLEIADKLYVDKTFTVLPEYINCARKFYTG |
| [Colinus | | GVEEVNFKTAABEARQLINSWVEKETNGQIKDLLVPSSVDFGTMMVFINTIYF |
| virginianus] | | KGIWKTAFNTEDTREMPFSMTKQESKPVQMMCLNDTFNMATLPAEKMRILE |
| | | LPYASGELSMIVLLPDEVSGLEQIEKAINFEKLREWTSTNAMEKKSMKVYLP |
| | | RMKIEEKYNLTSTLMALGMTDLFSRSANLTGISSVENLMISDAVHGAFMEVN |
| | | EEGTEAAGSTGAIGNIKHSVEFEEFRADHPFLFLIRYNPTNVILFFDNSEFTMGS |
| | | IGAVSTEFCFDVFKELRVHHANENIFYSPFTVISALAMVYLGAKDSTRTQINKV |
| | | VRFDKLPGFGDSIEAQCGTSANVHSSLRDILNQITKPNDIYSFSLASRLYADET |
| | | YTILPEYLQCVKELYRGGLESINFQTAADQARELINSWVESQTSGIIRNVLQPS |
| | | SVDSQTAMVLVNAIYFKGLWEKGFKDEDTQAMPFRVTEQENKSVQMMYQI |
| | | GTFKVASVASEKMKILELPFASGTMSMWVLLPDEVSGLEQLETTISIEKLTEW |
| | | TSSSVMEERKIKVFLPRMKMEEKYNLTSVLMAMGMTDLFSSSANLSGISSTLQ |
| | | KKGFRSQELGDKYAKPMLESPALTPQVTAWDNSWIVAHPAAIEPDLCYQIMB |
| | | QKWKPFDWPDFRLPMRVSCRFRTMEALNKANTSFALDFFKHECQEDDDENIL |
| | | FSPFSISSALATVYLGAKGNTADQMAKTEIGKSGNIHAGFKALDLEINQPTKN |
| | | YLLNSVNQLYGEKSLPFSKEYLQLAKKYYSAEPQSVDFLGKANEIRREINSRV |
| | | EHQTEGKIKNLLPPGSIDSLTRLVLVNALYFKGNWATKFEAEDTRHRPFRINM |
| | | HTTKQVPMMYLRDKFNWTYVESVQTDVLELPYVNNDLSMFILLPRDITGLQK |
| | | LINELTFEKLSAWTSPELMEKMKMEVYLPRFTVEKKYDMKSTLSKMGIEDAF |
| | | TKVDSCGVTNVDEITTHIVSSKCLELKHIQINKKLKCNKAVAMEQVSASIGNF |
| | | TIDLFNKLNETSRDKNIFFSPWSVSSALALTSLAAKQNTAREMAEDPENEQAE |
| | | NIHSGFKELMTALNKPRNTYSLKSANRIYVEKNYPLLPTYIQLSKKYYKAEPY |
| | | KVNFKTAPEQSRKEINNWVEKQTERKIKNFLSSDDVKNSTKSILVNAIYFKAE |
| | | WEEKFQAGNTDMQPFRMSKNKSKLVKMMYMRHTFPVLIMEKLNFKMIELP |
| | | YVKRELSMFILLPDDIKDSTTGLEQLERELTYEKLSEWADSKKMSVTLVDLHL |
| | | PKFSMEDRYDLKDALKSMGMASAFNSNADFSGMTGFQAVPMESLSASTNSF |
| | | TLDLYKKLDETSKGQNIFFASWSIATALAMVHLGAKGDTATQVAKGPEYEET |
| | | ENIHSGFKELLSAINKPRNTYLMKSANRLFGDKTYPLLPKFLELVARYYQAKP |
| | | QAVNFKTDAEQARAQINSWVENETESKIQNLLPAGSIDSHTVLVLVNAIYFKG |
| | | NWEKRFLEKDTSKMPFRISKTETKPVQMMFLKDTFLIHHERTMKFKIIELPYV |
| | | GNELSAFVLLPDDISDNTTGLELVERELTYEKLAEWSNSASMMKAKVELYLP |
| | | KLKMEENYDLKSVLSDMGIRSAFDPAQADFTRMSEKKDLFISKVIHKAFVEV |
| | | NEEDRIVQLASGRLTGRCRTLANKELSEKNRTKNLFFSPFSISSALSMILLGSK |
| | | GNTEAQIAKVLSLSKAEDAHNGYQSLLSEINNPDTKYILRTANRLYGEKTFEF |
| | | LSSFIDSSQKFYHAGLEQTDFKNASEDSRKQINGWVEEKTEGKIQKLLSEGIIN |
| | | SMTKLVLVNAIYFKGNWQEKFDKETTKEMPFKINKNETKPVQMMFRKGKYN |
| | | MTYIGDLETTVLEIPYVDNELSMIILLPDSIQDESTQLEKLERELTYEKLMDWI |
| | | NPNMMDSTEVRVSLPRFKLEENYELKPTLSTMGMPDAFDLRTADFSGISSGNE |
| | | LVLSEVVHKSFVEVNEEGTEAAAATAGIMLLRCAMIVANFTADHPFLFFIRHN |
| | | KTNSILFCGRFCSP |
| PREDICTED: | SEQ ID | MGSIGTASTEFCFDMFKEMKVQHANQNIIFSPLTHISALSMVYLGARDNTKAQ |
| Ovalbumin | NO: | MEKVIHFDKITGFGESVESQCGTSVSIHTSLKDMLSEITKPSDNYSLSLASRLY |
| isoform X2 | 105 | ABETYPILPEYLQCMKELYKGGLETVSFQTAADQARELINSWVESQTNGVIKN |
| [Apteryx australis | | FLQPGSVDPQTEMVLVNAIYFKGMWEKAFKDEDTQEVPFRITEQESKPVQM |
| mantelli] | | MYQVGSFKVATVAAEKMKILEIPYTHRELSMFVLLPDDISGLEQLETTISFEKL |
| | | TEWTSSNMMEERKVKVYLPHMKIEEKYNLTSVLMALGMTDLFSPSANLSGIS |
| | | TAQTLMMSEAIHGAYVEIYEAGREMASSTGVQVEVTSVLEEVRADKPFLFFIR |
| | | HNPTNSMVVFGRYMSP |
| Hypothetical | SEQ ID | MTSNTCHEADEFENIDFRMDSISVTNTKFCFDVFNEMKVHHVNENILYSPLSIL |
| protein | NO: | TALAMVYLGARGNTESQMKKALHFDSITGGGSTIDSQCGSSEYIHNLFKEFLT |
| ASZ78_006007 | 106 | EITRTNATYSLEIADKLYVDKIFTVLPEYINCARKFYTGGVEEVNFKTAAEEA |
| [Callipepla | | RQLMNSWVEKETNGQIKDLLVPSSVDFGTMMVFINTIYFKGIWKTAFNTEDT |
| squamata] | | REMPFSMTKQESKPVQMMCLNDTFNMVTLPAEKMRILELPYASGELSMLVL |
| | | LPDEVSGLERIEKAINFEKLREWTSTNAMEKKSMKVYLPRMKIEEKYNLTSTL |
| | | MALGMTDLFSRSANLTGISSVDNLMISDAVHGAFMEVNEEGTEAAGSTGAIG |
| | | NIKHSVEFEEFRADHPFLFLIRYNPTNVILFFDNSEFTMGSIGAVSTEFCFDVFK |
| | | ELRVHHANENIFYSPFTIISALAMVYLGAKDSTRTQINKVVRFDKLPGFGDSIE |
| | | AQCGTSANVHSSLRDILNQITKPNDIYSFSLASRLYADETYTILPEYLQCVKEL |
| | | YRGGLESINFQTAADQARELINSWVESQTSGIIRNVLQPSSVDSQTAMVLVNAI |
| | | YFKGLWEKGFKDEDTQAIPFRVTEQENKSVQMMYQIGTFKVASVASEKMKIL |
| | | ELPFASGTMSMWVLLPDEVSGLEQLETTISIEKLTEWTSSSVMEERKIKVFLPR |
| | | MKMEEKYNLTSVLMAMGMTDLFSSSANLSGISSTLQKKGFRSQELGDKYAK |
| | | PMLESPALTPQATAWDNSWIVAHPPAIEPDLYYQIMEQKWKPFDWPDFRLPM |
| | | RVSCRFRTMEALNKANTSFALDFFKHECQEDDSENILFSPFSISSALATVYLGA |
| | | KGNTADQMAKVLHFNEAEGARNVTTTIRMQVYSRTDQQRLNRRACFQKTEI |
| | | GKSGNIHAGFKGLNLEINQPTKNYLLNSVNQLYGEKSLPFSKEYLQLAKKYYS |
| | | AEPQSVDFVGTANEIRREINSRVEHQTEGKIKNLLPPGSIDSLTRLVLVNALYF |
| | | KGNWATKFEAEDTRHRPFRINTHTTKQVPMMYLSDKFNWTYVESVQTDVLE |
| | | LPYVNNDLSMFILLPRDITGLQKLINELTFEKLSAWTSPELMEKMKMEVYLPR |
| | | FTVEKKYDMKSTLSKMGIEDAFTKVDNCGVTNVDEITIHVVPSKCLELKHIQI |
| | | NKELKQNKAVAMEQVSASIGNFTIDLFNKLNETSRDKNIFFSPWSVSSALALT |
| | | SLAAKGNTAREMAEDPENEQAENIHSGFNELLTALNKPRNTYSLKSANRIYVE |
| | | KNYPLLPTYIQLSKKYYKAEPHKVNFKTAPEQSRKEINNWVEKQTERKIKNFL |
| | | SSDDVKNSTKLILVNAIYFKAEWEEKFQAGNTDMQPFRMSKNKSKLVKMMY |
| | | MRHTFPVLIMEKLNFKMIELPYVKRELSMFILLPDDIKDSTTGLEQLERELTYE |
| | | KLSEWADSKKMSVTLVDLHLPKFSMEDRYDLKDALRSMGMASAFNSNADFS |
| | | GMTGERDLVISKVCHQSFVAVDEKGTEAAAATAVIAEAVPMESLSASTNSFT |
| | | LDLYKKLDETSKGQNIFFASWSIATALTMVHLGAKGDTATQVAKGPEYEETE |
| | | NIHSGFKELLSALNKPRNTYSMKSANRLFGDKTYPLLPTKTKPVQMMFLKDT |
| | | FLIHHERTMKFKHIELPYMGNELSAFVLLPDDISDNTTGLELVERELTYEKLAE |
| | | WSNSASMMKVKVELYLPKLKMEENYDLKSALSDMGIRSAFDPAQADFTRMS |
| | | EKKDLFISKVIHKAFVEVNEEDRIVQLASGRLTGNTEAQIAKVLSLSKAEDAH |
| | | NGYQSLLSEINNPDTKYILRTANRLYGEKTFEFLSSFIDSSQKFYHAGLEQTDF |
| | | KNASEDSRKQINGWVEEKTEGKIQKLLSEGIINSMTKLVLVNAIYFKGNWQE |
| | | KFDKETTKEMPFKINKNETKPVQMMFRKGKYNMTYIGDLETTVLEIPYVDNE |
| | | LSMIILLPDSIQDESTGLEKLERELTYEKLMDWINPNMMDSTEVRVSLPRFKLE |
| | | ENYELKPTLSTMGMPDAFDLRTADFSGISSGNELVLSEVVHKSFVEVNEEGTE |
| | | AAAATAGIMLLRCAMIVANFTADHPFLFFIRHNKTNSILFCGRFCSP |
| PREDICTED: | SEQ ID | MASIGAASTEFCFDVFKELKTQHVKENIFYSPMAIISALSMVYIGARENTRAEI |
| Ovalbumin-like | NO: | DKVVHFDKITGFGNAVESQCGPSVSVHSSLKDLITQISKRSDNYSLSYASRIYA |
| [Mesitornis | 107 | EETYPILPEYLQCVKEVYKGGLESISFQTAADQARENINAWVESQINGMIKNI |
| unicolor] | | LQPSSVNPQTEMVLVNAIYLKGMWEKAFKDEDTQTMPFRVTQQESKPVQM |
| | | MYQIGSFKVAVIASEKMKILELPYTSGQLSMLVLLPDDVSGLEQVESAITAEK |
| | | LMEWTSPSIMEERTMKVYLPRMKMVEKYNLTSVLMALGMTDLFTSVANLSG |
| | | ISSAQGLKMSQAIHEAFVEIYEAGSEAVGSTGVGMEITSVSEEFKADLSFLFLIR |
| | | HNPTNSIIFFGRCISP |
| Ovalbumin, partial | SEQ ID | MGSIGAASTEFCFDVFRELRVQHVNENIFYSPFSIISALAMVYLGARDNTRTQI |
| [Anas | NO: | DKISQFQALSDEHLVLCIQQLGEFFVCTNRERREVTRYSEQTEDKTQDQNTGQ |
| platyrhynchos] | 108 | IHKIVDTCMLRQDILTQITKPSDNFSLSFASRLYAEETYAILPEYLQCVKELYK |
| | | GGLESISFQTAADQARELINSWVESQTNGIIKNILQPSSVDSQTTMVLVNAIYF |
| | | KGMWEKAFKDEDTQAMPFRMTEQESKPVQMMYQVGSFKVAMVTSEKMKI |
| | | LELPFASGMMSMFVLLPDEVSGLEQLESTISFEKLTEWTSSTMMEERRMKVY |
| | | LPRMKMEEKYNLTSVFMALGMTDLFSSSANMSGISSTVSLKMSEAVHAACV |
| | | EIFEAGRDVVGSAEAGMDVTSVSEEFRADHPFLFFIKHNPTNSILFFGRWMSP |
| PREDICTED: | SEQ ID | MGSIGAASAEFCLDIFKELKVQHVNENIIFSPMTIISALSLVYLGAKEDTRAQIE |
| Ovalbumin-like | NO: | KVVPFDKIPGFGEIVESQCPKSASVHSSIQDIFNQIIKRSDNYSLSLASRLYAEES |
| [Chaetura | 109 | YPIRPEYLQCVKELDKEGLETISFQTAADQARQLINSWVESQTNGMIKNILQPS |
| pelagica] | | SVNSQTEMVLVNAIYFRGLWQKAFKDEDTQAVPFRITEQESKPVQMMQQIGS |
| | | FKVABIASEKMKILELPYASGQLSMLVLLPDDVSGLEKLESSITVEKLIEWTSS |
| | | NLTEERNVKVYLPRLKIEEKYNLTSVLAALGITDLFSSSANLSGISTAESLKLSR |
| | | AVHESFVEIQEAGHEVEGPKEAGIEVTSALDEFRVDRPFLFVTKHNPTNSILFL |
| | | GRCLSP |
| PREDICTED: | SEQ ID | MGSISAASGEFCLDIFKELKVQHVNENIFYSPMVIVSALSLVYLGARENTRAQI |
| Ovalbumin-like | NO: | DKVIPFDKITGSSEAVESQCGTPVGAHISLKDVFAQIAKRSDNYSLSFVNRLYA |
| [Apaloderma | 110 | EETYPILPEYLQCVKELYKGGLETISFQTAADQAREIINSWVESQTDGKIKNIL |
| vittatum] | | QPSSVDPQTKMVLVSAIYFKGLWEKSFKDEDTQAVPFRVTEQESKPVQMMY |
| | | QIGSFKVAAIAAEKIKILELPYASEQLSMLVLLPDDVSGLEQLEKKISYEKLTE |
| | | WTSSSVMEEKKIKVYLPRMKIEEKYNLTSILMSLGITDLFSSSANLSGISSTKSL |
| | | KMSEAVHEASVEIYEAGSEASGITGDGMEATSVFGEFKVDHPFLFMIKHKPTN |
| | | SILFFGRCISP |
| Ovalbumin-like | SEQ ID | MGSIGPVSTEVCCDIFRELRSQSVQENVCYSPLLIISTLSMVYIGAKDNTKAQIE |
| [Corvus cornix | NO: | KAIHFDKIPGFGESTESQCGTSVSIHTSLKDIFTQITKPSDNYSISIARRLYAEE |
| cornix] | 111 | KVSSQTDMVLVSAIYFKGLWEKAFKEEDTQTIPFRITEQESKPVQMMSQIGTFK |
| | | VAEIPSEKCRILELPYASGRLSLWVLLPDDISGLEQLETAITFENLKEWTSSSKM |
| | | EERKIRVYLPRMKIEEKYNLTSVLKSLGITDLFSSSANLSGISSAESLKVSAAFH |
| | | EASVEIYEAGSKGVGSSEAGVDGTSVSEEIRADHPFLFLIKHNPSDSILFFGRCF |
| | | SP |
| PREDICTED: | SEQ ID | MGSIGAASTEFCFDVFKELKVQHVNENIIISPLSIISALSMVYLGAREDTRAQID |
| Ovalbumin-like | NO: | KVVHFDKITGFGEAIESQCPTSESVHASLKETFSQLTKPSDNYSLAFASRLYAE |
| [Calypte anna] | 112 | ETYPILPEYLQCVKELYKGGLETINFQTAAEQARQVINSWVESQTDGMIKSLL |
| | | QPSSVDPQTEMILVNAIYFRGLWERAFKDEDTQELPFRITEQESKPVQMMSQI |
| | | GSFKVAVVASEKVKILELPYASGQLSMLVLLPDDVSGLEQLESSITVEKLIEWI |
| | | SSNTKEERNIKVYLPRMKIEEKYNLTSVLVALGITDLFSSSANLSGISSAESLKI |
| | | SEAVHEAFVEIQEAGSEVVGSPGPEVEVTSVSEEWKADRPFLFLIKHNPTNSIL |
| | | FFGRYISP |
| PREDICTED: | SEQ ID | MGSIGPVSTEVCCDIFRELRSQSVQENVCYSPLLIISTLSMVYIGAKDNTKAQIE |
| Ovalbumin | NO: | KAIHFDKIPGFGESTESQCGTSVSIHTSLKDIFTQITKPSDNYSISIARRLYAEEK |
| [Corvus | 113 | YPILQEYIQCVKELYKGGLESISFQTAAEKSRELINSWVESQTNGTIKNILQPSS |
| brachyrhynchos] | | VSSQTDMVLVSAIYFKGLWEKAFKEEDTQTIPFRITEQESKPVQMMSQIGTFK |
| | | VAEIPSEKCRILELPYASGRLSLWVLLPDDISGLEQLETSITFENLKEWTSSSKM |
| | | EERKIRVYLPRMKIEEKYNLTSVLKSLGITDLFSSSANLSGISSAESLKVSAVFH |
| | | EASVEIYEAGSKQVGSSEAGVDGTSVSEEIRADHPFLFLIKHNPSDSILFFGRCF |
| | | SP |
| Hypothetical | SEQ ID | MLNLMHPKQFCCTMGSIGPVSTEVCCDIFRELRSQSVQENVCYSPLLIISTLSM |
| protein | NO: | VYIGAKDNTKAQIEKAIHFDKIPGFGESTESQCGTSVSIHTSLKDIFTQITKPSD |
| DUI87_08270 | 114 | NYSISIASRLYAEEKYPILPEYIQCVKELYKGGLESISFQTAAEKSRELINSWVE |
| [Hirundo rustica | | SQTNGTIKNILQPSSVSSQTDMVLVSAIYFKGLWEKAFKEEDTQTVPFRITEQE |
| rustica] | | SKPVQMMSQIGTFKVAEIPSEKCRILELPYASGRLSLWVLLPDDISGLEQLETAI |
| | | TSENLKEWTSSSKMEERKIKVYLPRMKIEEKYNLTSVLKSLGITDLFSSSANLS |
| | | GISSAESLKVSGAFHEAFVEIYEAGSKAVGSSGAGVEDTSVSEEIRADHPFLFFI |
| | | KHNPSDSILFFGRCFSP |
| Ostrich OVA | SEQ ID | EABAGSIGTASAEFCFDVFKELKVHHVNENIFYSPLSIISALSMVYLGARENTK |
| sequence as | NO: | TQMEKVIHFDKITGLGESMESQCGTGVSIHTALKDMLSEITKPSDNYSLSLASR |
| secreted from | 115 | LYAFQTYAILPEYLQCIKELYKESLETVSFQTAADQARELINSWIESQINGVIK |
| pichia | | NFLQPGSVDSQTELVLVNAIYFKGMWEKAFKDEDTQEVPFRITEQESRPVQM |
| | | MYQAGSFKVATVAAEKIKILELPYASGELSMLVLLPDDISGLEQLETTISFEKL |
| | | TEWTSSNMMEDRNMKVYLPRMKIEEKYNLTSVLIALGMTDLFSPAANLSGIS |
| | | AAESLKMSEAIHAAYVEIYEADSEIVSSAGVQVEVTSDSEEFRVDHPFLFLIKH |
| | | NPTNSVLFFGRCISP |
| Ostrich construct | SEQ ID | MRFPSIFTAVLFAASSALAAPVNTTTEDETAQIPAEAVIGYSDLEGDFDVAVLP |
| (secretion signal + | NO: | FSNSTNNGLLFINTTIASIAAKEEGVSLEKREAEAGSIGTASAEFCFDVFKELKV |
| mature protein) | 116 | HHVNENIFYSPLSIISALSMVYLGARENTKTQMEKVIHFDKITGLGESMESQCG |
| | | LETVSFQTAADQARELINSWIESQTNGVIKNFLQPGSVDSQTELVLVNAIYFKG |
| | | MWEKAFKDEDTQEVPFRITEQESRPVQMMYQAGSFKVATVAAEKIKILELPY |
| | | ASGELSMLVLLPDDISGLEQLETTISFEKLTEWTSSNMMEDRNMKVYLPRMKI |
| | | EEKYNLTSVLIALGMTDLFSPAANLSGISAAESLKMSEAIHAAYVEIYEADSEI |
| | | VSSAGVQVEVTSDSEEFRVDHPFLFLIKHNPTNSVLFFGRCISP |
| Duck OVA | SEQ ID | EABAGSIGAASTEFCFDVFRELRVQHVNENIFYSPFSIISALAMVYLGARDNTR |
| sequence as | NO: | TQIDKVVHFDKLPGFGESMEAQCGTSVSVHSSLRDILTQITKPSDNFSLSFASR |
| secreted from | 117 | LYAEETYAILPEYLQCVKELYKGGLESISFQTAADQARELINSWVESQTNGIIK |
| pichia | | NILQPSSVDSQTTMVLVNAIYFKGMWEKAFKDEDTQAMPFRMTEQESKPVQ |
| | | MMYQVGSFKVAMVTSEKMKILELPFASGMMSMFVLLPDEVSGLEQLESTISF |
| | | EKLTEWTSSTMMEERRMKVYLPRMKMEEKYNLTSVFMALGMTDLFSSSAN |
| | | MSGISSTVSLKMSEAVHAACVEIFEAGRDVVGSAEAGMDVTSVSEEFRADHP |
| | | FLFFIKHNPTNSILFFGRWMSP |
| Duck construct | SEQ ID | MRFPSIFTAVLFAASSALAAPVNTTTEDETAQIPAEAVIGYSDLEGDFDVAVLP |
| (secretion signal + | NO: | FSNSTNNGLLFINTTIASIAAKEEGVSLEKREAEAGSIGAASTEFCFDVFRELRV |
| mature protein) | 118 | QHVNENIFYSPFSIISALAMVYLGARDNTRTQIDKVVHFDKLPGFGESMEAQC |
| | | GTSVSVHSSLRDILTQITKPSDNFSLSFASRLYAEETYAILPEYLQCVKELYKG |
| | | GLESISFQTAADQARELINSWVESQTNGIIKNILQPSSVDSQTTMVLVNAIYFK |
| | | GMWEKAFKDEDTQAMPFRMTEQESKPVQMMYQVGSFKVAMVISEKMKILE |
| | | LPFASGMMSMFVLLPDEVSGLEQLESTISFEKLTEWTSSTMMEERRMKVYLP |
| | | RMKMEEKYNLTSVFMALGMTDLFSSSANMSGISSTVSLKMSEAVHAACVEIF |
| | | EAGRDVVGSAEAGMDVTSVSEEFRADHPFLFFIKHNPTNSILFFGRWMSP |
| OCH1:EndoH | SEQ ID | MAKADGSLLYYNPHNPPRRYYFYMAIFAVSVICVLYGPSQQLSSPKIDASAPA |
| fusion protein | NO: | PVKQGPTSVAYVEVNNNSMLNVGKYTLADGGGNAFDVAVIFAANINYDTGT |
| | 119 | KTAYLHFNENVQRVLDNAVTQIRPLQQQGIKVLLSVLGNHQGAGFANFPSQQ |
| | | AASAFAKQLSDAVAKYGLDGVDFDDEYABYGNNGTAQPNDSSFVHLVTALR |
| | | ANMPDKIISLYNIGPAASRLSYGGVDVSDKFDYAWNPYYGTWQVPGIALPKA |
| | | QLSPAAVEIGRTSRSTVADLARRTVDEGYGVYLTYNLDGGDRTADVSAFTRE |
| | | LYGSEAVRTP |

An rOVD or rOVA can include additional sequences. Expression of rOVD and rOVA in a host cell, for instance a Pichia species, a Saccharomyces species, a Trichoderma species, a Pseudomonas species may lead to an addition of peptides to the OVD or OVA sequence as part of post-transcriptional or post-translational modifications. Such peptides may not be part of the native OVD or OVA sequences. For instance, expressing an OVD sequence in a Pichia species, such as Komagataella phaffii and Komagataella pastoris may lead to addition of a peptide at the N-terminus or C-terminus. In some cases, a tetrapeptide EAEA (SEQ ID NO: 120) is added to the N-terminus of the OVD sequence upon expression in a host cell. In some embodiments, rOVD or rOVA or both include the amino acids EAEA at the N-terminus. An OVD or OVA protein sequence can include a signal sequence, such as for directing secretion from a host cell. In some cases, the signal sequence may be a native signal sequence. In some cases, a signal sequence may be a heterologous signal sequence. For instance, an alpha mating factor signal sequence can be fused to an OVD or OVA sequence for expression and secretion in a yeast cell such as a Pichia sp. In some cases, the signal sequence is removed in whole or in part when the protein, such as an rOVD or rOVA, is secreted from the host cell.

An rOVD and/or rOVA can be a non-naturally occurring variant of an OVD and/or OVA. Such variant can comprise one or more amino acid insertions, deletions, or substitutions relative to a native OVD or native OVA sequence.

Such an rOVD variant can have at least 70%, 75%, 80%, 85%, 90%, 95%, 96%, 97%, 98%, or 99% sequence identity to SEQ ID NOs: 1-44. An rOVA variant can have at least 70%, 75%, 80%, 85%, 90%, 95%, 96%, 97%, 98%, or 99% sequence identity to SEQ ID NOs: 45-118. The term “sequence identity” as used herein in the context of amino acid sequences is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared.

In some embodiments, a variant is one that confers additional features, such as reduced allergenicity. For example, an rOVD can include G162M and/or F167A (such as in SEQ ID NO: 3) relative to a wild type OVD sequence SEQ ID NO: 2 and have reduced allergenicity as compared to the wild type OVD sequence.

Depending on the host organism used to express the rOVD and/or rOVA, the rOVD and/or rOVA can have a glycosylation, acetylation, or phosphorylation pattern different from wild-type OVD (e.g., native OVD) or wild-type OVA (e.g., native OVA). For example, the rOVD and/or rOVA herein may or may not be glycosylated, acetylated, or phosphorylated. An rOVD and/or rOVA may have an avian, non-avian, microbial, non-microbial, mammalian, or non-mammalian glycosylation, acetylation, or phosphorylation pattern.

In some cases, rOVD and/or rOVA may be deglycosylated or modified in its glycosylation (e.g., chemically, enzymatically through endoglucanases (such as EndoH), endoglycosidases, mannosidases (such as alpha-1,2 mannosidase), PNGase F, O-Glycosidase, OCH1, Neuraminidase, β,1-4 Galactosidase, β-N-acetylglucosaminidases, etc.), deacetylated (e.g., protein deacetylase, histone deacetylase, sirtuin), or dephosphorylated (e.g., acid phosphatase, lambda protein phosphatase, calf intestinal phosphatase, alkaline phosphatase), Deglycosylation, deacetylation or dephosphorylation may produce a protein that is more uniform or is capable of producing a composition with less variation.

The present disclosure contemplates modifying glycosylation of the rOVD to alter or enhance one or more functional characteristics of the protein and/or its production. A host cell may comprise heterologous enzymes that modify the glycosylation pattern of ovomucoid. In some cases, one or more enzymes may be used for modifying the glycosylation of rOVD protein. The enzymes used modifying glycosylation of rOVD may be an enzyme or a fusion protein comprising an enzyme or active fragment of an enzyme, for example EndoH or a fusion of OCH1 to EndoH (such as to provide for Golgi retention of the EndoH enzyme) may be provided in a host cell.

Native ovomucoid (nOVD), such as isolated from a chicken or other avian egg, has a highly complex branched form of glycosylation. The glycosylation pattern comprises N-linked glycan structures such as N-acetylglucosamine units and N-linked mannose units. See, e.g., FIG. 1A (left-hand column). In some cases, the rOVD for use in a herein-disclosed composition and produced using the methods described herein has a glycosylation pattern which is different than the glycosylation pattern of nOVD. For example, when rOVD is produced in a Pichia sp., the protein may be highly glycosylated. FIG. 1B illustrates the glycosylation patterns of rOVD produced by P. pastoris, showing a complex branched glycosylation pattern. In some embodiments of the compositions and methods herein, rOVD is treated such that the glycosylation pattern is modified from that of nOVD and also modified as compared to rOVD produced by a Pichia sp. without such treatment. In some cases, the rOVD has no glycosylation. In other cases, the rOVD has reduced glycosylation. In some cases, the rOVD is modified by N-acetylglucosamine at one or more asparagine residues of the protein and lacks or is substantially devoid of N-linked mannosylation. See, e.g., FIG. 1A (right hand column). The changes in glycosylation described herein may lead to an increase in the solubility and clarity of rOVD as compared to other forms of protein such as whey proteins, soy proteins, pea proteins, and nOVD.

In some cases, an enzyme used for modifying glycosylation may be transformed into a host cell. In some cases, the enzyme used for modifying glycosylation may be transformed into the same host cell that produces rOVD. In some cases, the enzyme may be provided transiently to the host cell, such as by an inducible expression system. In some cases, when a host cell expresses an enzyme used for modifying glycosylation, the recombinant protein (e.g., rOVD and rOVA) is secreted from the host cell in the modified state.

In one example, a host cell producing OVD comprises a fusion of EndoH and OCH1 enzymes. An exemplary OCH1-EndoH protein sequence is provided as SEQ ID NO: 119. In such cases, an rOVD produced from the host cell comprises a glycosylation pattern substantially different from an rOVD which is produced in a cell without such enzymes. The rOVD produced in such cases is also substantially different as compared to a native OVD (e.g., produced by a chicken or other avian egg). FIG. 1A shows a comparison of nOVD (with mannose residues) and rOVD glycosylation patterns wherein the rOVD was treated with EndoH and comprises an N-acetylglucosamine residue at the asparagine but no mannose residues. FIG. 1C shows the glycosylation pattern of rOVD produced in a host cell such as P. pastoris and where rOVD was not treated with EndoH and has both N-acetylglucosamine resides as well as the chains of N-linked mannose residues. Modification of the glycosylation of rOVD may provide nutritional benefits to rOVD, such as a higher nitrogen to carbon ratio, and may improve the clarity and solubility of the protein. In some cases, the modification of the glycosylation of rOVD is performed within the host cell that produces rOVD before the rOVD is secreted from the host cell and/or before isolating the rOVD. In some cases, modification of the glycosylation of rOVD is performed after its secretion and/or after isolating rOVD from the host cell.

The molecular weight or rOVD may be different as compared to nOVD. The molecular weight of the protein may be less than the molecular weight of nOVD or less than rOVD produced by the host cell where the glycosylation of rOVD is not modified. In embodiments, the molecular weight of an rOVD may be from about 20 kDa to about 40 kDa. In some cases, an rOVD with modified glycosylation has a different molecular weight, such as compared to a native OVD (as produced by an avian host species) or as compared to a host cell that glycosylates the rOVD, such as where the rOVD includes N-linked mannosylation. In some cases, the molecular weight of rOVD is greater than the molecular weight of the rOVD that is completely devoid of post-translational modifications or an rOVD that lacks all forms of N-linked glycosylation.

The present disclosure contemplates modifying glycosylation of the rOVA to alter or enhance one or more functional characteristics of the protein and/or its production. In some embodiments, the change in rOVA glycosylation can be due to the host cell glycosylating the rOVA. In some embodiments, rOVA has a glycosylation pattern that is not identical to a native ovalbumin (nOVA), such as a nOVA from chicken egg. In some embodiments, rOVA is treated with a deglycosylating enzyme before it is used as an ingredient in an rOVA composition, or when rOVA is present in a composition. In some embodiments, the glycosylation of rOVA is modified or removed by expressing one or more enzymes in a host cell and exposing rOVA to the one or more enzymes. In some embodiments, rOVA and the one or more enzymes for modification or removal of glycosylation are co-expressed in the same host cell.

Native ovalbumin (nOVA), such as isolated from a chicken or another avian egg, has a highly complex branched form of glycosylation. The glycosylation pattern comprises N-linked glycan structures such as N-acetylglucosamine units, galactose and N-linked mannose units. See, e.g., FIG. 2A. In some cases, the rOVA for use in a herein disclosed composition and produced using the methods described herein has a glycosylation pattern which is different from the glycosylation pattern of nOVA. For example, when rOVA is produced in a Pichia sp., the protein may be glycosylated differently from the nOVA and lack galactose units in the N-linked glycosylation. FIG. 2B illustrates the glycosylation patterns of rOVA produced by P. pastoris, showing a complex branched glycosylation pattern. In some embodiments of the compositions and methods disclosed herein, rOVA is treated such that the glycosylation pattern is modified from that of nOVA and also modified as compared to rOVA produced by a Pichia sp. without such treatment. In some cases, the rOVA lacks glycosylation.

The molecular weight or rOVA may be different as compared to nOVA. The molecular weight of the protein may be less than the molecular weight of nOVA or less than rOVA produced by the host cell where the glycosylation of rOVA is not modified. In embodiments, the molecular weight of an rOVA may be from about 40 kDa to about 55 kDa. In some cases, an rOVA with modified glycosylation has a different molecular weight, such as compared to a native OVA (as produced by an avian host species) or as compared to a host cell that glycosylates the rOVA, such as where the rOVA includes N-linked mannosylation. In some cases, the molecular weight of rOVA is greater than the molecular weight of the rOVA that is completely devoid of post-translational modifications. or an rOVA that lacks all forms of N-linked glycosylation.

Expression of an rOVD or rOVA can be provided by an expression vector, a plasmid, nucleic acid integrated into the host genome or other means. For example, a vector for expression can include: (a) a promoter element, (b) a signal peptide, (c) a heterologous OVD or OVA sequence, and (d) a terminator element.

Expression vectors that can be used for expression of rOVD and rOVA include those containing an expression cassette with elements (a), (b), (c) and (d). In some embodiments, the signal peptide (c) need not be included in the vector. In general, the expression cassette is designed to mediate the transcription of the transgene when integrated into the genome of a cognate host microorganism.

To aid in the amplification of the vector prior to transformation into the host microorganism, a replication origin (e) may be contained in the vector (such as PUC_ORIC and PUC (DNA2.0)). To aide in the selection of microorganism stably transformed with the expression vector, the vector may also include a selection marker (f) such as URA3 gene and Zeocin resistance gene (ZeoR). The expression vector may also contain a restriction enzyme site (u) that allows for linearization of the expression vector prior to transformation into the host microorganism to facilitate the expression vectors stable integration into the host genome. In some embodiments the expression vector may contain any subset of the elements (b), (e), (f), and (g), including none of elements (b), (e), (f), and (g). Other expression elements and vector element known to one of skill in the art can be used in combination or substituted for the elements described herein.

Exemplary promoter elements (a) may include, but are not limited to, a constitutive promoter, inducible promoter, and hybrid promoter. Promoters include, but are not limited to, acu-5, adh1+, alcohol dehydrogenase (ADH1, ADH2, ADH4), AHSB4m, AINV, alcA, α-amylase, alternative oxidase (AOD), alcohol oxidase 1 (AOX1), alcohol oxidase 2 (AOX2), AXDH, B2, CaMV, cellobiohydrolase I (cbh1), ccg-1, cDNA1, cellular filament polypeptide (cfp), cpc-2, ctr4+, CUP1, dihydroxyacetone synthase (DAS), enolase (ENO, ENO1), formaldehyde dehydrogenase (FLD1), FMD, formate dehydrogenase (FMDH), G1, G6, GAA, GAL1, GAL2, GAL3, GAL4, GAL5, GAL6, GAL7, GAL8, GAL9, GAL10, GCW14, gdhA, gla-1, α-glucoamylase (glaA), glyceraldehyde-3-phosphate dehydrogenase (gpdA, GAP, GAPDH), phosphoglycerate mutase (GPM1), glycerol kinase (GUT1), HSP82, invl+, isocitrate lyase (ICL1), acetohydroxy acid isomeroreductase (ILV5), KAR2, KEX2, β-galactosidase (lac4), LEU2, melO, MET3, methanol oxidase (MOX), nmt1, NSP, pcbC, PET9, peroxin 8 (PEW, phosphoglycerate kinase (PGK, PGK1), pho1, PHO5, PHO89, phosphatidylinositol synthase (PIS1), PYK1, pyruvate kinase (pki1), RPS7, sorbitol dehydrogenase (SDH), 3-phosphoserine aminotransferase (SER1), SSA4, SV40, TEF, translation elongation factor 1 alpha (TEF1), THI11, homoserine kinase (THR1), tpi, TPS1, triose phosphate isomerase (TPI1), XRP2, YPT1, a sequence or subsequence chosen from SEQ ID Nos: 121 to 132, and any combination thereof. Illustrative inducible promoters include methanol-induced promoters, e.g., DAS1 and pPEX11.

A signal peptide (b), also known as a signal sequence, targeting signal, localization signal, localization sequence, signal peptide, transit peptide, leader sequence, or leader peptide, may support secretion of a protein or polynucleotide. Extracellular secretion of a recombinant or heterologously expressed protein from a host cell may facilitate protein purification. A signal peptide may be derived from a precursor (e.g., prepropeptide, preprotein) of a protein. Signal peptides can be derived from a precursor of a protein other than the signal peptides in native OVD and/or OVA.

Any nucleic acid sequence that encodes OVD and/or OVA can be used as (c). Preferably such sequence is codon optimized for the species/genus/kingdom of the host cell.

Exemplary transcriptional terminator elements include, but are not limited to, acu-5, adh1+, alcohol dehydrogenase (ADH1, ADH2, ADH4), AHSB4m, AINV, alcA, α-amylase, alternative oxidase (AOD), alcohol oxidase 1 (AOX1), alcohol oxidase 2 (AOX2), AXDH, B2, CaMV, cellobiohydrolase I (cbh1), ccg-1, cDNA1, cellular filament poly peptide (cfp), cpc-2, ctr4+, CUP1, dihydroxyacetone synthase (DAS), enolase (ENO, ENO1), formaldehyde dehydrogenase (FLD1), FMD, formate dehydrogenase (FMDH), G1, G6, GAA, GAL1, GAL2, GAL3, GAL4, GAL5, GAL6, GAL7, GAL8, GAL9, GAL10, GCW14, gdhA, gla-1, α-glucoamylase (glaA), glyceraldehyde-3-phosphate dehydrogenase (gpdA, GAP, GAPDH), phosphoglycerate mutase (GPM1), glycerol kinase (GUT1), HSP82, invl+, isocitrate lyase (ICL1), acetohydroxy acid isomeroreductase (ILV5), KAR2, KEX2, β-galactosidase (lac4), LEU2, melO, MET3, methanol oxidase (MOX), nmt1, NSP, pcbC, PET9, peroxin 8 (PEX8), phosphoglycerate kinase (PGK, PGK1), pho1, PHO5, PHO89, phosphatidylinositol synthase (PIS1), PYK1, pyruvate kinase (pki1), RPS7, sorbitol dehydrogenase (SDH), 3-phosphoserine aminotransferase (SER1), SSA4, SV40, TEF, translation elongation factor 1 alpha (TEF1), THI11, homoserine kinase (THR1), tpi, TPS1, triose phosphate isomerase (TPI1), XRP2, YPT1, and any combination thereof.

Exemplary selectable markers (f) may include but are not limited to: an antibiotic resistance gene (e.g. zeocin, ampicillin, blasticidin, kanamycin, nurseothricin, chloroamphenicol, tetracycline, triclosan, ganciclovir, and any combination thereof), an auxotrophic marker (e.g. ade1, arg4, his4, ura3, met2, and any combination thereof).

In one example, a vector for expression in Pichia sp. can include an AOX1 promoter operably linked to a signal peptide (alpha mating factor) that is fused in frame with a nucleic acid sequence encoding OVD and/or OVA, and a terminator element (AOX1 terminator) immediately downstream of the nucleic acid sequence encoding OVD and/or OVA.

In another example, a vector comprising a DAS1 promoter is operably linked to a signal peptide (alpha mating factor) that is fused in frame with a nucleic acid sequence encoding OVD and/or OVA and a terminator element (AOX1 terminator) immediately downstream of OVD and/or OVA.

A recombinant protein described herein may be secreted from the one or more host cells. In some embodiments, rOVD and/or rOVA protein is secreted from the host cell. The secreted rOVD and/or rOVA may be isolated and purified by methods such as centrifugation, fractionation, filtration, affinity purification and other methods for separating protein from cells, liquid and solid media components and other cellular products and byproducts. In some embodiments, rOVD and/or rOVA is produced in a Pichia Sp. and secreted from the host cells into the culture media. The secreted rOVD and/or rOVA is then separated from other media components for further use.

In some cases, multiple vectors comprising OVD may be transfected into one or more host cells. A host cell may comprise more than one copy of OVD. A single host cell may comprise 2, 3, 4, 5, 6, 7, 8, 9, 10, 11, 12, 13, 14, 15, 16, 17, 18, 19 or 20 copies of OVD. A single host cell may comprise one or more vectors for the expression of OVD. A single host cell may comprise 2, 3, 4, 5, 6, 7, 8, 9 or 10 vectors for OVD expression. Each vector in the host cell may drive the expression of OVD using the same promoter. Alternatively, different promoters may be used in different vectors for OVD expression.

An rOVD and/or rOVA is recombinantly expressed in one or more host cells. As used herein, a “host” or “host cell” denotes here any protein production host selected or genetically modified to produce a desired product. Exemplary hosts include fungi, such as filamentous fungi, as well as bacteria, yeast, plant, insect, and mammalian cells. A host cell may be Arxula spp., Arxula adeninivorans, Kluyveromyces spp., Kluyveromyces lactis, Komagataella phaffi, Pichia spp., Pichia angusta. Pichia pastoris, Saccharomyces spp., Saccharomyces cerevisiae, Schizosaccharomyces spp., Schizosaccharomyces pombe, Yarrowia spp., Yarrowia lipolytica, Agaricus spp., Agaricus bisporus, Aspergillus spp., Aspergillus awamori, Aspergillus fiimigatus, Aspergillus nidulans, Aspergillus niger. Aspergillus oryzae, Bacillus subtilhs, Colletotrichum spp., Colletotrichum gloeosporiodes, Endothia spp., Endothia parasitica, Escherichia coli, Fusarium spp., Fusarium graminearum. Fusarium solani, Mucor spp., Mucor miehei, Mucor pusillus, Myceliophthora spp., Myceliophthora thermophila, Neurospora spp., Neurospora crassa, Penicillium spp., Penicillium camemberti, Penicillium canescens, Penicillium chrysogenum. Penicillium (Talaromyces) emersonii, Penicillium funiculo sum. Penicillium purpurogenum, Penicillium roqueforti, Pleurotus spp., Pleurotus ostreatus, Rhizomucor spp., Rhizomucor miehei. Rhizomucor pusillus, Rhizopus spp., Rhizopus arrhizus. Rhizopus oligosporus. Rhizopus oryzae, Trichoderma spp., Trichoderma altroviride, Trichoderma reesei, or Trichoderma vireus. A host cell can be an organism that is approved as generally regarded as safe by the U.S. Food and Drug Administration.

A recombinant protein can be recombinantly expressed in yeast, filamentous fungi or a bacterium. In some embodiments, recombinant protein is recombinantly expressed in a Pichia species (Komagataella phaffii and Komagataella pastoris), a Saccharomyces species, a Trichoderma species, a Trichoderma species, a Pseudomonas species or an E. coli species.

A host cell may be transformed to include one or more expression cassettes. As examples, a host cell may be transformed to express one expression cassette, two expression cassettes, three expression cassettes or more expression cassettes. In one example, a host cell is transformed express a first expression cassette that encodes rOVA and express a second expression cassette that encodes rOVD. In another example, a first host cell is transformed to express a first expression cassette that encodes rOVA and a second host cell is transformed to express a second expression cassette that encodes rOVD.

The consumable products and rOVD and/or rOVA compositions herein can be essentially free of any microbial cells or microbial cell contaminants. For instance, rOVD and/or rOVA may be isolated from a culture comprising microbial growth.

Treated rOVD

The rOVD, included in a rOVA and rOVD containing composition, may be treated chemically or enzymatically before it is purified for use in a composition or protein mixture. Such treatments may be performed to reduce impurities in an rOVD protein composition. Such treatments may be performed to improve the sensory attributes of the rOVD protein composition. Treatments may include but are not limited to purification steps, filtration, chemical treatments, and enzymatic treatments.

In some cases, rOVD protein and compositions containing rOVD protein, including forms of rOVD with modified glycosylation (e.g., such forms with N-acetylglucosamine but lacking N-linked mannose residues) may be treated with oxidizing agent or an oxygen-generating agent to modify components of the rOVD composition, such as impurities. The oxidizing agent or oxygen-generating agent may comprise hydrogen peroxide, sodium percarbonate, activated chlorine dioxide, bubbled oxygen or ozone. The treatment may improve the solubility and clarity of an rOVD composition. The treatment may reduce the odor of an rOVD composition. The treatment may neutralize the color of an rOVD composition, for instance, the rOVD composition may lose color after a treatment, e.g., to a less intense/lighter coloration. In embodiments, the color may change form greenish to yellowish and/or from yellowish to essentially colorless.

In some examples, rOVD may be treated with an oxidizing agent or an oxygen-generating agent, e.g., hydrogen peroxide or sodium percarbonate, before it is purified for use in a composition. A culture medium comprising secreted or isolated rOVD may be treated with an oxygen-generating agent, e.g., hydrogen peroxide or sodium percarbonate. Using hydrogen peroxide as an example, a hydrogen peroxide treatment may be followed by one or more wash steps and/or filtration steps to remove hydrogen peroxide from the resulting rOVD compositions. Such steps may be performed following treatments with other oxygen-generating agents, e.g., sodium percarbonate.

In some cases, the concentration of hydrogen peroxide used for treating rOVD may be from 1% to 20%. The concentration of hydrogen peroxide used for treating rOVD may be at least 1% weight per total weight (w/w) and/or weight per total volume (w/v). The concentration of hydrogen peroxide used for treating rOVD may be at most 20% w/w or w/v. The concentration of hydrogen peroxide used for treating rOVD may be 1% to 2%, 1% to 5%, 1% to 7%, 1% to 10%, 1% to 12%, 1% to 15%, 1% to 17%, 1% to 20%, 2% to 5%, 2% to 7%, 2% to 10%, 2% to 12%, 2% to 15%, 2% to 17%, 2% to 20%, 5% to 7%, 5% to 10/o, 5% to 12%, 5% to 15%, 5% to 17%, 5% to 20%, 7% to 10%, 7% to 12%, 7% to 15%, 7% to 17%, 7% to 20%, 10% to 12%, 10% to 15%, 10% to 17%, 10% to 20%, 12% to 15%, 12% to 17%, 12% to 20%, 15% to 17%, 15% to 20%, or 17% to 20% w/w or w/v. The concentration of hydrogen peroxide used for treating rOVD may be about 1%, 2%, 5%, 7%, 10%, 12%, 15%, 17%, or 20% w/w or w/v. The concentration of hydrogen peroxide used for treating rOVD may be at least 1%, 2%, 5%, 7%, 10%, 12%, 15% or 17% w/w or w/v. The concentration of hydrogen peroxide used for treating rOVD may be at most 2%, 5%, 7%, 10%, 12%, 15%, 17%, or 20% w/w or w/v.

rOVD may be treated with hydrogen peroxide for a limited duration of time. For instance, rOVD may be exposed to hydrogen peroxide for at least 1 hour, 2 hours, 3 hours, 5 hours, 7 hours, 10 hours, 12 hours, 15 hours, 17 hours, 20 hours, 22 hours, 24 hours, 26 hours, 28 hours, 30 hours, 34 hours, 36 hours, 40 hours, 44 hours or 48 hours. Hydrogen peroxide may be added to the rOVD culture media throughout the culturing process.

rOVD may be treated with hydrogen peroxide at a pH of about 3 to 6. rOVD may be treated with hydrogen peroxide at a pH of about 3, 3.2, 3.4, 3.6, 3.8, 4, 4.1, 4.2, 4.4, 4.6, 4.8, 5, 5.2, 5.4, 5.6, 5.8 or 6. rOVD may treated with hydrogen peroxide at a pH of at least 3, 3.2, 3.4, 3.6, 3.8, 4, 4.1, 4.2, 4.4, 4.6, 4.8, 5, 5.2, 5.4, 5.6 or 5.8. rOVD may treated with hydrogen peroxide at a pH of at most 3.2, 3.4, 3.6, 3.8, 4, 4.1, 4.2, 4.4, 4.6, 4.8, 5, 5.2, 5.4, 5.6, 5.8 or 6.

rOVD may be filtered before treatment with an oxygen-generating agent. In some cases, rOVD may be filtered before and after treatment with an oxygen-generating agent.

### Definitions

The terminology used herein is for the purpose of describing particular cases only and is not intended to be limiting.

As used herein, unless otherwise indicated, the terms “a”. “an” and “the” are intended to include the plural forms as well as the single forms, unless the context clearly indicates otherwise.

The terms “comprise”, “comprising”, “contain,” “containing,” “including”, “includes”, “having”, “has”, “% with”, or variants thereof as used in either the present disclosure and/or in the claims, are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean 10% greater than or less than the stated value. In another example, “about” can mean within 1 or more than 1 standard deviation, per the practice in the given value. Where particular values are described in the application and claims, unless otherwise stated the term “about” should be assumed to mean an acceptable error range for the particular value.

The term “substantially” is meant to be a significant extent, for the most part, or essentially. In other words, the term substantially may mean nearly exact to the desired attribute or slightly different from the exact attribute. Substantially may be indistinguishable from the desired attribute. Substantially may be distinguishable from the desired attribute but the difference is unimportant or negligible.

The term “similar” is understood to be resembling up to and including identical. Therefore two (or more) items may be identical, substantially identical, comprise equivalent components, comprise substitutable components, comprise analogous components, comprise comparable components, comprise complementary components, comprise related components, comprise like components, and/or the differences between the two (or more items) are insubstantial and/or result in a composition having equivalent properties, identical properties, substantially identical properties, and the like.

As used herein, an “egg white substitute” may include products such as aquafaba, chia seeds, flax seeds, starches, apple sauce, banana puree, condensed milk, and other ingredients that are commonly used as egg white substitutes.

Any aspect or embodiment described herein can be combined with any other aspect or embodiment as disclosed herein.

### ADDITIONAL EMBODIMENTS

Embodiment 1. An egg white-like composition having a protein component comprising recombinantly-produced ovomucoid (rOVD) and recombinantly-produced ovalbumin (rOVA), wherein the composition has a higher foaming capacity than a control composition, wherein the control composition consists of the same contents by identity and quantity as the egg white-like composition, but the control's protein component is one of: a) chicken egg-white; b) ovomucoid; or c) ovalbumin.

Embodiment 2. An egg-white like composition having a protein component comprising recombinantly-produced ovomucoid (rOVD) and recombinantly-produced ovalbumin (rOVA), wherein the composition has a higher foam stability than a control composition, wherein the control composition is substantially similar to the egg-white like composition, but the control's protein component is one of: a) chicken egg-white; b) ovomucoid; or c) ovalbumin.

Embodiment 3. An emulsified composition having a protein component comprising recombinantly-produced ovomucoid (rOVD) and recombinantly-produced ovalbumin (rOVA), wherein the composition has a comparable or higher emulsion stability than a control composition, wherein the control composition is substantially similar to the emulsified composition, but the control's protein component is one of: a) chicken egg-white; b) ovomucoid; or c) ovalbumin.

Embodiment 4. A foam composition comprising essentially of a solvent and a protein component consisting of a recombinantly-produced ovomucoid (rOVD) and a recombinantly-produced ovalbumin (rOVA), wherein the composition has a higher foaming capacity and foam stability than a control composition, wherein the control composition consists of the same contents by identity and quantity as the egg white-like composition, but the control's protein component is one of: a) chicken egg-white; b) ovomucoid: or c) ovalbumin.

Embodiment 5. A salad dressing composition comprising essentially of an oil component, an acid component, and a protein component consisting of a recombinantly-produced ovomucoid (rOVD) and a recombinantly-produced ovalbumin (rOVA), wherein the salad dressing has a comparable or higher emulsion stability than a control composition, wherein the control composition is substantially similar to the salad dressing composition, but the control's protein component is one of: a) chicken egg-white; b) ovomucoid: or c) ovalbumin.

Embodiment 6. A liquid composition comprising essentially of a solvent and a protein component comprising a recombinantly-produced ovomucoid (rOVD) and a recombinantly-produced ovalbumin (rOVA), wherein the liquid composition has at least one of a comparable or higher emulsion stability, foaming capacity, foam stability, and time spent to generate foam as compared to a control composition, wherein the control composition is substantially similar to the liquid composition, but the control's protein component is one of: a) chicken egg-white; b) ovomucoid; or c) ovalbumin.

Embodiment 7. An egg white-like composition having a protein component comprising essentially of a recombinantly-produced ovomucoid (rOVD) and a recombinantly-produced ovalbumin (rOVA), wherein a ratio of rOVD to rOVA is form about 1:3 to about 3:1.

Embodiment 8. The egg white-like composition of any one of the previous Embodiments, wherein the ratio of rOVD to rOVA is 1:3, 1:2, 1:1, 2:1, or 3:1.

Embodiment 9. The composition of any one of Embodiments 1-8, wherein the rOVD has a glycosylation pattern different from the glycosylation pattern of a native chicken ovomucoid.

Embodiment 10. The composition of Embodiment 9, wherein the rOVD protein comprises at least one glycosylated asparagine residue and the rOVD is substantially devoid of N-linked mannosylation.

Embodiment 11. The composition of Embodiment 10, wherein each glycosylated asparagine comprises a single N-acetylglucosamine.

Embodiment 12. The composition of any one of Embodiments 9-11, wherein the rOVD comprises at least three glycosylated asparagine residues.

Embodiment 13. The composition of any one the previous Embodiments, wherein the rOVD provides protein fortification to the composition and provides an improvement to at least one additional feature selected from the group consisting of solubility, mouthfeel, texture, thickness, hardness, stability to heat treatment, and stability to pH.

Embodiment 14. The composition of any one of the previous Embodiments, wherein the protein component comprises at least 5% rOVD w/w.

Embodiment 15. The composition of any one of the previous Embodiments, wherein the composition comprises at least 1% rOVD w/w.

Embodiment 16. The composition of any one of the previous Embodiments, wherein the composition has sensory properties comparable to those of the control composition.

Embodiment 17. The composition of any one of the previous Embodiments, wherein the rOVA has a glycosylation pattern different from a native ovalbumin.

Embodiment 18. The composition of any one of the previous Embodiments, wherein the protein component comprises at least 5% rOVA w/w.

Embodiment 19. The composition of any one of the previous Embodiments, wherein the composition comprises at least 1% rOVA w/w.

Embodiment 20. The composition of any one of the previous Embodiments, wherein the pH of the rOVA when solubilized is from about 3.5 to about 7.0.

Embodiment 21. The composition of any one of the previous Embodiments, wherein the rOVA is in an amount from about 2% to about 15% (w/w) in the composition.

Embodiment 22. The composition of any one of the previous Embodiments, wherein the rOVA provides to an egg-less food item at least one egg-white characteristic selected from gelling, foaming, whipping, fluffing, binding, springiness, aeration, coating, film forming, emulsification, browning, thickening, texturizing, humectant, clarification, and cohesiveness.

Embodiment 23. The composition of any one of the previous Embodiments, wherein the rOVD and/or the rOVA is produced by a microbial host cell.

Embodiment 24. The composition of Embodiment 23, wherein the microbial host cell is a yeast, a filamentous fungus, or a bacterium.

Embodiment 25. The composition of Embodiment 23 or Embodiment 24, wherein the microbial host cell is a Pichia species, a Saccharomyces species, a Trichoderma species, a Pseudomonas species or an E. coli species.

Embodiment 26. The composition of any one of the previous Embodiments, wherein the protein component does not comprise any egg-white proteins other than rOVD and rOVA.

Embodiment 27. The composition of any one of the previous Embodiments, wherein the composition comprises one or more excipients.

Embodiment 28. The composition of any one of the previous Embodiments, wherein the composition comprises one or more solvents.

Embodiment 29. The composition of any one of Embodiments 1 to 28, wherein rOVD comprises an amino acid sequence of one of SEQ ID No. 1-44 or an amino acid sequence having at least 85% sequence identity with one of SEQ ID No. 1-44.

Embodiment 30. The composition of any one of Embodiments 1 to 28, wherein rOVA comprises an amino acid sequence of one of SEQ ID No. 45-118 or an amino acid sequence having at least 85% sequence identity with one of SEQ ID No. 45-118.

Embodiment 31. A food composition of Embodiments 1-28, wherein the foodstuff further comprises one or more non-egg white proteins.

Embodiment 32. A method of making a foam composition comprising mixing a recombinantly-produced ovomucoid and a recombinantly-produced ovalbumin in a solvent, wherein the composition needs less time to foam than a control composition, wherein the control composition consists of the same contents by identity and quantity as the foam composition, but the control's protein component is one of; a) chicken egg-white; b) ovomucoid; or c) ovalbumin.

### EXAMPLES

The following examples are given for the purpose of illustrating various embodiments of the invention and are not meant to limit the present invention in any fashion. The present examples, along with the methods described herein are presently illustrative of preferred embodiments, are exemplary, and are not intended as limitations on the scope of the invention. Changes therein and other uses which are encompassed within the spirit of the invention as defined by the scope of the claims will occur to those skilled in the art.

### Example 1: Expression Constructs, Transformation, Protein Purification and Processing

Two expression constructs were created for expression of OVD (SEQ ID NO: 1) in Pichia pastoris. The first construct included the Alcohol oxidase 1 (AOX1) promoter. An OVD coding sequenced was fused in-frame with the alpha mating factor signal sequence downstream of the promoter sequence. A transcriptional terminator from the AOX1 gene was placed downstream of the OVD sequence. The expression construct was placed into a Kpas-URA 3 vector.

A second expression construct was created containing the methanol-inducible DAS1 promoter (ATCC No. 28485) upstream of the alpha mating factor signal sequence fused in frame with a nucleic acid sequence encoding the same OVD protein sequence as in the first expression construct. A transcriptional terminator from the AOX1 gene was placed downstream of the OVD sequence.

In both expression constructs, the OVD sequence was that of chicken (Gallus gallus) having amino acid sequence of SEQ ID NO: 1.

Both expression constructs were transformed into Pichia pastoris. Successful integration of the two constructs were confirmed by genomic sequencing.

Fermentation: Recombinant OVD (rOVD) from each expression construct was produced in a bioreactor at ambient conditions. A seed train for the fermentation process began with the inoculation of shake flasks with liquid growth broth. The inoculated shake flasks were kept in a shaker after which the grown Pichia pastoris was transferred to a production scale reactor.

The culture was grown at 30° C., at a set pH and dissolved oxygen (DO). The culture was fed with a carbon source.

Secreted rOVD was purified by separating cells from the liquid growth broth, performing multiple filtration steps, performing chromatography using and drying the final protein product to produce pure rOVD powder.

### Example 2: Expression Construct, Transformation, Protein Purification and Processing

Three expression constructs were created for expression of a mature form of OVD (SEQ ID NO: 1) in Pichia pastoris. The first construct included the AOX1 promoter. An OVD coding sequenced was fused in-frame with the alpha mating factor signal sequence downstream of the promoter sequence (SEQ ID NO: 39). A transcriptional terminator from the AOX1 gene was placed downstream of the OVD sequence. The host cells had eleven copies of OVD, ten of which were in the hybrid promoter system, with five driven by a shortened pAOX1. The eleventh copy was driven by a full-sized pAOX1 promoter.

A second expression construct was created containing a nucleic acid encoding the P. pastoris transcription factor HAC1 under the control of a strong methanol-inducible promoter. A transcriptional terminator from the AOX1 gene was placed downstream of the HAC1 sequence.

A third expression construct was created encoding a fusion protein. The construct comprises a nucleic acid that encodes the first 48 residues of Pichia OCH1 protein fused to a catalytically active version of the Streptomyces coelicoflavus EndoH (SEQ ID NO.: 119) and under a strong methanol-inducible promoter, pPEX11. A transcriptional terminator from the AOX1 gene was placed downstream of the EndoH-OCH1 fusion protein sequence.

The P. pastoris strain was modified to remove cytoplasmic killer plasmids and then further modified to have a deletion in the AOX1 gene. This deletion generated a methanol-utilization slow (mutS) phenotype that reduces the strain's ability to consume methanol. This base strain was transformed with the three expression constructs.

Linear cassettes of methanol-inducible promoter: ScPrePro (Saccharomyces pre-pro sequence)::ovomucoid::AOX1term; linear cassettes of methanol-inducible promoter::HAC1::AOX1term; and a linear cassette of methanol-inducible promoter::EndoH-OCH1::AOX1term were introduced into the base P. pastoris strain using standard electroporation methods.

Fermentation: Recombinant OVD from each expression construct was produced in a bioreactor at ambient conditions. A seed train for the fermentation process began with the inoculation of shake flasks with liquid growth broth. The inoculated shake flasks were kept in a shaker after which the grown P. pastoris was transferred to a production-scale reactor.

The culture was grown at 30° C., at a set pH and dissolved oxygen (DO). The culture was fed with a carbon source.

To expand production, an rOVD P. pastoris seed strain is removed from cryo-storage and thawed to room temperature. Contents of the thawed seed vials are used to inoculate liquid seed culture media in baffled flasks which were grown at 30° C. in shaking incubators. These seed flasks are then transferred and grown in a series of larger and larger seed fermenters (number to vary depending on scale) containing a basal salt media, trace metals, and glucose. Temperature in the seed reactors are controlled at 30° C., pH at 5, and DO at 30%. pH is maintained by feeding ammonia hydroxide which also acts as a nitrogen source. Once sufficient cell mass is reached, the grown rOVD P. pastoris is inoculated in a production-scale reactor containing basal salt media, trace metals, and glucose. Like in the seed tanks, the culture is also controlled at 30° C., pH 5 and 30% DO throughout the process. pH is again maintained by feeding ammonia hydroxide. During the initial batch glucose phase, the culture is left to consume all glucose and subsequently-produced ethanol. Once the target cell density is achieved and glucose and ethanol concentrations are confirmed to be zero, the glucose fed-batch growth phase is initiated. In this phase, glucose is fed until the culture reaches a target cell density. Glucose is fed at a limiting rate to prevent ethanol from building up in the presence of non-zero glucose concentrations. In the final induction phase, the culture is co-fed glucose and methanol which induces it to produce rOVD. Glucose is fed at an amount to produce a desired growth rate, while methanol is fed to maintain the methanol concentration at 1% to ensure that expression is consistently induced. Regular samples are taken throughout the fermentation process for analyses of specific process parameters (e.g., cell density, glucose/methanol concentrations, product titer, and quality). After a designated amount of fermentation time, secreted rOVD is collected and transferred for downstream processing.

The rOVD products were purified by separating cells from the liquid growth broth, performing multiple filtration steps, performing chromatography, and/or drying the final protein product to produce pure rOVD powder.

Post-translation modification from the OCH1-EndoH fusion protein resulted in the removal of the alpha factor pre-pro sequence. N-terminal sequencing results showed imprecise cleavage of the N-terminal pro sequence by the Pichia host post-transcription machinery fusing an additional four amino acid residues (major) or 6 amino acid residues (minor) to the N-terminus of the produced rOVD (SEQ ID NO: 37) or (SEQ ID NO:38) in comparison to the amino acid sequence of mature OVD (SEQ ID NO:1).

The molecular weight of rOVD from Pichia was compared against native chicken ovomucoid (nOVD) using SDS-PAGE. The rOVD showed a difference in migration. To ascertain whether the difference in gel migration was due to differential post-translational glycosylation, deglycosylated native ovomucoid was treated with PNGase F, an enzyme that specifically deglycosylates proteins (BioLabs 2020), and compared to the rOVD sample. The deglycosylated native ovomucoid (nOVD+PNGaseF) displayed the same band patterns and molecular weight as three rOVD samples tested (FIG. 1C). The difference in glycosylation is attributed to the action of the OCH1-EndoH in the Pichia strain, such that rOVD has only the core N-acetylglucosamine unit attached to the Asn residue instead of the complex branched glycosylation (that includes mannose) of nOVD from chicken egg white (FIG. 1A and FIG. 1B).

Mass spectrometry analysis of rOVD expressed in Pichia without EndoH is shown to have eight different N-glycan structures (FIG. 1B). The structures include Man9 GlcNAc2, Man9 GlcNAc2 Hex, Man9 GlcNAc2Hex2, Man9 GlcNAc2Hex3, Man9 GlcNAc2Hex4, Man9 GlcNAc2 Hex5,v Man9 GlcNAc2Hex6, and Man9 GlcNAc2 Hex7. Table 2 below shows the percentage of N-linked glycans on the rOVD sample produced without endoglycosidase treatment.

TABLE 2
N-linked glycans from sample detected by MALDI TOF/TOF MS.

| Permethylated | | |
|---|---|---|
| mass (m/z)¹ | Text description of structures | Percentage |
| 2396.2 | Man₉ GlcNAc₂ | 5.6 |
| 2600.3 | Man₉ GlcNAc₂ Hex | 25.1 |
| 2804.4 | Man₉ GlcNAc₂ Hex₂ | 31.6 |
| 3008.5 | Man₉ GlcNAc₂ Hex₃ | 18.2 |
| 3212.6 | Man₉ GlcNAc₂ Hex₄ | 6.0 |
| 3416.7 | Man₉ GlcNAc₂ Hex₅ | 7.2 |
| 3620.8 | Man₉ GlcNAc₂ Hex₆ | 3.8 |
| 3824.9 | Man₉ GlcNAc₂ Hex₇ | 2.6 |

### Example 3: Comparison of Bovine Trypsin Inhibitory Activity

rOVD as produced in Example 2 was utilized in this Example. The trypsin inhibition activity was compared between native OVD (nOVD) and recombinant OVD (rOVD) in a standard assay (AACC #22-40.01) using bovine trypsin. A comparison of rOVD with nOVD is shown in Table 3. One trypsin unit is arbitrarily defined as an increase of 0.01 absorbance unit at 410 nm per 10 ml of reaction mixture under the conditions of the assay. Trypsin inhibitor activity is expressed in terms of trypsin inhibitor units (TIU). Three different batches of rOVD (samples 1-3) were compared to a native chicken ovomucoid.

TABLE 3
Comparison of trypsin inhibition activity

| Product | Trypsin inhibition activity |
|---|---|
| Sample 1 | 8190 TIU/g |
| Sample 2 | 8180 TIU/g |
| Sample 3 | 8649 TIU/g |
| Native chicken Ovomucoid | 13721 TIU/g |

### Example 4: Comparison of In Vitro Digestibility

The in vitro digestibility of rOVD samples was measured using the Protein Digestibility Assay procedure (Megazyme, Medallion Labs). A comparison of rOVD samples with nOVD is shown in Table 4. The data demonstrates equivalent in vitro digestibility between native ovomucoid and rOVD.

TABLE 4
Comparison in vitro digestibility

| Product | In-vitro digestibility |
|---|---|
| Sample 1 | 93% |
| Sample 2 | 93% |
| Sample 3 | 93% |
| Native chicken Ovomucoid | 92% |

### Example 5: Ovomucoid Specifications

Based upon the characterization of the produced rOVD compositions and the properties of native chicken ovomucoid, product specifications (Table 5) and quality control specifications (Table 6) were constructed for an rOVD of the present disclosure

Protein percentages were measured using AOAC 2006. See, Protein (crude) in animal feed, combustion method, 990.03. In: Official methods of analysis of AOAC International. 18th ed. Gaithersburg: ASA-SSA Inc. and AOAC 2006. Proximate Analysis and Calculations Crude Protein Meat and Meat Products Including Pet Foods—item 80. In: Official methods of analysis Association of Analytical Communities, Gaithersburg, MD, 17th edition, Reference data: Method 992.15 (39.1.16); NFNAP; NITR; NT.

Moisture percentages were measured using Association of Official Analytical Chemists. 1995. In Official Methods of Analysis.

Carbohydrate percentages were measured using methods described in J AOAC Int. 2012 September-October; 95(5):1392-7.

Fat by acid hydrolysis were measured using AOAC International. 2012. Official Method Fat (crude) or ether extraction in pet food. Gravimetric method, 954.02. In: Official Methods of Analysis of AOAC International, 19th ed., AOAC International, Gaithersburg. MD, USA, 2012.

Standard plate count was measured using AOAC International. 2005. Aerobic plate count in foods, dry rehydratable film, method 990.12. AOAC International, 17th ed. Gaithersburg, MD. Yeast and mold counts were measured using AOAC Official Method 997.02. Yeast and Mold Counts in Foods Dry Rehydratable Film Method (Petrifilm™ Method) First Action 1997 Final Action 2000 Salmonella was measured using AOAC International. 2005. Salmonella in selected foods, BAX automated system, method 2003.09. In Official methods of analysis of AOAC International, 17th ed., AOAC International, Gaithersburg, MD. Total coliform was measured using AOAC International. 2005. E. coli count in foods, dry rehydratable film, method 991.14. In: Official methods of analysis of AOAC International, 17th ed. AOAC International, Gaithersburg, MD.

TABLE 5
Specification for Ovomucoid produced by P. pastoris DFB-003

| Physical properties | Specification |
|---|---|
| Source | Yeast fermentation-derived |
| Appearance | White to off-white amorphous powder |
| Solubility | Soluble in water |

| | Specification | | Method |
|---|---|---|---|
| Chemical Properties | | | |
| (in powder as is) | | | |
| Protein | >75% | | AOAC 990.03^{1a} |
| | | | AOAC 992.15^{1b} |
| Moisture | Maximum 10.0% | | AOAC 925.09² |
| Carbohydrate | Maximum 20% | | Calculated |
| Ash | Maximum 2.0% | | AOAC 942.05³ |
| Fat by Acid Hydrolysis | <0.1% | | AOAC 954.02⁴ |
| Hg | <1 | ppm | ICP-AES⁵ |
| Pb | <1 | ppm | ICP-AES⁵ |
| As | <1 | ppm | ICP-AES⁵ |
| Cd | <1 | ppm | ICP-AES⁵ |
| Microbial Properties | | | |
| (in powder as is) | | | |
| Standard Plate Count | <10000 | CFU/g | AOAC 990.12⁶ |
| Yeast & Mold | <100 | CFU/g | AOAC 997.02⁷ |
| Salmonella | Not Detected/25 g | | AOAC 2003.09⁸ |
| E. coli | Not Detected/25 g | | AOAC 991.14⁹ |
| Total coliform | ≤30 | CFU/g | AOAC 991.14⁹ |

TABLE 6
Quality control results for three lots of Ovomucoid produced by P. pastoris DFB-003

| Analysis Parameter | Specification | | SOL19303 | SOL19317 | SOL19351 |
|---|---|---|---|---|---|
| Protein | >75% | | 75.31 | 75.06 | 79.94 |
| Protein (% dry weight | >80% | | 82.2 | 82.5 | 87.8 |
| powder) | | | | | |
| Moisture and Volatiles | <10% | | 8.4 | 9 | 9 |
| Carbohydrates, Calculated | <20% | | 15.53 | 15.28 | 11.06 |
| Ash | <2% | | 0.76 | 10.66 | <0.4 |
| Fat by Acid Hydrolysis | <0.1% | | <0.10 | <0.10 | <0.10 |
| Arsenic (As) | <1 | mg/kg | <0.010 | <0.010 | <0.010 |
| Mercury (Hg) | <1 | mg/kg | <0.010 | <0.010 | <0.010 |
| Lead (Pb) | <1 | mg/kg | 0.03 | 0.063 | 10.168 |
| Cadmium (Cd) | <1 | mg/kg | <0.010 | <0.010 | <0.010 |
| Aerobic Plate Count | <10000 | CFU/g | <10 | <10 | <10 |
| Molds | <100 | CFU/g | <10 | <10 | <10 |
| Yeast | <100 | CFU/g | <10 | <10 | <10 |
| Salmonella | Not Detected/25 g | | Not Detected | Not Detected | Not Detected |
| Escherichia Coli | Not Detected/25 g | | Not Detected | Not Detected | Not Detected |
| Coliforms | <10 | CFU/g | <10 | <10 | <10 |

| | | | | |
|---|---|---|---|---|
| Absence of source organism | Not detected */ | Not detected | Not detected | Not detected |
| from product | mg sample | | | |
| Absence of encoding DNA | Not detected **/ | Not detected | Not detected | Not detected |
| from product | mg sample | | | |

* Limit of detection for source organism = 11 CFU/mg sample
** Limit of detection for encoding DNA = 10 femtogram

### Example 6: Absence of Production Organism and DNA in rOVD Preparations

rOVD powder was plated on PGA plates and if samples yielded colonies, these were re-streaked and analyzed by PCR for the presence of the Pichia organism. This procedure was applied to three lots of rOVD powder produced from the recombinant strain. No manufacturing organism was detected in any of the lots (Table 6).

PCR analysis was used to confirm that no encoding pieces of recombinant DNA was present in the rOVD preparation using primers for the rOVD cassette. OVD plasmid DNA was used as a positive control, producing a 570 bp band corresponding the OVD PCR product. This band was absent in all three rOVD powder lots tested.

### Example 7: Fermentation and Purification of rOVD

An rOVD P. pastoris seed strain was removed from cryo-storage and thawed to room temperature. Contents of the thawed seed vials were used to inoculate liquid culture media in the primary fermenter and grown at process temperature until target cell density was reached. Then, the grown rOVD P. pastoris was transferred to a production-scale reactor. The culture was grown in the production bioreactor at target fermentation conditions and fed a series of substrates. The fermentation was analyzed for culture purity at multiple times during the process.

The recombinant OVD was purified by separating the cells from the liquid medium by centrifugation, followed by microfiltration. Fermentation broth was first brought to pH 3 and diluted with DI water. Cells were removed using bucket centrifugation. The collected supernatant was brought to pH 7 using sodium hydroxide and a 0.2 μm filtration was performed followed by diafiltration with five volumes of deionized water. The permeates of the 0.2 μm were adjusted to pH 5 and then concentrated via 5 kDa TFF membrane. The 5 kDa retentate was precipitated using 65% saturation ammonium sulfate. After salt addition, the pH was adjusted to pH 4-4.1 with phosphoric acid. The mixture was incubated with agitation at room temperature overnight. The next day, precipitates were spun down using bucket centrifugation. The rOVD precipitates were dissolved in DI water and pH adjusted to 5 using sodium hydroxide. The rOVD solution was then diafiltered and then the retentate was passed through 0.2 μm bottle filters.

A spray dryer was used to dehydrate the rOVD solution into rOVD powder.

### Example 8: Hydrogen Peroxide Treatment During rOVD Purification

Liquid rOVD was concentrated to 50-60 g/L using a 5 kDa TFF membrane. The rOVD solution was passed through a 0.2 μm filter to remove microbes. Hydrogen peroxide, an oxygen-generating agent, in an amount to equal 10% volume of the solution was slowly added to the rOVD solution while stirring. The mixture was incubated with agitation and monitored to ensure color change from a dark green-brown color before treatment to a pale-yellow color after treatment. After 1.5 hours, diafiltration was performed via 5 kDa TFF membrane with 5 volumes of DI water. The rOVD in the 5 kDa diafiltration retentate was precipitated using ammonium sulfate at 65% salt saturation at room temperature. After addition of salt, the pH was adjusted to pH4-4.1 with phosphoric acid. The mixture was incubated with agitation overnight to form precipitates. The next day, the precipitates were spun down using bucket centrifugation. The precipitates were removed, dissolved in deionized water and pH adjusted to 5 using sodium hydroxide. Five kDa TFF membranes were cleaned and diafiltration was performed using volumes of DI water until a retentate conductivity of less than 2.0 mS was achieved. The retentate was passed through 0.2 μm bottle filters. The filtered rOVD solution was then spray dried and stored.

### Example 9: Reprocessed rOVD Treated with Hydrogen Peroxide

OVD powder was dissolved in deionized water to 50-60 g/L and filtered through a hollow fiber 0.2 μm tangential flow filter, then through a 0.2 μm bottle filter. Hydrogen peroxide in an amount to provide a 10% solution was slowly stirred into the rOVD solution and incubated for thirty minutes. The treated solution was washed through a 5 kDa membrane using 5 volumes of DI water.

Ammonium sulfate was slowly added to the retentate solution and the pH changed to between 4 to 4.1 using phosphoric acid. After overnight incubation with medium agitation, the solution was centrifuged, and supernatants discarded. Precipitates were collected, dissolved in DI water, and brought to pH 5 using sodium hydroxide. The protein solution was desalted with a 5 kDa membrane and filtered through a 0.2 μm bottle filter. Then, the protein solution was spray dried to produce rOVD powder.

### Example 10: Preparation of Recombinant Ovalbumin

A Gallus gallus OVA coding sequence was fused in-frame with the alpha mating factor signal sequence downstream of the promoter sequence (SEQ ID NO:45). A promoter was placed upstream of the signal sequence OVA coding sequence and a transcriptional terminator was placed downstream of the OVA sequence. The expression construct was placed into a Kpas-URA 3 vector.

The expression constructs were transformed into Pichia pastoris. Successful integration was confirmed by genomic sequencing.

Fermentation: Recombinant OVA was produced in a bioreactor at ambient conditions. A seed train for the fermentation process begins with the inoculation of shake flasks with liquid growth broth using 2 ml cryovials of Pichia pastoris which are stored at −80° C. and thawed at room temperature prior to inoculation.

The inoculated shake flasks were kept in a shaker at 30° C. for 24 hours, after which the grown Pichia pastoris was transferred to a production scale reactor.

The culture was grown at 30° C., at a set pH and dissolved oxygen (DO). The culture was fed with a carbon source. At the end of the fermentation, the target OVA protein was harvested from the supernatant.

Cell debris was removed, protein was purified and lyophilized to a dry powder. The OVA produced was used in the examples described below.

### Example 11: Fermentation and Production of rOVA

Fermentation: Strains for fermenting recombinant OVA (rOVA) were each cultured in a bioreactor at ambient conditions. A seed train for the fermentation process began with the inoculation of shake flasks with liquid growth broth. The inoculated shake flasks were kept in a shaker after which the grown P. pastoris was transferred to a production-scale reactor.

To expand production, a seed vial of rOVA P. pastoris seed strain was removed from cryo-storage and thawed to room temperature. Contents of the thawed seed vials were used to inoculate liquid seed culture media in baffled flasks which were grown at 30° C. in shaking incubators. These seed flasks were then transferred and grown in a series of larger and larger seed fermenters (number to vary depending on scale) containing a basal salt media, trace metals, and glucose. Temperature in the seed reactors was controlled at 30° C., pH at 5, and dissolved oxygen (DO) at 30%. pH was maintained by feeding ammonia hydroxide, which also acted as a nitrogen source. Once sufficient cell mass was reached, the grown rOVA P. pastoris was inoculated into a production-scale reactor containing basal salt media, trace metals, and glucose.

Like in the seed tanks, the culture was also controlled at 30° C., pH5 and 30% DO throughout the process. pH was again maintained by feeding ammonia hydroxide. During the initial batch glucose phase, the culture was left to consume all glucose and subsequently-produced ethanol. Once the target cell density was achieved and glucose and ethanol concentrations were confirmed to be zero, the glucose fed-batch growth phase was initiated. In this phase, glucose was fed until the culture reached a target cell density. Glucose was fed at a limiting rate to prevent ethanol from building up in the presence of non-zero glucose concentrations. In the final induction phase, the culture was co-fed glucose and methanol which induced it to produce rOVA via the pAOX promoters. Glucose was fed at an amount to produce a desired growth rate, while methanol was fed to maintain the methanol concentration at 1% to ensure that expression was consistently induced. Regular samples were taken throughout the fermentation process for analyses of specific process parameters (e.g., cell density, glucose/methanol concentrations, product titer, and quality). After a designated amount of fermentation time, secreted rOVA was collected and transferred for downstream processing.

The fermentation broth containing the secreted rOVA was subjected to centrifugation at 12,000 rpm. The supernatant was clarified using microfiltration. To concentrate the protein and remove excess water, ultrafiltration at room temperature was used. An appropriately sized filter was used to retain the target rOVA while the compounds, salts, and water smaller than rOVA passed through the filter. To reduce the final salt content and conductivity in preparation for chromatography, the concentrated rOVA retentate was dialyzed at pH 3.5 until the final conductivity of the material was 1.7 mS/cm. The bulk of the purification was done using cation exchange chromatography at pH 3.5. Citrate buffer containing a high salt concentration of sodium chloride was used to elute the bound rOVA from the resin. To remove the excess salts, the eluant was finally dialyzed to make a final protein solution containing about 5-10% protein and 85-95% water. The final solution was sterilized by passing it through a 0.2 um bioburden filter. The water was evaporated using a spray dryer/lyophilizer at appropriate temperatures to produce a final powder containing about 80% protein. Duck and ostrich OVAs were produced in similar systems.

### Example 12: Preparation of Solubilized rOVA

In this example, hydrophobic recombinant chicken rOVA was solubilized and passed through a 0.2 μm filter.

Recombinant rOVA was purified through ion exchange chromatography at pH 3.5 and was found to be insoluble. Sodium hydroxide was added to the solution to change the pH to 12.5 and solubilize the rOVA. The rOVA solution at pH 12.5 was passed through a 0.2 μm filter. Following filtration, the pH was returned to 6.5 using hydrochloric acid and the rOVA was spray dried or lyophilized. This dried chicken rOVA was then used in the Examples below.

### Example 13: Glycosylation of Gallus gallus rOVA

In this example, Pichia-secreted rOVA was analyzed for glycosylation patterns.

Native ovalbumin (nOVA) has two potential N-linked glycosylation sites (FIG. 2A). A single site of glycosylation at Asn-292 is found in the egg white. MALDI-TOF analysis has shown that the typical glycans on native OVA are organized as (Man)5(GlcNAc)5(Gal)1 (FIG. 2A) (Harvey et al., 2000). Analysis of glycans on rOVA showed a typical glycosylation pattern shown in (FIG. 2B).

Pichia secreted chicken rOVA from the above Example was analyzed by gel electrophoresis migration and observed in three distinct forms (three white arrows pointing to rOVA in the “Input” lane below a) glycosylation-free, b) mono-glycosylated and c) di-glycosylated. Both the mono- and di-glycosylated glycosyl chains were cleaved from the mature rOVA protein using either of the endoglycanases EndoH or PNGaseF. Both the “denatured” or “native” deglycosylation protocols were used (as described in the NEB catalog). The green arrow indicates exogenous EndoH and the purple arrow indicates exogenous PNGaseF added to the in vitro reactions (FIG. 2C).

Pichia secreted chicken rOVA was subjected to standard analysis using Mass spectrometry. It was found to have five versions of N-linked Glycans (ManGlcNAc): high-mannose glycans of Man9 (˜40%), Man10 (˜47%) or Man11 (˜13%) type of N-glycan structures (FIG. 2D).

### Example 14: Comparison of Foaming rOVA and rOVD Solutions

Recombinant chicken ovomucoid (rOVD) and recombinant chicken ovalbumin (rOVA) were each expressed and purified as shown in the above examples. rOVA, rOVD and combinations of rOVA and rOVD were compared to fresh egg white and evaluated for properties of foaming ability and foam retention.

Lyophilized protein samples were blended into aqueous solution (distilled water) at different concentrations and pHs. Protein solutions were created for each 4% w/w OVA, 4% w/w OVA+8% w/w OVD, 7% nOVD, 7% rOVD, 7% w/w OVA, 7% w/w OVA+5% w/w OVD, 12% nOVD, 12% rOVD, Fresh Egg White (12% w/w protein), and 12% w/w OVA. The solutions' foaming ability and foaming retention was assessed.

The samples were divided into 5 mL aliquots to be tested for foam capacity and stability. Each 5 mL aliquot was pipetted into a beaker and whipped using the Dremel® on speed 3. After a stiff foam was achieved, the foaming time was recorded as well as the initial volume of the foam. Foam capacity was determined by measuring the initial volume of foam following the whipping and compare against the initial volume of 5 mL. Foam Capacity (%)=(volume of foam/initial volume)*100.

The drainage was measured in 10-minute increments for 30 minutes to gather data for foam stability. The drained volume after 30 minutes was compared to the initial liquid volume (5 mL). Foam Stability (%): (Initial volume−drained volume)/initial volume*100.

Results for foaming time, capacity and stability for rOVD, rOVA, various combinations of rOVD and rOVA and egg white are shown in Table 7 below. The combinations of rOVD and rOVA outperformed not only rOVD alone and rOVA alone, they also performed better than the chicken egg white. The combinations of rOVD and rOVA showed a higher foaming capacity and a higher foam stability and they needed less time to generate the foam. The combinations of rOVD and rOVA presented herein are not found naturally in an egg white and thus show the unexpected effect of combining two or more recombinant egg white proteins.

TABLE 7
Foaming Parameters

| | | Foaming | Foam | Time Spent |
|---|---|---|---|---|
| | | Capacity | Stability | Foaming |
| Protein Combination | pH | (%) | (%) | (s) |
| 4% OVA | 6.05 | 333 | 57 | 25 |
| 4% OVA + 8% OVD | 6 | 444 | 84 | 19 |
| 7% nOVD | 6.00 | 437.5 ± 17.7 | 25 ± 0 | 16 ± 1.4 |
| 7% rOVD | 6.01 | 450 | 8 | 15 |
| 7% OVA | 6.03 | 333 | 66 | 19 |
| 7% OVA + 5% OVD | 6.05 | 438 | 90 | 17 |
| 12% OVA | 6.05 | 313 | 69 | 18 |
| 12% nOVD | 6.01 | 362.5 ± 17.7 | 10 ± 7.1 | 14.5 ± 0.7 |
| 12% rOVD | 6.01 | 444 | 11 | 16 |
| Fresh Egg White | 9.01 | 268 | 77 | 67 |
| Fresh Egg White | 9.01 | 313 | 64 | 30 |
| (12% protein) | | | | |

### Example 15: Comparison of Emulsified Products

In this example, recombinant chicken ovomucoid (rOVD) and recombinant chicken ovalbumin (rOVA) were made using the above-described examples. rOVA, rOVD and combinations of rOVA and rOVD were compared to fresh egg white and evaluated for emulsification functionality in a salad dressing application.

Water, vinegar and the protein powder of interest were mixed in a kitchen mixer for 30 seconds. The proteins of interest included, egg-white protein powder, rOVD alone, rOVA alone, and the combination of rOVA and rOVD. Oil was added gradually over 30 seconds into this mixture while mixing. Mixing was continued for additional 2.5 minutes. Using a L5M-A Silverson mixer, the mixture was homogenized with the Square Hole shear head (SQSH) mixer for 9 min at 4000 rpm. List of some of the illustrative ingredients and their respective concentrations are provided in Table 8 below.

TABLE 8
List of Ingredients.

| | | | | 4% | |
|---|---|---|---|---|---|
| | Egg | | | rOVA | |
| | white | | | & | |
| | protein | rOVA | rOVD | 4% | Negative |
| | 8% | 8% | 8% | rOVD | control |
| Ingredient | % | % | % | % | % |
| Canola oil | 30 | 30 | 30 | 30 | 30 |
| Water | 54.67 | 55.30 | 55.21 | 55.26 | 64 |
| Vinegar | 6 | 6 | 6 | 6 | 6 |
| Emulsifier | 9.33 | 8.70 | 8.79 | 8.75 | 0 |
| Protein | | | | | |
| Total | 100 | 100 | 100 | 100 | 100 |

Emulsion stability was assessed visually by capturing the observed separation of phases pictorially. The samples were refrigerated at a temperature (4° C.) for 3 days. Results are shown in FIG. 3. On day 0, all samples except the negative control showed good emulsification properties. Thereafter, the samples were refrigerated to monitor stability. All samples showed separation of the phases including egg-white protein powder. Combinations of rOVD and rOVA showed emulsion properties comparable to egg-white protein powder for all time points.

On day 0, all samples except the negative control showed good emulsification properties. Thereafter, the samples were refrigerated to monitor stability. All samples showed separation of the phases including egg-white protein powder. rOVD outperformed both nOVD and fresh egg whites in foaming capacity at least. Combinations of rOVD and rOVA showed emulsion properties comparable to egg-white protein powder for all time points.

### Example 16: Comparison of Film Formation

In this example, recombinant chicken ovomucoid (rOVD) and recombinant chicken ovalbumin (rOVA) were made using the above-described examples. rOVA, rOVD and combinations of rOVA and rOVD were compared to egg white and evaluated for film formation and sheen formation in a bread application were evaluated.

In a small container, yeast and sugar were mixed together and warm water (85-95 F) was added. Water was then added in a kitchen aid bowl. Mixture was mixed until a dough was formed. Dough was kneaded until the desired consistency was achieved. Dough was greased and placed in a bowl and left to rise for 45 minutes at 80F proofing temperature (1st proof). Dough was turned out onto a floured board and knead out air (fold 7 times). A mini loaf was shaped and placed in a greased mini pan. The dough was covered and risen for 30 minutes at room temperature (2nd proof). Appropriate wash was applied on top of the dough balls at a 3% level.

TABLE 9
List of Ingredients and their proportions used in bread formulation:

| Ingredients | % |
|---|---|
| DI Water | 41.77 |
| Granulated Sugar | 2.94 |
| Bakers Yeast | 1 |
| All-Purpose Flour | 53.62 |
| Salt | 0.67 |
| Total | 100.00 |

3% wash of total bread dough weight was added on top. 25 g samples each were used (total egg wash = 0.75 g).

The following formulations were used for protein of interest: (protein at 8% usage level was tested).

TABLE 10
List of Ingredients and their proportions used in wash formulation:

| | EWP (egg | | | | |
|---|---|---|---|---|---|
| Sample/ | white | | | | |
| Ingredient | powder) | rOVD | rOVA | Water | Total |
| Egg white | 9.33 | | | 90.67 | 100 |
| powder | | | | | |
| 8% rOVD | | 8.79 | | 91.21 | 100 |
| 8% rOVA | | 8.7 | | 91.30 | 100 |
| 4% rOVD | | 4.4 | 4.35 | 91.26 | 100 |
| 4% rOVA | | | | | |
| 5% rOVD | | 5.49 | 3.26 | 91.24 | 100 |
| 3% rOVA | | | | | |
| 7% rOVD | | 7.69 | 1.09 | 91.22 | 100 |
| 1% rOVA | | | | | |

For samples with commercial egg white sheen, 0.75 g of each sample was applied to the dough surface. Bake at 350 F for 8 minutes or until golden brown (switch the location of the bread at 4 min to achieve even baking on all samples). Individual sample pictures were analyzed for color data in the RGB spectrum using the ColorName application. Sample values were generated using a 2×2 cm cross-section taken from the center of the bread surface. RGB data was then converted to a CIELAB system using the online software www.colormine.org. CIELAB model is a color space system that expresses color in 3 values:
1) L* for the lightness from black (0) to white (100)
2) a* from green (−) to red (+),
3) b* from blue (−) to yellow (+)

TABLE 11
CIELAB results for bread post baking:

| | L* | a* | b* |
|---|---|---|---|
| Negative Control | 63.669 | 1.10972 | 25.4527 |
| Commercial egg | 68.349 | 0.04763 | 34.7033 |
| white substitute | | | |
| 8% Egg white protein | 76.831 | 2.58977 | 31.1123 |
| 8% rOVD | 83.591 | 4.58532 | 42.2485 |
| 8% rOVA | 80.135 | 3.24212 | 31.53948 |
| 4% rOVD | 86.945 | 0.94292 | 30.1951 |
| 4% rOVA | | | |
| 5% rOVD | 82.04 | 4.44288 | 39.6797 |
| 3% rOVA | | | |
| 7% rOVD | 82.43 | −8.31015 | 20.2132 |
| 1% rOVA | | | |

rOVD and egg white protein samples showed a higher L* value suggesting higher brightness or luminance. Control (no egg wash), egg white protein samples showed a low a* value suggesting lower redness or brownness as compared to rOVD samples. Higher inclusion of rOVD provides better sheen and better a* browning values as seen in 8% rOVD, followed by 5% rOVD-3% rOVA and 4% rOVD-4% rOVA. FIG. 4 shows illustrative samples for comparing film forming agents in a bread dough application.

The control sample looked pale, wrinkly and had no shine. The commercial egg wash substitute, 8% rOVD and 8% rOVA samples showed browning and sheen comparable to the 5% rOVD+3% rOVA and 4% rOVD+4% rOVA samples.

### Example 17: Comparison of Pound Cake Application

In this example, recombinant chicken ovomucoid (rOVD) and recombinant chicken ovalbumin (rOVA) were made using the above-described examples. rOVA, rOVD and combinations of rOVA and rOVD were compared to fresh whole egg in the classic pound cake system. In this experiment the ratio of rOVA to rOVD is 1.4.

TABLE 12
List of Ingredients and their proportions used to create pound cake.

| | Percentage in 100 g recipe | | |
|---|---|---|---|
| | Fresh | | |
| | whole | | rOVA + |
| Ingredients | egg | rOVA | rOVD |
| Unbleached all-purpose flour | 23.34 | 22.93 | 22.93 |
| (includes hard red wheat flour, | | | |
| Malted barley flour; 11.7% protein) | | | |
| Granulated Sugar | 23.34 | 22.93 | 22.93 |
| Unsalted butter (includes cream | 23.34 | 22.93 | 22.93 |
| from milk, Natural flavorings) | | | |
| Fresh whole egg | 23.34 | 0 | 0 |
| Sour cream (includes cultured | 5.2 | 5.10 | 5.10 |
| cream) | | | |
| Baking powder | 1.25 | 1.22 | 1.22 |
| (includes Monocalcium Phosphate, | | | |
| Na Bicarbonate, Cornstarch) | | | |
| Salt (includes Salt, Ca silicate) | 0.18 | 0.18 | 0.18 |
| rOVA | 0 | 3.40 | 1.98 |
| rOVD | 0 | 0 | 1.44 |
| Emfix K 02 (modified potato starch) | 0 | 1.36 | 1.36 |
| Keltrol F (xanthan gum) | 0 | 0.15 | 0.15 |
| Sunlec 25 (Sunflower lecithin) | 0 | 0.34 | 0.34 |
| Pineapple yellow AET color | 0 | 0.095 | 0.095 |
| Water | 0 | 19.33 | 19.33 |
| Total | 100 | 100 | 100 |

Batter preparation was as follows: Preheated the oven to 325° F. Butter (it has to be at was beat at room temperature, but not too soft: around 70F) and sugar was added until incorporating more air into the mix and becoming pale (about 1 minute at high speed (7); time of whipping was adjusted based on the batter volume). In the pound cake with rOVA/rOVD, Sunlec 25 was added in this step. Reduced the speed to medium (5) and gradually eggs and/or reconstituted the rOVA. Emfix K 02 and Keltrol F in DI water (color is added to the water) and beat thoroughly until creating a creamy emulsion to hold the air bubbles (around 1:30 minutes).

In a bowl, whisked flour, baking powder and salt. Reduced the speed to low (1). To maintain emulsion from breaking add flour mixture in 2 batches (alternating with sour cream), beating until just incorporated (about 1 minute). Baked for 24 minutes at 325° F. and around 3-4 minutes at 320° F. or until a toothpick inserted into the center of each cake came out clean (with a few crumbs), and cakes looked golden brown. Transferred pans to a wire rack to cool around 10 minutes. Then, turned out cakes onto the rack to cool completely.

The texture of the cakes was evaluated using Brookfield CT3 Texture Analyzer. The cake samples were cut into 15×15×20 mm cubes. Upper dome, bottom layer and crust sides were removed from all samples before analysing. A double-cycle program was used to compress the samples at a test speed of 1 mm/s with 5 mm distance and 10 g trigger load. In terms of sensory analysis, two sensory attributes (appearance and aroma) were evaluated. Results are presented in Table 13 and FIG. 5.

TABLE 13
The texture analysis and sensory results.

| | Fresh whole egg | ROVA | rOVA + rOVD |
|---|---|---|---|
| Hardness | 79.8 ± 2.14 a | 95.3 ± 32.7 a | 108.4 ± 12.1 a |
| Resilience | 0.39 ± 0.02 a | 0.28 ± 0.02 b | 0.31 ± 0.01cb |
| Cohesiveness | 0.69 ± 0.01 a | 0.54 ± 0.4 b | 0.59 ± 0.02cb |
| Springness | 3.82 ± 0.06 a | 3.18 ± 0.4 b | 3.58 ± 0.11ab |
| chewiness | 2.05 ± 0.08 a | 1.64 ± 0.8 a | 2.24 ± 0.15a |
| Weight loss % | 15.21 ± 0.36a | 12.47 ± 0.23b | 12.64 ± 0.7cb |
| batter density (g/ml) | 1.1 | 1.07 | 1.08 |
| Cake height (mm) | 32.53 ± 0.87a | 32.34 ± 0.86a | 31.6 ± 0.920a |
| Appearance | ice pale yellow crumb | open pores structure but | open pores structure, |
| | color, light brown | slightly looks pasty (needs | slightly pasty interior |
| | crust color, open | to be cooked more), | (needs to be cooked more), |
| | pores structure (a few | crumb: slightly intense | slightly intense yellow |
| | large air bubbles) | yellow color, crust: pale | color, pale brown crust, |
| | | brown color on surface, | more cohesive than rOV |
| | | looks less cohesive, fall | cakeA, more moist than |
| | | apart | egg |
| Aroma | Eggy, cakey | sweet, buttery, lack of | sweet, buttery cakey smell |
| | | eggy smell | |

*Data that does not share the same letter for a specific attribute, is significantly different from
each other (p < 0.05) using Tukey-HSD test; Mean ± Std Dev.

In terms of moisture loss during baking, cakes formulated with rOVA and rOVA/rOVA indicated significantly lower weight loss compared to the egg control.

The average of three standing height points on the cakes (measuring by EZ Cal caliper) indicated no significant difference between the samples.

In terms of hardness and chewiness, no significant differences were found between the cakes formulated with egg and proteins.

Cake made by fresh whole eggs showed higher levels of resilience; however, the resilience values for the rOVA and rOVA/rOVD cakes were similar.

Numerically, cake with rOVA/rOVD was more cohesive and springier compared to rOVA cake.

### Example 18: Comparison of Meringue Application

In this example, recombinant chicken ovomucoid (rOVD) and recombinant chicken ovalbumin (rOVA) were made using the above-described examples. rOVA, rOVD and combinations of rOVA and rOVD were compared to fresh whole egg in a meringue system.

Egg whites were separated from the egg yolk carefully at the refrigerator temperature and let egg whites get to the room temperature before whipping. rOVA powder. Sodium lauryl sulfate (SLS). Xanthan gum and Triethyl Citrate (TEC) were reconstituted in DI water at the room temperature. Transferred to the mixer bowl and whipped for 30 seconds at speed 5 (to obtain a homogeneous solution), then mixed at speed 8 until soft peaks formed. While beating constantly, sugar was added gradually and beat at high speed after each addition until sugar was dissolved before adding the next. Mixing was continued until a glossy and firm peak was formed. Preheated oven (Breville BOV800XL Smart Electric Oven) to 200° F.; meringues were baked for 70 minutes (or until light and crisp but not brown. After cooling, meringues were stored tan airtight container. Whipping time to produce firm foam for each protein solution was recorded. Results are presented in Table 15 and FIG. 6.

TABLE 14
Formulations

| | | rOVA8.3% + SLS + | | rOVA8.3% + TEC + | | rOVA 7% + rOVD 5% | |
|---|---|---|---|---|---|---|---|
| Fresh egg white | | Xanthan gum | | Xanthan gum | | (w/w based on protein) | |
| Ingredients | % | Ingredients | % | Ingredients | % | Ingredients | % |
| Fresh egg white | 38.1 | rOVA | 9.5 | rOVA | 9.5 | rOVA | 7.0 |
| Sugar | 29.4 | Sugar | 29 | Sugar | 29 | rOVD | 5.0 |
| — | — | Water | 61.3 | Water | 61.3 | sugar | 29 |
| — | — | Xanthan gum | 0.1 | Xanthan gum | 0.1 | water | 59 |
| — | — | SLS | 0.1 | TEC | 0.048 | — | — |
| Total weight | 100 | Total weight | 100 | Total weight | 100 | Total weight | 100 |

TABLE 15
Results:

| | | rOVA 8.3% + | rOVA 8.3% + | |
|---|---|---|---|---|
| | | SLS + Xanthan | TEC + | rOVA 7% + |
| Parameter | Fresh egg white | gum | Xanthan gum | roVD 5% |
| weight loss % | 60 ± 2a* | 60 ± 1.1a | 58 ± 2.5a | 57 ± 2a |
| volume (ml) | 7 ± 1.5 | 7.3 ± 1.5 | 7.9 ± 2 | |
| foam density (g/ml) | 0.19 | 0.2 | 0.22 | 0.3 |
| Meringue density | 0.056 ± 0.014a | 0.074 ± 0.02a | 0.064 ± 0.018a | 0.04 ± 0.007a |
| (g/ml) | | | | |

*Similar letters indicate that there is no significant difference between the values (p < 0.05; mean ± std dev). n = 6

rOVA and a combination of rOVD and rOVA produced meringue that is comparable to fresh egg w % bite sample in terms of physical parameters.

The appearance of rOVA and rOVD+rOVA meringues were visually better than fresh egg white controls. The ridges were more well defined in rOVA containing meringues and the samples were whiter compared to the fresh egg white control.

### Example 19: Foaming Capabilities of Recombinant Compositions

The following experiment was performed to compare foam capacity and stabilities of recombinant proteins versus egg white protein and an egg-white powder (EWP) substitute.

Materials:
a. OVA and/or OVD protein powder
b. DI water
c. 25 or 30 mL beaker
d. 25 mL beakers (one for each repetition; two repetitions per sample)
e. 15 mL Falcon tubes, conical (one for each repetition)
f. Timers (one for timing Dremel, another for timing repetitions)
g. pH meter
h. pH calibration tubes
i. Micropipette, p1000
j. Dremel 3000 variable speed rotary tool

### Protein Solution Preparation

For each sample set, a 15 mL solution was made with the required % of protein.
Gently mix with a thin spatula until completely dissolved.
pH and conductivity of the protein solution were measured for each sample set.
Transfer 4 mL of the protein solution into each 25 mL beaker.

### Dremel® Operation

For each repetition: Submerge the Dremel to the bottom of the 25 mL beaker and turn on speed 3. Immediately start a 25 second timer. Keeping the Dremel directly off the bottom of the beaker, spin the Dremel in medium sized circles for 25 seconds.
After the 25 seconds, start a timer for 10 minutes (time until first drainage measurement).
Squeeze the top and bottom of the Dremel wings against the rim of the beaker to recover as much foam as possible.
Using the Dremel wings, push down on the foam gently a couple of times to flatten the foam. Use a q tip to carefully prod the center of the foam to ensure there are no large air bubbles trapped inside. Flatten the foam as much as possible and clean the beaker walls down to the level of the foam.
Record the volume of the foam.
Take a picture of the beaker at eye level against a black background.
During the 10-minute interval of the first repetition, repeat steps 1-6 with the second repetition. There should be an independent timer going for each repetition.
   a. Clean the Dremel between repetitions. Spin into a beaker of DI water, then wipe down with a paper towel.

Foam Capacity(%)=foam volume×100/initial volume of solution

### Drainage

After 10 minutes has passed, flip the beaker over 1800 and feed drainage into a falcon tube for 30 seconds. To streamline this, restart the same timer for another 10 minutes, and then use the first 30 seconds that elapse to keep track of drainage time.
Record the volume of the drainage. Read the volume of drainage at the meniscus (may help to hold up against/toward a light source). Use the labeled standard as reference.
Repeat steps 1-2 after each 10-minute interval has passed. Continue until data for 30 minutes have been recorded.

Foam Stability(%)=(initial solution volume−volume drained at 30 min)×100/initial solution volume

TABLE 16
Proteins Used

| Protein | Protein content |
|---|---|
| OVA | 87.3% |
| OVD | 90.4% |
| Ostrich OVA (oOVA) | 81% |
| Duck OVA (dOVA) | 87.3% |

The average and standard deviation of each sample set was analyzed. Results are presented in Tables 17 (foam capacity) and 18 (foam stability) and FIG. 7.

TABLE 17
Foam Capacity

| | Average Foam | STD | | |
|---|---|---|---|---|
| Sample | Capacity (%) | DEV | pH | Conductivity |
| OVA 12% | 416.67 | 14.43 | | |
| OVA 11% + OVD 1% | 562.5 | 53.03 | 6.07 | 1.974 |
| OVA 10% + OVD 2% | 625 | 0 | 5.91 | 1.781 |
| OVA 9% + OVD 3% | 600 | 0 | 5.5 | 1.753 |
| OVA 8% + OVD 4% | 625 | 0 | 5.25 | 1.619 |
| OVA 7% + OVD 5% | 600 | 0 | 5.25 | 1.323 |
| OVA 6% + OVD 6% | 637.5 | 17.68 | 5.07 | 1.338 |
| OVA 5% + OVD 7% | 650 | 0 | 4.95 | 1.266 |
| OVA 4% + OVD 8% | 600 | 35.36 | 4.85 | 1.154 |
| OVA 3% + 9% | 662.5 | 17.68 | 4.71 | 1.02 |
| OVA 2% + OVD 10% | 712.5 | 53.03 | 4.61 | 0.933 |
| OVA 1% + OVD 11% | 712.5 | 53.03 | 4.51 | 0.821 |
| OVD 12% | 600 | 35.36 | 4.51 | 0.734 |
| Fresh Egg White | 312.5 | 0.18 | 9.13 | |
| EWP 12% | 350 | 0 | 6.82 | |
| OVA 20% | 162.5 | 17.68 | 6.31 | 1.84 |
| OVA 15% | 462.5 | 53.03 | 6.25 | 1.732 |
| OVA 6% | 425 | 70.71 | 6.05 | 1.016 |
| OVD 20% | 412.5 | 17.68 | 4.25 | 0.896 |
| OVD 15% | 587.5 | 17.68 | 4.35 | 0.804 |
| OVD 6% | 700 | 0 | 4.3 | 0.47 |
| oOVA 6% | 600 | 35.36 | 3.05 | 0,618 |
| dOVA 6% | 475 | 35.36 | 3.17 | 0.667 |
| oOVA 3% + OVD 3% | 712.5 | 17.68 | 3.7 | 0.531 |
| dOVA 3% + OVD 3% | 662.5 | 53.03 | 3.71 | 0.518 |
| OVA 9% + OVD 6% | 625 | 0 | 5.04 | 1.512 |
| OVD 2% + OVA 2% | 637.5 | 17.68 | 4.93 | 0.661 |
| OVD 3% + OVA 3% | 662.5 | 17.68 | 4.88 | 0.966 |
| OVD 10% + OVA 10% | 525 | 0 | 4.9 | 0.968 |

TABLE 18
Foam Stability

| Sample | Average Foam Stability (%) | STD DEV |
|---|---|---|
| OVA 12% | 61.67 | 0.04 |
| OVA 11% + OVD 1% | 97.5 | 0.04 |
| OVA 10% + OVD 2% | 100 | 0 |
| OVA 9% + OVD 3% | 100 | 0 |
| OVA 8% + OVD 4% | 100 | 0 |
| OVA 7% + OVD 5% | 100 | 0 |
| OVA 6% + OVD 6% | 100 | 0 |
| OVA 5% + OVD 7% | 100 | 0 |
| OVA 4% + OVD 8% | 100 | 0 |
| OVA 3% + 9% | 93.75 | 0.09 |
| OVA 2% + OVD 10% | 78.13 | 0.04 |
| OVA 1% + OVD 11% | 80 | 0.04 |
| OVD 12% | 0 | 0 |
| Fresh Egg White | 66.25 | 0.02 |
| EWP 12% | 72.5 | 0.04 |
| OVA 20% | 100 | 0 |
| OVA 15% | 100 | 0 |
| OVA 6% | 45 | 0.11 |
| OVD 20% | 0 | 0 |
| OVD 15% | 0 | 0 |
| OVA 6% | 0 | 0 |
| oOVA 6% | 48.75 | 0.02 |
| dOVA 6% | 52.5 | 0 |
| oOVA 3% + OVD 3% | 56.25 | 0.09 |
| dOVA 3% + OVD 3% | 88.75 | 0.02 |
| OVA 9% + OVD 6% | 100 | 0 |
| OVD 2% + OVA 2% | 97.5 | 0.04 |
| OVD 3% + OVA 3% | 100 | 0 |
| OVD 10% + OVA 10% | 88.75 | 0.02 |

Foam capacity and stability of fresh egg white were normalized to 100% and the other data points were calculated as fold foam capacity and fold foam stability relative to fresh egg white. FIG. 5 illustrates the results.

Surprisingly, all concentrations tested for rOVD had a substantially higher foam capacity as compared to fresh egg-white or EWP. Even at concentrations as low as 6% (nearly half of the concentration of total protein in a natural egg white) the foam capacity was more than double of the foam capacity of fresh egg white. Additionally, the foam produced with rOVD alone showed no foam stability.

The results also show that up to 15% the foam capacity of rOVA alone was also higher than the foam capacity of fresh egg white. Unexpectedly, the rOVA solution shows reduced foam capacity at 20% but a higher foam stability of the foam produced. These concentrations of OVA and OVD are not naturally found and are not expected to show properties at such different concentrations. Furthermore, the combinations of these two proteins illustrate an unexpected synergy at all concentrations tested. rOVD alone provides little to no foam stability but in combination with rOVA the foam stability of the solution increased significantly. At concentrations as low as 4% the foam produced was 1.5 folds more stable than fresh egg white and presented a 2 hold higher foam capacity. The foam capacity and stability of rOVD+rOVA solutions remain higher than fresh egg whites. Adding 1% rOVD to a solution comprising 11% rOVA not only increased the foam capacity significantly but also improved foam stability. Similar results were shown for duck and ostrich OVA where the foam capacity and stability were higher even than the chicken OVA.

Compositions comprising rOVD and rOVA in combination or alone may be used as ingredients to produce various types of compositions such as described herein and provide improved properties as compared to fresh egg white or egg white substitutes.

### Example 20: Foaming Capabilities of Recombinant Compositions

The following experiment was performed to compare density of compositions comprising recombinant proteins versus egg white protein and an egg-white powder (EWP) substitute. Recombinant chicken OVD and OVA protein solutions or fresh egg white (4 mL) were foamed for 25 sec using Dremel machine at setting 3. The foam formed was scooped into a cylinder (35 mL capacity) with minimal air pockets and smoothed to the rim. The tared weight of the foam was recorded. Foam density was calculated as follows:

Foam density(g/mL)=foam weight(g)/35(mL)

Results of density measurements are provided in Table 19 below:

TABLE 19
Density measurements

| Sample | Foam Density (g/mL) |
|---|---|
| 6% OVA 6% OVD | 20.43 |
| 3% OVA 3% OVD | 20.95 |
| 2% OVA 2% OVD | 21.50 |
| Fresh Egg White | 38.02 |

As shown in Table 19, the foam density of rOVD and rOVA protein solutions was less than the foam density of fresh egg white. As Example 19 describes, the foam stability and capacity of rOVD and rOVA protein solutions were higher and provided a less dense foam.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A foam composition comprising a protein component, wherein the protein component comprises a mixture of recombinantly produced ovomucoid (rOVD) protein and recombinantly produced ovalbumin (rOVA) protein, wherein the foam composition has a foam capacity and a foam stability comparable to or higher than the foam capacity and the foam stability of a control composition that comprises similar contents by identity and quantity as the foam composition except the control composition's protein component is one of: chicken egg-white or an egg white substitute; ovomucoid alone; or ovalbumin alone.

2. The foam composition of claim 1, wherein the protein component consists essentially of a mixture of the rOVD and the rOVA.

3. The foam composition of claim 1, wherein the protein component comprises from about 2% to about 30% w/w of the foam composition, about 4% to about 25% w/w of the foam composition, about 4% to about 20% w/w of the foam composition, or about 3% to about 20% w/w of the foam composition.

4-6. (canceled)

7. The foam composition of claim 1, wherein the protein component comprises from about 0.1% to about 99.5% rOVD w/w of the protein component.

8. The foam composition of claim 1, wherein the protein component comprises from about 0.1% to about 99.5% rOVA w/w of the protein component.

9. The foam composition of claim 1, wherein rOVD is from about 0.1% to about 20% w/w of the foam composition, about 0.1% to about 10% w/w of the foam composition, about 0.1% to about 5% w/w of the foam composition, about 0.1% to about 2% w/w of the foam composition, about 0.1% to about 1% w/w of the foam composition, about 0.1% to about 20% w/w of the foam composition, about 0.1% to about 10% w/w of the foam composition, about 0.1% to about 5% w/w of the foam composition, about 0.1% to about 2% w/w of the foam composition, or about 0.1% to about 1% w/w of the foam composition.

10-18. (canceled)

19. The foam composition of claim 1, wherein the foam composition comprises at least 1% rOVD w/w and at least 1% rOVA w/w.

20. (canceled)

21. The foam composition of claim 1, wherein a ratio of rOVD to rOVA in the protein component is from 1:20 to 20:1, 1:15 to 15:1, 1:10 to 10:1, 1:5 to 5:1, 1:3 to 3:1, 1:2 to 2:1, or 1:1.

22. The foam composition of claim 1, wherein the foam composition comprises a solvent wherein the solvent is water or another consumable liquid.

23-24. (canceled)

25. The foam composition of claim 22, wherein the other consumable liquid is a beverage.

26. The foam composition of claim 1, wherein the foam composition comprises a solvent, a protein component, and one or more components selected from a preservative, flavorant, salt, sweetener, acid, alcohol, fat or oil, stabilizer, and colorant.

27. The foam composition of claim 1, wherein the rOVD has a glycosylation pattern different from the glycosylation pattern of an ovomucoid obtained from a chicken egg, and/or wherein the rOVD protein comprises at least one glycosylated asparagine residue and the rOVD is substantially devoid of N-linked mannosylation, and wherein each glycosylated asparagine comprises a single N-acetylglucosamine.

28-29. (canceled)

30. The foam composition of claim 27, wherein the rOVD comprises at least three glycosylated asparagine residues.

31. The foam composition of claim 1, wherein the rOVD provides protein fortification to the foam composition and provides an improvement to at least one additional feature selected from the group consisting of solubility, mouthfeel, texture, thickness, stability to heat treatment, and stability to pH relative to the control composition.

32. The foam composition of claim 1, wherein the foam composition has sensory properties comparable to those of the control composition.

33. The foam composition of claim 1, wherein the rOVA has a glycosylation pattern different from an ovalbumin obtained from a chicken egg.

34. (canceled)

35. The foam composition of claim 1, wherein the rOVD and/or the rOVA is produced by a microbial host cell, wherein the microbial host cell is a yeast cell, a filamentous fungal cell, or a bacterial cell.

36. (canceled)

37. The foam composition of claim 35, wherein the microbial host cell is from a Pichia species, a Saccharomyces species, a Trichoderma species, a Pseudomonas species or an E. coli species.

38. The foam composition of claim 1, wherein the protein component comprises one or more non-egg white proteins.

39. The foam composition of claim 1, wherein the protein component does not comprise any egg white proteins other than rOVD and rOVA.

40. The foam composition of claim 1, wherein the rOVD has an amino acid sequence selected from any one of SEQ ID NOs: 1-44 and the rOVA has an amino acid sequence selected from any one of SEO ID NOs: 45-118.

41. (canceled)

42. An edible composition, wherein the edible composition comprises the foam composition of claim 1, wherein the edible composition comprises at least 0.1% of the foam composition w/w, wherein the composition is selected from: a coffee-drink, an alcoholic drink, a whipped cream composition, a frozen composition, or a dessert composition.

43-44. (canceled)

45. A method for making a foam composition, the method comprising
combining a solvent with the protein component as recited in claim 1 to obtain a liquid composition; and
aerating the liquid composition to obtain the foam composition.

46-62. (canceled)

63. A powder composition comprising a mixture of a recombinantly produced ovomucoid (rOVD) protein and a recombinantly produced ovalbumin (rOVA) protein, wherein the powder composition is capable of being solubilized and aerated to produce a foam composition that has a foam capacity and a foam stability comparable to or higher than the foam capacity and the foam stability of a control composition that comprises similar contents by identity and quantity as a control composition except the control composition's protein component is one of: chicken egg-white or an egg white substitute; ovomucoid alone; or ovalbumin alone.

64. The powder composition of claim 63, wherein the foam composition has a protein concentration of less than 20% w/w.

65. The powder composition of claim 63, wherein the powder has a protein component that consists essentially of rOVD and rOVA.

66. The powder composition of claim 63, wherein the powder comprises one or more additives, wherein the one or more additives are selected from: a filler or bulking agent, a flavorant, colorant, preservative, pH adjuster, powdered beverage mix, powdered juice mix, a sweetener, an amino acid, a protein, acidulant, dehydrated soup mix, dehydrated nutritional mix, dehydrated milk powder, caffeinated powder, or any combination thereof.

67. (canceled)

68. The powder composition of claim 63, wherein a protein content of the powder is at least 1% w/w.

69. The powder composition of claim 63, wherein a protein content of the powder is at most 99%.

70. The powder composition of claim 63, wherein rOVD is at least 5% w/w of the protein component at least 8% w/w of the protein component, at least 10% w/w of the protein component, at least 20% w/w of the protein component, at least 30% w/w of the protein component, at least 50% w/w of the protein component, at least 80% w/w of the protein component, or at least 90% w/w of the protein component.

71-77. (canceled)

78. The powder composition of claim 63, wherein rOVA is at least 5% w/w of the protein component at least 8% w/w of the protein component, at least 10% w/w of the protein component, at least 20% w/w of the protein component, at least 30% w/w of the protein component, at least 50% w/w of the protein component, at least 80% w/w of the protein component, or at least 90% w/w of the protein component.

79-85. (canceled)

86. The powder composition of claim 63, wherein a ratio of rOVD to rOVA in the protein component is from 1:20 to 20:1, 1:15 to 15:1, 1:10 to 10:1, 1:5 to 5:1, 1:3 to 3:1, 1:2 to 2:1, or 1:1.

87. The powder composition of claim 63, wherein the rOVD has a glycosylation pattern different from the glycosylation pattern of an ovomucoid obtained from a chicken egg and/or the the rOVD protein comprises at least one glycosylated asparagine residue and the rOVD is substantially devoid of N-linked mannosylation, and wherein each glycosylated asparagine comprises a single N-acetylglucosamine.

88-89. (canceled)

90. The powder composition of claim 87, wherein the rOVD comprises at least three glycosylated asparagine residues.

91. The powder composition of claim 63, wherein the powder composition has sensory properties comparable to those of the control composition.

92. The powder composition of claim 63, wherein the rOVA has a glycosylation pattern different from an ovalbumin obtained from a chicken egg.

93. (canceled)

94. The powder composition of claim 63, wherein the rOVD and/or the rOVA is produced by a microbial host cell, wherein the microbial host cell is a yeast cell, a filamentous fungal cell, or a bacterial cell.

95. (canceled)

96. The powder composition of claim 94, wherein the microbial host cell is from a Pichia species, a Saccharomyces species, a Trichoderma species, a Pseudomonas species or an E. coli species.

97. The powder composition of claim 63, wherein the protein component does not comprise any egg white proteins other than rOVD and rOVA.

98. The powder composition of claim 63, wherein the rOVD has an amino acid sequence selected from any one of SEQ ID NOs: 1-44 and the the rOVA has an amino acid sequence selected from any one of SEO ID NOs: 45-118.

99. (canceled)

100. The foam composition of claim 1, wherein the foam composition has a foam density that is less than a foam density of a control composition that comprises similar contents by identity and quantity as a control composition except the control composition's protein component is one of: chicken egg-white or an egg white substitute; ovomucoid alone; or ovalbumin alone.

101. The foam composition of claim 100, wherein the foam density is less than about 30 g/ml, is less than about 25 g/ml, or is less than about 20 g/ml.

102-104. (canceled)

105. An animal-free egg-white like composition having a protein component comprising a recombinantly-produced ovomucoid (rOVD) and a recombinantly-produced ovalbumin (rOVA), wherein the composition has a higher foam stability than a control composition that comprises similar contents by identity and quantity as the animal-free egg-like composition except the control composition's protein component is one of: chicken egg-white or an egg white substitute; ovomucoid alone; or ovalbumin alone.


SEQUENCE LISTING (120 sequences)

SEQ ID NO: 1 - protein, 186 aa, synthetic construct
  REGION 1..186: note = Synthetic polypeptide
AEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 2 - protein, 186 aa, synthetic construct
  REGION 1..186: note = Synthetic polypeptide
AEVDCSRFPNATDMEGKDVLVCNKDLRPICGTDGVTYTNDCLLCAYSVEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 3 - protein, 186 aa, synthetic construct
  REGION 1..186: note = Synthetic polypeptide
AEVDCSRFPNATDMEGKDVLVCNKDLRPICGTDGVTYTNDCLLCAYSVEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYMNKCNACNAVVESNGTLTLSHFGKC

SEQ ID NO: 4 - protein, 210 aa, synthetic construct
  REGION 1..210: note = Synthetic polypeptide
MAMAGVFVLFSFVLCGFLPDAAFGAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 5 - protein, 210 aa, synthetic construct
  REGION 1..210: note = Synthetic polypeptide
MAMAGVFVLFSFVLCGFLPDAVFGAEVDCSRFPNATDMEGKDVLVCNKDLRPICGTDGVTYTNDCLLCAYSVEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 6 - protein, 208 aa, synthetic construct
  REGION 1..208: note = Synthetic polypeptide
MAMAGVFVLFSFVLCGFLPDAAFGAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVDCSEYPKPDCTAEDRPLCGSDNKTYGNKCNFCNAVVESNGTLTLSHFGKC

SEQ ID NO: 7 - protein, 186 aa, synthetic construct
  REGION 1..186: note = Synthetic polypeptide
AEVDCSRFPNATDKEGKDVLVCNKDLRPICGTDGVTYNNECLLCAYSIEFGTNISKEHDGECKETVPMNCSSYANTTSEDGKVMVLCNRAFNPVCGTDGVTYDNECLLCAHKVEQGASVDKRHDGECRKELAAVSVDCSEYPKPDCTAEDRPLCGSDNKTYGNKCNFCNAVVESNGTLTLSHFGKC

SEQ ID NO: 8 - protein, 208 aa, synthetic construct
  REGION 1..208: note = Synthetic polypeptide
MAMAGVFVLFSFALCGFLPDAAFGVEVDCSRFPNATNEEGKDVLVCTEDLRPICGTDGVTYSNDCLLCAYNIEYGTNISKEHDGECREAVPVDCSRYPNMTSEEGKVLILCNKAFNPVCGTDGVTYDNECLLCAHNVEQGTSVGKKHDGECRKELAAVDCSEYPKPACTMEYRPLCGSDNKTYDNKCNFCNAVVESNGTLTLSHFGKC

SEQ ID NO: 9 - protein, 239 aa, synthetic construct
  REGION 1..239: note = Synthetic polypeptide
MQTITWRQPQGDHLRSRAPAATCRAGQYLTMAMAGIFVLFSFALCGFLPDAAFGVEVDCSRFPNTTNEEGKDVLVCTEDLRPICGTDGVTHSECLLCAYNIEYGTNISKEHDGECREAVPMDCSRYPNTTNEEGKVMILCNKALNPVCGTDGVTYDNECVLCAHNLEQGTSVGKKHDGGCRKELAAVSVDCSEYPKPACTLEYRPLCGSDNKTYGNKCNFCNAVVESNGTLTLSHFGKC

SEQ ID NO: 10 - protein, 185 aa, synthetic construct
  REGION 1..185: note = Synthetic polypeptide
VEVDCSRFPNTTNEEGKDVLVCTEDLRPICGTDGVTHSECLLCAYNIEYGTNISKEHDGECREAVPMDCSRYPNTTSEEGKVMILCNKALNPVCGTDGVTYDNECVLCAHNLEQGTSVGKKHDGECRKELAAVSVDCSEYPKPACTLEYRPLCGSDNKTYGNKCNFCNAVVESNGTLTLSHFGKC

SEQ ID NO: 11 - protein, 237 aa, synthetic construct
  REGION 1..237: note = Synthetic polypeptide
MQTITWRQPQGDHLRSRAPAATCRAGQYLTMAMAGIFVLFSFALCGFLPDAAFGVEVDCSRFPNTTNEEGKDVLVCTEDLRPICGTDGVTHSECLLCAYNIEYGTNISKEHDGECREAVPMDCSRYPNTTNEEGKVMILCNKALNPVCGTDGVTYDNECVLCAHNLEQGTSVGKKHDGGCRKELAAVDCSEYPKPACTLEYRPLCGSDNKTYGNKCNFCNAVVESNGTLTLSHFGKC

SEQ ID NO: 12 - protein, 128 aa, synthetic construct
  REGION 1..128: note = Synthetic polypeptide
EYGTNISIKHNGECKETVPMDCSRYANMTNEEGKVMMPCDRTYNPVCGTDGVTYDNECQLCAHNVEQGTSVDKKHDGVCGKELAAVSVDCSEYPKPECTAEERPICGSDNKTYGNKCNFCNAVVYVQP

SEQ ID NO: 13 - protein, 195 aa, synthetic construct
  REGION 1..195: note = Synthetic polypeptide
VDCSRFPNTTNEEGKDVLACTKELHPICGTDGVTYSNECLLCYYNIEYGTNISKEHDGECTEAVPVDCSRYPNTTSEEGKVLIPCNRDFNPVCGSDGVTYENECLLCAHNVEQGTSVGKKHDGGCRKEFAAVSVDCSEYPKPDCTLEYRPLCGSDNKTYASKCNFCNAVVIWEQEKNTRHHASHSVFFISARLVC

SEQ ID NO: 14 - protein, 339 aa, synthetic construct
  REGION 1..339: note = Synthetic polypeptide
MLPLGLREYGTNTSKEHDGECTEAVPVDCSRYPNTTSEEGKVRILCKKDINPVCGTDGVTYDNECLLCSHSVGQGASIDKKHDGGCRKEFAAVSVDCSEYPKPACMSEYRPLCGSDNKTYVNKCNFCNAVVYVQPWLHSRCRLPPTGTSFLGSEGRETSLLTSRATDLQVAGCTAISAMEATRAAALLGLVLLSSFCELSHLCFSQASCDVYRLSGSRNLACPRIFQPVCGTDNVTYPNECSLCRQMLRSRAVYKKHDGRCVKVDCTGYMRATGGLGTACSQQYSPLYATNGVIYSNKCTFCSAVANGEDIDLLAVKYPEEESWISVSPTPWRMLSAGA

SEQ ID NO: 15 - protein, 238 aa, synthetic construct
  REGION 1..238: note = Synthetic polypeptide
MSWWGIKPALERPSQEQSTSGQPVDSGSTSTTTMAGIFVLLSLVLCCFPDAAFGVEVDCSRFPNTTNEEGKEVLLCTKDLSPICGTDGVTYSNECLLCAYNIEYGTNISKDHDGECKEAVPVDCSTYPNMTNEEGKVMLVCNKMFSPVCGTDGVTYDNECMLCAHNVEQGTSVGKKYDGKCKKEVATVDCSDYPKPACTVEYMPLCGSDNKTYDNKCNFCNAVVDSNGTLTLSHFGKC

SEQ ID NO: 16 - protein, 284 aa, synthetic construct
  REGION 1..284: note = Synthetic polypeptide
MSSQNQLHRRRRPLPGGQDLNKYYWPHCTSDRFSWLLHVTAEQFRHCVCIYLQPALERPSQEQSTSGQPVDSGSTSTTTMAGIFVLLSLVLCCFPDAAFGVEVDCSRFPNTTNEEGKEVLLCTKDLSPICGTDGVTYSNECLLCAYNIEYGTNISKDHDGECKEAVPVDCSTYPNMTNEEGKVMLVCNKMFSPVCGTDGVTYDNECMLCAHNVEQGTSVGKKYDGKCKKEVATVDCSDYPKPACTVEYMPLCGSDNKTYDNKCNFCNAVVDSNGTLTLSHFGKC

SEQ ID NO: 17 - protein, 186 aa, synthetic construct
  REGION 1..186: note = Synthetic polypeptide
VEVDCSRFPNTTNEEGKDEVVCPDELRLICGTDGVTYNHECMLCFYNKEYGTNISKEQDGECGETVPMDCSRYPNTTSEDGKVTILCTKDFSFVCGTDGVTYDNECMLCAHNVVQGTSVGKKHDGECRKELAAVSVDCSEYPKPACPKDYRPVCGSDNKTYSNKCNFCNAVVESNGTLTLNHFGKC

SEQ ID NO: 18 - protein, 210 aa, synthetic construct
  REGION 1..210: note = Synthetic polypeptide
MAMAGVFLLFSFALCGFLPDAAFGVEVDCSRFPNTTNEEGKDEVVCPDELRLICGTDGVTYNHECMLCFYNKEYGTNISKEQDGECGETVPMDCSRYPNTTSEDGKVTILCTKDFSFVCGTDGVTYDNECMLCAHNIVQGTSVGKKHDGECRKELAAVSVDCSEYPKPACPKDYRPVCGSDNKTYSNKCNFCNAVVESNGTLTLNHFGKC

SEQ ID NO: 19 - protein, 205 aa, synthetic construct
  REGION 1..205: note = Synthetic polypeptide
MAGVFVLLSLVLCCFPDAAFGVEVDCSRFPNTTNEEGKDVLLCTKELSPVCGTDGVTYSNECLLCAYNIEYGTNISKDHDGECKEAVPADCSMYPNMTNEEGKMTLLCNKMFSPVCGTDGVTYDNECMLCAHNVEQGTSVGKKYDGKCKKEVATVDCSGYPKPACTMEYMPLCGSDNKTYGNKCNFCNAVVDSNGTLTLSHFGEC

SEQ ID NO: 20 - protein, 171 aa, synthetic construct
  REGION 1..171: note = Synthetic polypeptide
QVDCSRFPNTTNEEGKEVLLCTKELSPVCGTDGVTYSNECLLCAYNIEYGTNISKDHDGECKEAVPADCSMYPNMTNEEGKMTLLCNKMFSPVCGTDGVTYDNECMLCAHNVEQGTSVGKKYDGKCKKEVATVSVDCSGYPKPACTMEYMPLCGSDNKTYGNKCNFCNAVV

SEQ ID NO: 21 - protein, 207 aa, synthetic construct
  REGION 1..207: note = Synthetic polypeptide
MTMPGAFVVLSFVLCCFPDATFGVEVDCSTYPNTTNEEGKEVLVCSKILSPICGTDGVTYSNECLLCANNIEYGTNISKYHDGECKEFVPVNCSRYPNTTNEEGKVMLICNKDLSPVCGTDGVTYDNECLLCAHNLEPGTSVGKKYDGECKKEIATVDCSDYPKPVCSLESMPLCGSDNKTYSNKCNFCNAVVDSNETLTLSHFGKC

SEQ ID NO: 22 - protein, 207 aa, synthetic construct
  REGION 1..207: note = Synthetic polypeptide
MTMAGVFVLLSFALCCFPDAAFGVEVDCSTYPNTTNEEGKEVLVCTKILSPICGTDGVTYSNECLLCAYNIEYGTNVSKDHDGECKEVVPVDCSRYPNSTNEEGKVVMLCSKDLNPVCGTDGVTYDNECVLCAHNVESGTSVGKKYDGECKKETATVDCSDYPKPACTLEYMPFCGSDSKTYSNKCNFCNAVVDSNGTLTLSHFGKC

SEQ ID NO: 23 - protein, 207 aa, synthetic construct
  REGION 1..207: note = Synthetic polypeptide
MTTAGVFVLLSFALCSFPDAAFGVEVDCSTYPNTTNEEGKEVLVCTKILSPICGTDGVTYSNECLLCAYNIEYGTNVSKDHDGECKEFVPVDCSRYPNTTNEDGKVVLLCNKDLSPICGTDGVTYDNECLLCARNLEPGTSVGKKYDGECKKEIATVDCSDYPKPVCSLEYMPLCGSDSKTYSNKCNFCNAVVDSNGTLTLSHFGKC

SEQ ID NO: 24 - protein, 207 aa, synthetic construct
  REGION 1..207: note = Synthetic polypeptide
MTTAGVFVLLSFTLCSFPDAAFGVEVDCSPYPNTTNEEGKEVLVCNKILSPICGTDGVTYSNECLLCAYNLEYGTNISKDYDGECKEVAPVDCSRHPNTTNEEGKVELLCNKDLNPICGTNGVTYDNECLLCARNLESGTSIGKKYDGECKKEIATVDCSDYPKPVCTLEEMPLCGSDNKTYGNKCNFCNAVVDSNGTLTLSHFGKC

SEQ ID NO: 25 - protein, 207 aa, synthetic construct
  REGION 1..207: note = Synthetic polypeptide
MTTAVVFVLLSFALCCFPDAAFGVEVDCSTYPNSTNEEGKDVLVCPKILGPICGTDGVTYSNECLLCAYNIQYGTNVSKDHDGECKEIVPVDCSRYPNTTNEEGKVVFLCNKNFDPVCGTDGDTYDNECMLCARSLEPGTTVGKKHDGECKREIATVDCSDYPKPTCSAEDMPLCGSDSKTYSNKCNFCNAVVDSNGTLTLSRFGKC

SEQ ID NO: 26 - protein, 207 aa, synthetic construct
  REGION 1..207: note = Synthetic polypeptide
MTMTGVFVLLSFAICCFPDAAFGVEVDCSTYPNTTNEEGKEVLVCTKILSPICGTDGVTYSNECLLCAYNIEYGTNVSKDHDGECKEVVPVDCSKYPNTTNEEGKVVLLCSKDLSPVCGTDGVTYDNECLLCARNLEPGSSVGKKYDGECKKEIATIDCSDYPKPVCSLEYMPLCGSDSKTYDNKCNFCNAVVDSNGTLTLSHFGKC

SEQ ID NO: 27 - protein, 207 aa, synthetic construct
  REGION 1..207: note = Synthetic polypeptide
MTTAGVFVLLSFVLCCFPDAVFGVEVDCSTYPNTTNEEGKEVLVCTKILSPICGTDGVTYSNECLLCAYNIEYGTNVSKDHDGECKEVVPVNCSRYPNTTNEEGKVVLRCSKDLSPVCGTDGVTYDNECLMCARNLEPGAVVGKNYDGECKKEIATVDCSDYPKPVCSLEYMPLCGSDSKTYSNKCNFCNAVVDSNGTLTLSHFGKC

SEQ ID NO: 28 - protein, 207 aa, synthetic construct
  REGION 1..207: note = Synthetic polypeptide
MTTAGVFVLLSIALCCFPDAAFGVEVDCSAYSNTTSEEGKEVLSCTKILSPICGTDGVTYSNECLLCAYNIEYGTNISKDHDGECKEVVSVDCSRYPNTTNEEGKAVLLCNKDLSPVCGTDGVTYDNECLLCAHNLEPGTSVGKKYDGACKKEIATVDCSDYPKPVCTLEYLPLCGSDSKTYSNKCDFCNAVVDSNGTLTLSHFGKC

SEQ ID NO: 29 - protein, 206 aa, synthetic construct
  REGION 1..206: note = Synthetic polypeptide
MTTAGVFVLLSFALCCFPDAAFGVEVDCSTYPNTTNEEGKEVLVCTKILSPICGTDGTTYSNECLLCAYNIEYGTNVSKDHDGECKVVPVDCSKYPNTTNEDGKVVLLCNKALSPICGTDRVTYDNECLMCAHNLEPGTSVGKKHDGECQKEVATVDCSDYPKPVCSLEYMPLCGSDGKTYSNKCNFCNAVVNSNGTLTLSHFEKC

SEQ ID NO: 30 - protein, 213 aa, synthetic construct
  REGION 1..213: note = Synthetic polypeptide
MTTAGVFVLLSFVLCCFFPDAAFGVEVDCSTYPNTTNEEGKEVLVCAKILSPVCGTDGVTYSNECLLCAHNIENGTNVGKDHDGKCKEAVPVDCSRYPNTTDEEGKVVLLCNKDVSPVCGTDGVTYDNECLLCAHNLEAGTSVDKKNDSECKTEDTTLAAVSVDCSDYPKPVCTLEYLPLCGSDNKTYSNKCRFCNAVVDSNGTLTLSRFGKC

SEQ ID NO: 31 - protein, 207 aa, synthetic construct
  REGION 1..207: note = Synthetic polypeptide
MTTAGVFVLLSFALCCSPDAAFGVEVDCSTYPNTTNEEGKEVLACTKILSPICGTDGVTYSNECLLCAYNMEYGTNVSKDHDGKCKEVVPVDCSRYPNTTNEEGKVVLLCNKDLSPVCGTDGVTYDNECLLCARNLEPGASVGKKYDGECKKEIATVDCSDYPKPVCSLEHMPLCGSDSKTYSNKCTFCNAVVDSNGTLTLSHFGKC

SEQ ID NO: 32 - protein, 207 aa, synthetic construct
  REGION 1..207: note = Synthetic polypeptide
MTTAGVFVLLSFALCCFPDAAFGVEVDCSTYPNTTNEEGREVLVCTKILSPICGTDGVTYSNECLLCAYNIEYGTNVSKDHDGECKEVAPVGCSRYPNTTNEEGKVVLLCNKDLSPVCGTDGVTYDNECLLCARHLEPGTSVGKKYDGECKKEIATVDCSDYPKPVCSLEYMPLCGSDSKTYSNKCNFCNAVLDSNGTLTLSHFGKC

SEQ ID NO: 33 - protein, 207 aa, synthetic construct
  REGION 1..207: note = Synthetic polypeptide
MTTAGVFVLLSFALCCFPDAVFGVEVDCSTYPNTTNEEGKEVLVCTKILSPICGTDGVTYSNECLLCAYNIEYGTNVSKDHDGECKEVVPVDCSRYPNTTNEEGKVVLRCNKDLSPVCGTDGVTYDNECLMCARNLEPGAIVGKKYDGECKKEIATVDCSDYPKPVCSLEYMPLCGSDSKTYSNKCNFCNAVVDSNGTLILSHFGKC

SEQ ID NO: 34 - protein, 207 aa, synthetic construct
  REGION 1..207: note = Synthetic polypeptide
MTTAGVFVLLSFVLCCFPDAVFGVEVDCSTYPNTTNEEGKEVLVCTKILSPICGTDGVTYSNECLLCAYNIEYGTNVSKDHDGECKEVVPVDCSRYPNTTNEEGKVVLRCSKDLSPVCGTDGVTYDNECLMCARNLEPGAVVGKNYDGECKKEIATVDCSDYPKPVCSLEYMPLCGSDSKTYSNKCNFCNAVVDSNGTLTLSHFGKC

SEQ ID NO: 35 - protein, 282 aa, synthetic construct
  REGION 1..282: note = Synthetic polypeptide
MSSQNQLPSRCRPLPGSQDLNKYYQPHCTGDRFCWLFYVTVEQFRHCICIYLQLALERPSHEQSGQPADSRNTSTMTTAGVFVLLSFALCCFPDAVFGVEVDCSTYPNTTNEEGKEVLVCTKILSPICGTDGVTYSNECLLCAYNIEYGTNVSKDHDGECKEVVPVDCSRYPNTTNEEGKVVLRCNKDLSPVCGTDGVTYDNECLMCARNLEPGAIVGKKYDGECKKEIATVDCSDYPKPVCSLEYMPLCGSDSKTYSNKCNFCNAVVDSNGTLILSHFGKC

SEQ ID NO: 36 - protein, 193 aa, synthetic construct
  REGION 1..193: note = Synthetic polypeptide
MTTAVVFVLLSFALCCFPDAAFGVEVDCSTYPNSTNEEGKDVLVCPKILGPICGTDGVTYSNECLLCAYNIQYGTNVSKDHDGECKEIVPVDCSRYPNTTNEEGKVVFLCNKNFDPVCGTDGDTYDNECMLCARSLEPGTTVGKKHDGECKREIATVDCSDYPKPTCSAEDMPLCGSDSKTYSNKCNFCNAVV

SEQ ID NO: 37 - protein, 190 aa, synthetic construct
  REGION 1..190: note = Synthetic polypeptide
EAEAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 38 - protein, 199 aa, synthetic construct
  REGION 1..199: note = Synthetic polypeptide
EEGVSLEKREAEAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 39 - protein, 275 aa, synthetic construct
  REGION 1..275: note = Synthetic polypeptide
MRFPSIFTAVLFAASSALAAPVNTTTEDETAQIPAEAVIGYSDLEGDFDVAVLPFSNSTNNGLLFINTTIASIAAKEEGVSLEKREAEAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 40 - protein, 273 aa, synthetic construct
  REGION 1..273: note = Synthetic polypeptide
MRFPSIFTAVLFAASSALAAPVNTTTEDETAQIPAEAVIGYSDLEGDFDVAVLPFSNSTNNGLLFINTTIASIAAKEEGVSLEKREAEAVEVDCSTYPNTTNEEGKEVLVCTKILSPICGTDGVTYSNECLLCAYNIEYGTNVSKDHDGECKEFVPVDCSRYPNTTNEDGKVVLLCNKDLSPICGTDGVTYDNECLLCARNLEPGTSVGKKYDGECKKEIATVDCSDYPKPVCSLEYMPLCGSDSKTYSNKCNFCNAVVDSNGTLTLSHFGKC

SEQ ID NO: 41 - protein, 188 aa, synthetic construct
  REGION 1..188: note = Synthetic polypeptide
EAEAVEVDCSTYPNTTNEEGKEVLVCTKILSPICGTDGVTYSNECLLCAYNIEYGTNVSKDHDGECKEFVPVDCSRYPNTTNEDGKVVLLCNKDLSPICGTDGVTYDNECLLCARNLEPGTSVGKKYDGECKKEIATVDCSDYPKPVCSLEYMPLCGSDSKTYSNKCNFCNAVVDSNGTLTLSHFGKC

SEQ ID NO: 42 - protein, 206 aa, synthetic construct
  REGION 1..206: note = Synthetic polypeptide
MTMAGVFVLLSFILCCFPDTAFGVEVDCSIYPNTTSEEGKEVLVCTETLSPICGSDGVTYNNECQLCAYNVEYGTNVSKDHDGECKEIVPVDCSRYPNTTEEGRVVMLCNKALSPVCGTDGVTYDNECLLCARNLESGTSVGKKFDGECKKEIATVDCTDYPKPVCSLDYMPLCGSDSKTYSNKCNFCNAVMDSNGTLTLNHFGKC

SEQ ID NO: 43 - protein, 272 aa, synthetic construct
  REGION 1..272: note = Synthetic polypeptide
MRFPSIFTAVLFAASSALAAPVNTTTEDETAQIPAEAVIGYSDLEGDFDVAVLPFSNSTNNGLLFINTTIASIAAKEEGVSLDKREAEAVEVDCSIYPNTTSEEGKEVLVCTETLSPICGSDGVTYNNECQLCAYNVEYGTNVSKDHDGECKEIVPVDCSRYPNTTEEGRVVMLCNKALSPVCGTDGVTYDNECLLCARNLESGTSVGKKFDGECKKEIATVDCTDYPKPVCSLDYMPLCGSDSKTYSNKCNFCNAVMDSNGTLTLNHFGKC

SEQ ID NO: 44 - protein, 187 aa, synthetic construct
  REGION 1..187: note = Synthetic polypeptide
EAEAVEVDCSIYPNTTSEEGKEVLVCTETLSPICGSDGVTYNNECQLCAYNVEYGTNVSKDHDGECKEIVPVDCSRYPNTTEEGRVVMLCNKALSPVCGTDGVTYDNECLLCARNLESGTSVGKKFDGECKKEIATVDCTDYPKPVCSLDYMPLCGSDSKTYSNKCNFCNAVMDSNGTLTLNHFGKC

SEQ ID NO: 45 - protein, 474 aa, synthetic construct
  REGION 1..474: note = Synthetic polypeptide
MRFPSIFTAVLFAASSALAAPVNTTTEDETAQIPAEAVIGYSDLEGDFDVAVLPFSNSTNNGLLFINTTIASIAAKEEGVSLDKREAEAGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 46 - protein, 389 aa, synthetic construct
  REGION 1..389: note = Synthetic polypeptide
EAEAGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 47 - protein, 419 aa, synthetic construct
  REGION 1..419: note = Synthetic polypeptide
MRVPAQLLGLLLLWLPGARCGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPLEIKRAAAHHHHHH

SEQ ID NO: 48 - protein, 406 aa, synthetic construct
  REGION 1..406: note = Synthetic polypeptide
MTSGFANELGPRLMGKLTMGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKTFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPSR

SEQ ID NO: 49 - protein, 451 aa, synthetic construct
  REGION 1..451: note = Synthetic polypeptide
MGGRRVRWEVYISRAGYVNRQIAWRRHHRSLTMRVPAQLLGLLLLWLPGARCGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPLEIKRAAAHHHHHH

SEQ ID NO: 50 - protein, 386 aa, synthetic construct
  REGION 1..386: note = Synthetic polypeptide
MGSIGAVSMEFCFDVFKELKVHHANENIFYSPFTIISALAMVYLGAKDSTRTQINKVVRFDKLPGFGDSVEAQCGTSVNVHSSLRDILNQITKPNDVYSFSLASRLYAEETYPILPEYLQCVKELYRGGLESINFQTAADQARGLINSWVESQTNGMIKNVLQPSSVDSQTAMVLVNAIVFKGLWEKAFKDEDTQAIPFRVTEQESKPVQMMYQIGLFKVASMASEKMKILELPFASGTMSMWVLLPDEVSGLEQLETTISFEKMTEWISSNIMEERRIKVYLPRMKMEEKYNLTSVLMAMGITDLFSSSANLSGISSAGSLKISQAVHAAYAEIYEAGREVIGSAEAGADATSVSEEFRVDHPFLYCIKHNLTNSILFFGRCISP

SEQ ID NO: 51 - protein, 386 aa, synthetic construct
  REGION 1..386: note = Synthetic polypeptide
MGSIGAVSMEFCFDVFKELKVHHANENIFYSPFTIISALAMVYLGAKDSTRTQINKVVRFDKLPGFGDSVEAQCGTSVNVHSSLRDILNQITKPNDVYSFSLASRLYAEETYPILPEYLQCVKELYRGGLESINFQTAADQARGLINSWVESQTNGMIKNVLQPSSVDSQTAMVLVNAIVFKGLWEKAFKDEDTQAIPFRVTEQESKPVQMMYQIGLFKVASMASEKMKILELPFASGTMSMWVLLPDEVSGLEQLETTISFEKMTEWISSNIMEERRIKVYLPRMKMEEKYNLTSVLMAMGITDLFSSSANLSGISSAGSLKISQAAHAAYAEIYEAGREVIGSAEAGADATSVSEEFRVDHPFLYCIKHNLTNSILFFGRCISP

SEQ ID NO: 52 - protein, 417 aa, synthetic construct
  REGION 1..417: note = Synthetic polypeptide
YYRVPCMVLCTAFHPYIFIVLLFALDNSEFTMGSIGAVSMEFCFDVFKELRVHHPNENIFFCPFAIMSAMAMVYLGAKDSTRTQINKVIRFDKLPGFGDSTEAQCGKSANVHSSLKDILNQITKPNDVYSFSLASRLYADETYSIQSEYLQCVNELYRGGLESINFQTAADQARELINSWVESQTNGIIRNVLQPSSVDSQTAMVLVNAIVFRGLWEKAFKDEDTQTMPFRVTEQESKPVQMMYQIGSFKVASMASEKMKILELPLASGTMSMLVLLPDEVSGLEQLETTISFEKLTEWTSSNVMEERKIKVYLPRMKMEEKYNLTSVLMAMGITDLFRSSANLSGISLAGNLKISQAVHAAHAEINEAGRKAVSSAEAGVDATSVSEEFRADRPFLFCIKHIATKVVFFFGRYTSP

SEQ ID NO: 53 - protein, 383 aa, synthetic construct
  REGION 1..383: note = Synthetic polypeptide
MGSIGAASMEFCFDVFKELKVHHANDNMLYSPFAILSTLAMVFLGAKDSTRTQINKVVHFDKLPGFGDSIEAQCGTSVNVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMYQIGSFKVASMASEKMKILELPFASGTMSMLVLLPDDVSGLEQLESIISFEKLTEWTSSSIMEERKVKVYLPRMKMEEKYNLTSLLMAMGITDLFSSSANLSGISSVGSLKISQAVHAAHAEINEAGRDVVGSAEAGVDATEEFRADHPFLFCVKHIETNAILLFGRCVSP

SEQ ID NO: 54 - protein, 386 aa, synthetic construct
  REGION 1..386: note = Synthetic polypeptide
MASIGAVSTEFCVDVYKELRVHHANENIFYSPFTIISTLAMVYLGAKDSTRTQINKVVRFDKLPGFGDSIEAQCGTSVNVHSSLRDILNQITKPNDVYSFSLASRLYAEETYPILPEYLQCVKELYRGGLESINFQTAADQARELINSWVESQTSGIIKNVLQPSSVNSQTAMVLVNAIYFKGLWERAFKDEDTQAIPFRVTEQESKPVQMMSQIGSFKVASVASEKVKILELPFVSGTMSMLVLLPDEVSGLEQLESTISTEKLTEWTSSSIMEERKIKVFLPRMRMEEKYNLTSVLMAMGMTDLFSSSANLSGISSAESLKISQAVHAAYAEIYEAGREVVSSAEAGVDATSVSEEFRVDHPFLLCIKHNPTNSILFFGRCISP

SEQ ID NO: 55 - protein, 411 aa, synthetic construct
  REGION 1..411: note = Synthetic polypeptide
MALCKAFHPYIFIVLLFDVDNSAFTMASIGAVSTEFCVDVYKELRVHHANENIFYSPFTIISTLAMVYLGAKDSTRTQINKVVRFDKLPGFGDSIEAQCGTSVNVHSSLRDILNQITKPNDVYSFSLASRLYAEETYPILPEYLQCVKELYRGGLESINFQTAADQARELINSWVESQTSGIIKNVLQPSSVNSQTAMVLVNAIYFKGLWERAFKDEDTQAIPFRVTEQESKPVQMMSQIGSFKVASVASEKVKILELPFVSGTMSMLVLLPDEVSGLEQLESTISTEKLTEWTSSSIMEERKIKVFLPRMRMEEKYNLTSVLMAMGMTDLFSSSANLSGISSAESLKISQAVHAAYAEIYEAGREVVSSAEAGVDATSVSEEFRVDHPFLLCIKHNPTNSILFFGRCISP

SEQ ID NO: 56 - protein, 383 aa, synthetic construct
  REGION 1..383: note = Synthetic polypeptide
MGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKISQAVHAAYAEINEAGRDVVGSAEAGVDATEEFRADHPFLFCVKHIETNAILLFGRCVSP

SEQ ID NO: 57 - protein, 408 aa, synthetic construct
  REGION 1..408: note = Synthetic polypeptide
MGLCTAFHPYIFIVLLFALDNSEFTMGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKISQAVHAAYAEINEAGRDVVGSAEAGVDATEEFRADHPFLFCVKHIETNAILLFGRCVSP

SEQ ID NO: 58 - protein, 383 aa, synthetic construct
  REGION 1..383: note = Synthetic polypeptide
MGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKIPQAVHAAYAEINEAGRDVVGSAEAGVDATEEFRADHPFLFCVKHIETNAILLFGRCVSP

SEQ ID NO: 59 - protein, 386 aa, synthetic construct
  REGION 1..386: note = Synthetic polypeptide
MGSIGAASTEFCFDVFRELRVQHVNENIFYSPFSIISALAMVYLGARDNTRTQIDKVVHFDKLPGFGESMEAQCGTSVSVHSSLRDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 60 - protein, 386 aa, synthetic construct
  REGION 1..386: note = Synthetic polypeptide
MGSIGAASTEFCFDVFRELKVQHVNENIFYSPLSIISALAMVYLGARDNTRTQIDQVVHFDKIPGFGESMEAQCGTSVSVHSSLRDILTEITKPSDNFSLSFASRLYAEETYTILPEYLQCVKELYKGGLESISFQTAADQARELINSWVESQTNGIIKNILQPSSVDSQTTMVLVNAIYFKGMWEKAFKDEDTQTMPFRMTEQESKPVQMMYQVGSFKLATVTSEKVKILELPFASGMMSMCVLLPDEVSGLEQLETTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPSNSILFFGRWISP

SEQ ID NO: 61 - protein, 386 aa, synthetic construct
  REGION 1..386: note = Synthetic polypeptide
MGSIGAASTEFCFDVFKELKVQHVNENIFYSPLTIISALSMVYLGARENTRAQIDKVLHFDKMPGFGDTIESQCGTSVSIHTSLKDMFTQITKPSDNYSLSFASRLYAEETYPILPEYLQCVKELYKGGLETISFQTAAEQARELINSWVESQTNGMIKNILQPSSVDPQTKMVLVNAIYFKGVWEKAFKDEDTQEVPFRVTEQESKPVQMMYQIGSFKVAVMASEKMKILELPYASGQLSMLVLLPDDVSGLEQLESAITFEKLMAWTSSTTMEERKMKVYLPRMKIEEKYNLTSVLMALGVTDLFSSSANLSGISSAESLKISKAVHEAFVEIYEAGSEVVGSTEAGMEVTSVSEEFRADHPFLFLIKHNPTNSILFFGRCFSP

SEQ ID NO: 62 - protein, 386 aa, synthetic construct
  REGION 1..386: note = Synthetic polypeptide
MGSIGAASTEFCFDVFKELKVQHVNENIFYSPLTIISALSMVYLGARENTRTQIDKVLHFDKMTGFGDTVESQCGTSVSIHTSLKDIFTQITKPSDNYSLSLASRLYAEETYPILPEYLQCVKELYKGGLETVSFQTAAEQARELINSWVESQTNGMIKNILQPSSVDPQTKMVLVNAIYFKGVWEKAFKDEDTQEVPFRVTEQESKPVQMMYQIGSFKVAVMASEKMKILELPYASGQLSMLVLLPDDVSGLEQLESAITSEKLMEWTSSTTMEERKMKVYLPRMKIEEKYNLTSVLMALGVTDLFSSSADLSGISSAESLKISKAVHEAFVEIYEAGSEVVGSTEGGMEVTSVSEEFRADHPFLFLIKHKPTNSILFFGRCFSP

SEQ ID NO: 63 - protein, 386 aa, synthetic construct
  REGION 1..386: note = Synthetic polypeptide
MGSIGAASTEFCFDVFKELKVQHVNENIFYSPLTIISALSMVYLGARENTRTQIDKVLHFDKMTGFGDTVESQCGTSVSIHTSLKDIFTQITKPSDNYSLSLASRLYAEETYPILPEYLQCVKELYKGGLETVSFQTAAEQARELINSWVESQTNGMIKNILQPSSVDPQTKMVLVNAIYFKGVWEKAFKDEDTQEVPFRVTEQESKPVQMMYQIGSFKVAVMASEKMKILELPYASGQLSMLVLLPDDVSGLEQLESAITSEKLMEWTSSTTMEERKMKVYLPRMKIEEKYNLTSVLMALGVTDLFSSSADLSGISSAESLKISKAVHEAFVEIYEAGSEVVGSTEGGMEVTSFSEEFRADHPFLFLIKHKPTNSILFFGRCFSP

SEQ ID NO: 64 - protein, 386 aa, synthetic construct
  REGION 1..386: note = Synthetic polypeptide
MGSIGAASTEFCFDVFKELKVQHVNENIFYSPLSIISALSMVYLGARENTRAQIDKVVHFDKITGFGETIESQCGTSVSVHTSLKDMFTQITKPSDNYSLSFASRLYAEETYPILPEYLQCVKELYKGGLETTSFQTAADQARELINSWVESQTNGMIKNILQPGSVDPQTEMVLVNAIYFKGMWEKAFKDEDTQAVPFRMTEQESKTVQMMYQIGSFKVAVMASEKMKILELPYASGELSMLVMLPDDVSGLEQLETAITFEKLMEWTSSNMMEERKMKVYLPRMKMEEKYNLTSVLMALGVTDLFSSSANLSGISSAESLKMSEAVHEAFVEIYEAGSEVVGSTGAGMEVTSVSEEFRADHPFLFLIKHNPTNSILFFGRCFSP

SEQ ID NO: 65 - protein, 386 aa, synthetic construct
  REGION 1..386: note = Synthetic polypeptide
MGSIGAASTEFCFDVFKELRVQHVNENVCYSPLIIISALSLVYLGARENTRAQIDKVVHFDKITGFGESIESQCGTSVSVHTSLKDMFNQITKPSDNYSLSVASRLYAEERYPILPEYLQCVKELYKGGLESISFQTAADQAREAINSWVESQTNGMIKNILQPSSVDPQTEMVLVNAIYFKGMWQKAFKDEDTQAVPFRISEQESKPVQMMYQIGSFKVAVMAAEKMKILELPYASGELSMLVLLPDEVSGLEQLENAITVEKLMEWTSSSPMEERIMKVYLPRMKIEEKYNLTSVLMALGITDLFSSSANLSGISAEESLKMSEAVHQAFAEISEAGSEVVGSSEAGIDATSVSEEFRADHPFLFLIKHNATNSILFFGRCFSP

SEQ ID NO: 66 - protein, 386 aa, synthetic construct
  REGION 1..386: note = Synthetic polypeptide
MGSISAASTEFCFDVFKELKVQHVNENIFYSPLSIISALSMVYLGARENTRAQIEKVVHFDKITGFGESIESQCSTSVSVHTSLKDMFTQITKPSDNYSLSFASRFYAEETYPILPEYLQCVKELYKGGLETINFRTAADQARELINSWVESQTNGMIKNILQPGSVDPQTDMVLVNAIYFKGMWEKAFKDEDTQALPFRVTEQESKPVQMMYQIGSFKVAVLASEKVKILELPYASGQLSMLVLLPDDVSGLEQLETAITVEKLMEWTSSNNMEERKIKVYLPRIKIEEKYNLTSVLMALGITDLFSSSANLSGISSAESLKVSEAIHEAFVEIYEAGSEVAGSTEAGIEVTSVSEEFRADHPFLFLIKHNATNSILFFGRCFSP

SEQ ID NO: 67 - protein, 386 aa, synthetic construct
  REGION 1..386: note = Synthetic polypeptide
MVSIGAASTEFCFDVFKELKVQHVNENIFYSPLSIISALSMVYLGARENTRAQIDKVVHFDKITGFEETIESQCSTSVSVHTSLKDMFTQITKPSDNYSLSFASRLYAEETYPILPEYLQCVKELYKGGLETISFQTAADQARELINSWVESQTDGMIKNILQPGSVDPQTEMVLVNAIYFKGMWEKAFKDEDTQAVPFRMTEQESKPVQMMYQIGSFKVAVMASEKMKILELPYASGGMSMLVMLPDDVSGLEQLETAITFEKLMEWTSSNMMEERKMKVYLPRMKMEEKYNLTSVLMALGMTDLFSSSANLSGISSAESLKMSEAVHEAFVEIYEAGSEAVGSTGAGMEVTSVSEEFRADHPFLFLIKHNPTNSILFFGRCFSP

SEQ ID NO: 68 - protein, 386 aa, synthetic construct
  REGION 1..386: note = Synthetic polypeptide
MGSIGAASTEFCFDVFKELKVQHVNENIFYSPLSIISALSMVYLGARENTRAQIDKVVHFDKITGFGEPIESQCGISVSVHTSLKDMITQITKPSDNYSLSFASRLYAEETYPILPEYLQCVKELYKGGLETISFQTAADQARELINSWVENQTNGMIKNILQPGSVDPQTEMVLVNAVYFKGMWEKAFKDEDTQAVPFRMTEQESKPVQMMYQIGSFKVAVMASEKIKILELPYASGELSMLVLLPDDVSGLEQLETAITLDKLTEWTSSNAMEERKMKVYLPRMKIEKKYNLTSVLIALGMTDLFSSSANLSGISSAESLKMSEAIHEAFLEIYEAGSEVVGSTEAGMEVTSVSEEFRADHPFLFLIKHNPTNSILFFGRCLSP

SEQ ID NO: 69 - protein, 386 aa, synthetic construct
  REGION 1..386: note = Synthetic polypeptide
MGSIGAASTEFCFDVFKELKVQHVNENIFYSPLTIISALSMVYLGARENTRAQIDKVVHFDKIPGFGDTTESQCGTSVSVHTSLKDMFTQITKPSDNYSVSFASRLYAEETYPILPEFLECVKELYKGGLESISFQTAADQARELINSWVESQTNGMIKNILQPGSVDSQTEMVLVNAIYFKGMWEKAFKDEDTQTVPFRMTEQETKPVQMMYQIGTFKVAVMPSEKMKILELPYASGELCMLVMLPDDVSGLEELESSITVEKLMEWTSSNMMEERKMKVFLPRMKIEEKYNLTSVLMALGMTDLFSSSANLSGISSAEPLKMSEAVHEAFIEIYEAGSEVVGSTGAGMEITSVSEEFRADHPFLFLIKHNPTNSILFFGRCVSP

SEQ ID NO: 70 - protein, 386 aa, synthetic construct
  REGION 1..386: note = Synthetic polypeptide
MGSIGAVSTEFCFDVFKELKVQHVNENIFYSPLSIISALSMVYLGARENTRAQIDKVVHFDKITGSGETIEAQCGTSVSVHTSLKDMFTQITKPSENYSVGFASRLYADETYPIIPEYLQCVKELYKGGLEMISFQTAADQARELINSWVESQTNGMIKNILQPGSVDPQTEMILVNAIYFKGVWEKAFKDEDTQAVPFRMTEQESKPVQMMYQFGSFKVAAMAAEKMKILELPYASGALSMLVLLPDDVSGLEQLESAITFEKLMEWTSSNMMEEKKIKVYLPRMKMEEKYNFTSVLMALGMTDLFSSSANLSGISSADSLKMSEVVHEAFVEIYEAGSEVVGSTGSGMEAASVSEEFRADHPFLFLIKHNPTNSILFFGRCFSP

SEQ ID NO: 71 - protein, 391 aa, synthetic construct
  REGION 1..391: note = Synthetic polypeptide
MVSIGAASTEFCFDVFKELKVQHVNENIFYSPLSIISALSMVYLGARENTRAQIDKVVHFDKITGFEETIESQVQKKQCSTSVSVHTSLKDMFTQITKPSDNYSLSFASRLYAEETYPILPEYLQCVKELYKGGLETISFQTAADQARELINSWVESQTDGMIKNILQPGSVDPQTEMVLVNAIYFKGMWEKAFKDEDTQAVPFRMTEQESKPVQMMYQIGSFKVAVMASEKMKILELPYASGGMSMLVMLPDDVSGLEQLETAITFEKLMEWTSSNMMEERKMKVYLPRMKMEEKYNLTSVLMALGMTDLFSSSANLSGISSAESLKMSEAVHEAFVEIYEAGSEAVGSTGAGMEVTSVSEEFRADHPFLFLIKHNPTNSILFFGRCFSP

SEQ ID NO: 72 - protein, 386 aa, synthetic construct
  REGION 1..386: note = Synthetic polypeptide
MGSIGAASGEFCFDVFKELKVQHVNENIFYSPLSIISALSMVYLGARENTRAQIDKVVHFDKIIGFGESIESQCGTSVSVHTSLKDMFAQITKPSDNYSLSFASRLYAEETFPILPEYLQCVKELYKGGLETLSFQTAADQARELINSWVESQTNGMIKDILQPGSVDPQTEMVLVNAIYFKGVWEKAFKDEDTQTVPFRMTEQESKPVQMMYQIGSFKVAVVAAEKIKILELPYASGALSMLVLLPDDVSSLEQLETAITFEKLTEWTSSNIMEERKIKVYLPRMKIEEKYNLTSVLMDLGITDLFSSSANLSGISSAESLKVSEAIHEAIVDIYEAGSEVVGSSGAGLEGTSVSEEFRADHPFLFLIKHNPTSSILFFGRCFSP

SEQ ID NO: 73 - protein, 386 aa, synthetic construct
  REGION 1..386: note = Synthetic polypeptide
MGSIGAASTEFCFDVFKELKVQHVNENIFYSPLSIISALSMVYLGARENTRAQIDKVVHFDKITGSGEAIESQCGTSVSVHISLKDMFTQITKPSDNYSLSFASRLYAEETYPILPEYLQCVKELYKEGLATISFQTAADQAREFINSWVESQTNGMIKNILQPGSVDPQTQMVLVNAIYFKGVWEKAFKDEDTQAVPFRMTKQESKPVQMMYQIGSFKVAVMASEKMKILELPYASGQLSMLVMLPDDVSGLEQIENAITFEKLMEWTNPNMMEERKMKVYLPRMKMEEKYNLTSVLMALGMTDLFSSSANLSGISSAESLKMSEAVHEAFVEIYEAGSEVVGSTGAGIEVTSVSEEFRADHPFLFLIKHNPTNSILFFGRCFSP

SEQ ID NO: 74 - protein, 386 aa, synthetic construct
  REGION 1..386: note = Synthetic polypeptide
MGSIGEASTEFCIDVFRELKVQHVNENIFYSPLSIISALSMVYLGARENTRAQIDQVVHFDKITGFGDTVESQCGSSLSVHSSLKDIFAQITQPKDNYSLNFASRLYAEETYPILPEYLQCVKELYKGGLETISFQTAADQARELINSWVESQTNGMIKNILQPSSVDPQTEMVLVNAIYFKGVWEKAFKDEETQAVPFRITEQENRPVQIMYQFGSFKVAVVASEKIKILELPYASGQLSMLVLLPDEVSGLEQLENAITFEKLTEWTSSDIMEEKKIKVFLPRMKIEEKYNLTSVLVALGIADLFSSSANLSGISSAESLKMSEAVHEAFVEIYEAGSEVVGSSGAGIEAASDSEEFRADHPFLFLIKHKPTNSILFFGRCFSP

SEQ ID NO: 75 - protein, 386 aa, synthetic construct
  REGION 1..386: note = Synthetic polypeptide
MGSIGAASTEFCFDIFNELKVQHVNENIFYSPLSIISALSMVYLGARENTKAQIDKVVHFDKITGFGESIESQCSTSASVHTSFKDMFTQITKPSDNYSLSFASRLYAEETYPILPEYSQCVKELYKGGLESISFQTAADQARELINSWVESQTNGMIKNILQPGSVDPQTELVLVNAIYFKGTWEKAFKDKDTQAVPFRVTEQESKPVQMMYQIGSYKVAVIASEKMKILELPYASGELSMLVLLPDDVSGLEQLETAITFEKLMEWTSSNMMEERKVKVYLPRMKIEEKYNLTSVLMALGMTDLFSPSANLSGISSAESLKMSEAIHEAFVEIYEAGSEVVGSTEAGMEVTSVSEEFRADHPFLFLIKCNLTNSILFFGRCFSP

SEQ ID NO: 76 - protein, 385 aa, synthetic construct
  REGION 1..385: note = Synthetic polypeptide
MGSISTASTEFCFDVFKELKVQHVNENIFYSPLSIISALSMVYLGARENTRAQIEKVVHFDKITGFGESIESQCGTSVSVHTSLKDMLIQISKPSDNYSLSFASKLYAEETYPILPEYLQCVKELYKGGLESINFQTAADQARQLINSWVESQTNGMIKDILQPSSVDPQTEMVLVNAIYFKGIWEKAFKDEDTQEVPFRITEQESKPVQMMYQIGSFKVAVIASEKIKILELPYASGELSMLIVLPDDVSGLEQLETAITFEKLIEWTSPSIMEERKTKVYLPRMKIEEKYNLTSVLMALGMTDLFSPSANLSGISSAESLKMSEAIHEAFVEIYEAGSEVVGSAEAGMEATSVSEFRVDHPFLFLIKHNPANIILFFGRCVSP

SEQ ID NO: 77 - protein, 386 aa, synthetic construct
  REGION 1..386: note = Synthetic polypeptide
MGSIGAASTEFCFDVFKELKVQHVNENIFYSPLTIISALSLVYLGARENTRAQIDKVFHFDKISGFGETTESQCGTSVSVHTSLKEMFTQITKPSDNYSVSFASRLYAEDTYPILPEYLQCVKELYKGGLETISFQTAADQAREVINSWVESQTNGMIKNILQPGSVDSQTEMVLVNAIYFKGMWEKAFKDEDTQTMPFRITEQERKPVQMMYQAGSFKVAVMASEKMKILELPYASGEFCMLIMLPDDVSGLEQLENSFSFEKLMEWTTSNMMEERKMKVYIPRMKMEEKYNLTSVLMALGMTDLFSSSANLSGISSAETLKMSEAVHEAFMEIYEAGSEVVGSTGSGAEVTGVYEEFRADHPFLFLVKHKPTNSILFFGRCVSP

SEQ ID NO: 78 - protein, 386 aa, synthetic construct
  REGION 1..386: note = Synthetic polypeptide
MGSIGAASTEFCFDIFNELKVQHVNENIFYSPLSIISALSMVYLGARENTKAQIDKVVHFDKITGFGETIESQCSTSVSVHTSLKDTFTQITKPSDNYSLSFASRLYAEETYPILPEYSQCVKELYKGGLETISFQTAADQARELINSWVESQTNGMIKNILQPGSVDPQTELVLVNAIYFKGTWEKAFKDKDTQAVPFRVTEQESKPVQMMYQIGSYKVAVIASEKMKILELPYASRELSMLVLLPDDVSGLEQLETAITFEKLMEWTSSNMMEERKVKVYLPRMKIEEKYNLTSVLMALGMTDLFSPSANLSGISSAESLKMSEAVHEAFVEIYEAGSEVVGSTGAGMEVTSVSEEFRADHPFLFLIKCNPTNSILFFGRCFSP

SEQ ID NO: 79 - protein, 386 aa, synthetic construct
  REGION 1..386: note = Synthetic polypeptide
MGSISAASAEFCLDVFKELKVQHVNENIFYSPLSIISALSMVYLGARENTRAQIDKVVHFDKITGSGETIEFQCGTSANIHPSLKDMFTQITRLSDNYSLSFASRLYAEERYPILPEYLQCVKELYKGGLETISFQTAADQARELINSWVESQTNGMIKNILQPGSVNPQTEMVLVNAIYFKGLWEKAFKDEDTQTVPFRMTEQESKPVQMMYQVGSFKVAVMASDKIKILELPYASGELSMLVLLPDDVTGLEQLETSITFEKLMEWTSSNVMEERTMKVYLPHMRMEEKYNLTSVLMALGVTDLFSSSANLSGISSAESLKMSEAVHEAFVEIYESGSQVVGSTGAGTEVTSVSEEFRVDHPFLFLIKHNPTNSILFFGRCFSP

SEQ ID NO: 80 - protein, 385 aa, synthetic construct
  REGION 1..385: note = Synthetic polypeptide
MGSIGAASVEFCFDVFKELKVQHVNENIFYSPLSIISALSMVYLGARENTKAQIDKVVHFDKIAGFGEAIESQCVTSASIHSLKDMFTQITKPSDNYSLSFASRLYAEEAYSILPEYLQCVKELYKGGLETISFQTAADQARDLINSWVESQTNGMIKNILQPGAVDLETEMVLVNAIYFKGMWEKAFKDEDTQTVPFRMTEQESKPVQMMYQVGSFKVAVMASDKIKILELPYASGQLSMVVVLPDDVSGLEQLEASITSEKLMEWTSSSIMEEKKIKVYFPHMKIEEKYNLTSVLMALGMTDLFSSSANLSGISSAEKLKVSEAVHEAFVEISEAGSEVVGSTEAGTEVTSVSEEFKADHPFLFLIKHNPTNSILFFGRCFSP

SEQ ID NO: 81 - protein, 386 aa, synthetic construct
  REGION 1..386: note = Synthetic polypeptide
MGSIGAASSEFCFDIFKELKVQHVNENIFYSPLSIISALSMVYLGARENTRAQIDKVVPFDKITASGESIESQCSTSVSVHTSLKDIFTQITKSSDNHSLSFASRLYAEETYPILPEYLQCVKELYEGGLETISFQTAADQARELINSWIESQTNGRIKNILQPGSVDPQTEMVLVNAIYFKGMWEKAFKDEDTQAVPFRMTEQESKPVQVMHQIGSFKVAVLASEKIKILELPYASGELSMLVLLPDDVSGLEQLETAITFEKLMEWTSPNIMEERKIKVFLPRMKIEEKYNLTSVLMALGITDLFSPLANLSGISSAESLKMSEAIHEAFVEISEAGSEVIGSTEAEVEVTNDPEEFRADHPFLFLIKHNPTNSILFFGRCFSP

SEQ ID NO: 82 - protein, 386 aa, synthetic construct
  REGION 1..386: note = Synthetic polypeptide
MGSIGAASTEFCFDVFKELKAQYVNENIFYSPMTIITALSMVYLGSKENTRAQIAKVAHFDKITGFGESIESQCGASASIQFSLKDLFTQITKPSGNHSLSVASRIYAEETYPILPEYLECMKELYKGGLETINFQTAANQARELINSWVERQTSGMIKNILQPSSVDSQTEMVLVNAIYFRGLWEKAFKVEDTQATPFRITEQESKPVQMMHQIGSFKVAVVASEKIKILELPYASGRLTMLVVLPDDVSGLKQLETTITFEKLMEWTTSNIMEERKIKVYLPRMKIEEKYNLTSVLMALGLTDLFSSSANLSGISSAESLKMSEAVHEAFVEIYEAGSEVVASAEAGMDATSVSEEFRADHPFLFLIKDNTSNSILFFGRCFSP

SEQ ID NO: 83 - protein, 386 aa, synthetic construct
  REGION 1..386: note = Synthetic polypeptide
MGSIGAASTEFCFDVFKELKGQHVNENIFFCPLSIVSALSMVYLGARENTRAQIVKVAHFDKIAGFAESIESQCGTSVSIHTSLKDMFTQITKPSDNYSLNFASRLYAEETYPIIPEYLQCVKELYKGGLETISFQTAADQAREIINSWVESQTNGMIKNILRPSSVHPQTELVLVNAVYFKGTWEKAFKDEDTQAVPFRITEQESKPVQMMYQIGSFKVAAVTSEKMKILEVPYASGELSMLVLLPDDVSGLEQLETAITAEKLIEWTSSTVMEERKLKVYLPRMKIEEKYNLTTVLTALGVTDLFSSSANLSGISSAQGLKMSNAVHEAFVEIYEAGSEVVGSKGEGTEVSSVSDEFKADHPFLFLIKHNPTNSIVFFGRCFSP

SEQ ID NO: 84 - protein, 386 aa, synthetic construct
  REGION 1..386: note = Synthetic polypeptide
MGSIGAASTEFCFDVFKELKVHHVNENILYSPLAIISALSMVYLGAKENTRDQIDKVVHFDKITGIGESIESQCSTAVSVHTSLKDVFDQITRPSDNYSLAFASRLYAEKTYPILPEYLQCVKELYKGGLETIDFQTAADQARQLINSWVEDETNGMIKNILRPSSVNPQTKIILVNAIYFKGMWEKAFKDEDTQEVPFRITEQETKSVQMMYQIGSFKVAEVVSDKMKILELPYASGKLSMLVLLPDDVYGLEQLETVITVEKLKEWTSSIVMEERITKVYLPRMKIMEKYNLTSVLTAFGITDLFSPSANLSGISSTESLKVSEAVHEAFVEIHEAGSEVVGSAGAGIEATSVSEEFKADHPFLFLIKHNPTNSILFFGRCFSP

SEQ ID NO: 85 - protein, 386 aa, synthetic construct
  REGION 1..386: note = Synthetic polypeptide
MGSIGAASTEFCLDVFKELKVQHVNENIFYSPLSIISALSMVYLGARENTRAQIDKVVHFDKITGFEDSIESQCGTSVSVHTSLKDMFTQITKPSDNYSVGFASRLYAAETYQILPEYSQCVKELYKGGLETINFQKAADQATELINSWVESQTNGMIKNILQPSSVDPQTQIFLVNAIYFKGMWQRAFKEEDTQAVPFRISEKESKPVQMMYQIGSFKVAVIPSEKIKILELPYASGLLSMLVILPDDVSGLEQLENAITLEKLMQWTSSNMMEERKIKVYLPRMRMEEKYNLTSVFMALGITDLFSSSANLSGISSAESLKMSDAVHEASVEIHEAGSEVVGSTGSGTEASSVSEEFRADHPYLFLIKHNPTDSIVFFGRCFSP

SEQ ID NO: 86 - protein, 386 aa, synthetic construct
  REGION 1..386: note = Synthetic polypeptide
MGSIGAASTEFCFDVFKELKFQHVDENIFYSPLTIISALSMVYLGARENTRAQIDKVVHFDKIAGFEETVESQCGTSVSVHTSLKDMFAQITKPSDNYSLSFASRLYAEETYPILPEYLQCVKELYKGGLETISFQTAADQARDLINSWVESQTNGMIKNILQPSSVGPQTELILVNAIYFKGMWQKAFKDEDTQEVPFRMTEQQSKPVQMMYQTGSFKVAVVASEKMKILALPYASGQLSLLVMLPDDVSGLKQLESAITSEKLIEWTSPSMMEERKIKVYLPRMKIEEKYNLTSVLMALGITDLFSPSANLSGISSAESLKMSQAVHEAFVEIYEAGSEVVGSTGAGMEDSSDSEEFRVDHPFLFFIKHNPTNSILFFGRCFSP

SEQ ID NO: 87 - protein, 386 aa, synthetic construct
  REGION 1..386: note = Synthetic polypeptide
MGSIGPLSVEFCCDVFKELRIQHPRENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPINFQTAAEQARELINSWVESQTNGMIKNILQPSSVNPETDMVLVNAIYFKGLWEKAFKDEDIQTVPFRITEQESKPVQMMFQIGSFRVAEITSEKIRILELPYASGQLSLWVLLPDDISGLEQLETAITFENLKEWTSSTKMEERKIKVYLPRMKIEEKYNLTSVLTSLGITDLFSSSANLSGISSAESLKVSSAFHEASVEIYEAGSKVVGSTGAEVEDTSVSEEFRADHPFLFLIKHNPSNSIFFFGRCFSP

SEQ ID NO: 88 - protein, 386 aa, synthetic construct
  REGION 1..386: note = Synthetic polypeptide
MGSIGTASAEFCFDVFKELKVHHVNENIFYSPLSIISALSMVYLGARENTKTQMEKVIHFDKITGLGESMESQCGTGVSIHTALKDMLSEITKPSDNYSLSLASRLYAEQTYAILPEYLQCIKELYKESLETVSFQTAADQARELINSWIESQTNGVIKNFLQPGSVDSQTELVLVNAIYFKGMWEKAFKDEDTQEVPFRITEQESRPVQMMYQAGSFKVATVAAEKIKILELPYASGELSMLVLLPDDISGLEQLETTISFEKLTEWTSSNMMEDRNMKVYLPRMKIEEKYNLTSVLIALGMTDLFSPAANLSGISAAESLKMSEAIHAAYVEIYEADSEIVSSAGVQVEVTSDSEEFRVDHPFLFLIKHNPTNSVLFFGRCISP

SEQ ID NO: 89 - protein, 386 aa, synthetic construct
  REGION 1..386: note = Synthetic polypeptide
MGSIGAVSTEFSCDVFKELRIHHVQENIFYSPVTIISALSMIYLGARDSTKAQIEKAVHFDKIPGFGESIESQCGTSLSIHTSIKDMFTKITKASDNYSIGIASRLYAEEKYPILPEYLQCVKELYKGGLESISFQTAAEQAREIINSWVESQTNGMIKNILQPSSVDPQTDIVLVNAIYFKGLWEKAFRDEDTQTVPFKITEQESKPVQMMYQIGSFKVAEITSEKIKILEVPYASGQLSLWVLLPDDISGLEKLETAITFENLKEWTSSTKMEERKIKVYLPRMKIEEKYNLTSVLTALGITDLFSSSANLSGISSAESLKVSEAFHEAIVEISEAGSKVVGSVGAGVDDTSVSEEFRADHPFLFLIKHNPTSSIFFFGRCFSP

SEQ ID NO: 90 - protein, 381 aa, synthetic construct
  REGION 1..381: note = Synthetic polypeptide
MGSIGAASTEFCFDVFKELKVQHVNENIFYSPLSIISALSMVYLGARENTRAQIDKVVHFDKIAGFGESTESQCGTSVSAHTSLKDMSNQITKLSDNYSLSFASRLYAEETYPILPEYSQCVKELYKGGLESISFQTAAYQARELINAWVESQTNGMIKDILQPGSVDSQTKMVLVNAIYFKGIWEKAFKDEDTQEVPFRMTEQETKPVQMMYQIGSFKVAVIAAEKIKILELPYASGQLSMLVILPDDVSGLEQLETAITFEKLTEWTSASVMEERKIKVYLPRMSIEEKYNLTSVLIALGVTDLFSSSANLSGISSAESLRMSEAIHEAFVETYEAGSTESGTEVTSASEEFRVDHPFLFLIKHKPTNSILFFGRCFSP

SEQ ID NO: 91 - protein, 391 aa, synthetic construct
  REGION 1..391: note = Synthetic polypeptide
MGSIGAASSEFCFDIFKELKVQHVNENIFYSPLSIISALSMVYLGARENTRAQIDKVVPFDKITASGESIESQVQKIQCSTSVSVHTSLKDIFTQITKSSDNHSLSFASRLYAEETYPILPEYLQCVKELYEGGLETISFQTAADQARELINSWIESQTNGRIKNILQPGSVDPQTEMVLVNAIYFKGMWEKAFKDEDTQAVPFRMTEQESKPVQVMHQIGSFKVAVLASEKIKILELPYASGELSMLVLLPDDVSGLEQLETAITFEKLMEWTSPNIMEERKIKVFLPRMKIEEKYNLTSVLMALGITDLFSPLANLSGISSAESLKMSEAIHEAFVEISEAGSEVIGSTEAEVEVTNDPEEFRADHPFLFLIKHNPTNSILFFGRCFSP

SEQ ID NO: 92 - protein, 383 aa, synthetic construct
  REGION 1..383: note = Synthetic polypeptide
MGSIGPLSVEFCCDVFKELRIQHARENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPISFQTAAEQARELINSWVESQTNGIIKNILQPSSVNPETDMVLVNAIYFKGLWEKAFKDEGTQTVPFRITEQESKPVQMMFQIGSFRVAEIASEKIRILELPYASGQLSLWVLLPDDISGLEQLETAITFENLKEWTSSTKMEERKIKVYLPRMKIEEKYNLTSVLTSLGITDLFSSSANLSGISSAERLKVSSAFHEASMEINEAGSKVVGAGVDDTSVSEEFRVDRPFLFLIKHNPSNSIFFFGRCFSP

SEQ ID NO: 93 - protein, 386 aa, synthetic construct
  REGION 1..386: note = Synthetic polypeptide
MGSIGAASTEFCFDMFKELKVHHVNENIIYSPLSIISILSMVFLGARENTKTQMEKVIHFDKITGFGESLESQCGTSVSVHASLKDILSEITKPSDNYSLSLASKLYAEETYPVLPEYLQCIKELYKGSLETVSFQTAADQARELINSWVETQTNGVIKNFLQPGSVDPQTEMVLVDAIYFKGTWEKAFKDEDTQEVPFRITEQESKPVQMMYQAGSFKVATVAAEKMKILELPYASGELSMFVLLPDDISGLEQLETTISIEKLSEWTSSNMMEDRKMKVYLPHMKIEEKYNLTSVLVALGMTDLFSPSANLSGISTAQTLKMSEAIHGAYVEIYEAGSEMATSTGVLVEAASVSEEFRVDHPFLFLIKHNPSNSILFFGRCIFP

SEQ ID NO: 94 - protein, 392 aa, synthetic construct
  REGION 1..392: note = Synthetic polypeptide
MGSIGAASTEFCFDMFKELKVHHVNENIIYSPLSIISILSMVFLGARENTKTQMEKVIHFDKITGFGESLESQCGTSVSVHASLKDILSEITKPSDNYSLSLASKLYAEETYPVLPEYLQCIKELYKGSLETVSFQTAADQARELINSWVETQTNGVIKNFLQPGSVDPQTEMVLVDAIYFKGTWEKAFKDEDTQEVPFRITEQESKPVQMMYQAGSFKVATVAAEKMKILELPYASGELSMFVLLPDDISGLEQLETTISIEKLSEWTSSNMMEDRKMKVYLPHMKIEEKYNLTSVLVALGMTDLFSPSANLSGISTAQTLKMSEAIHGAYVEIYEAGSEMATSTGVLVEAASVSEEFRVDHPFLFLIKHNPSNSILFFGRCIFPHHHHHH

SEQ ID NO: 95 - protein, 386 aa, synthetic construct
  REGION 1..386: note = Synthetic polypeptide
MGSIGPLSVEFCCDVFKELRIQHARENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPISFQTAAEQARELINSWVESQTNGMIKNILQPSAVNPETDMVLVNAIYFKGLWEKAFKDEGTQTVPFRITEQESKPVQMMFQIGSFRVAEITSEKIRILELPYASGQLSLWVLLPDDISGLEQLETAITFENLKEWTSSTKMEERKIKVYLPRMKIEEKYNLTSVLTSLGITDLFSSSANLSGISSAERLKVSSAFHEASMEIYEAGSKVVGSTGAGVDDTSVSEEFRVDRPFLFLIKHNPSNSIFFFGRCFSP

SEQ ID NO: 96 - protein, 395 aa, synthetic construct
  REGION 1..395: note = Synthetic polypeptide
  SITE 99: note = misc_feature - Xaa can be any naturally occurring amino acid
  SITE 233: note = misc_feature - Xaa can be any naturally occurring amino acid
  SITE 341: note = misc_feature - Xaa can be any naturally occurring amino acid
  SITE 379: note = misc_feature - Xaa can be any naturally occurring amino acid
MEDQRGNTGFTMGSIGAASTEFCIDVFRELRVQHVNENIFYSPLTIISALSMVYLGARENTRAQIDQVVHFDKIAGFGDTVESQCGSSPSVHNSLKTVXAQITQPRDNYSLNLASRLYAEESYPILPEYLQCVKELYNGGLETVSFQTAADQARELINSWVESQTNGIIKNILQPSSVDPQTEMVLVNAIYFKGLWEKAFKDEETQAVPFRITEQENRPVQMMYQFGSFKVAXVASEKIKILELPYASGQLSMLVLLPDEVSGLEQNAITFEKLTEWTSSDLMEERKIKVFFPRVKIEEKYNLTAVLVSLGITDLFSSSANLSGISSAENLKMSEAVHEAXVEIYEAGSEVAGSSGAGIEVASDSEEFRVDHPFLFLIXHNPTNSILFFGRCFSP

SEQ ID NO: 97 - protein, 386 aa, synthetic construct
  REGION 1..386: note = Synthetic polypeptide
MGSIGAASTEFCIDVFRELRVQHVNENIFYSPLSIISALSMVYLGARENTRAQIDEVFHFDKIAGFGDTVDPQCGASLSVHKSLQNVFAQITQPKDNYSLNLASRLYAEESYPILPEYLQCVKELYNEGLETVSFQTGADQARELINSWVENQTNGVIKNILQPSSVDPQTEMVLVNAIYFKGLWQKAFKDEETQAVPFRITEQENRPVQMMYQFGSFKVAVVASEKVKILELPYASGQLSMWVLLPDEVSGLEQLENAITFEKLTEWTSSDLTEERKIKVFLPRVKIEEKYNLTAVLMALGVTDLFSSSANFSGISAAENLKMSEAVHEAFVEIYEAGSEVVGSSGAGIEAPSDSEEFRADHPFLFLIKHNPTNSILFFGRCFSP

SEQ ID NO: 98 - protein, 386 aa, synthetic construct
  REGION 1..386: note = Synthetic polypeptide
MGSIGPLSVEFCCDVFKELRIQHARDNIFYSPVTIISALSMVYLGARDNTKAQIEKAVHFDKIPGFGESIESQCGTSLSVHTSLKDIFTQITKPRENYTVGIASRLYAEEKYPILPEYLQCIKELYKGGLEPISFQTAAEQARELINSWVESQTNGMIKNILQPSSVNPETDMVLVNAIYFKGLWKKAFKDEGTQTVPFRITEQESKPVQMMFQIGSFRVAEITSEKIRILELPYASGQLSLWVLLPDDISGLEQLESAITFENLKEWTSSTKMEERKIKVYLPRMKIEEKYNLTSVLTSLGITDLFSSSANLSGISSAEKLKVSSAFHEASMEIYEAGNKVVGSTGAGVDDTSVSEEFRVDRPFLFLIKHNPSNSIFFFGRCFSP

SEQ ID NO: 99 - protein, 385 aa, synthetic construct
  REGION 1..385: note = Synthetic polypeptide
MGSIGAASAEFCVDVFKELKDQHVNNIVFSPLMIISALSMVNIGAREDTRAQIDKVVHFDKITGYGESIESQCGTSIGIYFSLKDAFTQITKPSDNYSLSFASKLYAEETYPILPEYLKCVKELYKGGLETISFQTAADQARELINSWVESQTNGMIKNILQPSSVDPQTEMVLVNAIYFKGLWEKAFKDEDTQAVPFRITEQESKPVQMMYQIGSFKVAVIASEKIKILELPYASGQLSLLVLLPDDVSGLEQLESAITSEKLLEWTNPNIMEERKTKVYLPRMKIEEKYNLTSVLVALGITDLFSSSANLSGISSAEGLKLSDAVHEAFVEIYEAGREVVGSSEAGVEDSSVSEEFKADRPFIFLIKHNPTNGILYFGRYISP

SEQ ID NO: 100 - protein, 386 aa, synthetic construct
  REGION 1..386: note = Synthetic polypeptide
MGSIGAANTDFCFDVFKELKVHHANENIFYSPLSIVSALAMVYLGARENTRAQIDKALHFDKILGFGETVESQCDTSVSVHTSLKDMLIQITKPSDNYSFSFASKIYTEETYPILPEYLQCVKELYKGGVETISFQTAADQAREVINSWVESHTNGMIKNILQPGSVDPQTKMVLVNAVYFKGIWEKAFKEEDTQEMPFRINEQESKPVQMMYQIGSFKLTVAASENLKILEFPYASGQLSMMVILPDEVSGLKQLETSITSEKLIKWTSSNTMEERKIRVYLPRMKIEEKYNLKSVLMALGITDLFSSSANLSGISSAESLKMSEAVHEAFVEIYEAGSEVTSSTGTEMEAENVSEEFKADHPFLFLIKHNPTDSIVFFGRCMSP

SEQ ID NO: 101 - protein, 383 aa, synthetic construct
  REGION 1..383: note = Synthetic polypeptide
MGSIGPLSVEFCCDVFKELRIQHARENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPISFQTAAEQARELINSWVESQTNGMIKNILQPSSVNPETDMVLVNAIYFKGLWEKAFKDESTQTVPFRITEQESKPVQMMFQIGSFRVAEIASEKIRILELPYASGQLSLWVLLPDDISGLEQLETAITFENLKEWTSSTKMEERKIKVYLPRMKIEEKYNLTSVLTSLGITDLFSSSANLSGISSAERLKVSSAFHEASMEIYEAGSRVVEAGVDDTSVSEEFRVDRPFLFLIKHNPSNSIFFFGRCFSP

SEQ ID NO: 102 - protein, 386 aa, synthetic construct
  REGION 1..386: note = Synthetic polypeptide
MGSIGPVSTEFCCDIFKELRIQHARENIIYSPVTIISALSMVYLGARDNTKAQIEKAVHFDKIPGFGESIESQCGTSLSIHTSLKDILTQITKPSDNYTVGIASRLYAEEKYPILSEYLQCIKELYKGGLEPISFQTAAEQARELINSWVESQTNGMIKNILQPSSVNPETDMVLVNAIYFKGLWEKAFKDEGTQTVPFRITEQESKPVQMMFQIGSFKVAEITSEKIRILELPYASGKLSLWVLLPDDISGLEQLETAITFENLKEWTSSTRMEERKIKVYLPRMKIEEKYNLTSVLTSLGITDLFSSSANLSGISSAERLKVSSAFHEVFVEIYEAGSKVEGSTGAGVDDTSVSEEFRADHPFLFLVKHNPSNSIIFFGRCYLP

SEQ ID NO: 103 - protein, 386 aa, synthetic construct
  REGION 1..386: note = Synthetic polypeptide
MGSTGAASMEFCFALFRELKVQHVNENIFFSPVTIISALSMVYLGARENTRAQLDKVAPFDKITGFGETIGSQCSTSASSHTSLKDVFTQITKASDNYSLSFASRLYAEETYPILPEYLQCVKELYKGGLESISFQTAADQARELINSWVESQTNGMIKDILRPSSVDPQTKIILITAIYFKGMWEKAFKEEDTQAVPFRMTEQESKPVQMMYQIGSFKVAVIPSEKLKILELPYASGQLSMLVILPDDVSGLEQLETAITTEKLKEWTSPSMMKERKMKVYFPRMRIEEKYNLTSVLMALGITDLFSPSANLSGISSAESLKVSEAVHEASVDIDEAGSEVIGSTGVGTEVTSVSEEIRADHPFLFLIKHKPTNSILFFGRCFSP

SEQ ID NO: 104 - protein, 2174 aa, synthetic construct
  REGION 1..2174: note = Synthetic polypeptide
MEHAQLTQLVNSNMTSNTCHEADEFENIDFRMDSISVTNTKFCFDVFNEMKVHHVNENILYSPLSILTALAMVYLGARGNTESQMKKALHFDSITGAGSTTDSQCGSSEYIHNLFKEFLTEITRTNATYSLEIADKLYVDKTFTVLPEYINCARKFYTGGVEEVNFKTAAEEARQLINSWVEKETNGQIKDLLVPSSVDFGTMMVFINTIYFKGIWKTAFNTEDTREMPFSMTKQESKPVQMMCLNDTFNMATLPAEKMRILELPYASGELSMLVLLPDEVSGLEQIEKAINFEKLREWTSTNAMEKKSMKVYLPRMKIEEKYNLTSTLMALGMTDLFSRSANLTGISSVENLMISDAVHGAFMEVNEEGTEAAGSTGAIGNIKHSVEFEEFRADHPFLFLIRYNPTNVILFFDNSEFTMGSIGAVSTEFCFDVFKELRVHHANENIFYSPFTVISALAMVYLGAKDSTRTQINKVVRFDKLPGFGDSIEAQCGTSANVHSSLRDILNQITKPNDIYSFSLASRLYADETYTILPEYLQCVKELYRGGLESINFQTAADQARELINSWVESQTSGIIRNVLQPSSVDSQTAMVLVNAIYFKGLWEKGFKDEDTQAMPFRVTEQENKSVQMMYQIGTFKVASVASEKMKILELPFASGTMSMWVLLPDEVSGLEQLETTISIEKLTEWTSSSVMEERKIKVFLPRMKMEEKYNLTSVLMAMGMTDLFSSSANLSGISSTLQKKGFRSQELGDKYAKPMLESPALTPQVTAWDNSWIVAHPAAIEPDLCYQIMEQKWKPFDWPDFRLPMRVSCRFRTMEALNKANTSFALDFFKHECQEDDDENILFSPFSISSALATVYLGAKGNTADQMAKTEIGKSGNIHAGFKALDLEINQPTKNYLLNSVNQLYGEKSLPFSKEYLQLAKKYYSAEPQSVDFLGKANEIRREINSRVEHQTEGKIKNLLPPGSIDSLTRLVLVNALYFKGNWATKFEAEDTRHRPFRINMHTTKQVPMMYLRDKFNWTYVESVQTDVLELPYVNNDLSMFILLPRDITGLQKLINELTFEKLSAWTSPELMEKMKMEVYLPRFTVEKKYDMKSTLSKMGIEDAFTKVDSCGVTNVDEITTHIVSSKCLELKHIQINKKLKCNKAVAMEQVSASIGNFTIDLFNKLNETSRDKNIFFSPWSVSSALALTSLAAKGNTAREMAEDPENEQAENIHSGFKELMTALNKPRNTYSLKSANRIYVEKNYPLLPTYIQLSKKYYKAEPYKVNFKTAPEQSRKEINNWVEKQTERKIKNFLSSDDVKNSTKSILVNAIYFKAEWEEKFQAGNTDMQPFRMSKNKSKLVKMMYMRHTFPVLIMEKLNFKMIELPYVKRELSMFILLPDDIKDSTTGLEQLERELTYEKLSEWADSKKMSVTLVDLHLPKFSMEDRYDLKDALKSMGMASAFNSNADFSGMTGFQAVPMESLSASTNSFTLDLYKKLDETSKGQNIFFASWSIATALAMVHLGAKGDTATQVAKGPEYEETENIHSGFKELLSAINKPRNTYLMKSANRLFGDKTYPLLPKFLELVARYYQAKPQAVNFKTDAEQARAQINSWVENETESKIQNLLPAGSIDSHTVLVLVNAIYFKGNWEKRFLEKDTSKMPFRLSKTETKPVQMMFLKDTFLIHHERTMKFKIIELPYVGNELSAFVLLPDDISDNTTGLELVERELTYEKLAEWSNSASMMKAKVELYLPKLKMEENYDLKSVLSDMGIRSAFDPAQADFTRMSEKKDLFISKVIHKAFVEVNEEDRIVQLASGRLTGRCRTLANKELSEKNRTKNLFFSPFSISSALSMILLGSKGNTEAQIAKVLSLSKAEDAHNGYQSLLSEINNPDTKYILRTANRLYGEKTFEFLSSFIDSSQKFYHAGLEQTDFKNASEDSRKQINGWVEEKTEGKIQKLLSEGIINSMTKLVLVNAIYFKGNWQEKFDKETTKEMPFKINKNETKPVQMMFRKGKYNMTYIGDLETTVLEIPYVDNELSMIILLPDSIQDESTGLEKLERELTYEKLMDWINPNMMDSTEVRVSLPRFKLEENYELKPTLSTMGMPDAFDLRTADFSGISSGNELVLSEVVHKSFVEVNEEGTEAAAATAGIMLLRCAMIVANFTADHPFLFFIRHNKTNSILFCGRFCSP

SEQ ID NO: 105 - protein, 386 aa, synthetic construct
  REGION 1..386: note = Synthetic polypeptide
MGSIGTASTEFCFDMFKEMKVQHANQNIIFSPLTIISALSMVYLGARDNTKAQMEKVIHFDKITGFGESVESQCGTSVSIHTSLKDMLSEITKPSDNYSLSLASRLYAEETYPILPEYLQCMKELYKGGLETVSFQTAADQARELINSWVESQTNGVIKNFLQPGSVDPQTEMVLVNAIYFKGMWEKAFKDEDTQEVPFRITEQESKPVQMMYQVGSFKVATVAAEKMKILEIPYTHRELSMFVLLPDDISGLEQLETTISFEKLTEWTSSNMMEERKVKVYLPHMKIEEKYNLTSVLMALGMTDLFSPSANLSGISTAQTLMMSEAIHGAYVEIYEAGREMASSTGVQVEVTSVLEEVRADKPFLFFIRHNPTNSMVVFGRYMSP

SEQ ID NO: 106 - protein, 2104 aa, synthetic construct
  REGION 1..2104: note = Synthetic polypeptide
MTSNTCHEADEFENIDFRMDSISVTNTKFCFDVFNEMKVHHVNENILYSPLSILTALAMVYLGARGNTESQMKKALHFDSITGGGSTTDSQCGSSEYIHNLFKEFLTEITRTNATYSLEIADKLYVDKTFTVLPEYINCARKFYTGGVEEVNFKTAAEEARQLMNSWVEKETNGQIKDLLVPSSVDFGTMMVFINTIYFKGIWKTAFNTEDTREMPFSMTKQESKPVQMMCLNDTFNMVTLPAEKMRILELPYASGELSMLVLLPDEVSGLERIEKAINFEKLREWTSTNAMEKKSMKVYLPRMKIEEKYNLTSTLMALGMTDLFSRSANLTGISSVDNLMISDAVHGAFMEVNEEGTEAAGSTGAIGNIKHSVEFEEFRADHPFLFLIRYNPTNVILFFDNSEFTMGSIGAVSTEFCFDVFKELRVHHANENIFYSPFTIISALAMVYLGAKDSTRTQINKVVRFDKLPGFGDSIEAQCGTSANVHSSLRDILNQITKPNDIYSFSLASRLYADETYTILPEYLQCVKELYRGGLESINFQTAADQARELINSWVESQTSGIIRNVLQPSSVDSQTAMVLVNAIYFKGLWEKGFKDEDTQAIPFRVTEQENKSVQMMYQIGTFKVASVASEKMKILELPFASGTMSMWVLLPDEVSGLEQLETTISIEKLTEWTSSSVMEERKIKVFLPRMKMEEKYNLTSVLMAMGMTDLFSSSANLSGISSTLQKKGFRSQELGDKYAKPMLESPALTPQATAWDNSWIVAHPPAIEPDLYYQIMEQKWKPFDWPDFRLPMRVSCRFRTMEALNKANTSFALDFFKHECQEDDSENILFSPFSISSALATVYLGAKGNTADQMAKVLHFNEAEGARNVTTTIRMQVYSRTDQQRLNRRACFQKTEIGKSGNIHAGFKGLNLEINQPTKNYLLNSVNQLYGEKSLPFSKEYLQLAKKYYSAEPQSVDFVGTANEIRREINSRVEHQTEGKIKNLLPPGSIDSLTRLVLVNALYFKGNWATKFEAEDTRHRPFRINTHTTKQVPMMYLSDKFNWTYVESVQTDVLELPYVNNDLSMFILLPRDITGLQKLINELTFEKLSAWTSPELMEKMKMEVYLPRFTVEKKYDMKSTLSKMGIEDAFTKVDNCGVTNVDEITIHVVPSKCLELKHIQINKELKCNKAVAMEQVSASIGNFTIDLFNKLNETSRDKNIFFSPWSVSSALALTSLAAKGNTAREMAEDPENEQAENIHSGFNELLTALNKPRNTYSLKSANRIYVEKNYPLLPTYIQLSKKYYKAEPHKVNFKTAPEQSRKEINNWVEKQTERKIKNFLSSDDVKNSTKLILVNAIYFKAEWEEKFQAGNTDMQPFRMSKNKSKLVKMMYMRHTFPVLIMEKLNFKMIELPYVKRELSMFILLPDDIKDSTTGLEQLERELTYEKLSEWADSKKMSVTLVDLHLPKFSMEDRYDLKDALRSMGMASAFNSNADFSGMTGERDLVISKVCHQSFVAVDEKGTEAAAATAVIAEAVPMESLSASTNSFTLDLYKKLDETSKGQNIFFASWSIATALTMVHLGAKGDTATQVAKGPEYEETENIHSGFKELLSALNKPRNTYSMKSANRLFGDKTYPLLPTKTKPVQMMFLKDTFLIHHERTMKFKIIELPYMGNELSAFVLLPDDISDNTTGLELVERELTYEKLAEWSNSASMMKVKVELYLPKLKMEENYDLKSALSDMGIRSAFDPAQADFTRMSEKKDLFISKVIHKAFVEVNEEDRIVQLASGRLTGNTEAQIAKVLSLSKAEDAHNGYQSLLSEINNPDTKYILRTANRLYGEKTFEFLSSFIDSSQKFYHAGLEQTDFKNASEDSRKQINGWVEEKTEGKIQKLLSEGIINSMTKLVLVNAIYFKGNWQEKFDKETTKEMPFKINKNETKPVQMMFRKGKYNMTYIGDLETTVLEIPYVDNELSMIILLPDSIQDESTGLEKLERELTYEKLMDWINPNMMDSTEVRVSLPRFKLEENYELKPTLSTMGMPDAFDLRTADFSGISSGNELVLSEVVHKSFVEVNEEGTEAAAATAGIMLLRCAMIVANFTADHPFLFFIRHNKTNSILFCGRFCSP

SEQ ID NO: 107 - protein, 386 aa, synthetic construct
  REGION 1..386: note = Synthetic polypeptide
MASIGAASTEFCFDVFKELKTQHVKENIFYSPMAIISALSMVYIGARENTRAEIDKVVHFDKITGFGNAVESQCGPSVSVHSSLKDLITQISKRSDNYSLSYASRIYAEETYPILPEYLQCVKEVYKGGLESISFQTAADQARENINAWVESQTNGMIKNILQPSSVNPQTEMVLVNAIYLKGMWEKAFKDEDTQTMPFRVTQQESKPVQMMYQIGSFKVAVIASEKMKILELPYTSGQLSMLVLLPDDVSGLEQVESAITAEKLMEWTSPSIMEERTMKVYLPRMKMVEKYNLTSVLMALGMTDLFTSVANLSGISSAQGLKMSQAIHEAFVEIYEAGSEAVGSTGVGMEITSVSEEFKADLSFLFLIRHNPTNSIIFFGRCISP

SEQ ID NO: 108 - protein, 420 aa, synthetic construct
  REGION 1..420: note = Synthetic polypeptide
MGSIGAASTEFCFDVFRELRVQHVNENIFYSPFSIISALAMVYLGARDNTRTQIDKISQFQALSDEHLVLCIQQLGEFFVCTNRERREVTRYSEQTEDKTQDQNTGQIHKIVDTCMLRQDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 109 - protein, 386 aa, synthetic construct
  REGION 1..386: note = Synthetic polypeptide
MGSIGAASAEFCLDIFKELKVQHVNENIIFSPMTIISALSLVYLGAKEDTRAQIEKVVPFDKIPGFGEIVESQCPKSASVHSSIQDIFNQIIKRSDNYSLSLASRLYAEESYPIRPEYLQCVKELDKEGLETISFQTAADQARQLINSWVESQTNGMIKNILQPSSVNSQTEMVLVNAIYFRGLWQKAFKDEDTQAVPFRITEQESKPVQMMQQIGSFKVAEIASEKMKILELPYASGQLSMLVLLPDDVSGLEKLESSITVEKLIEWTSSNLTEERNVKVYLPRLKIEEKYNLTSVLAALGITDLFSSSANLSGISTAESLKLSRAVHESFVEIQEAGHEVEGPKEAGIEVTSALDEFRVDRPFLFVTKHNPTNSILFLGRCLSP

SEQ ID NO: 110 - protein, 386 aa, synthetic construct
  REGION 1..386: note = Synthetic polypeptide
MGSISAASGEFCLDIFKELKVQHVNENIFYSPMVIVSALSLVYLGARENTRAQIDKVIPFDKITGSSEAVESQCGTPVGAHISLKDVFAQIAKRSDNYSLSFVNRLYAEETYPILPEYLQCVKELYKGGLETISFQTAADQAREIINSWVESQTDGKIKNILQPSSVDPQTKMVLVSAIYFKGLWEKSFKDEDTQAVPFRVTEQESKPVQMMYQIGSFKVAAIAAEKIKILELPYASEQLSMLVLLPDDVSGLEQLEKKISYEKLTEWTSSSVMEEKKIKVYLPRMKIEEKYNLTSILMSLGITDLFSSSANLSGISSTKSLKMSEAVHEASVEIYEAGSEASGITGDGMEATSVFGEFKVDHPFLFMIKHKPTNSILFFGRCISP

SEQ ID NO: 111 - protein, 386 aa, synthetic construct
  REGION 1..386: note = Synthetic polypeptide
MGSIGPVSTEVCCDIFRELRSQSVQENVCYSPLLIISTLSMVYIGAKDNTKAQIEKAIHFDKIPGFGESTESQCGTSVSIHTSLKDIFTQITKPSDNYSISIARRLYAEEKYPILPEYIQCVKELYKGGLESISFQTAAEKSRELINSWVESQTNGTIKNILQPSSVSSQTDMVLVSAIYFKGLWEKAFKEEDTQTIPFRITEQESKPVQMMSQIGTFKVAEIPSEKCRILELPYASGRLSLWVLLPDDISGLEQLETAITFENLKEWTSSSKMEERKIRVYLPRMKIEEKYNLTSVLKSLGITDLFSSSANLSGISSAESLKVSAAFHEASVEIYEAGSKGVGSSEAGVDGTSVSEEIRADHPFLFLIKHNPSDSILFFGRCFSP

SEQ ID NO: 112 - protein, 386 aa, synthetic construct
  REGION 1..386: note = Synthetic polypeptide
MGSIGAASTEFCFDVFKELKVQHVNENIIISPLSIISALSMVYLGAREDTRAQIDKVVHFDKITGFGEAIESQCPTSESVHASLKETFSQLTKPSDNYSLAFASRLYAEETYPILPEYLQCVKELYKGGLETINFQTAAEQARQVINSWVESQTDGMIKSLLQPSSVDPQTEMILVNAIYFRGLWERAFKDEDTQELPFRITEQESKPVQMMSQIGSFKVAVVASEKVKILELPYASGQLSMLVLLPDDVSGLEQLESSITVEKLIEWISSNTKEERNIKVYLPRMKIEEKYNLTSVLVALGITDLFSSSANLSGISSAESLKISEAVHEAFVEIQEAGSEVVGSPGPEVEVTSVSEEWKADRPFLFLIKHNPTNSILFFGRYISP

SEQ ID NO: 113 - protein, 386 aa, synthetic construct
  REGION 1..386: note = Synthetic polypeptide
MGSIGPVSTEVCCDIFRELRSQSVQENVCYSPLLIISTLSMVYIGAKDNTKAQIEKAIHFDKIPGFGESTESQCGTSVSIHTSLKDIFTQITKPSDNYSISIARRLYAEEKYPILQEYIQCVKELYKGGLESISFQTAAEKSRELINSWVESQTNGTIKNILQPSSVSSQTDMVLVSAIYFKGLWEKAFKEEDTQTIPFRITEQESKPVQMMSQIGTFKVAEIPSEKCRILELPYASGRLSLWVLLPDDISGLEQLETSITFENLKEWTSSSKMEERKIRVYLPRMKIEEKYNLTSVLKSLGITDLFSSSANLSGISSAESLKVSAVFHEASVEIYEAGSKGVGSSEAGVDGTSVSEEIRADHPFLFLIKHNPSDSILFFGRCFSP

SEQ ID NO: 114 - protein, 399 aa, synthetic construct
  REGION 1..399: note = Synthetic polypeptide
MLNLMHPKQFCCTMGSIGPVSTEVCCDIFRELRSQSVQENVCYSPLLIISTLSMVYIGAKDNTKAQIEKAIHFDKIPGFGESTESQCGTSVSIHTSLKDIFTQITKPSDNYSISIASRLYAEEKYPILPEYIQCVKELYKGGLESISFQTAAEKSRELINSWVESQTNGTIKNILQPSSVSSQTDMVLVSAIYFKGLWEKAFKEEDTQTVPFRITEQESKPVQMMSQIGTFKVAEIPSEKCRILELPYASGRLSLWVLLPDDISGLEQLETAITSENLKEWTSSSKMEERKIKVYLPRMKIEEKYNLTSVLKSLGITDLFSSSANLSGISSAESLKVSGAFHEAFVEIYEAGSKAVGSSGAGVEDTSVSEEIRADHPFLFFIKHNPSDSILFFGRCFSP

SEQ ID NO: 115 - protein, 389 aa, synthetic construct
  REGION 1..389: note = Synthetic polypeptide
EAEAGSIGTASAEFCFDVFKELKVHHVNENIFYSPLSIISALSMVYLGARENTKTQMEKVIHFDKITGLGESMESQCGTGVSIHTALKDMLSEITKPSDNYSLSLASRLYAEQTYAILPEYLQCIKELYKESLETVSFQTAADQARELINSWIESQTNGVIKNFLQPGSVDSQTELVLVNAIYFKGMWEKAFKDEDTQEVPFRITEQESRPVQMMYQAGSFKVATVAAEKIKILELPYASGELSMLVLLPDDISGLEQLETTISFEKLTEWTSSNMMEDRNMKVYLPRMKIEEKYNLTSVLIALGMTDLFSPAANLSGISAAESLKMSEAIHAAYVEIYEADSEIVSSAGVQVEVTSDSEEFRVDHPFLFLIKHNPTNSVLFFGRCISP

SEQ ID NO: 116 - protein, 474 aa, synthetic construct
  REGION 1..474: note = Synthetic polypeptide
MRFPSIFTAVLFAASSALAAPVNTTTEDETAQIPAEAVIGYSDLEGDFDVAVLPFSNSTNNGLLFINTTIASIAAKEEGVSLEKREAEAGSIGTASAEFCFDVFKELKVHHVNENIFYSPLSIISALSMVYLGARENTKTQMEKVIHFDKITGLGESMESQCGTGVSIHTALKDMLSEITKPSDNYSLSLASRLYAEQTYAILPEYLQCIKELYKESLETVSFQTAADQARELINSWIESQTNGVIKNFLQPGSVDSQTELVLVNAIYFKGMWEKAFKDEDTQEVPFRITEQESRPVQMMYQAGSFKVATVAAEKIKILELPYASGELSMLVLLPDDISGLEQLETTISFEKLTEWTSSNMMEDRNMKVYLPRMKIEEKYNLTSVLIALGMTDLFSPAANLSGISAAESLKMSEAIHAAYVEIYEADSEIVSSAGVQVEVTSDSEEFRVDHPFLFLIKHNPTNSVLFFGRCISP

SEQ ID NO: 117 - protein, 389 aa, synthetic construct
  REGION 1..389: note = Synthetic polypeptide
EAEAGSIGAASTEFCFDVFRELRVQHVNENIFYSPFSIISALAMVYLGARDNTRTQIDKVVHFDKLPGFGESMEAQCGTSVSVHSSLRDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 118 - protein, 474 aa, synthetic construct
  REGION 1..474: note = Synthetic polypeptide
MRFPSIFTAVLFAASSALAAPVNTTTEDETAQIPAEAVIGYSDLEGDFDVAVLPFSNSTNNGLLFINTTIASIAAKEEGVSLEKREAEAGSIGAASTEFCFDVFRELRVQHVNENIFYSPFSIISALAMVYLGARDNTRTQIDKVVHFDKLPGFGESMEAQCGTSVSVHSSLRDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 119 - protein, 321 aa, synthetic construct
  REGION 1..321: note = Synthetic polypeptide
MAKADGSLLYYNPHNPPRRYYFYMAIFAVSVICVLYGPSQQLSSPKIDASAPAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTP

SEQ ID NO: 120 - protein, 4 aa, synthetic construct
  REGION 1..4: note = Synthetic polypeptide
EAEA